(19) United States
(12) Patent Application Publication
(10) Pub. No.: US 2024/0391896 A1
(43) Pub. Date: Nov. 28, 2024

(54) SMALL MOLECULE COMPOUND TARGETING BCL9/BETA-CATENIN INTERACTION

(71) Applicant: Nantong Jutai Biotech Co., Ltd., Nantong City, Jiangsu Province (CN)

(72) Inventor: Yiming CHEN, Nantong City, Jiangsu Province (CN)

(21) Appl. No.: 18/577,053

(22) PCT Filed: Jul. 5, 2022
(86) PCT No.: PCT/CN2022/103988

§ 371 Date: Jun. 6, 2024

(30) Foreign Application Priority Data
Jul. 5, 2021 (CN) 2021107571615

(51) Int. Cl. C07D 401/12; A61K 31/496; A61K 31/5377; C07D 471/04

(52) U.S. Cl. CPC C07D 401/12; A61K 31/496; A61K 31/5377; C07D 471/04

(57) ABSTRACT

A small molecule compound targeting BCL9/β-catenin interaction is provided herein. Specifically, provided is a compound of formula (I) or a pharmaceutically acceptable salt thereof. The compound of formula (I) has excellent ability to inhibit BCL9/β-catenin interaction.

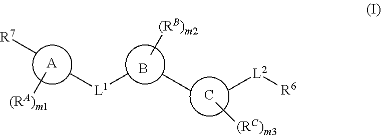

### TECHNICAL FIELD

The invention belongs to the pharmaceutical field, in particular relates to a small molecule compound targeting BCL9 (B-cell lymphoma 9)/β-catenin interaction.

### BACKGROUND

Wnt/β-catenin signal transduction is essential for normal embryonic development and throughout life. In addition, aberrant Wnt signaling is associated with various diseases, especially cancer. Recent studies have shown that direct targeting the β-catenin/B-cell lymphoma 9 (BCL9) protein-protein interaction (PPI) is a promising strategy to block the Wnt pathway. Advances in understanding the cocrystal complex of β-catenin/BCL9 interaction and its mechanism of action have facilitated the discovery process of its inhibitors, but only a few inhibitors have been reported.

Canonical Wnt signaling is a highly conserved developmental signal transduction pathway that regulates cell proliferation, differentiation, and survival. β-catenin is generally considered to be a key effector of Wnt signaling. In the absence of Wnt mono selective (Wntoff), the cytoplasmic pool of β-catenin binds to glycogen synthase kinase 3β(GSK3β), casein kinase 1α(CK1α), the scaffold protein AXIN, and the tumor suppressor adenomatous polyposis coli (APC) to regulate phosphorylation, followed by degradation of β-catenin by the proteasome. β-catenin recruits coactivators, including BCL9 or B-cell lymphoma 9-like (B9L), Pygo, CREB-binding protein (CBP), and the like, to promote the transcription of cell proliferation, migration, and survival genes, such as cyclin D1, c-myc, Survivin, and LEF1. The occurrence and progression of many types of cancers are closely related to these Wnt target genes, including colorectal cancer, breast cancer, lung cancer, hepatocellular carcinoma, leukemia, and multiple myeloma.

Development of use of the β-catenin/BCL9 complex follow robust biochemical assays and developments in drug discovery strategies provide further understanding of interactions, which may lead to the discovery of new anticancer drugs. To date, several different classes of β-catenin/BCL9 PPI inhibitors have been reported. These inhibitors can be mainly divided into two main categories: peptide inhibitors and non-peptide small molecule inhibitors. However, the exploration of β-catenin/BCL9 PPI inhibitors, especially non-peptide small molecule inhibitors, is still in the primary stage of research and exploration.

Summing up, there is an urgent need to develop small molecule compounds targeting the BCL9 (B-cell lymphoma 9)/β-catenin interaction.

### SUMMARY OF THE INVENTION

The purpose of the present invention is to provide a new class of small molecule compounds targeting BCL9/β-catenin interaction.

In the first aspect of the present invention, provided is a compound or a pharmaceutically acceptable salt thereof, or an isomer, solvate, crystal form, or prodrug thereof, the compound is of Formula I.

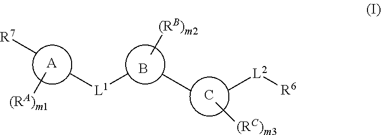

wherein,
R⁷ is an optionally substituted group selected from the group consisting of consisting of: optionally substituted C₁₋₆ alkyl, C₃₋₁₀ cycloalkyl, 4 to 10-membered heterocycloalkyl, C₃₋₁₀ cycloalkenyl, 4 to 10-membered heterocycloalkenyl, C₆₋₁₀ aryl, and 5 to 10-membered heteroaryl; preferably, R⁷ is an optionally substituted group selected from the group consisting of: optionally substituted C₁₋₆ alkyl, C₃₋₁₀ cycloalkyl, 4 to 10-membered heterocycloalkyl, C₆₋₁₀ aryl, and 5 to 10-membered heteroaryl;
Ring A is an optionally substituted ring selected from the group consisting of: C₆₋₁₀aryl; 5 to 10-membered heteroaryl; C₆₋₁₀ aryl substituted with C₃₋₁₀ cycloalkyl, 4 to 10-membered heterocycloalkyl, C₃₋₁₀cycloalkenyl, 4 to 10-membered heterocycloalkenyl, C₆₋₁₀ aryl, or 5 to 10-membered heteroaryl; 5 to 10-membered heteroaryl substituted with C₃₋₁₀ cycloalkyl, 4 to 10-membered heterocycloalkyl, C₃₋₁₀cycloalkenyl, 4 to 10-membered heterocycloalkenyl, C₆₋₁₀ aryl, or 5 to 10-membered heteroaryl; C₆₋₁₀ aryl fused with C₃₋₁₀ cycloalkyl, 4 to 10-membered heterocycloalkyl, C₃₋₁₀cycloalkenyl, 4 to 10-membered heterocycloalkenyl, C₆₋₁₀ aryl, or 5 to 10-membered heteroaryl; 5 to 10-membered heteroaryl fused with C₃₋₁₀ cycloalkyl, 4 to 10-membered heterocycloalkyl, C₃₋₁₀cycloalkenyl, 4 to 10-membered heterocycloalkenyl, C₆₋₁₀ aryl, or 5 to 10-membered heteroaryl;
m1=0, 1, 2, 3 or 4;
each R^{A} is independently R^{A1} or R^{s};
each R^{A1} is independently selected from the group consisting of: halogen, optionally substituted C₁₋₆ alkyl, optionally substituted C₁₋₆ haloalkyl, optionally substituted C₁₋₆ alkoxyl, and optionally substituted C₁₋₆ alkylthio;
L¹ is a linker group of —(W¹)ₙ₁—;
each W¹ is independently selected from the group consisting of: —O—, —S—, C(O)—, —S(O), —S(O)₂, —N(R′)—, —CH(R⁸)— and —C(R^{s})₂—;
n1=1, 2, 3, 4, or 5;
each R¹ and R⁸ are independently selected from the group consisting of: H, optionally substituted C₁₋₆ alkyl, optionally substituted C₃₋₆ cycloalkyl, halogen, optionally substituted C₁₋₆ haloalkyl, optionally substituted C₁₋₆ alkoxyl, optionally substituted C₁₋₆ haloalkoxyl (—O—C₁₋₆ haloalkyl), optionally substituted C₁₋₆ alkyl-O—C₁₋₆ alkylene, optionally substituted C₁₋₆ haloalkoxyl-O—C₁₋₆ alkylene, optionally substituted C₁₋₆ haloalkyl-S—C₁₋₆ alkylene, optionally substituted C₁₋₆ aminoalkyl, optionally substituted C₃₋₁₀ cycloalkyl, optionally substituted 4-10-membered heterocycloalkyl, optionally substituted C₆₋₁₀ aryl, optionally substituted 5 to 10-membered heteroaryl, optionally substituted C₃₋₁₀cycloalkenyl, optionally substituted 4 to 10-membered heterocycloalkenyl, optionally substituted C₃₋₁₀ cycloalkyl-C₁₋₄ alkylene, optionally substituted 4 to 10-membered heterocycloalkyl-C₁₋₄ alkylene, optionally substituted C-₆₋₁₀ aryl-C₁₋₄ alkylene, optionally substituted 5 to 10-membered heteroaryl-C₁₋₄ alkylene, optionally substituted C₃₋₁₀cycloalkenyl-C₁₋₄ alkylene, optionally substituted 4 to 10-membered heterocycloalkenyl-C₁₋₄ alkylene; or, R¹ or R⁸ together with the R⁸ on ring A to form an optionally substituted C₄₋₁₀ cycloalkyl or 4 to 10-membered heterocycloalkyl;
Ring B is an optionally substituted ring selected from the group consisting of: C₃₋₁₂ cycloalkyl, and 4- to 12-membered heterocycloalkyl;
m2=0, 1, 2, 3 or 4;
each R^{B} is independently R^{B1} or R^{s};
each R^{B1} is independently selected from the group consisting of: halogen, hydroxyl, cyano, optionally substituted C₁₋₆ alkyl, optionally substituted C₁₋₆ alkoxyl, optionally substituted C₁₋₆ alkylthio, optionally substituted C₃₋₁₀ cycloalkyl, optionally substituted 4 to 10-membered heterocycloalkyl, optionally substituted C₃₋₁₀cycloalkenyl, optionally substituted 4 to 10-membered heterocycloalkenyl, optionally substituted C₆₋₁₀ aryl, and optionally substituted 5 to 10-membered heteroaryl;
Ring C is an optionally substituted ring selected from the group consisting of: C₆₋₁₀ aryl, and 5 to 10-membered heteroaryl;
m3=0, 1, 2, 3 or 4;
each R^{C} is independently R^{C1} or R^{s};
each R^{C1} is independently selected from the group consisting of: halogen, optionally substituted C₁₋₆ alkyl, optionally substituted C₁₋₆ haloalkyl, hydroxyl and optionally substituted C₁₋₆ alkoxy, optionally substituted C₁₋₆ haloalkoxy;
L² is a linker group of —(W²)ₙ₂—;
each W² is independently selected from the group consisting of: —O—, —S—, —C(O)—, —S(O), —S(O)₂, —N(R^{s})—, and —CR²R³—;
subscript n2=1, 2, 3, 4, or 5;
R² and R³ are each independently selected from the group consisting of: H, optionally substituted C₁₋₄ alkyl, halogen, cyano, optionally substituted C₁₋₆ haloalkyl, optionally substituted C₁₋₆ alkyl-O—C₁₋₆alkylene, optionally substituted C₁₋₆ haloalkyl-O—C₁₋₆alkylene, optionally substituted C₁₋₆ haloalkyl-S—C₁₋₆alkylene, optionally substituted C₃₋₁₀ cycloalkyl, optionally substituted 4 to 10-membered heterocycloalkyl, optionally substituted C₆₋₁₀ aryl, optionally substituted 5 to 10-membered heteroaryl, optionally substituted C₃₋₁₀cycloalkenyl, optionally substituted 4 to 10-membered heterocycloalkenyl, optionally substituted C₃₋₁₀ cycloalkyl-C₁₋₄alkylene, optionally substituted 4 to 10-membered heterocycloalkyl-C₁₋₄alkylene, optionally substituted C₆₋₁₀ aryl-C₁₋₄alkylene, optionally substituted 5 to 10-membered heteroaryl-C₁₋₄alkylene, optionally substituted C₃₋₁₀ cycloalkenyl-C₁₋₄alkylene, optionally substituted 4 to 10-membered heterocycloalkenyl-C₁₋₄alkylene; or, R² and R³, together with the carbon atoms to which they are attached to form a group selected from the group consisting of: optionally substituted C₃₋₁₀ cycloalkyl, optionally substituted 4 to 10-membered heterocycloalkyl, optionally substituted C₃₋₁₀cycloalkenyl, optionally substituted 4 to 10-membered heterocycloalkenyl;
R⁶ is selected from the group consisting of: —OH, C₃₋₁₂ cycloalkyl, 4 to 10-membered heterocycloalkyl attached to the rest via a carbon atom in the ring, and —NR⁴R⁵;
R⁴ and R⁵ are each independently selected from the group consisting of: H, optionally substituted C₁₋₆ alkyl, optionally substituted C₃₋₁₀ cycloalkyl, optionally substituted 4 to 8-membered heterocycloalkyl, optionally substitute C₆₋₁₀ aryl, optionally substituted 5 to 10-membered heteroaryl, optionally substituted C₃₋₁₀cycloalkenyl, optionally substituted 4 to 10-membered heterocycloalkenyl (preferably, R⁴ and R⁵ are each independently selected from the group consisting of: optionally substituted C₁₋₆ alkyl, optionally substituted C₃₋₁₀ cycloalkyl, optionally substituted 4 to 8-membered heterocycloalkyl, optionally substituted C₆₋₁₀ aryl, optionally substituted 5 to 10-membered heteroaryl, optionally substituted C₃₋₁₀ cycloalkenyl, optionally substituted 4 to 10-membered heterocycloalkyl); or, R⁴ and R⁵ together with the nitrogen atom to which they are connected to form a ring selected from the group consisting of: optionally substituted 4 to 10-membered heterocycloalkyl, optionally substituted 4 to 10-membered heterocycloalkenyl, or optionally substituted 5 to 10-membered heteroaryl;
each R⁸ is independently an H or optionally substituted C₁₋₄ alkyl group;
unless otherwise defined, said optionally substituted means unsubstituted or means that one or more (such as 1, 2, 3, or 4) hydrogen atoms in the group are substituted with a substituent selected from the group consisting of: D, halogen, C₁₋₆ alkyl, C₁₋₆ haloalkyl, C₁₋₆ hydroxyalkyl, C₂₋₆ alkenyl, C₂₋₆ alkynyl, —CN, —OR′, —NO₂, —NR′R″, —SR′, —OC(O)R′, —C(O)R′, —CO₂R′, —CONR′, —OC(O)NR′R″, —NR″C(O)R′, —NR″—C(O)NR′R″, —NR″C(O)₂R′, —S(O)R′, —S(O)₂R′, —S(O)₂NR′R″, —NR″S(O)₂R′, C₃₋₁₀ cycloalkyl optionally substituted with one or more R″′, 4 to 10-membered heterocycloalkyl optionally substituted with one or more R″′, C₆₋₁₀ aryl optionally substituted with one or more R″′, 5 to 10-membered heteroaryl optionally substituted with one or more R″′, —C₁₋₄ alkylene-C₃₋₁₀ cycloalkyl optionally substituted with one or more R″′, —C₁₋₄ alkylene-4 to 10-membered heterocycloalkyl optionally substituted with one or more R″′, —C₁₋₄ alkylene-C₆₋₁₀ aryl optionally substituted with one or more R″′, and —C₁₋₄ alkylene-5 to 10-membered heteroaryl optionally substituted with one or more R″′;
each R′ is independently selected from the group consisting of: H, D, C₁₋₆ alkyl, C₁₋₆ haloalkyl, C₃₋₁₀ cycloalkyl optionally substituted with one or more R″′, 4 to 10 heterocycloalkyl optionally substituted with one or more R″′, C₆₋₁₀ aryl optionally substituted with one or more R″′, 5 to 10 heteroaryl optionally substituted with one or more R″′, —C₁₋₄ alkylene-C₃₋₁₀ cycloalkyl optionally substituted with one or more R″′, —C₁₋₄ alkylene-4 to 10-membered heterocycloalkyl optionally substituted with one or more R′″, —C₁₋₄ alkylene-C₆₋₁₀ aryl optionally substituted with one or more R″′, and —C₁₋₄ alkylene-5 to 10-membered heteroaryl optionally substituted with one or more R″′; each R″ is selected from the group consisting of: H, D, C₁₋₄ alkyl, C₁₋₄haloalkyl, and C₃₋₄ cycloalkyl; each R″′ is independently selected from the group consisting of: D, halogen, hydroxyl, nitro, CN, C₁₋₆ alkyl, and C₁₋₆ haloalkyl.

In another preferred embodiment, R⁷ is an optionally substituted group selected from the group consisting of: optionally substituted C₁₋₆ alkyl, C₃₋₁₀ cycloalkyl, 4 to 10-membered heterocycloalkyl, C₆₋₁₀ aryl, and 5 to 10-membered heteroaryl; and, R⁴ and R⁵ are each independently selected from the group consisting of: optionally substituted C₁₋₆alkyl, optionally substituted C₃₋₁₀ cycloalkyl, optionally substituted 4- to 8-membered heterocycloalkyl, optionally substituted C₆₋₁₀ aryl, optionally substituted 5 to 10-membered heteroaryl, optionally substituted C₃₋₁₀cycloalkenyl, optionally substituted 4 to 10-membered heterocycloalkenyl; or, R⁴ and R⁵ and the nitrogen atom to which they are connected combined together to form a ring selected from the group consisting of: optionally substituted 4 to 10-membered heterocycloalkyl, optionally substituted 4 to 10-membered heterocycloalkenyl, or optionally substituted 5 to 10-membered heteroaryl.

In another preferred embodiment, the pharmaceutically acceptable salt is an acid addition salt, preferably hydrochloride or trifluorocarbamate salt.

In another preferred embodiment, R⁷ is an optionally substituted group selected from the group consisting of: C₆₋₁₀ alkyl, and 5 to 10-membered heteroaryl.

In another preferred embodiment, in R⁷, the heteroaryl comprises 1, 2 or 3 nitrogen heteroatoms as ring atoms, and the remaining ring atoms in the heteroaryl are carbon atoms.

In another preferred embodiment, R⁷ is optionally substituted C₃₋₁₀cycloalkenyl or optionally substituted 5 to 10-membered heteroaryl.

In another preferred embodiment, R⁷ is optionally substituted C₃₋₁₀cycloalkenyl; preferably, R⁷ is optionally substituted C₃₋₆ cycloalkenyl. In another preferred embodiment, R⁷ is optionally substituted 5 to 10-membered heteroaryl.

In another preferred embodiment, R⁷ is optionally substituted 5-membered heteroaryl.

In another preferred embodiment, R⁷ is an optionally substituted group that the group is selected from the group consisting of:

isopropyl,

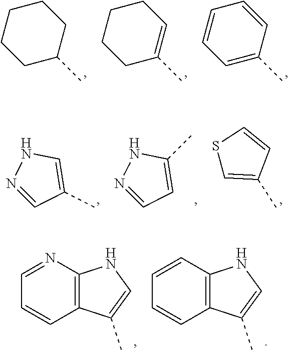

In another preferred embodiment, R⁷ is an optionally substituted group that is selected from the group consisting of:

isopropyl,

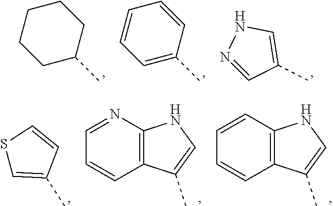

In another preferred embodiment, R⁷ is optionally substituted

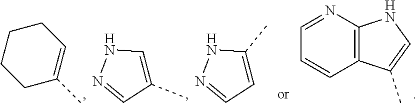

In another preferred embodiment, R⁷ is optionally substituted

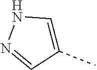

In another preferred embodiment, R⁷ is optionally substituted

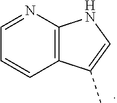

In another preferred embodiment, R⁷ is optionally substituted

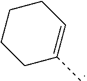

In another preferred embodiment, in R⁷, the optionally substituted means unsubstituted or means that one or more (such as 1, or 2) hydrogen in the group is substituted with a substituent selected from the group consisting of: D, halogen, C₁₋₆ alkyl, —NR′R″; wherein, each R′ is independently selected from the group consisting of: H, D, C₁₋₆ alkyl; and each R″ is selected from the group consisting of: H, D, C₁₋₄ alkyl.

In another preferred embodiment, in R⁷, the optionally substituted means unsubstituted or means that one or more (such as 1, or 2) hydrogen in the group is substituted with a substituent selected from the group consisting of: methyl, —NR′R″; wherein each R′ is independently selected from the group consisting of: H and each R″ is selected from the group consisting of: H.

In another preferred embodiment, R⁷ is optionally substituted

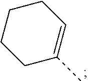

and in R⁷ the optionally substituted means unsubstituted or means that 1 or 2 hydrogen in the group is substituted with a substituent selected from the group consisting of: C₁₋₆ alkyl (such as methyl).

In another preferred embodiment, R⁷ is optionally substituted

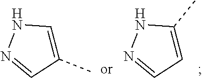

and in R⁷, the optionally substituted means unsubstituted or means that one or more (such as 1, or 2) hydrogen in the group is substituted with a substituent selected from the group consisting of: methyl, —NR′R″; wherein each R′ is independently selected from the group consisting of: H and each R″ is selected from the group consisting of: H.

In another preferred embodiment, Ring A is a ring selected from the group consisting of:

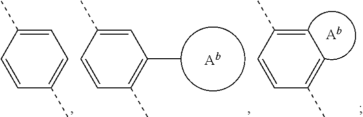

wherein, Ring A^{a} and Ring A^{b} are each independently selected from the group consisting of: C₃₋₁₀ cycloalkyl, C₃₋₁₀cycloalkenyl, 4 to 10-membered heterocycloalkyl, 4 to 10-membered heterocycloalkenyl, C₆₋₁₀ aryl, or 5 to 10-membered heteroaryl.

In another preferred embodiment, Ring A^{a} and Ring A^{b} are each independently selected from the group consisting of: C₅₋₆ cycloalkyl, C₅₋₆ cycloalkenyl, 5 to 6-membered heterocycloalkyl, 5 to 6-membered heterocycloalkenyl, phenyl, or 5 to 6-membered heteroaryl.

In another preferred embodiment, Ring A is selected from the group consisting of:

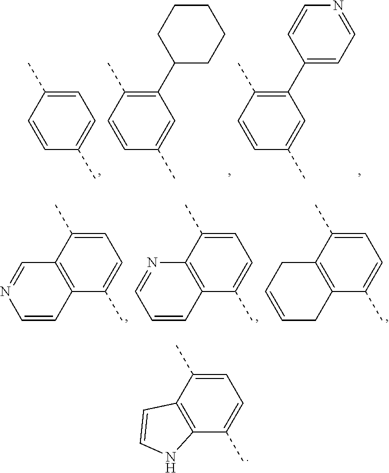

In another preferred embodiment, Ring A is

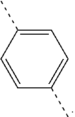

In another preferred embodiment, m1=0, 1 or 2; preferably, m1=0 or 1.

In another preferred embodiment, R^{A} is H or R^{A1}; and R^{A1} is selected from the group consisting of: halogen, optionally substituted C₁₋₆alkyl, optionally substituted C₁₋₆ haloalkyl, and optionally substituted C₁₋₆ alkoxy (preferably, R^{A1} is halogen).

In another preferred embodiment, m1=0.

In another preferred embodiment, m1=1, 2, 3, or 4. In another In another preferred embodiment, m1=1, 2, 3, or 4, and at least one of R^{A} is R^{A1}.

In another preferred embodiment,

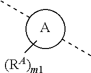

is

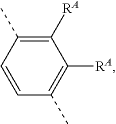

and each R^{A} is R^{s} (preferably, each R^{A} is H).

In another preferred embodiment, and where

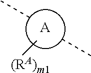

is

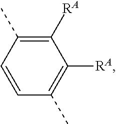

and wherein at least one of R^{A} is R^{A1}; preferably, each R^{A1} is independently selected from the group consisting of: halogen, optionally substituted C₁₋₆ haloalkyl, and optionally substituted C₁₋₆ alkoxy group; more preferably, each R^{A1} is independently selected from the group consisting of: Cl, —OCH₃, —CF₃.

In another preferred embodiment,

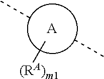

is

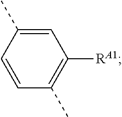

preferably, R^{A1} is selected from the group consisting of: halogen, optionally substituted C-₁₋₆haloalkyl, and optionally substituted C-₁₋₆alkoxy; more preferably, R^{A1} is selected from the group consisting of: Cl, —OCH₃, —CF₃.

In another preferred embodiment,

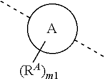

is

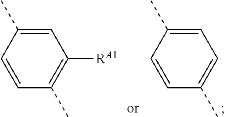

and wherein R^{A1} is selected from the group consisting of: halogen (e.g., Cl), optionally substituted C₁₋₆ alkyl, optionally substituted C₁₋₆ haloalkyl, and optionally substituted C₁₋₆ alkoxy; more preferably, R^{A1} is a halogen such as Cl.

In another preferred embodiment, n1=3.

In another preferred embodiment, at least one of W¹ group is —N(R¹)—.

In another preferred embodiment, at least one of W¹ group is —CH(R⁸)—.

In another preferred embodiment, L¹ is —CH(R⁸)—N(R¹)—W¹—W¹— (preferably, wherein the CH(R⁸) terminal is connected to Ring A).

In another preferred embodiment, L¹ is —CH(R⁸)—N(R¹)—C(O)—W¹— (preferably, wherein the CH(R⁸) terminal is connected to Ring A).

In another optimization example, L¹ is —CH(R⁸)—N(R¹)—C(O)—N(R¹)— (preferably, wherein CH(R⁸) terminal is connected to Ring A).

In another preferred embodiment, L¹ is —CH(R⁸)—N(R¹)—C(O)—NH— (preferably, wherein the CH(R⁸) terminal is attached to ring A).

In another preferred embodiment, L¹ is —CH(R⁸)—N(R¹)—W¹— (preferably, wherein the CH(R⁸) terminal is attached to ring A).

In another preferred embodiment, L¹ is —CH(R⁸)—N(R¹)—C(O)— (preferably, wherein the CH(R⁸) terminal is attached to ring A). In another preferred embodiment, R¹ is selected from the group consisting of: halogen, optionally substituted C₁₋₆ haloalkyl, optionally substituted C₁₋₆ haloalkyl-O—C₁₋₆ alkylene, optionally substituted C₁₋₆ haloalkyl-S—C₁₋₆ alkylene, optionally substituted C₆₋₁₀ aryl, optionally substituted 5 to 10 membered heteroaryl, optionally substituted C₃₋₁₀cycloalkenyl, optionally substituted 4 to 10-membered heterocycloalkenyl.

In another preferred embodiment, R¹ is optionally substituted C₃₋₆ cycloalkyl; preferably, optionally substituted cyclopropyl.

In another preferred embodiment, R⁸ is selected from the group consisting of: H, optionally substituted C₁₋₆ alkyl (preferably, C₁₋₄ alkyl, more preferably, methyl, ethyl, and isopropyl, most preferably, methyl), optionally substituted C₁₋₆ aminoalkyl, optionally substituted C₁₋₆ alkyl-O—C₁₋₆ alkylene (preferably, —(CH₂)₂OCH₂CH₃), optionally substituted C₃₋₆ cycloalkyl (preferably, cyclobutyl

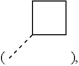

cyclopentyl, cyclohexyl), and optionally substituted C₃₋₁₀ cycloalkyl-C₁₋₄ alkylene (preferably cyclopropyl-methyl (—CH₂-cyclopropyl)).

In another preferred embodiment, R⁸ is selected from the group consisting of: H, C₁₋₆ alkyl, and C₃₋₆ cycloalkyl.

In another preferred embodiment, L¹ is —CH(R⁸)—N(R¹)—W¹— (preferably, —CH(R⁸)—N(R¹)—C(O)—), wherein the CH(R⁸) terminal is attached to Ring A; and wherein, R⁸ together with R⁸ on ring A forms an optionally substituted 4-10 heterocycloalkyl (preferably, a 5 or 6-membered heterocycloalkyl).

In another preferred embodiment, L¹ is —CH(R⁸)—N(R¹)—W¹—W¹— (preferably, —CH(R⁸)—N(R¹)—C(O)—NH—), wherein the CH(R⁸) terminal is attached to Ring A; and wherein, R¹ is selected from the group consisting of: halogen, optionally substituted C₁₋₆ haloalkyl, optionally substituted C₁₋₆ haloalkyl-O—C₁₋₆alkylene, optionally substituted C₁₋₆haloalkyl-S—C₁₋₆alkylene, optionally substituted C₆₋₁₀aryl, optionally substituted 5 to 10-membered heteroaryl, optionally substituted C₃₋₁₀cycloalkenyl, and optionally substituted 4 to 10-membered heterocycloalkenyl, and R⁸ is H.

In another preferred embodiment, L¹ is —CH(R⁸)—N(R¹)—W¹— (preferably, is —CH(R⁸)—N(R¹)—C(O)—), wherein the CH(R⁸) terminal is attached to Ring A; and wherein, R¹ is selected from the group consisting of: halogen, optionally substituted C₁₋₆ haloalkyl, optionally substituted C₁₋₆haloalkyl-O—C₁₋₆alkylene, optionally substituted C₁₋₆haloalkyl-S—C₁₋₆alkylene, optionally substituted C₆₋₁₀aryl, optionally substituted 5 to 10-membered heteroaryl, optionally substituted C₃₋₁₀cycloalkenyl, and optionally substituted 4 to 10-membered heterocycloalkenyl, and R⁸ is H.

In another preferred embodiment, L¹ is —CH(R⁸)—N(R¹)—W¹—W¹— (preferably, —CH(R⁸)—N(R¹)—C(O)—NH—), wherein the CH(R⁸) terminal is attached to Ring A; and wherein R¹ is optionally substituted C₃₋₆ cycloalkyl (preferably, optionally substituted cyclopropyl), and R⁸ is selected from the group consisting of: H, optionally substituted C₁₋₆ alkyl (preferably, C₁₋₄ alkyl, more preferably, methyl or ethyl, most preferably, methyl), and optionally substituted C₃₋₆ cycloalkyl (preferably, cyclobutyl).

In another preferred embodiment, L¹ is —CH(R⁸)—N(R¹)—W¹— (preferably, —CH(R⁸)—N(R¹)—C(O)—), wherein the CH(R⁸) terminal is attached to Ring A; and wherein R¹ is optionally substituted C₃₋₆ cycloalkyl (preferably, optionally substituted cyclopropyl), and R⁸ is selected from the group consisting of: H, optionally substituted C₁₋₆ alkyl (preferably, C₁₋₄ alkyl, more preferably, methyl or ethyl, most preferably, methyl), and optionally substituted C₃₋₆ cycloalkyl (preferably, cyclobutyl).

In another preferred embodiment, L¹ is —CH(R⁸)—N(R¹)—W¹— (preferably, —CH(R⁸)—N(R¹)—C(O)—) or —CH(R⁸)—N(R¹)—W¹—W¹— (preferably, —CH(R⁸)—N(R¹)—C(O)—NH—), wherein the CH(R⁸) terminal is attached to Ring A; and wherein R¹ is optionally substituted C₃₋₆ cycloalkyl (preferably, optionally substituted cyclopropyl), and R⁸ is selected from the group consisting of: H, optionally substituted C₁₋₆ alkyl (preferably, R⁸ is H).

In another preferred embodiment, Ring B is

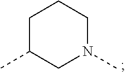

preferably, the N in Ring B is attached to ring C.

In another preferred embodiment, m2=0

In another preferred embodiment, R^{B} is all R^{s}; preferably, R^{B} is all H.

In another preferred embodiment, m2=1 or 2.

In another preferred embodiment, m2=1.

In another preferred embodiment, at least one of R^{B} is R^{B1} In another preferred embodiment, each R^{B1} is independently selected from the group consisting of: halogen, hydroxyl, cyano, optionally substituted C₁₋₆ alkyl, optionally substituted C₁₋₆ alkanoxy, optionally substituted C₃₋₁₀ cycloalkyl, optionally substituted 4 to 10-membered heterocycloalkyl, optionally substituted C₆₋₁₀ aryl, and optionally substituted 5 to 10-membered heteroaryl.

In another preferred embodiment, R^{B1} is selected from the group consisting of: —OH, Cl, methoxy, cyano, methyl, ethyl, n-propyl, isopropyl, cyclohexyl, pyridyl, and phenyl.

In another preferred embodiment,

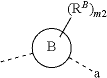

is

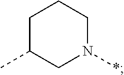

wherein * refers to the attachment to Ring C.

In another preferred embodiment,

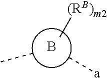

is selected from the group consisting of

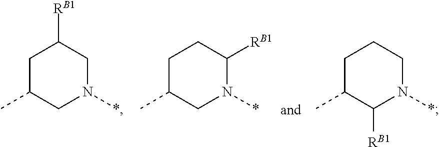

wherein * refers to the attachment to Ring C.

In another preferred embodiment,

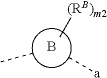

is

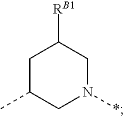

wherein * refers to the attachment to Ring C.

In another preferred embodiment,

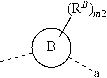

is

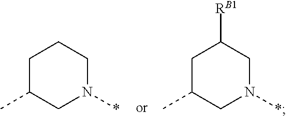

wherein, * refers to the attachment to Ring C; and wherein R^{B1} is selected from the group consisting of: optionally substituted C₃₋₁₀ cycloalkyl, optionally substituted 4 to 10-membered heterocycloalkyl, optionally substituted C₆₋₁₀ aryl, and optionally substituted 5 to 10-membered heteroaryl (preferably, R^{B1} is selected from the group consisting of: cyclohexyl and phenyl).

In another preferred embodiment, Ring C is phenyl or pyridyl, preferably, phenyl.

In another preferred embodiment, Ring C is

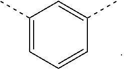

In another preferred embodiment, m3=0.

In another preferred embodiment, m3=1, 2, 3, or 4.

In another preferred embodiment, R^{C1} is selected from the group consisting of: halogen (preferably, F, Cl), C₁₋₆ haloalkyl (preferably, trifluoromethyl), and C₁₋₆ alkoxy (preferably, methoxy).

In another preferred embodiment, Ring C is

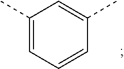

m3=0, 1 or 2; R^{C} is H or R^{C1}; and R^{C1} is selected from the group consisting of: halogen preferably, F, Cl), C₁₋₆ haloalkyl (preferably, trifluoromethyl), and C₁₋₆ alkoxy (preferably, methoxy); preferably, R^{C1} is a halogen.

In another preferred embodiment,

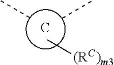

is

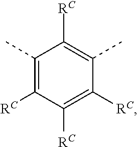

and wherein R^{C} are all R^{s}; preferably, R^{C} are all H.

In another preferred embodiment,

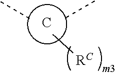

is

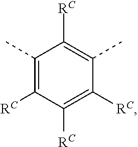

and wherein at least one of R^{C} is R^{C1}.

In another preferred embodiment,

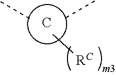

is

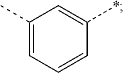

wherein, * refers to the attachment to L².

In another preferred embodiment,

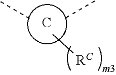

is

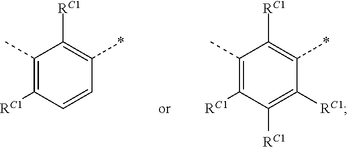

wherein * refers to the attachment to L².

In another preferred embodiment, at least one of W² group is —C(R²R³)—.

In another preferred embodiment, n2=3.

In another preferred embodiment, L² is —W²—CR²R³—W²—.

In another preferred embodiment, L² is W²—CR²R³—C(O)— and W² is selected from the group consisting of: —O—, —S—, —N(R^{s})— (preferably, W² is selected from the group consisting of: —O—, —N(R^{s})—).

In another preferred embodiment, L² is —O—CR²R³—C(O)—.

In another preferred embodiment, both R² and R³ are optionally substituted C₁₋₄ alkyl.

In another preferred embodiment, one of R² and R³ is H, and the other is a group other than H as defined above.

In another preferred embodiment, R² and R³ together with the carbon atom to which they are attached to form a group selected from the group consisting of: optionally substituted C₃₋₁₀ cycloalkyl, optionally substituted 4 to 10-membered heterocycloalkyl, optionally substituted C₃₋₁₀cycloalkenyl, optionally substituted 4 to 10-membered heterocycloalkenyl.

In another preferred embodiment, L² is —W²—CR²R³—W²— (preferably —O—CR²R³—C(O)—), and R² and R³ are each independently optionally substituted C-₁₋₄alkyl; preferably, L² is —O—CR²R³—C(O)— and both R² and R³ are methyl.

In another preferred embodiment, L² is —O—C(CH₃)₂—C(O)— (wherein the C(O) terminal is attached to R⁶).

In another preferred embodiment, L² is —W²—CR²R³—W²— (preferably —O—CR²R³—C(O)—), and R² and R³ are each independently selected from the group consisting of: H, halogen, cyano, optionally substituted C₁₋₆ haloalkyl, optionally substituted C₁₋₆ alkyl-O—C₁₋₆ alkylene, optionally substituted C₁₋₆ haloalkyl-O—C₁₋₆ alkylene, optionally substituted C₁₋₆ haloalkyl-S—C₁₋₆ alkylene, optionally substituted C₃₋₁₀ cycloalkyl, optionally substituted 4 to 10-membered heterocycloalkyl, optionally substituted 5 to 10-membered heteroaryl, optionally substituted C₃₋₁₀cycloalkenyl, optionally substituted 4 to 10-membered heterocycloalkenyl, optionally substituted C₃₋₁₀ cycloalkyl-C₁₋₄ alkylene, optionally substituted 4 to 10-membered heterocycloalkyl-C₁₋₄ alkylene, optionally substituted 5 to 10-membered heteroaryl-C₁₋₄ alkylene, optionally substituted C₃₋₁₀cycloalkenyl-C₁₋₄ alkylene, optionally substituted 4 to 10-membered heterocycloalkenyl-C₁₋₄ alkylene; or, R² and R³ together with the carbon atom to which they are attached to form a group selected from the group consisting of: optionally substituted 4 to 10-membered heterocycloalkyl, optionally substituted C₃₋₁₀ cycloalkenyl, optionally substituted 4 to 10-membered heterocycloalkenyl. In another preferred embodiment, R⁴ and R⁵ are each independently selected from the group consisting of: H, optionally substituted C₁₋₆ alkyl; and wherein the optionally substituted means that one hydrogen in the group is substituted with a substituent selected from the group consisting of: —OR′, —NR′R″; wherein R′ is independently selected from the group consisting of: H, D, C₁₋₆ alkyl, and R″ is selected from the group consisting of: H, D, C₁₋₄ alkyl (preferably, R′ is H and R″ is H).

In another preferred embodiment, R⁴ and R⁵ are each independently selected from the group consisting of: H, optionally substituted C₁₋₆ alkyl group; and wherein the optionally substituted means that one hydrogen in the group is substituted with a substituent selected from the group consisting of: —NR′R″; wherein R′ is independently selected from the group consisting of: H, D, C₁₋₆ alkyl, and R″ is selected from the group consisting of: H, D, C₁₋₄ alkyl (preferably, R′ is H and R″ is H).

In another preferred embodiment, —NR⁴R⁵ is 4 to 10 membered heterocycloalkyl with at least one —O— present on the ring; preferably, —NR⁴R⁵ is a 4 to 10 membered heterocycloalkyl with one —O— present on the ring.

In another preferred embodiment, —NR⁴R⁵ is

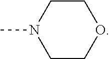

In another preferred embodiment, —NR⁴R⁵ is a 4 to 10 membered heterocycloalkyl with at least one —NH₂⁺— or —NH— present on the ring; preferably, —NR⁴R⁵ is 4 to 10 membered heterocycloalkyl with one —NH₂⁺— or —NH— present on the ring.

In another preferred embodiment, —NR⁴R⁵ is or

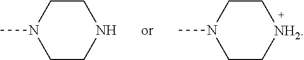

In another preferred embodiment, R⁶ is —NR⁴R⁵.

In another preferred embodiment, R⁶ is —NR⁴R⁵; wherein,

R⁴ and R⁵ are each independently selected from the group consisting of: H, optionally substituted C₁₋₆ alkyl; and wherein the optionally substituted means that one hydrogen in the group is substituted with a substituent selected from the group consisting of: —OR′, —NR′R″; wherein R′ is independently selected from the group consisting of: H, D, C₁₋₆ alkyl, and R″ is selected from the group consisting of: H, D, C₁₋₄ alkyl (preferably, R′ is H and R″ is H); or, —NR⁴R⁵ is 4 to 10-membered heterocycloalkyl with at least one —O— present in the ring; or, —NR⁴R⁵ is 4 to 10 membered heterocycloalkyl with at least one —NH₂⁺— or —NH— present on the ring.

In another preferred embodiment, R⁶ is —NR⁴R⁵, and R⁴ and R⁵ are each independently selected from the group consisting of: optionally substituted C₁₋₆ alkyl, optionally substituted C₃₋₁₀ cycloalkyl, optionally substituted 4 to 8-membered heterocycloalkyl, optionally substituted C₆₋₁₀ aryl, optionally substituted 5 to 10-membered heteroaryl, optionally substituted C₃₋₁₀cycloalkenyl, optionally substituted 4 to 10-membered heterocycloalkenyl; or, R⁴ and R⁵ together with the nitrogen atom to which they are attached to form a ring selected from the group consisting of: an optionally substituted 4 to 10-membered heterocycloalkenyl or optionally substituted 5 to 10-membered heteroaryl.

In another preferred embodiment, the compound is as shown in Formula V, Formula Va or Vb.

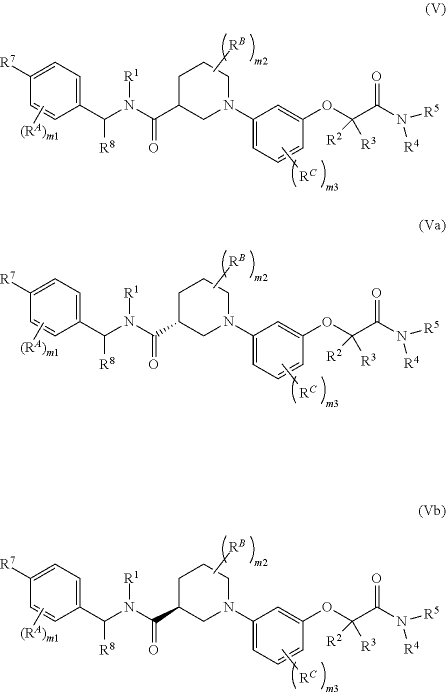

Preferably, R⁷ is

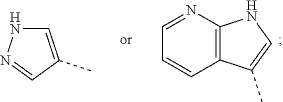

R^{A}, R^{B}, R^{C}, R¹, R², R³, R⁴, R⁵, R⁷, R⁸, subscript m1, subscript m2, and subscript m3 as defined above.

In another preferred embodiment, the compound is selected from Table I:

TABLE I

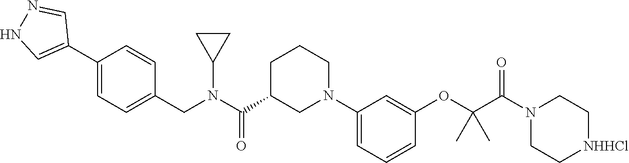

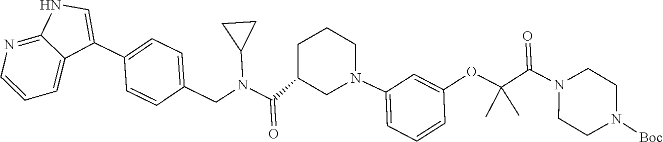 [SMILES: CC(C)(C)OC(=O)N1CCN(C(=O)C(C)(C)Oc2cccc(N3CCC[C@@H](C(=O)N(Cc4ccc(-c5c[nH]c6ncccc56)cc4)C4CC4)C3)c2)CC1]

In another preferred embodiment, the compound is as shown in Formula II.

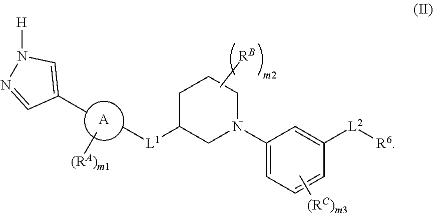

In another preferred embodiment, the compound is as shown in Formula III.

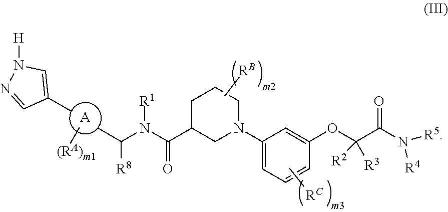

In another preferred embodiment, the shown compound is as shown in Formula IIIa or Formula IIIb.

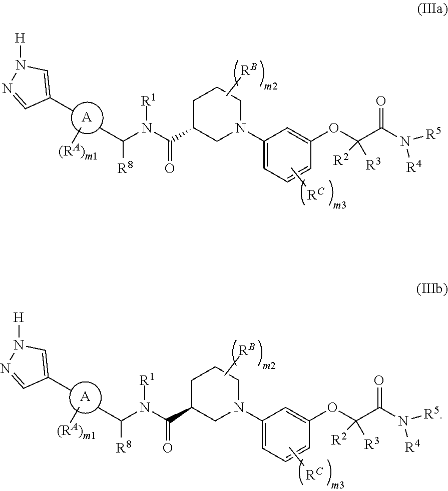

In another preferred embodiment, the compound is selected from Table A1:

TABLE A1

| | |
|---|---|
| A-022 | 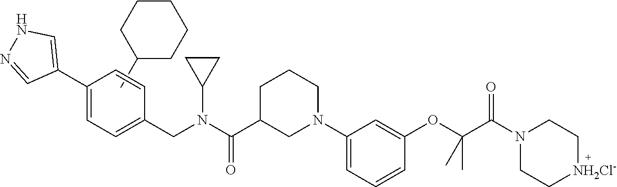 |
| A-023 | 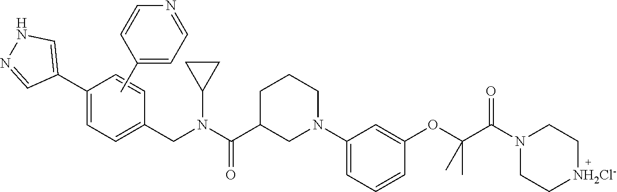 |
| A-024 | 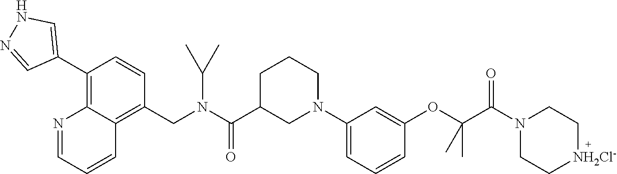 |
| A-025 | 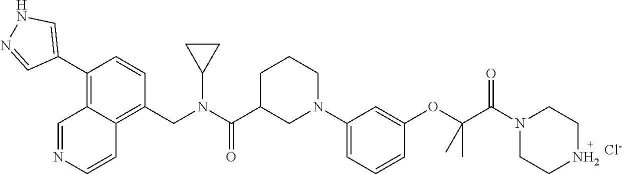 [SMILES: CC(C)(Oc1cccc(N2CCCC(C(=O)N(Cc3ccc(-c4cn[nH]c4)c4cnccc34)C3CC3)C2)c1)C(=O)N1CCNCC1 ; [Cl-]] |
| A-026 | 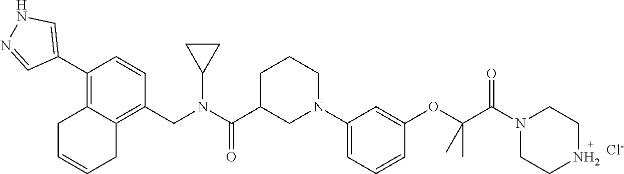 [SMILES: CC(C)(Oc1cccc(N2CCCC(C(=O)N(Cc3ccc(-c4cn[nH]c4)c4c3CC=CC4)C3CC3)C2)c1)C(=O)N1CCNCC1 ; [Cl-]] |
| A-027 | 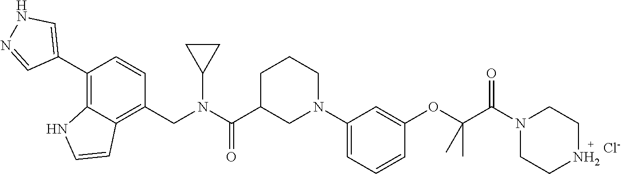 [SMILES: CC(C)(Oc1cccc(N2CCCC(C(=O)N(Cc3ccc(-c4cn[nH]c4)c4[nH]ccc34)C3CC3)C2)c1)C(=O)N1CCNCC1 ; [Cl-]] |

In another preferred embodiment, the compound is selected from Table A2: Table A2

TABLE A2

| | |
|---|---|
| A-001 | 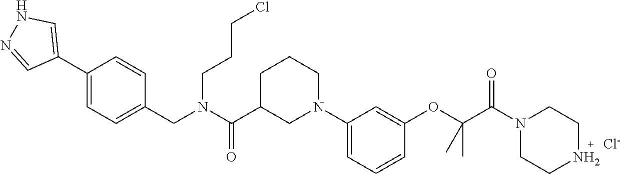 [SMILES: CC(C)(Oc1cccc(N2CCCC(C(=O)N(CCCCl)Cc3ccc(-c4cn[nH]c4)cc3)C2)c1)C(=O)N1CCNCC1 ; [Cl-]] |
| A-002 | 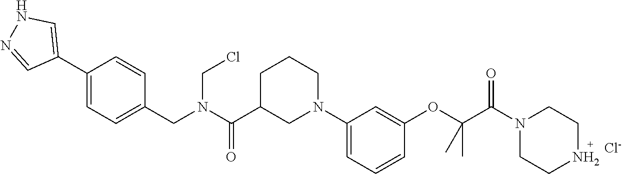 [SMILES: CC(C)(Oc1cccc(N2CCCC(C(=O)N(CCl)Cc3ccc(-c4cn[nH]c4)cc3)C2)c1)C(=O)N1CCNCC1 ; [Cl-]] |
| A-003 | 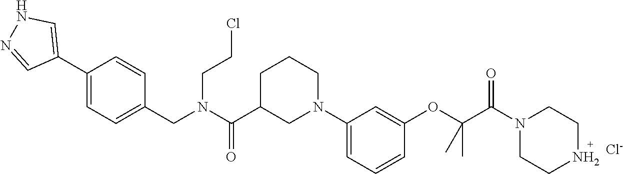 [SMILES: CC(C)(Oc1cccc(N2CCCC(C(=O)N(CCCl)Cc3ccc(-c4cn[nH]c4)cc3)C2)c1)C(=O)N1CCNCC1 ; [Cl-]] |
| A-004 | 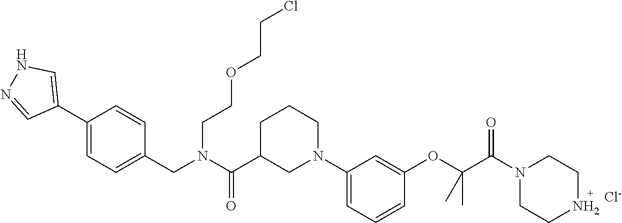 [SMILES: CC(C)(Oc1cccc(N2CCCC(C(=O)N(CCOCCCl)Cc3ccc(-c4cn[nH]c4)cc3)C2)c1)C(=O)N1CCNCC1 ; [Cl-]] |
| A-005 | 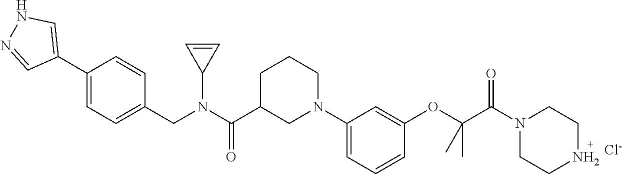 [SMILES: CC(C)(Oc1cccc(N2CCCC(C(=O)N(Cc3ccc(-c4cn[nH]c4)cc3)C3C=C3)C2)c1)C(=O)N1CCNCC1 ; [Cl-]] |
| A-006 | 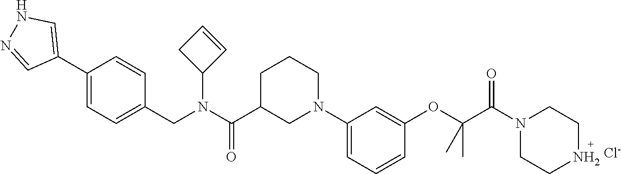 [SMILES: CC(C)(Oc1cccc(N2CCCC(C(=O)N(Cc3ccc(-c4cn[nH]c4)cc3)C3C=CC3)C2)c1)C(=O)N1CCNCC1 ; [Cl-]] |
| A-007 | 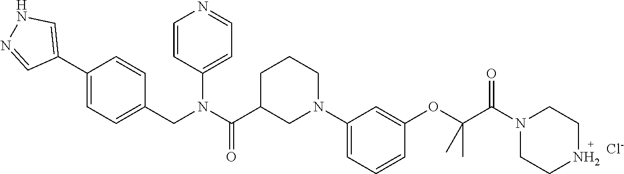 [SMILES: CC(C)(Oc1cccc(N2CCCC(C(=O)N(Cc3ccc(-c4cn[nH]c4)cc3)c3ccncc3)C2)c1)C(=O)N1CCNCC1 ; [Cl-]] |
| A-008 | 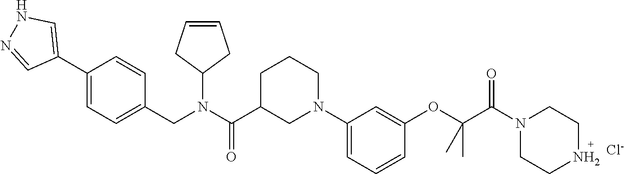 [SMILES: CC(C)(Oc1cccc(N2CCCC(C(=O)N(Cc3ccc(-c4cn[nH]c4)cc3)C3CC=CC3)C2)c1)C(=O)N1CCNCC1 ; [Cl-]] |
| A-009 | 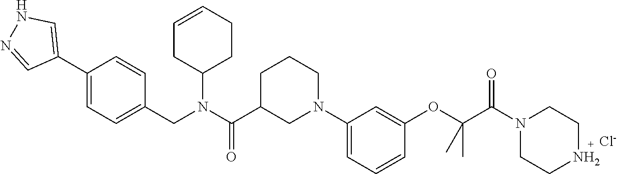 [SMILES: CC(C)(Oc1cccc(N2CCCC(C(=O)N(Cc3ccc(-c4cn[nH]c4)cc3)C3CC=CCC3)C2)c1)C(=O)N1CCNCC1 ; [Cl-]] |

In another preferred embodiment, the compound is selected from Table A3:

TABLE A3

| | |
|---|---|
| A-034 | 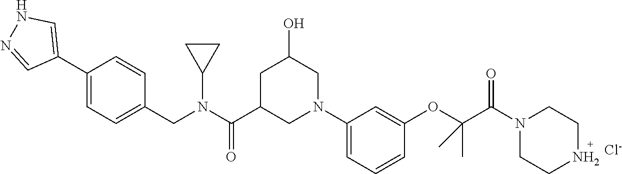 [SMILES: CC(C)(Oc1cccc(N2CC(O)CC(C(=O)N(Cc3ccc(-c4cn[nH]c4)cc3)C3CC3)C2)c1)C(=O)N1CCNCC1 ; [Cl-]] |
| A-035 | 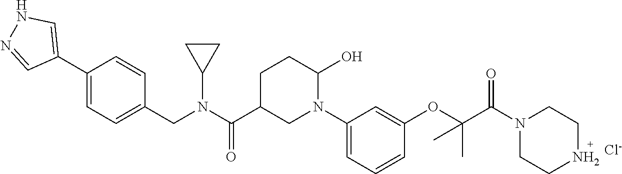 [SMILES: CC(C)(Oc1cccc(N2CC(C(=O)N(Cc3ccc(-c4cn[nH]c4)cc3)C3CC3)CCC2O)c1)C(=O)N1CCNCC1 ; [Cl-]] |
| A-036 | 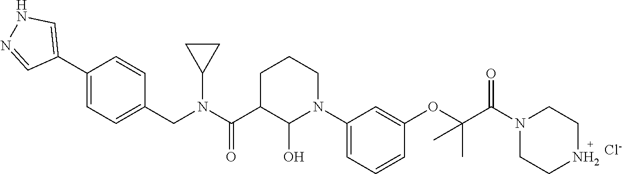 [SMILES: CC(C)(Oc1cccc(N2CCCC(C(=O)N(Cc3ccc(-c4cn[nH]c4)cc3)C3CC3)C2O)c1)C(=O)N1CCNCC1 ; [Cl-]] |
| A-037 | 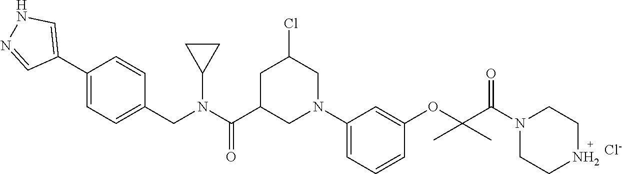 [SMILES: CC(C)(Oc1cccc(N2CC(Cl)CC(C(=O)N(Cc3ccc(-c4cn[nH]c4)cc3)C3CC3)C2)c1)C(=O)N1CCNCC1 ; [Cl-]] |
| A-038 | 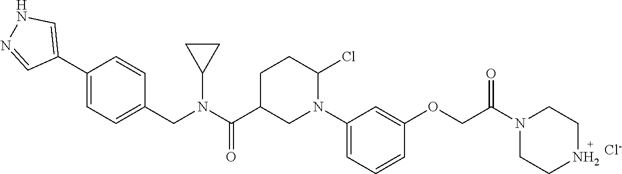 [SMILES: O=C(COc1cccc(N2CC(C(=O)N(Cc3ccc(-c4cn[nH]c4)cc3)C3CC3)CCC2Cl)c1)N1CCNCC1 ; [Cl-]] |
| A-039 | 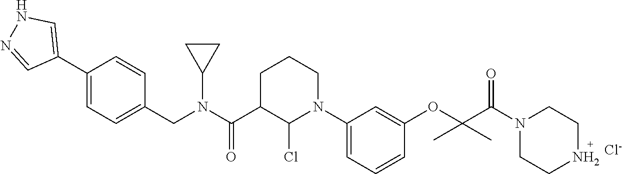 [SMILES: CC(C)(Oc1cccc(N2CCCC(C(=O)N(Cc3ccc(-c4cn[nH]c4)cc3)C3CC3)C2Cl)c1)C(=O)N1CCNCC1 ; [Cl-]] |
| A-040 | 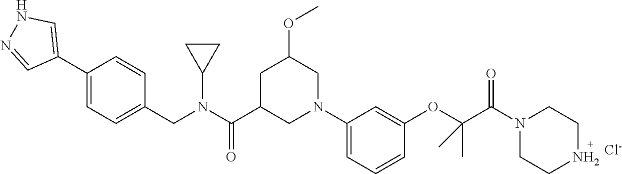 [SMILES: COC1CC(C(=O)N(Cc2ccc(-c3cn[nH]c3)cc2)C2CC2)CN(c2cccc(OC(C)(C)C(=O)N3CCNCC3)c2)C1 ; [Cl-]] |
| A-041 | 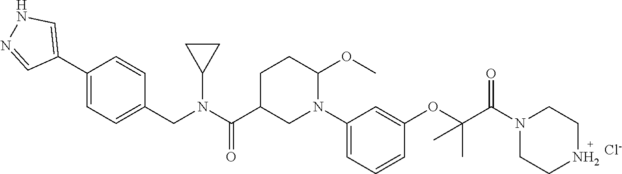 [SMILES: COC1CCC(C(=O)N(Cc2ccc(-c3cn[nH]c3)cc2)C2CC2)CN1c1cccc(OC(C)(C)C(=O)N2CCNCC2)c1 ; [Cl-]] |
| A-042 | 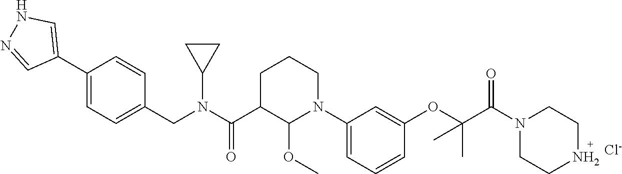 [SMILES: COC1C(C(=O)N(Cc2ccc(-c3cn[nH]c3)cc2)C2CC2)CCCN1c1cccc(OC(C)(C)C(=O)N2CCNCC2)c1 ; [Cl-]] |
| A-043 | 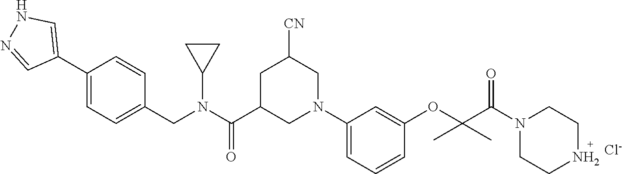 [SMILES: CC(C)(Oc1cccc(N2CC(C#N)CC(C(=O)N(Cc3ccc(-c4cn[nH]c4)cc3)C3CC3)C2)c1)C(=O)N1CCNCC1 ; [Cl-]] |
| A-044 | 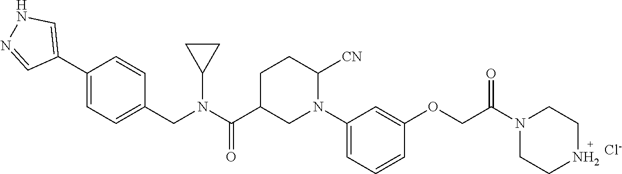 [SMILES: N#CC1CCC(C(=O)N(Cc2ccc(-c3cn[nH]c3)cc2)C2CC2)CN1c1cccc(OCC(=O)N2CCNCC2)c1 ; [Cl-]] |
| A-045 | 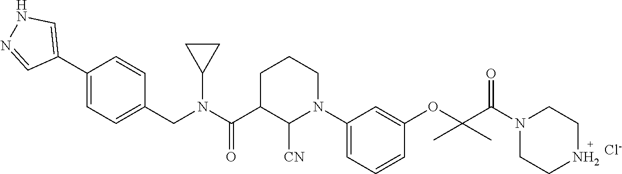 [SMILES: CC(C)(Oc1cccc(N2CCCC(C(=O)N(Cc3ccc(-c4cn[nH]c4)cc3)C3CC3)C2C#N)c1)C(=O)N1CCNCC1 ; [Cl-]] |
| A-046 | 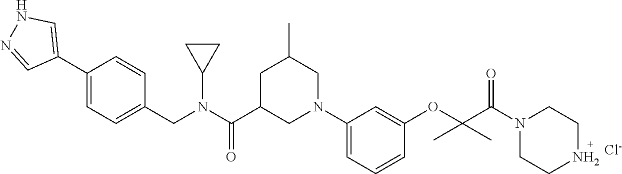 [SMILES: CC1CC(C(=O)N(Cc2ccc(-c3cn[nH]c3)cc2)C2CC2)CN(c2cccc(OC(C)(C)C(=O)N3CCNCC3)c2)C1 ; [Cl-]] |
| A-047 | 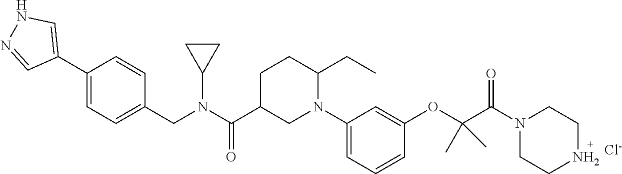 [SMILES: CCC1CCC(C(=O)N(Cc2ccc(-c3cn[nH]c3)cc2)C2CC2)CN1c1cccc(OC(C)(C)C(=O)N2CCNCC2)c1 ; [Cl-]] |
| A-048 | 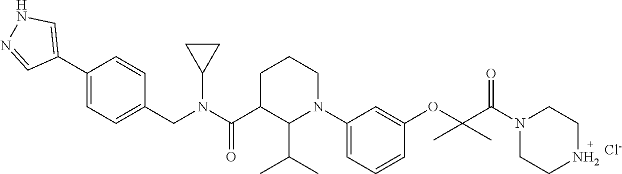 [SMILES: CC(C)C1C(C(=O)N(Cc2ccc(-c3cn[nH]c3)cc2)C2CC2)CCCN1c1cccc(OC(C)(C)C(=O)N2CCNCC2)c1 ; [Cl-]] |
| A-049 | 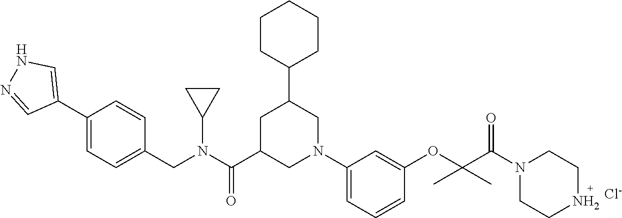 [SMILES: CC(C)(Oc1cccc(N2CC(C(=O)N(Cc3ccc(-c4cn[nH]c4)cc3)C3CC3)CC(C3CCCCC3)C2)c1)C(=O)N1CCNCC1 ; [Cl-]] |
| A-050 | 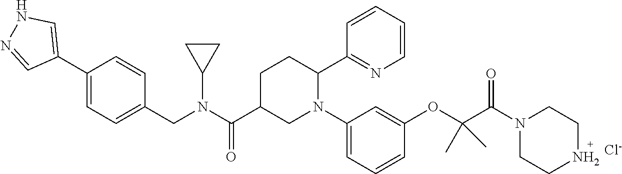 [SMILES: CC(C)(Oc1cccc(N2CC(C(=O)N(Cc3ccc(-c4cn[nH]c4)cc3)C3CC3)CCC2c2ccccn2)c1)C(=O)N1CCNCC1 ; [Cl-]] |

In another preferred embodiment, the compound is selected from Table A4:

TABLE A4

| | |
|---|---|
| A-019 | 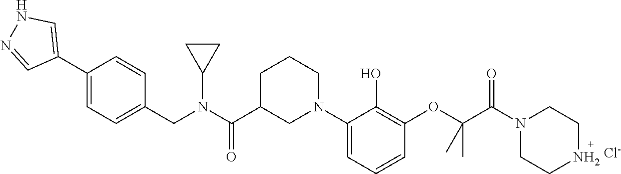 [SMILES: CC(C)(Oc1cccc(N2CCCC(C(=O)N(Cc3ccc(-c4cn[nH]c4)cc3)C3CC3)C2)c1O)C(=O)N1CCNCC1 ; [Cl-]] |
| A-020 | 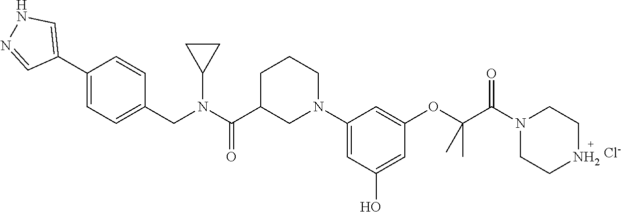 [SMILES: CC(C)(Oc1cc(O)cc(N2CCCC(C(=O)N(Cc3ccc(-c4cn[nH]c4)cc3)C3CC3)C2)c1)C(=O)N1CCNCC1 ; [Cl-]] |
| A-021 | 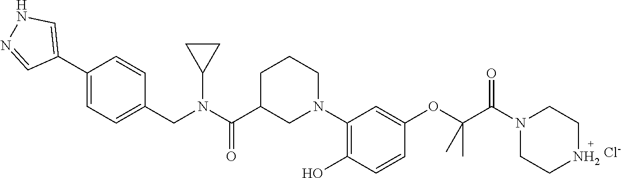 [SMILES: CC(C)(Oc1ccc(O)c(N2CCCC(C(=O)N(Cc3ccc(-c4cn[nH]c4)cc3)C3CC3)C2)c1)C(=O)N1CCNCC1 ; [Cl-]] |

In another preferred embodiment, the compound is selected from Table A5:

TABLE A5

| | |
|---|---|
| A-028 | 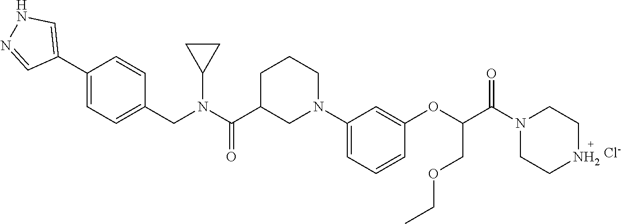 [SMILES: CCOCC(Oc1cccc(N2CCCC(C(=O)N(Cc3ccc(-c4cn[nH]c4)cc3)C3CC3)C2)c1)C(=O)N1CCNCC1 ; [Cl-]] |
| A-029 | 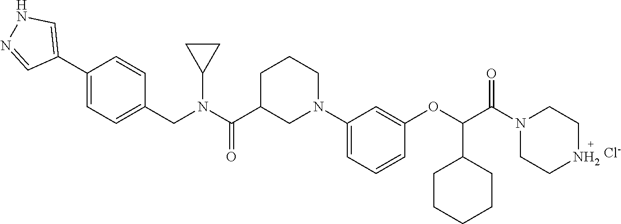 [SMILES: O=C(C(Oc1cccc(N2CCCC(C(=O)N(Cc3ccc(-c4cn[nH]c4)cc3)C3CC3)C2)c1)C1CCCCC1)N1CCNCC1 ; [Cl-]] |
| A-030 | 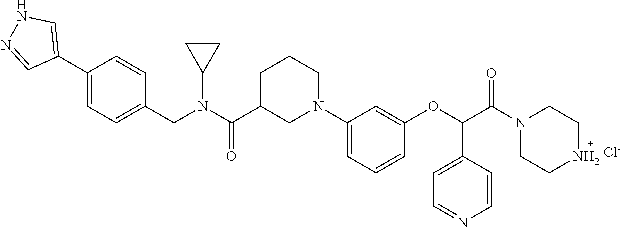 [SMILES: O=C(C(Oc1cccc(N2CCCC(C(=O)N(Cc3ccc(-c4cn[nH]c4)cc3)C3CC3)C2)c1)c1ccncc1)N1CCNCC1 ; [Cl-]] |
| A-031 | 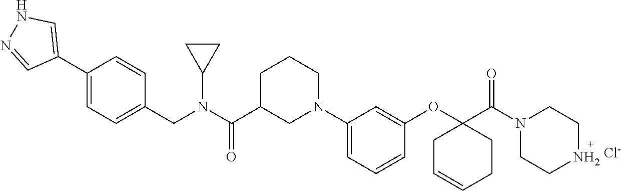 [SMILES: O=C(C1CCCN(c2cccc(OC3(C(=O)N4CCNCC4)CC=CCC3)c2)C1)N(Cc1ccc(-c2cn[nH]c2)cc1)C1CC1 ; [Cl-]] |
| A-032 | 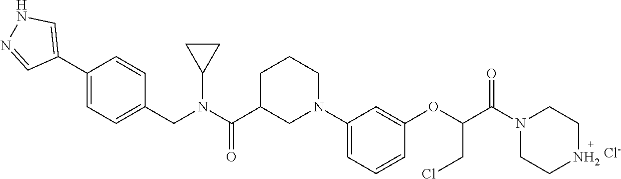 [SMILES: O=C(C(CCl)Oc1cccc(N2CCCC(C(=O)N(Cc3ccc(-c4cn[nH]c4)cc3)C3CC3)C2)c1)N1CCNCC1 ; [Cl-]] |
| A-033 | 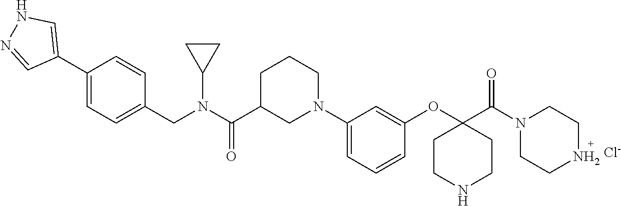 [SMILES: O=C(C1CCCN(c2cccc(OC3(C(=O)N4CCNCC4)CCNCC3)c2)C1)N(Cc1ccc(-c2cn[nH]c2)cc1)C1CC1 ; [Cl-]] |

In another preferred embodiment, the compound is selected from Table A6:

TABLE A6

| | |
|---|---|
| A-010 | 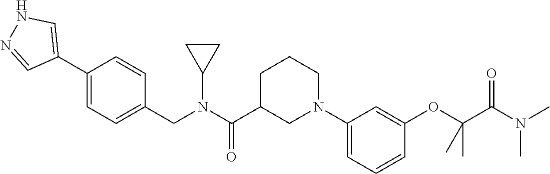 [SMILES: CN(C)C(=O)C(C)(C)Oc1cccc(N2CCCC(C(=O)N(Cc3ccc(-c4cn[nH]c4)cc3)C3CC3)C2)c1] |
| A-011 | 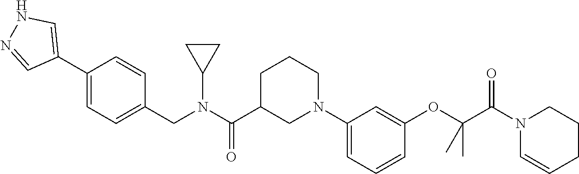 [SMILES: CC(C)(Oc1cccc(N2CCCC(C(=O)N(Cc3ccc(-c4cn[nH]c4)cc3)C3CC3)C2)c1)C(=O)N1C=CCCC1] |
| A-012 | 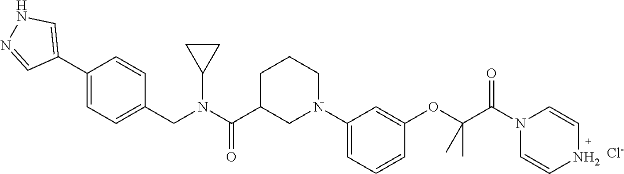 [SMILES: CC(C)(Oc1cccc(N2CCCC(C(=O)N(Cc3ccc(-c4cn[nH]c4)cc3)C3CC3)C2)c1)C(=O)N1C=CNC=C1 ; [Cl-]] |
| A-013 | 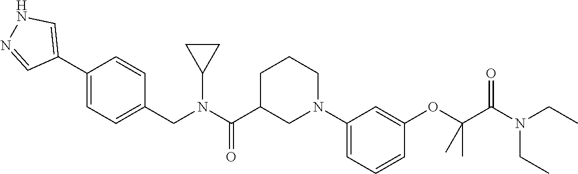 [SMILES: CCN(CC)C(=O)C(C)(C)Oc1cccc(N2CCCC(C(=O)N(Cc3ccc(-c4cn[nH]c4)cc3)C3CC3)C2)c1] |
| A-014 | 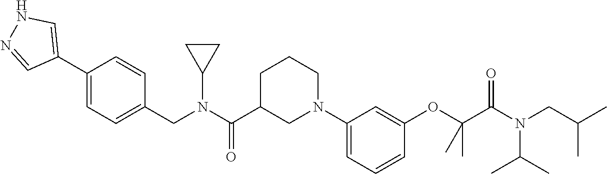 [SMILES: CC(C)CN(C(=O)C(C)(C)Oc1cccc(N2CCCC(C(=O)N(Cc3ccc(-c4cn[nH]c4)cc3)C3CC3)C2)c1)C(C)C] |
| A-015 | 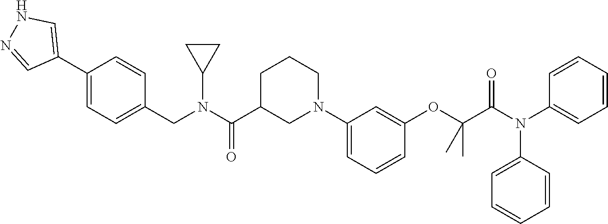 [SMILES: CC(C)(Oc1cccc(N2CCCC(C(=O)N(Cc3ccc(-c4cn[nH]c4)cc3)C3CC3)C2)c1)C(=O)N(c1ccccc1)c1ccccc1] |
| A-016 | 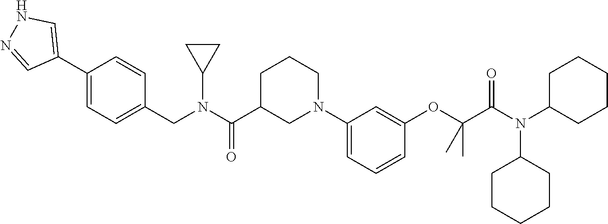 [SMILES: CC(C)(Oc1cccc(N2CCCC(C(=O)N(Cc3ccc(-c4cn[nH]c4)cc3)C3CC3)C2)c1)C(=O)N(C1CCCCC1)C1CCCCC1] |
| A-017 | 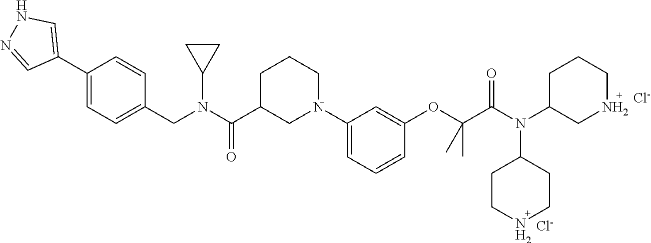 [SMILES: CC(C)(Oc1cccc(N2CCCC(C(=O)N(Cc3ccc(-c4cn[nH]c4)cc3)C3CC3)C2)c1)C(=O)N(C1CCNCC1)C1CCCNC1 ; [Cl-] ; [Cl-]] |
| A-018 | 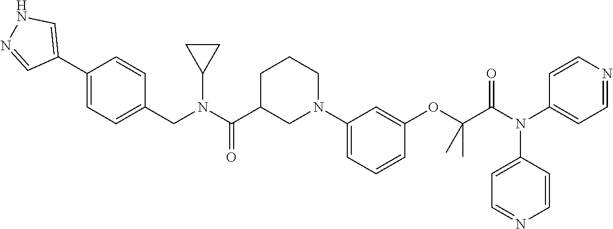 [SMILES: CC(C)(Oc1cccc(N2CCCC(C(=O)N(Cc3ccc(-c4cn[nH]c4)cc3)C3CC3)C2)c1)C(=O)N(c1ccncc1)c1ccncc1] |

In another preferred embodiment, the compound is as shown in Formula IV;

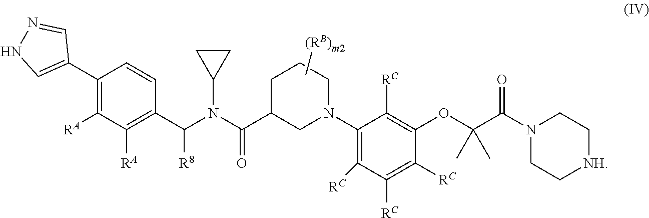

In another preferred embodiment, at least one of R^{A} is R^{A1}

In another preferred embodiment, R^{A} at the ortho position of the —C(R⁸)— group is R^{A1}, and R^{A} at the meta-position of the —C(R⁸)— group is H.

In another preferred embodiment, the compound is as shown in Formula IVa or Formula IVb;

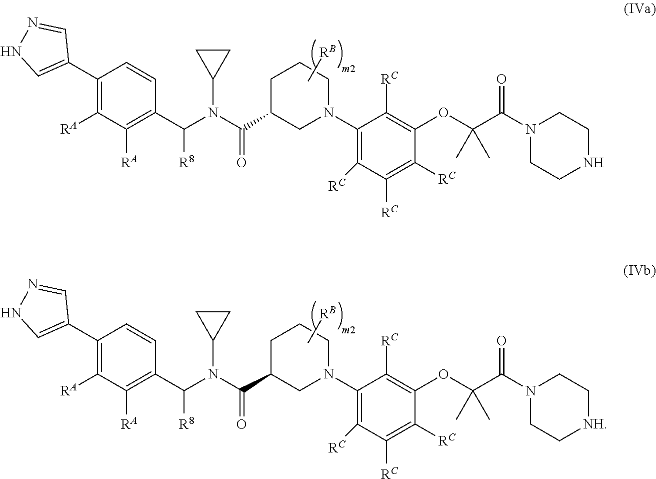

In another preferred embodiment, the compound is as shown in Formula IV-1 or Formula IV-2;

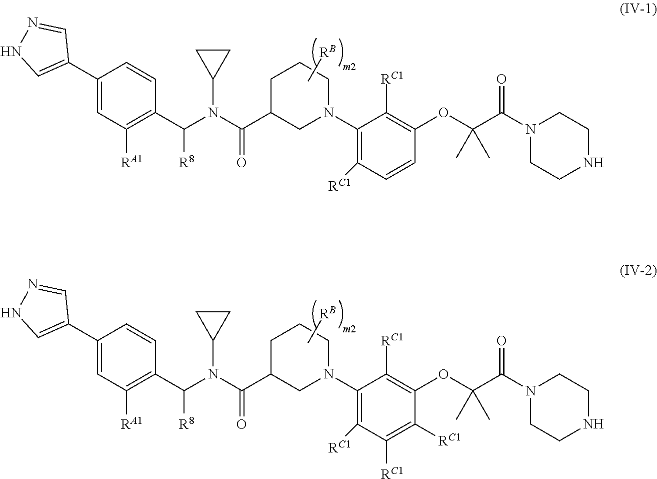

In another preferred embodiment, the compound is as shown in Formula IV-1a, IV-1b, IV-2a, or Formula IV-2b;

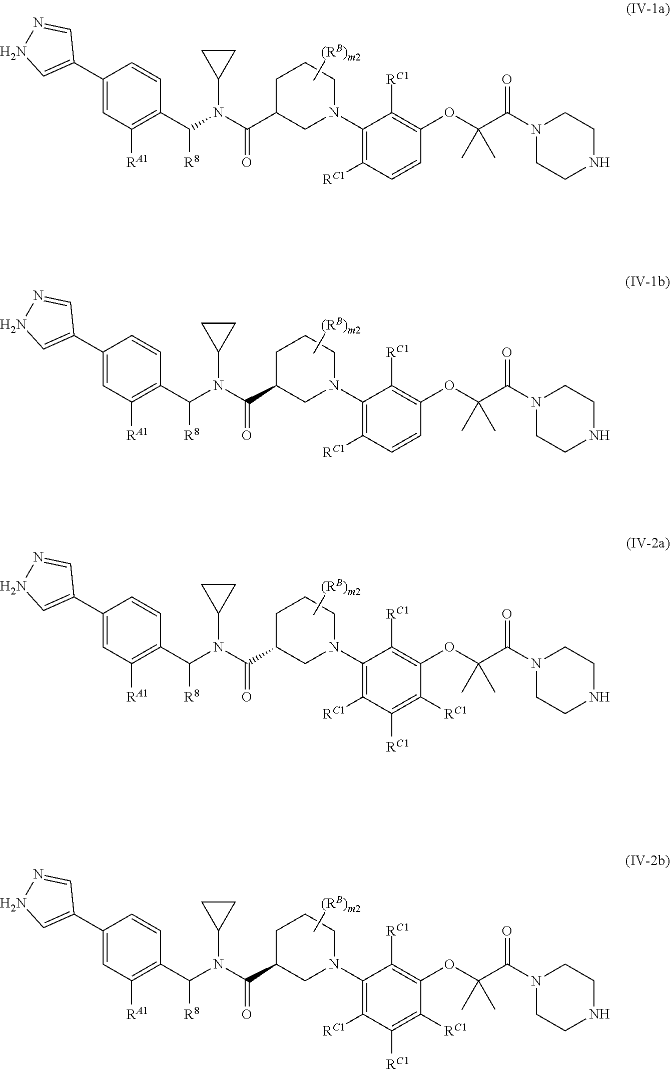

In another preferred embodiment, R^{A1} is selected from the group consisting of: halogen (preferably, Cl), C₁₋₆ haloalkyl (preferably, trifluoromethyl), C₁₋₆ alkoxy (preferably, methoxy).

In another preferred embodiment, R^{C1} is each independently selected from the group consisting of: halogen (preferably, Cl), C₁₋₆ haloalkyl (preferably, trifluoromethyl), C₁₋₆ alkoxy (preferably, methoxy).

In another preferred embodiment, R^{C1} is the same or different group.

In another preferred embodiment, the compound or the pharmaceutically acceptable salt thereof is selected from the following table:

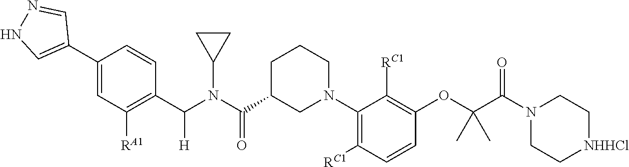

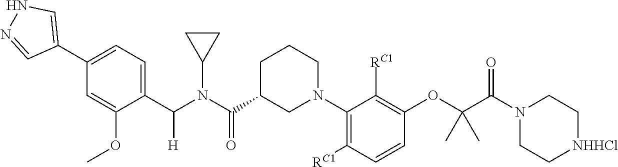

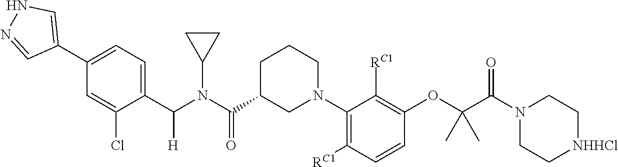

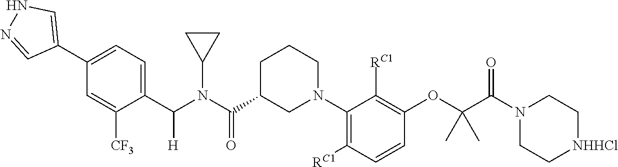

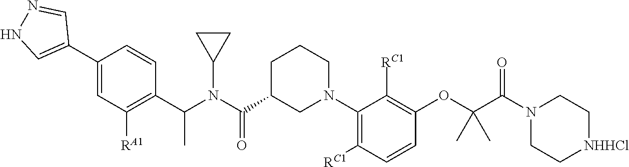

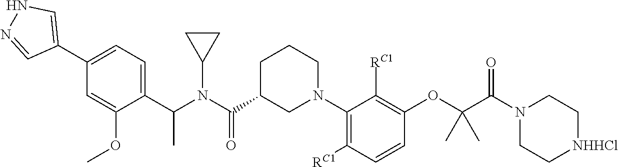

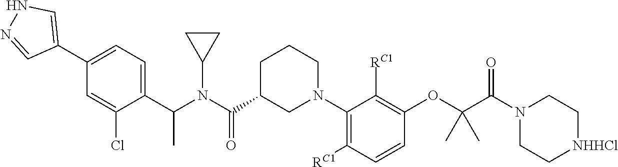

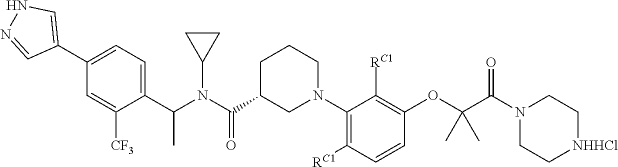

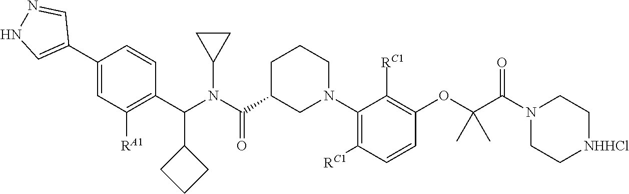

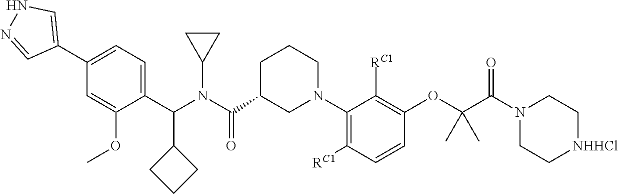

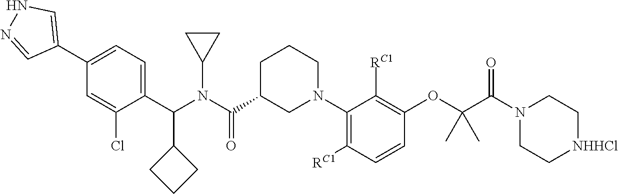

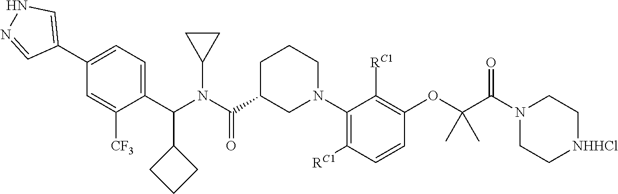

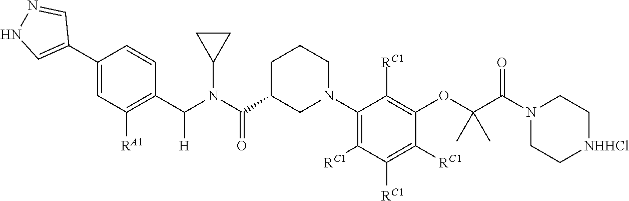

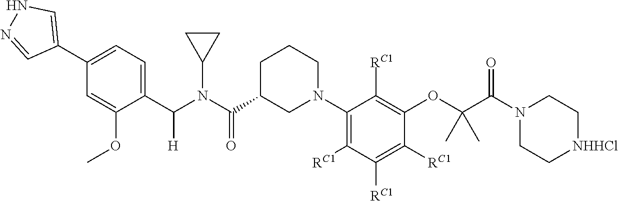

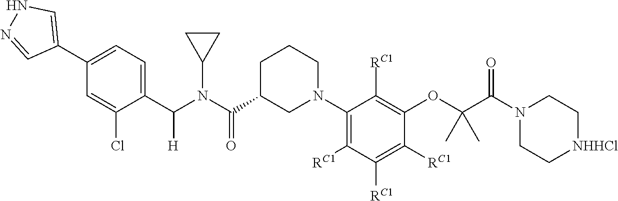

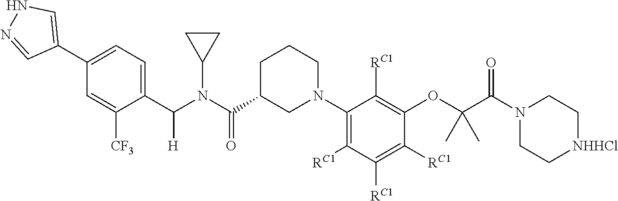

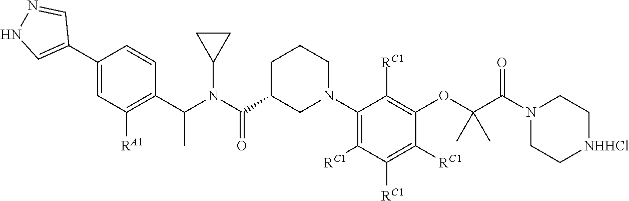

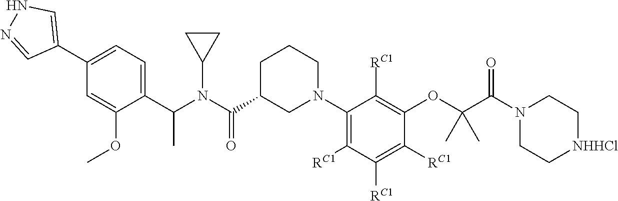

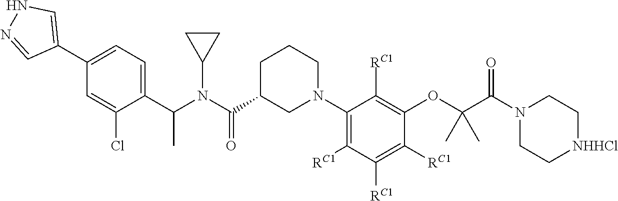

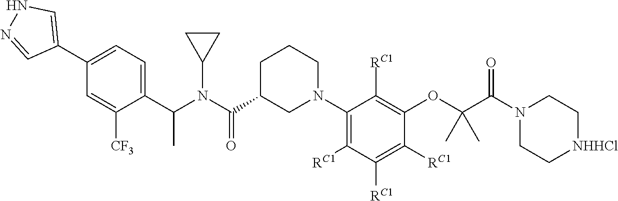

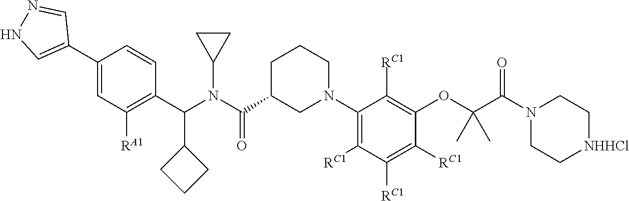

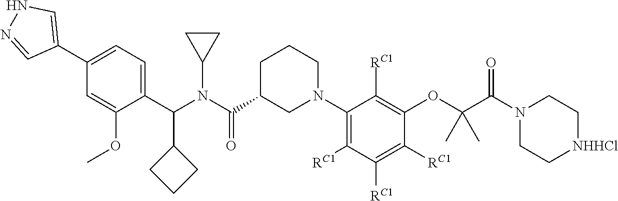

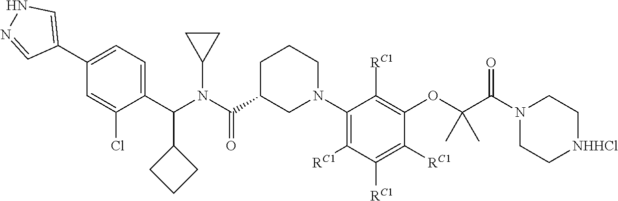

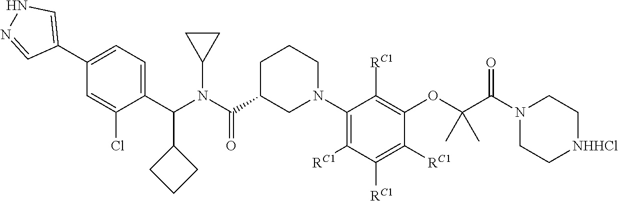

wherein R^{A1} and R^{C1} are as defined above.

In another preferred embodiment, the compound or a pharmaceutically acceptable salt thereof is selected from Table B below.

TABLE B

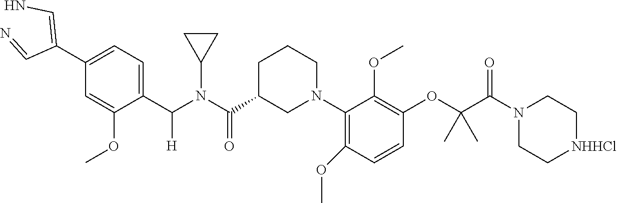

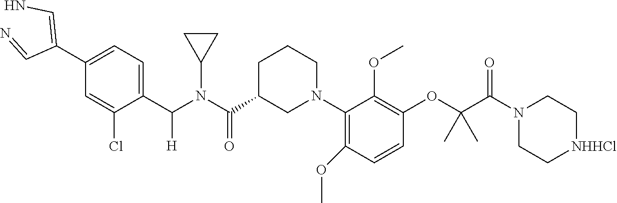

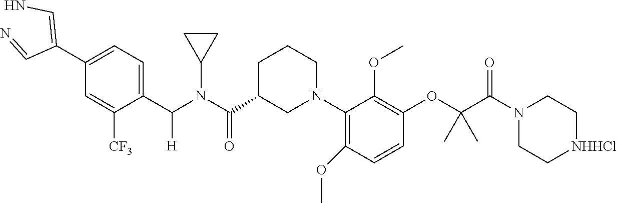

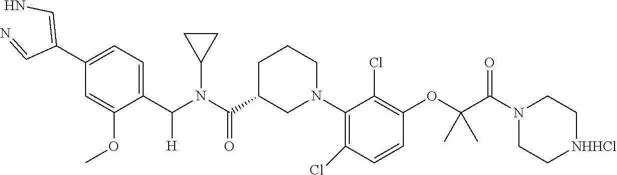

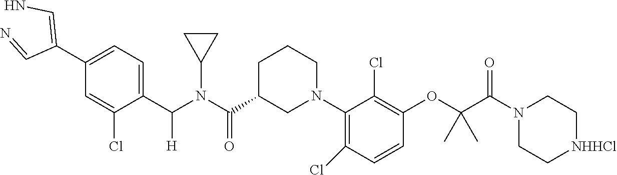

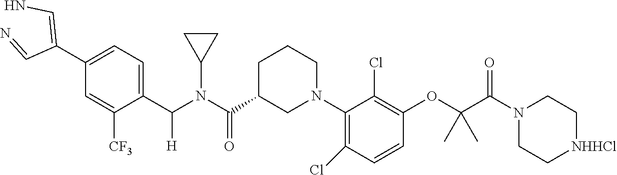

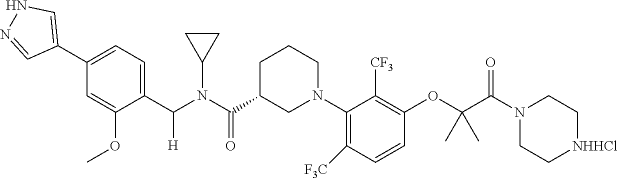

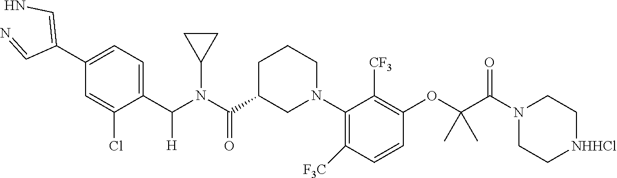

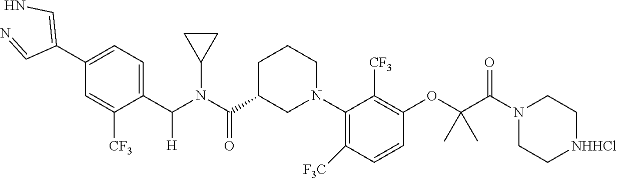

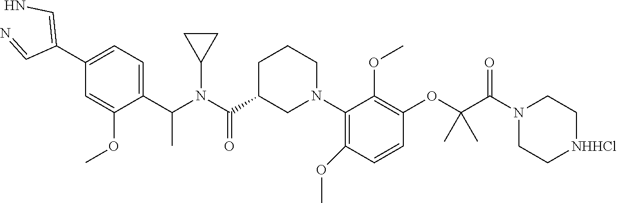

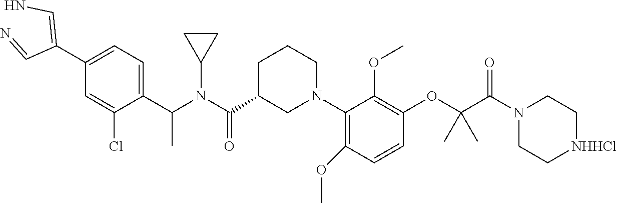

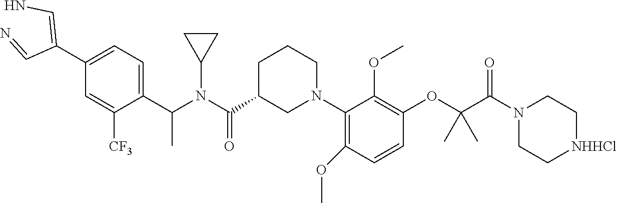

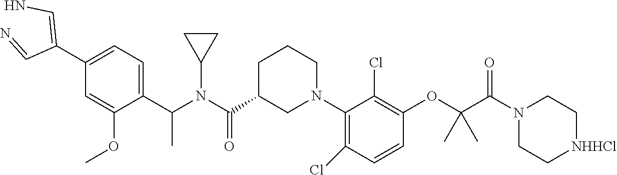

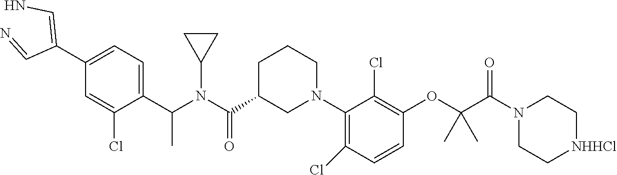

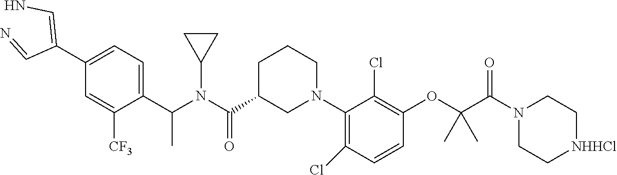

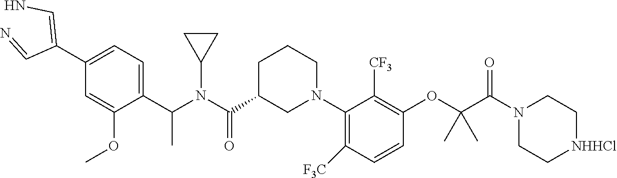

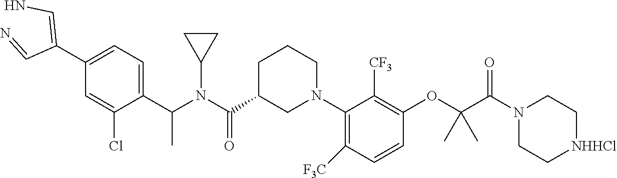

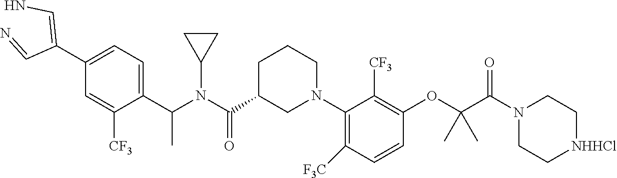

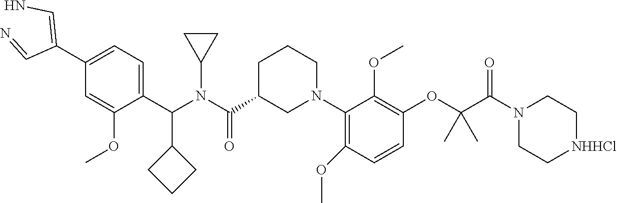

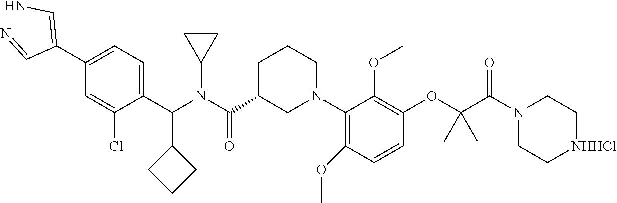

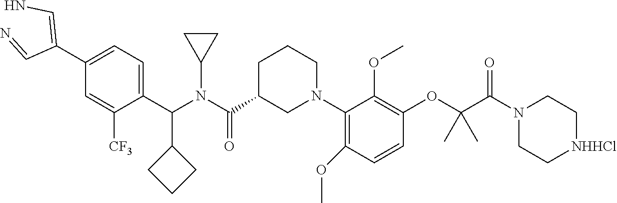

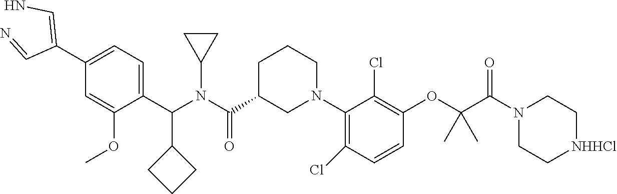

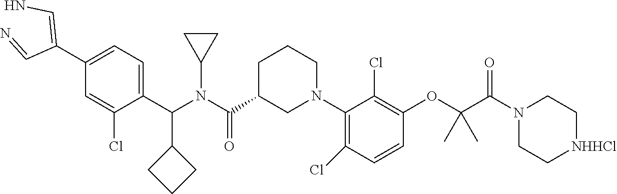

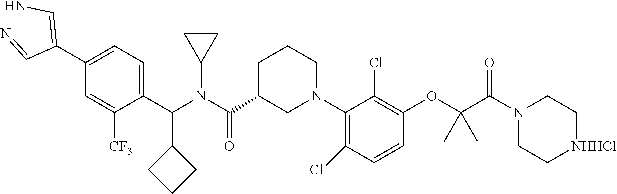

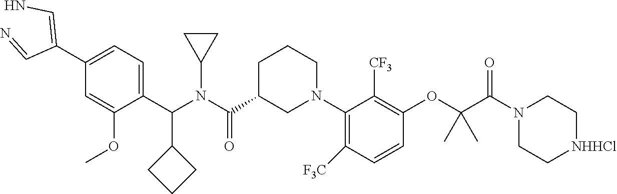

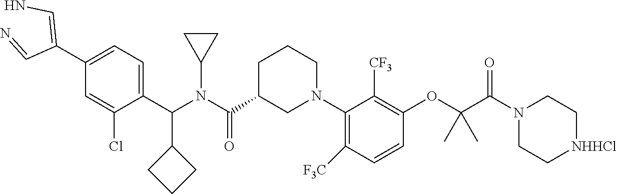

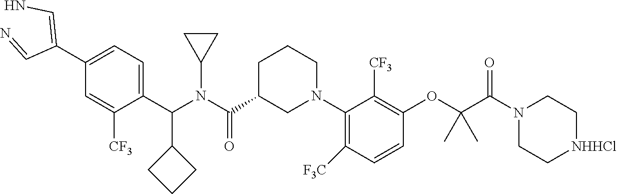

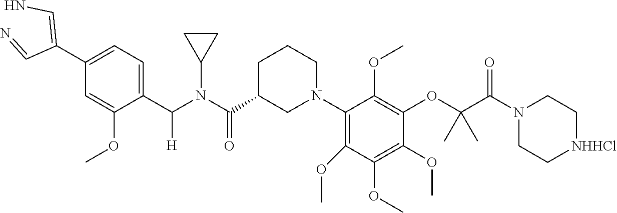

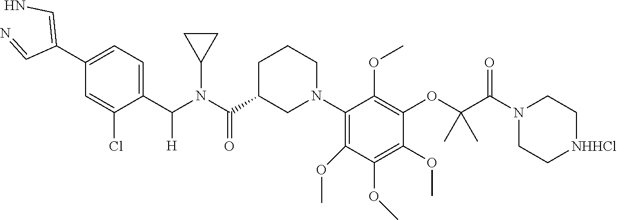

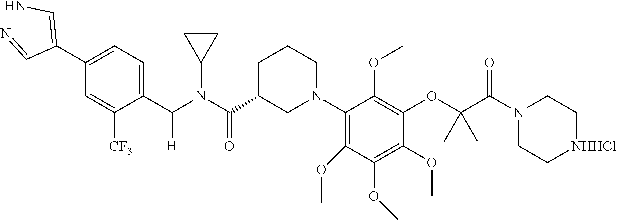

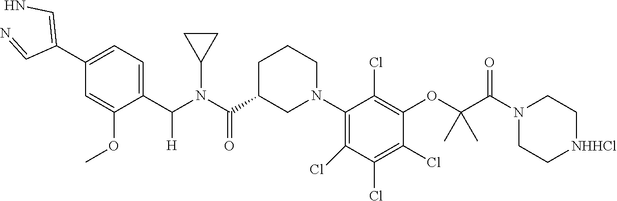

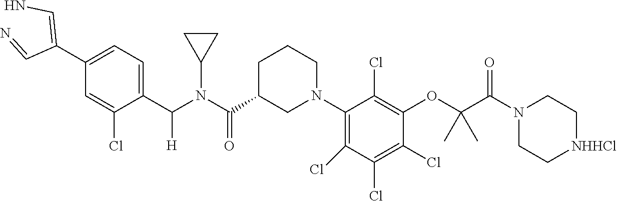

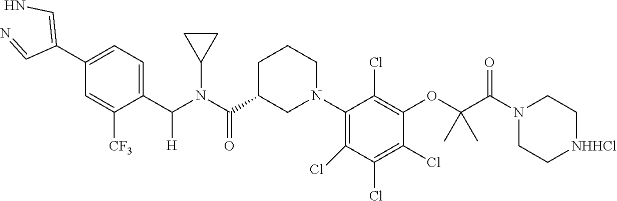

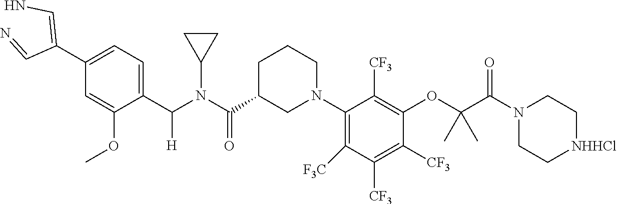

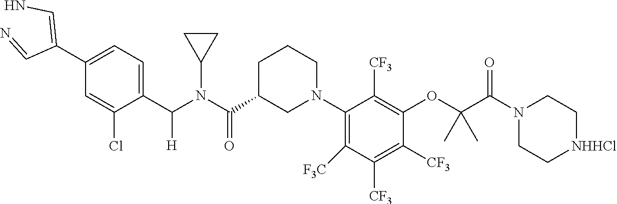

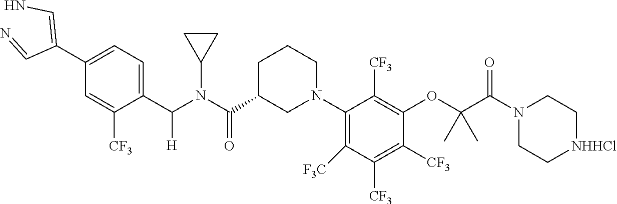

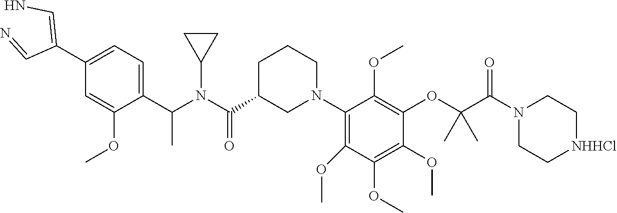

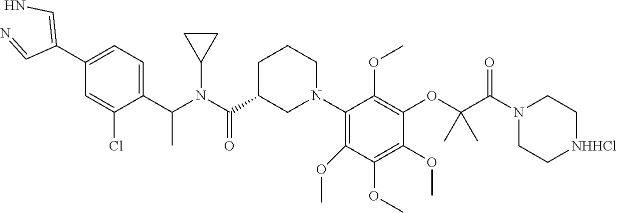

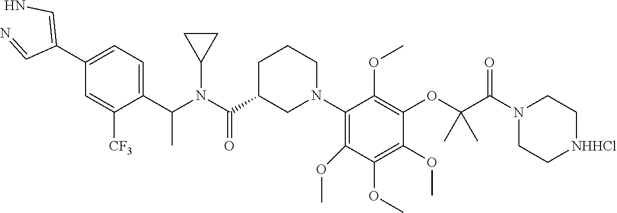

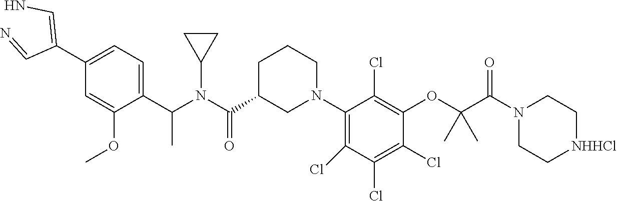

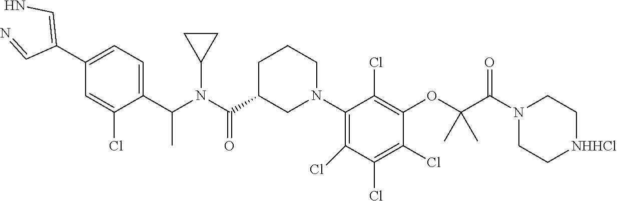

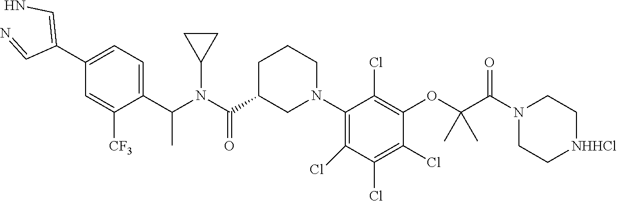

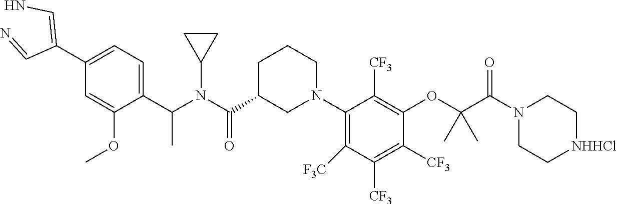

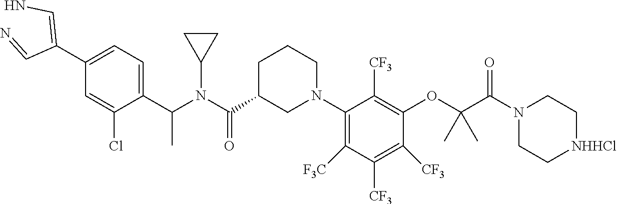

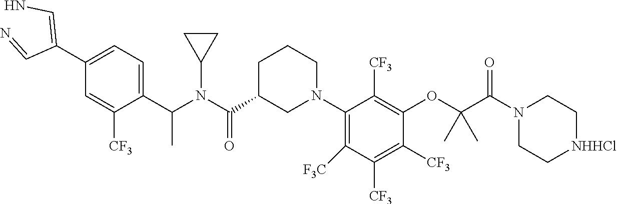

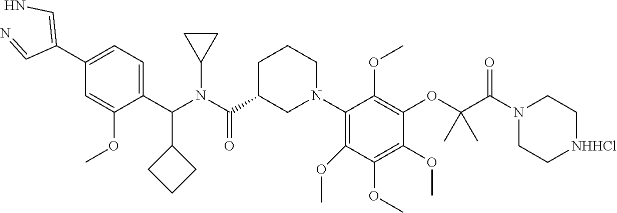

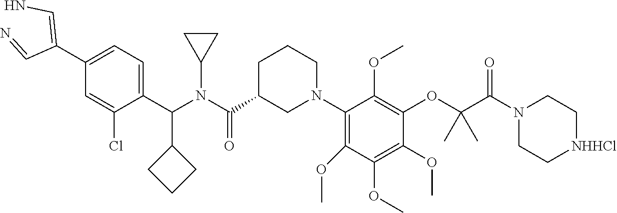

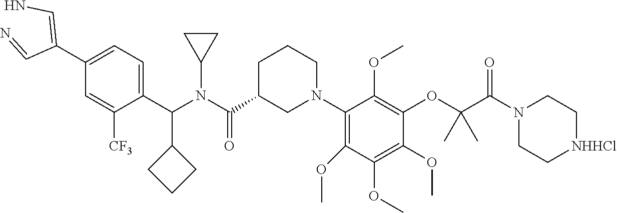

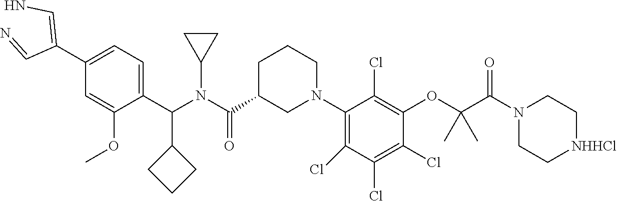

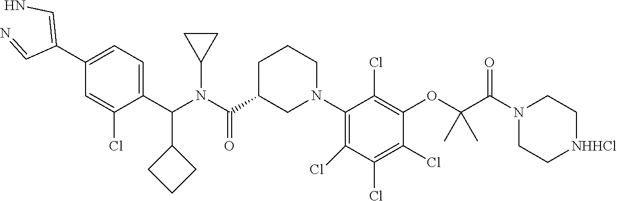

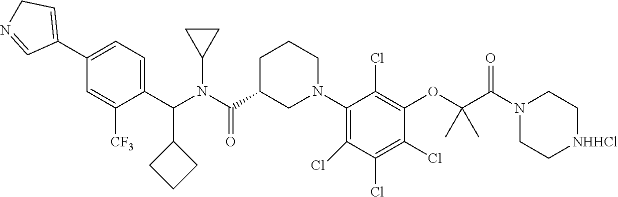

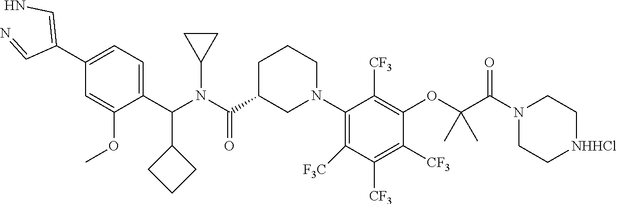

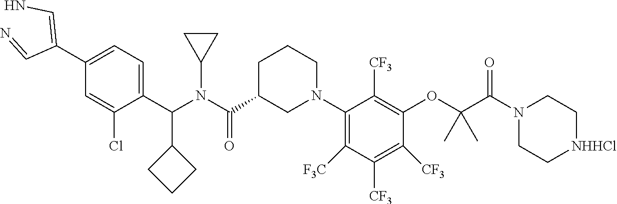

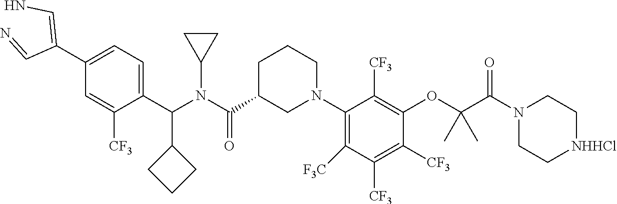

In another preferred embodiment, the compound is as shown in formula IV-3, IV-3a, IV-3b.

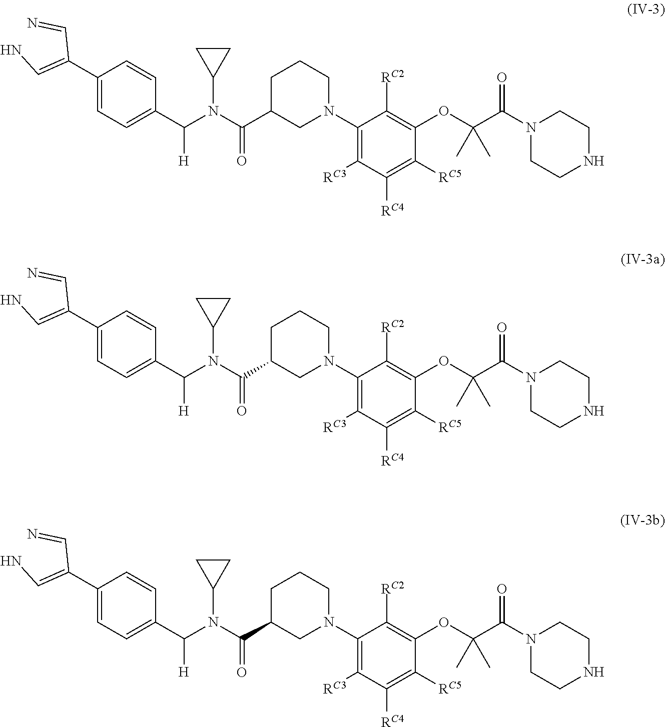

wherein, R^{C2}, R^{C3}, R^{C4}, and R^{C5} are defined as R^{C}.

In another preferred embodiment, at least one of R^{C2}, R^{C3}, R^{C4} and R^{C5} is R^{C1}, and the rest is R^{C1} or R^{s}.

In another preferred embodiment, the compound is selected from Table C below.

TABLE C

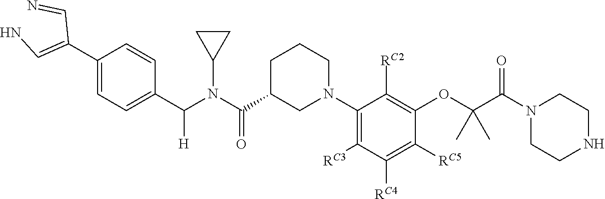

| | RC2 | RC3 | RC4 | RC5 |
|---|---|---|---|---|
| C020 | Me | H | H | H |
| C021 | H | Me | H | H |
| C022 | H | H | Me | H |
| C023 | H | H | H | Me |
| C024 | OMe | H | H | H |
| C025 | H | OMe | H | H |
| C026 | H | H | OMe | H |
| C027 | H | H | H | OMe |
| C028 | CF3 | H | H | H |
| C029 | H | CF3 | H | H |
| C030 | H | H | CF3 | H |
| C031 | H | H | H | CF3 |
| C032 | OCF3 | H | H | H |
| C033 | H | OCF3 | H | H |
| C034 | H | H | OCF3 | H |
| C035 | H | H | H | OCF3 |
| C036 (i.e. compound 7) | C1 | H | H | H |
| C037 (i.e. compound 9) | H | C1 | H | H |

In another preferred embodiment,
R⁷ is an optionally substituted C₃₋₁₀cycloalkenyl or optionally substituted 5-10 membered heteroaryl;
Ring A is

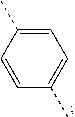

m1=0 or 1;
R^{A} is H or R^{A1}; and R^{A1} is selected from the group consisting of: halogen, optionally substituted C₁₋₆ haloalkyl, and optionally substituted C₁₋₆ alkoxy group (preferably, R^{A1} is halogen);
L¹ is-CH(R⁸)—N(R¹)—C(O)— or —CH(R⁸)—N(R¹)—C(O)—NH—, wherein the CH(R⁸) terminal is attached to Ring A; wherein, R¹ is optionally substituted C₃₋₆ cycloalkyl, R⁸ is selected from the group consisting of: H, optionally substituted C₁₋₆ alkyl;

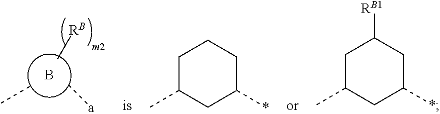

wherein * refers to the attachment to Ring C; and wherein R^{B1} is selected from the group consisting of: optionally substituted C₃₋₁₀ cycloalkyl, optionally substituted 4 to 10-membered heterocycloalkyl, optionally substituted C₆₋₁₀ aryl, and optionally substituted 5 to 10-membered heteroaryl (preferably, R^{B1} is selected from the group consisting of: cyclohexyl and phenyl);
Ring C is

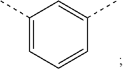

m3=0, 1, or 2;
R^{C} is H, C₁₋₄ alkyl or R^{C1}; and R^{C1} is selected from the group consisting of: halogen (preferably, F, Cl), C₁₋₆ haloalkyl (preferably, trifluoromethyl), and C₁₋₆ alkoxy (preferably, methoxy); preferably, R^{C1} is a halogen;
L² is —W²—CR²R³—C(O)— and W² is selected from the group consisting of: —O—, —S—, —N(R^{s})—; wherein both R² and R³ are optionally substituted C₁₋₄ alkyl (preferably, both R² and R³ are methyl);

In another preferred embodiment, R⁶ is —NR⁴R⁵; wherein,
R⁴ and R⁵ are independently selected from the group consisting of: H, optionally substituted C₁₋₆ alkyl; and wherein the optionally substituted means that one hydrogen in the group is substituted with a substituent selected from the group consisting of: —OR′, —NR′R″; wherein R′ is independently selected from the group consisting of: H, D, C₁₋₆ alkyl, and R″ is selected from the group consisting of: H, D, C₁₋₄ alkyl (preferably, R′ is H and R″ is H); or, —N⁴R⁵ is a 4 to 10-membered heterocycloalkyl with at least one —O— present in the ring; or, —NR⁴R⁵ is 4 to 10 membered heterocycloalkyl with at least one —NH₂⁺— or —NH— present on the ring.

In another preferred embodiment, L¹ is —CH(R⁸)—N(R¹)—C(O)—NH—.

In another preferred embodiment, the compound is selected from Table D below.

TABLE D

| | |
|---|---|
| Compound 1 i.e., the free base of A-049, C37-001 | 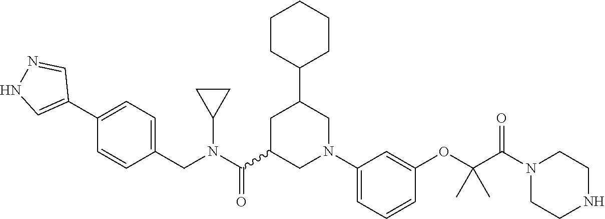 [SMILES: CC(C)(Oc1cccc(N2CC(C(=O)N(Cc3ccc(-c4cn[nH]c4)cc3)C3CC3)CC(C3CCCCC3)C2)c1)C(=O)N1CCNCC1] |
| Compound 2 i.e., the free base of A-052, C37-002 | 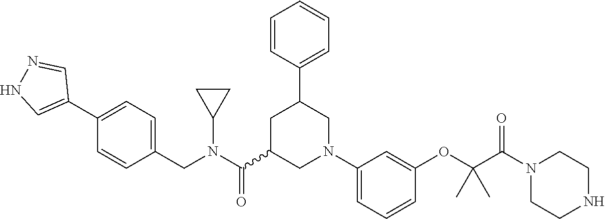 [SMILES: CC(C)(Oc1cccc(N2CC(C(=O)N(Cc3ccc(-c4cn[nH]c4)cc3)C3CC3)CC(c3ccccc3)C2)c1)C(=O)N1CCNCC1] |
| Compound 3 | 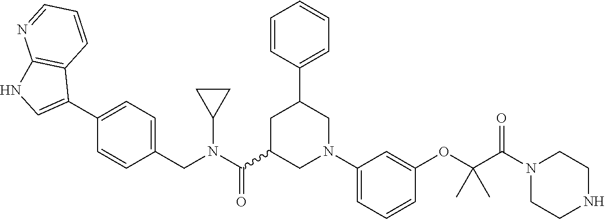 [SMILES: CC(C)(Oc1cccc(N2CC(C(=O)N(Cc3ccc(-c4c[nH]c5ncccc45)cc3)C3CC3)CC(c3ccccc3)C2)c1)C(=O)N1CCNCC1] |
| Compound 4 | 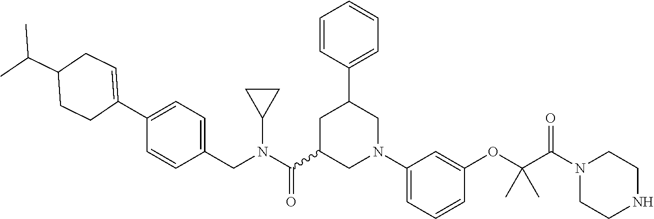 [SMILES: CC(C)C1CC=C(c2ccc(CN(C(=O)C3CC(c4ccccc4)CN(c4cccc(OC(C)(C)C(=O)N5CCNCC5)c4)C3)C3CC3)cc2)CC1] |
| Compound 5 | 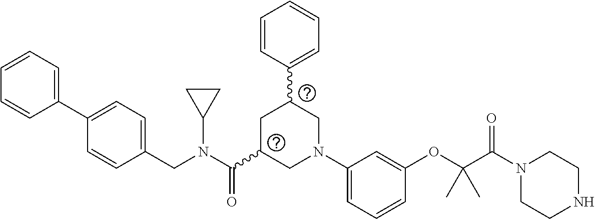 [SMILES: CC(C)(Oc1cccc(N2CC(C(=O)N(Cc3ccc(-c4ccccc4)cc3)C3CC3)CC(c3ccccc3)C2)c1)C(=O)N1CCNCC1] |
| Compound 6 | 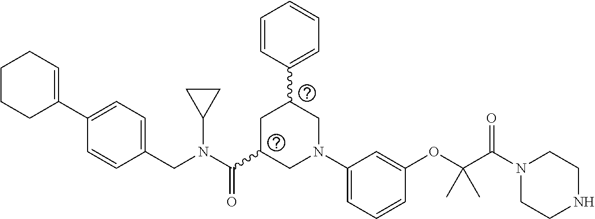 [SMILES: CC(C)(Oc1cccc(N2CC(C(=O)N(Cc3ccc(C4=CCCCC4)cc3)C3CC3)CC(c3ccccc3)C2)c1)C(=O)N1CCNCC1] |
| Compound i.e., C036, C37-008 | 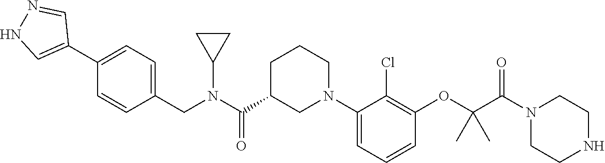 [SMILES: CC(C)(Oc1cccc(N2CCC[C@@H](C(=O)N(Cc3ccc(-c4cn[nH]c4)cc3)C3CC3)C2)c1Cl)C(=O)N1CCNCC1] |
| Compound 8 | 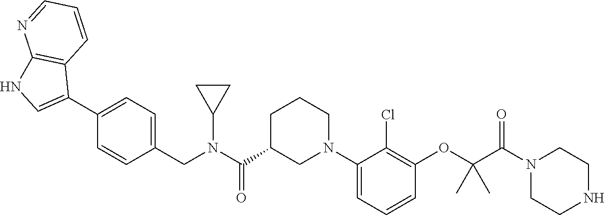 [SMILES: CC(C)(Oc1cccc(N2CCC[C@@H](C(=O)N(Cc3ccc(-c4c[nH]c5ncccc45)cc3)C3CC3)C2)c1Cl)C(=O)N1CCNCC1] |
| Compound 9 i.e., C037, C37-009 | 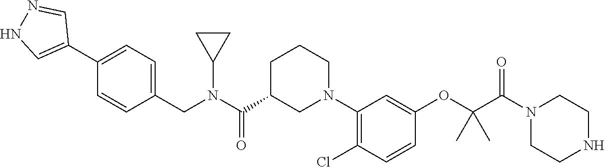 [SMILES: CC(C)(Oc1ccc(Cl)c(N2CCC[C@@H](C(=O)N(Cc3ccc(-c4cn[nH]c4)cc3)C3CC3)C2)c1)C(=O)N1CCNCC1] |
| Compound 10 | 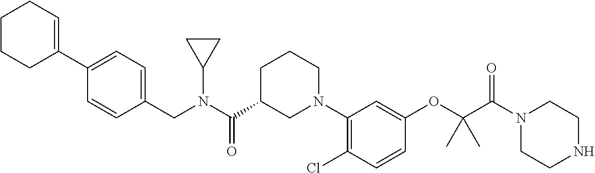 [SMILES: CC(C)(Oc1ccc(Cl)c(N2CCC[C@@H](C(=O)N(Cc3ccc(C4=CCCCC4)cc3)C3CC3)C2)c1)C(=O)N1CCNCC1] |
| Compound 11 | 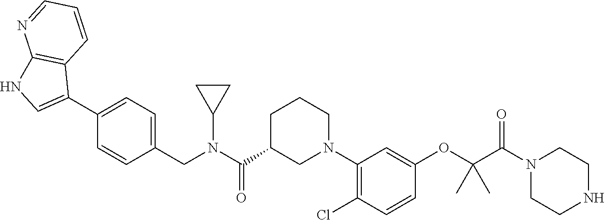 [SMILES: CC(C)(Oc1ccc(Cl)c(N2CCC[C@@H](C(=O)N(Cc3ccc(-c4c[nH]c5ncccc45)cc3)C3CC3)C2)c1)C(=O)N1CCNCC1] |
| Compound 12 C37-007 | 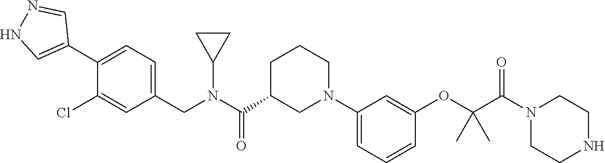 [SMILES: CC(C)(Oc1cccc(N2CCC[C@@H](C(=O)N(Cc3ccc(-c4cn[nH]c4)c(Cl)c3)C3CC3)C2)c1)C(=O)N1CCNCC1] |
| Compound 13 | 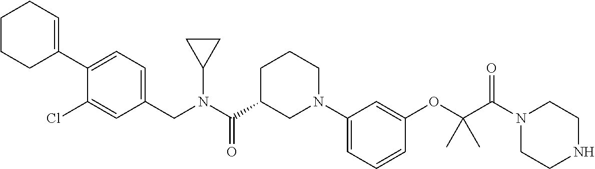 [SMILES: CC(C)(Oc1cccc(N2CCC[C@@H](C(=O)N(Cc3ccc(C4=CCCCC4)c(Cl)c3)C3CC3)C2)c1)C(=O)N1CCNCC1] |
| Compound 14 | 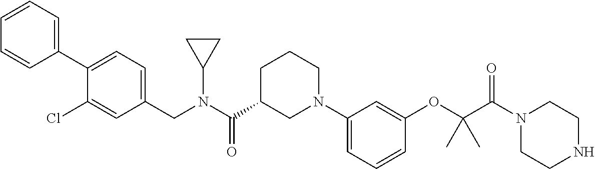 [SMILES: CC(C)(Oc1cccc(N2CCC[C@@H](C(=O)N(Cc3ccc(-c4ccccc4)c(Cl)c3)C3CC3)C2)c1)C(=O)N1CCNCC1] |
| Compound 15 i.e., C37-010 | 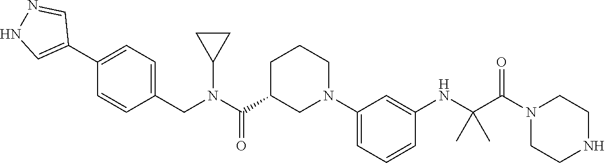 [SMILES: CC(C)(Nc1cccc(N2CCC[C@@H](C(=O)N(Cc3ccc(-c4cn[nH]c4)cc3)C3CC3)C2)c1)C(=O)N1CCNCC1] |
| Compound 16 i.e., C37-011 | 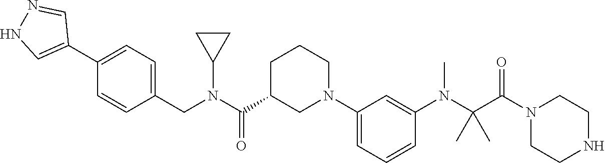 [SMILES: CN(c1cccc(N2CCC[C@@H](C(=O)N(Cc3ccc(-c4cn[nH]c4)cc3)C3CC3)C2)c1)C(C)(C)C(=O)N1CCNCC1] |
| Compound 17 i.e., C37-012 | 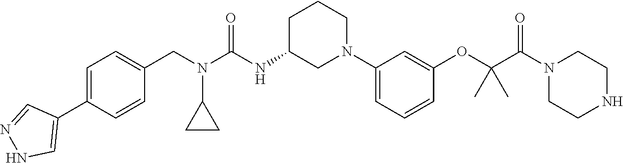 [SMILES: CC(C)(Oc1cccc(N2CCC[C@@H](NC(=O)N(Cc3ccc(-c4cn[nH]c4)cc3)C3CC3)C2)c1)C(=O)N1CCNCC1] |
| Compound 18 i.e., C37-013 | 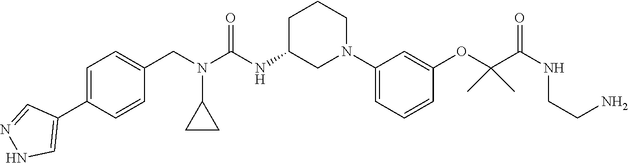 [SMILES: CC(C)(Oc1cccc(N2CCC[C@@H](NC(=O)N(Cc3ccc(-c4cn[nH]c4)cc3)C3CC3)C2)c1)C(=O)NCCN] |
| Compound 19 i.e., C37-014 | 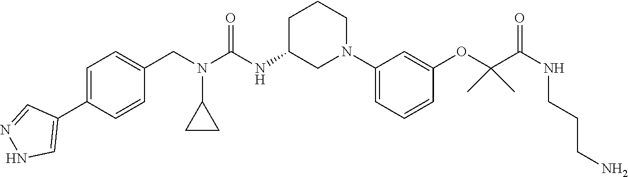 [SMILES: CC(C)(Oc1cccc(N2CCC[C@@H](NC(=O)N(Cc3ccc(-c4cn[nH]c4)cc3)C3CC3)C2)c1)C(=O)NCCCN] |
| Compound 20 | 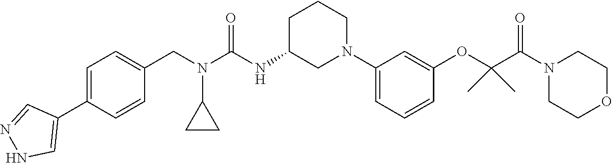 [SMILES: CC(C)(Oc1cccc(N2CCC[C@@H](NC(=O)N(Cc3ccc(-c4cn[nH]c4)cc3)C3CC3)C2)c1)C(=O)N1CCOCC1] |

In another preferred embodiment, the compound is selected from Table E below.

TABLE E

| Molecular | | Molecular | Molecular |
|---|---|---|---|
| numbering | Structural formula | weight | formula |
| C37-005 | 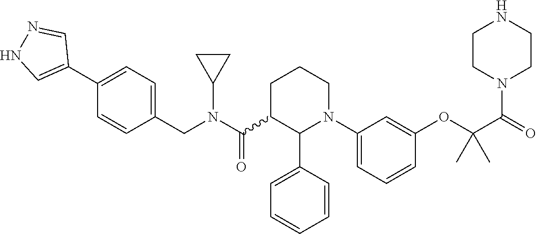 [SMILES: CC(C)(Oc1cccc(N2CCCC(C(=O)N(Cc3ccc(-c4cn[nH]c4)cc3)C3CC3)C2c2ccccc2)c1)C(=O)N1CCNCC1] | 646.84 | C39H46N603 |
| C37-015 | 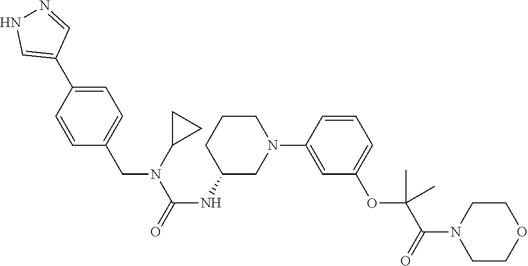 [SMILES: CC(C)(Oc1cccc(N2CCC[C@@H](NC(=O)N(Cc3ccc(-c4cn[nH]c4)cc3)C3CC3)C2)c1)C(=O)N1CCOCC1] | 586.74 | C33H42N604 |
| C37-016 | 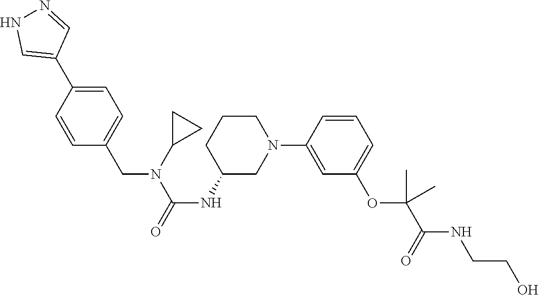 [SMILES: CC(C)(Oc1cccc(N2CCC[C@@H](NC(=O)N(Cc3ccc(-c4cn[nH]c4)cc3)C3CC3)C2)c1)C(=O)NCCO] | 560.7 | C31H40N604 |
| C37-018 | 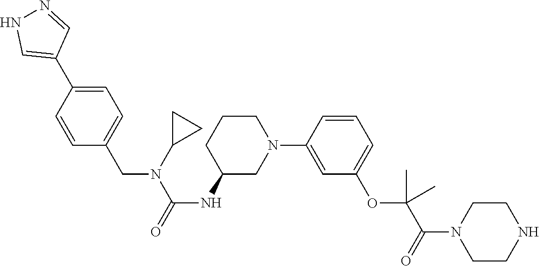 [SMILES: CC(C)(Oc1cccc(N2CCC[C@H](NC(=O)N(Cc3ccc(-c4cn[nH]c4)cc3)C3CC3)C2)c1)C(=O)N1CCNCC1] | 585.75 | C33H43N703 |
| C37-019 | 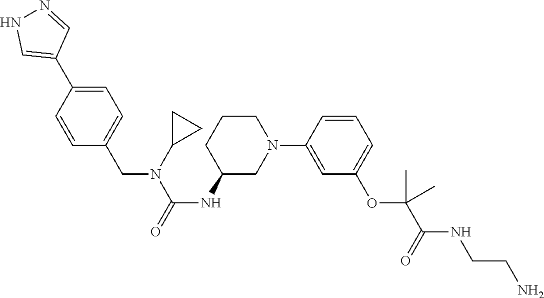 [SMILES: CC(C)(Oc1cccc(N2CCC[C@H](NC(=O)N(Cc3ccc(-c4cn[nH]c4)cc3)C3CC3)C2)c1)C(=O)NCCN] | 559.72 | C31H41N703 |
| C37-020 | 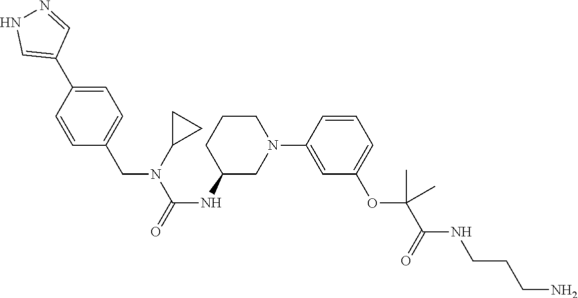 [SMILES: CC(C)(Oc1cccc(N2CCC[C@H](NC(=O)N(Cc3ccc(-c4cn[nH]c4)cc3)C3CC3)C2)c1)C(=O)NCCCN] | 573.74 | C32H43N703 |
| C37-021 | 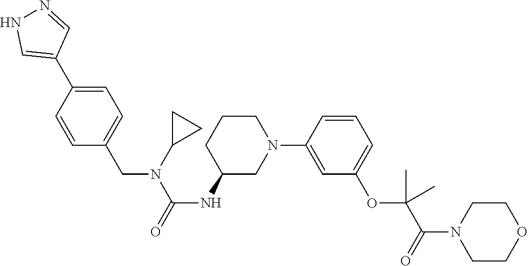 [SMILES: CC(C)(Oc1cccc(N2CCC[C@H](NC(=O)N(Cc3ccc(-c4cn[nH]c4)cc3)C3CC3)C2)c1)C(=O)N1CCOCC1] | 586.74 | C32H42N604 |
| C37-022 | 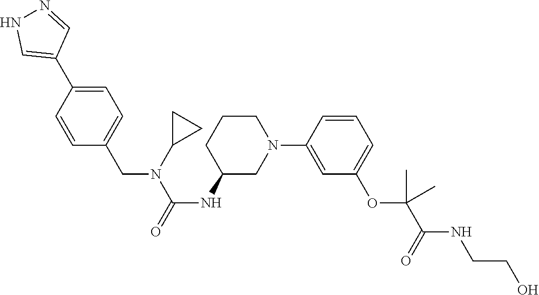 [SMILES: CC(C)(Oc1cccc(N2CCC[C@H](NC(=O)N(Cc3ccc(-c4cn[nH]c4)cc3)C3CC3)C2)c1)C(=O)NCCO] | 560.7 | C31H40N604 |
| C37-032 | 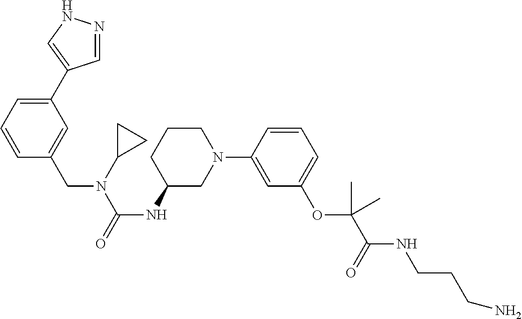 [SMILES: CC(C)(Oc1cccc(N2CCC[C@H](NC(=O)N(Cc3cccc(-c4cn[nH]c4)c3)C3CC3)C2)c1)C(=O)NCCCN] | 573.74 | C32H43N703 |
| C37-033 | 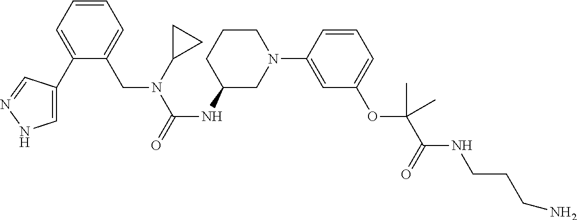 [SMILES: CC(C)(Oc1cccc(N2CCC[C@H](NC(=O)N(Cc3ccccc3-c3cn[nH]c3)C3CC3)C2)c1)C(=O)NCCCN] | 573.74 | C32H43N703 |
| C37-035 | 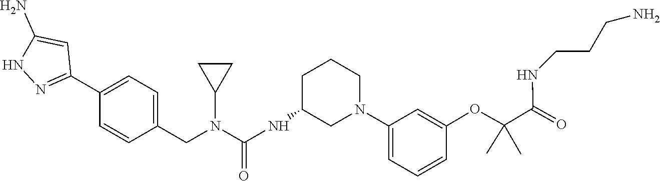 [SMILES: CC(C)(Oc1cccc(N2CCC[C@@H](NC(=O)N(Cc3ccc(-c4cc(N)[nH]n4)cc3)C3CC3)C2)c1)C(=O)NCCCN] | 588.76 | C32H44N803 |
| C37-036 | 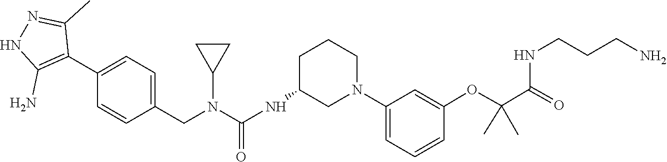 [SMILES: Cc1n[nH]c(N)c1-c1ccc(CN(C(=O)N[C@@H]2CCCN(c3cccc(OC(C)(C)C(=O)NCCCN)c3)C2)C2CC2)cc1] | 602.78 | C33H46N803 |
| C37-043 | 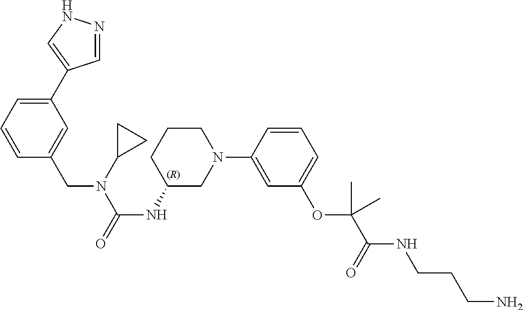 | 573.74 | C32H43N703 |
| C37-044 | 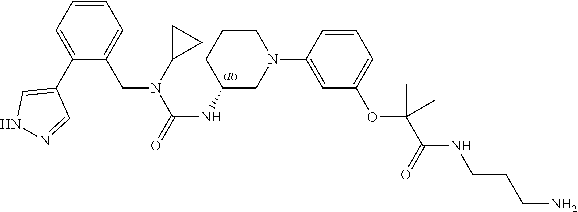 | 573.74 | C32H43N703 |
| C37-045 | 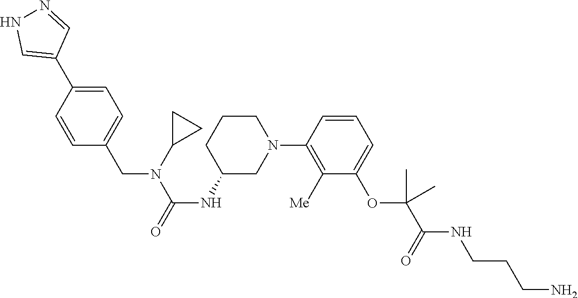 [SMILES: Cc1c(OC(C)(C)C(=O)NCCCN)cccc1N1CCC[C@@H](NC(=O)N(Cc2ccc(-c3cn[nH]c3)cc2)C2CC2)C1] | 587.77 | C33H45N703 |
| C37-046 | 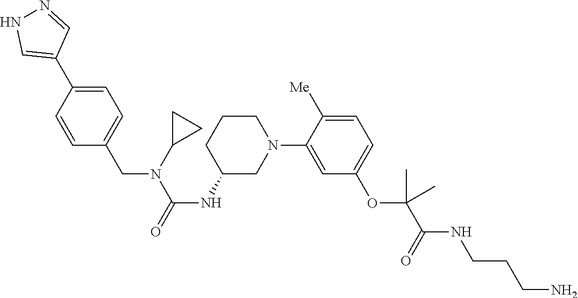 [SMILES: Cc1ccc(OC(C)(C)C(=O)NCCCN)cc1N1CCC[C@@H](NC(=O)N(Cc2ccc(-c3cn[nH]c3)cc2)C2CC2)C1] | 587.77 | C33H45N703 |

In another preferred embodiment, Ring A, Ring B, Ring C, L¹, L², W¹, W², subscript n1, subscript n2, R^{A}, R^{B}, R^{C}, R^{A1}, R^{B1}, R^{C1}, R¹, R², R³, R⁴, R⁵, R⁶, R⁷, R⁸, R^{s}, subscript m1, subscript m2, and subscript m3 are each independently corresponding groups in example compounds or specific compounds in tables A1, A2, A3, A4, A5, A6, Tables B, C, D, and E.

In a second aspect of the present invention, provided is a compound or a pharmaceutically acceptable salt thereof, or an isomer, solvate, crystal form, or prodrug thereof, the compound of Formula I:

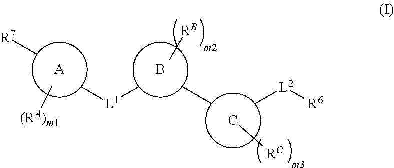

wherein Ring A, Ring B, Ring C, L¹, L², R^{A}, R^{B}, R^{C}, R⁶, R^{7s}, subscript m1, subscript m2 and subscript m3 are as defined in the first aspect;
and the compound is not a compound selected from Table I (or the pharmaceutically acceptable salt thereof).

In another preferred embodiment, the compound is not a specific compound disclosed in WO2021055936 (such as the inhibitor 1-112 therein).

In a third aspect of the present invention, provided is a pharmaceutical composition, comprising:
(i) the compound of the first or second aspect, or the pharmaceutically acceptable salt thereof, or the isomer, solvate, crystal form, or prodrug thereof; and
(ii) a pharmaceutically acceptable carrier or excipient.

In a fourth aspect of the invention, provided is a use of the compound of the first or second aspect, or the pharmaceutically acceptable salt thereof, or the isomer, solvate, crystal form, or prodrug thereof in preparing a drug for treating or preventing a disease associated with BCL9/β-catenin interaction.

In another preferred embodiment, the disease associated with BCL9/β-catenin interaction comprises cancer, and tumor.

In a fifth aspect of the invention, provided is a method for treating or preventing a disease associated with BCL9/β-catenin interaction, comprising a step of: administering a therapeutically effective amount of a compound of the first or second aspect or the pharmaceutically acceptable salt thereof, or the isomer, solvate, crystal form or prodrug thereof, or the pharmaceutical composition of the third aspect to a subject in need thereof.

In another preferred embodiment, the disease associated with BCL9/β-catenin interaction comprises: cancer, and tumor.

In a sixth aspect of the invention, provided is a method for treating or preventing cancer, comprising a step of administering a therapeutically effective amount of a compound of the first aspect or the second aspect or the pharmaceutically acceptable salt thereof, or the isomer, solvate, crystal form or prodrug thereof, or the pharmaceutical composition of the third aspect, to a subject in need thereof.

In a seventh aspect of the invention, provided is a use of the compound of the first aspect, or the pharmaceutically acceptable salt thereof, or the isomer, solvate, crystal form, or prodrug thereof in the preparing a drug for treating or preventing fibrosis or a related disease thereof.

In another preferred embodiment, the fibrosis or the related disease thereof comprises pulmonary fibrosis, hepatic fibrosis, nonalcoholic hepatic steatohepatitis, bone fibrosis, or a combination thereof.

In an eighth aspect of the invention, provided is a method for treating or preventing a fibrosis-related disease, comprising a step of administering a therapeutically effective amount of the compound of the first aspect or the pharmaceutically acceptable salt thereof, or the isomer, solvate, crystal form or prodrug thereof, or the pharmaceutical composition of the third aspect to a subject in need thereof.

In another preferred embodiment, fibrosis or the related disease thereof comprises pulmonary fibrosis, hepatic fibrosis, non-alcoholic hepatic steatohepatitis, bone fibrosis, or a combination thereof.

In another preferred embodiment, L¹ is —CH(R⁸)—N(R¹)—C(O)—NH—, wherein the CH(R⁸) end is attached to Ring A.

In a ninth aspect of the invention, provided is a method for inhibiting the binding of BCL9 to β-catenin in a subject; and/or regulating Wnt/β-catenin signaling in a subject; and/or reducing the survival of regulatory T cell in a subject; and/or reducing VEGF expression in the tumor in a subject; and/or increasing CD4+ and CD8+ T cells that infiltrate into the tumor in a subject; and/or increasing T helper 17 (Th17) cells that get into the tumorin a subject; and/or decreasing dendritic cells in the tumor in a subject; and/or making a half-life (T 112) greater than at least 2 hours when administered to a subject; and/or inducing a tumor microenvironment in a subject that is conducive to an immune response; and/or inhibiting tumor growth in a subject; and/or inhibiting the proliferation of cancer stem cells in a subject; and/or inhibiting tumor metastasis in a subject, including a step of: administering the compound according to the first or second aspect or the pharmaceutically acceptable salt thereof, or the isomer, solvate, crystal form or prodrug thereof, or the pharmaceutical composition according to the third aspect to the subject, or contracting the subject with the compound according to the first aspect or the pharmaceutically acceptable salt thereof, or the isomer, solvate, crystal form or prodrug thereof.

In another preferred embodiment, the subject is a mammal, preferably, a human being.

In another preferred embodiment, the subject is a cell.

In another preferred embodiment, the method is non-therapeutic.

It will be understood that within the scope of the present invention, each of the above technical features of the present invention and each of the technical features specifically described below (e.g., examples) may be combined with each other to form a new or preferred technical solution. Due to space limitations, it will not be repeated herein.

### DESCRIPTION OF THE DRAWINGS

FIGS. 1A-1T show the chromatographic analysis results of compounds 1-20 synthesized in Preparation Example 2.

### EMBODIMENTS FOR CARRYING OUT THE INVENTION

After extensive and in-depth research, the inventors unexpectedly found a class of small molecule compounds with a novel structure that has excellent activity for inhibiting the interaction between BCL9 and β-catenin. In addition, the inventors found that this class of compounds has excellent therapeutic and preventive effects in fibrosis and related diseases. Based on this, the inventors have completed the present invention.

### TERMS

Unless otherwise specified, each term or abbreviation used herein has the usual meaning understood by those skilled in the art.

Unless otherwise noted, as used herein, when a single bond in the structure of a compound indicated by a dotted line ( [symbol] ), the single bond represents the position of connection to the rest of the molecule.

As used herein, the terms “containing”, “comprising”, or “including” mean that various components may be used together in the mixture or composition of the present invention. Accordingly, the terms “mainly consisting of . . . ” and “consisting of . . . ” are within the term “comprising”.

Unless otherwise stated, the term “alkyl” group, by itself or as part of another substituent, refers to a straight or branched hydrocarbon group having a specified number of carbon atoms (i.e., C₁₋₆ denotes 1-6 carbons). Examples of alkyl groups include methyl, ethyl, n-propyl, isopropyl, n-butyl, tert-butyl, isobutyl, sec-butyl, n-pentyl, n-hexyl, n-heptyl, n-octyl, etc.

The term “alkenyl” refers to an unsaturated alkyl group having one or more double bonds. Similarly, the term “alkynyl” refers to an unsaturated alkyl group with one or more triple bonds. In general, the alkenyl group has 1-6 carbon atoms (i.e., C₁₋₆ alkenyl), and the alkynyl group has 1-6 carbon atoms (i.e., C₁₋₆ alkynyl). Examples of this kind of unsaturated alkyl groups include vinyl, 2-propenyl, crotonyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The terms “alkoxy”, “alkoamino”, and “alkothyl” (or thioalkoxy) are used in their conventional meanings, refer to those alkyl groups attached to the rest of the molecule by an oxygen atom, an amino group, or a sulfur atom, respectively. Furthermore, for dialkylamino groups, the alkyl portions can be the same or different or can also be combined to form a 3- to 7-membered ring with the nitrogen atoms to which each is attached. Thus, the group represented by —NR^{a}R^{b} is meant to include piperidinyl, pyrrolidinyl, morpholinyl, azetidinyl, etc.

As used herein, the term “alkylene”, by itself or as part of another substituent, refers to a divalent group derived from an alkane, such as —CH₂—, —CH₂CH₂—.

As used herein, the term “aminoalkyl” refers to an alkyl group as defined above having a specified number of carbon atoms with one or two hydrogens being substituted by an amino group. For example, —(CH₂)₂NH₂.

As used herein, the term “cycloalkyl” refers to a saturated hydrocarbon ring having a specified number of ring atoms (e.g., C₃₋₁₀cycloalkyl, preferably C₃₋₆cycloalkyl). “Cycloalkyl” may be a single ring (e.g., cyclopropyl, cyclobutyl, cyclohexyl, etc.), and may also refer to a bicyclic and polycyclic hydrocarbon ring (including fused ring, spiro ring, bridge ring, etc.), such as bicyclo[2.2.1]heptane, bicyclo[2.2.2]octane, etc. The term “heterocycloalkyl” refers to a cycloalkyl group containing one to five (preferably 1, 2, 3, or 4) heteroatoms selected from N, O, and S, wherein the nitrogen and sulfur atoms are optionally oxidized and the nitrogen atom is optionally quaternized. The Heterocycloalkyl may be a monocyclic, a bicyclic or a polycylic system (including fused ring, spiro ring, bridge ring, etc.). In general, the heterocycloalkyl typically includes 4 to 10 ring atoms (i.e., 4 to 10 membered heterocycloalkyl), preferably includes 4 to 7 (e.g., 4, 5, 6) ring atoms (i.e., 4 to 7 membered heterocycloalkyl, or 4 to 6 membered heterocycloalkyl) and contains 1, 2, 3, or 4 (preferably 1 or 2) heterocyclic atoms. Non-limiting examples of heterocycloalkyl groups include pyrrolidine, imidazolidine, pyrazolide, butyrolactam, valerolactam, imidazolidinone, hydantoin, dioxolane, phthalimide, piperidine, 1,4-dioxane, morpholine, thiomorpholine-S-oxide, thiomorpholine-S,S-oxide, piperazine, pyran, pyridinone, 3-pyrroline thiopyran, pyrone, tetrahydrofuran, tetrahydrothiophene, quinuclidine, etc. The heterocycloalkyl group can be attached to the rest of the molecule via a ring carbon or a heteroatom such as a ring nitrogen.

As used herein, the term “cycloalkenyl”, used alone or as part of a group, refers to a cyclic hydrocarbon having a specified number of ring atoms (e.g., C₃₋₁₀cycloalkenyl, or C₃₋₆cycloalkenyl), and having 1 or 2 double bonds (preferably, only 1 double bond) between the ring vertices. Cycloalkenyl may be a monocyclic ring or may also refer to a bicyclic or a polycylic hydrocarbon ring (including fused ring, spiro ring, bridge ring, etc.). Examples of cycloalkenyl include, for example, cyclopropene, cyclobutene, cyclopentene, cyclopentadiene, etc. Similarly, the term “heterocycloalkenyl” refers to a cycloalkenyl group containing one to five heteroatoms (preferably 1, 2, 3, or 4) selected from N, O, and S, wherein nitrogen and sulfur atoms are optionally oxidized and nitrogen atom is optionally quaternized. The heterocycloalkenyl group may be a monocyclic, bicyclic, or polycyclic system (including fused ring, spiro ring, bridge ring, etc.). In general, the heterocyenyl group typically contains 4-10 ring atoms (i.e., 4 to 10 membered heterocycloalkyl), preferably, 4-7 ring atoms (i.e., 4 to 7 membered heterocycloalkyl, or 4 to 6 membered heterocycloalkyl) and contains 1, 2, 3, or 4 (preferably 1 or 2) heterocyclic atoms.

For terms such as cycloalkylalkyl (alkylene) and heterocycloalkylalkyl (alkylene), it is meant that the cycloalkyl or heterocycloalkyl group is attached to the rest of the molecule via an alkyl or alkylene. For example, cyclobutylmethyl- is a cyclobutyl ring attached to a methylene linker to the rest of the molecule.

Unless otherwise stated, the term “aryl” means a polyunsaturated (typically aromatic) hydrocarbon group, which may be a single ring or a multiple ring (up to three rings) fused together or linked covalently. Generally, the aryl group has 6-10 ring atoms. The term “heteroaryl” refers to an aryl group (or ring) containing one to five heteroatoms selected from N, O, and S, wherein nitrogen and sulfur atoms are optionally oxidized and the nitrogen atom is optionally quaternized. Generally, the heteroaryl group has 5-10 ring atoms i.e., 5-10 membered heteroaryl, preferably 5-6 ring atoms i.e., 5-6 membered heteroaryl, and contains 1, 2, 3, or 4 heteroatoms. A heteroaryl group may be attached to the rest of the molecule through a heteroatom. Non-limiting examples of aryl include phenyl, naphthyl, and biphenyl, while non-limiting examples of heteroaryl include pyridinyl, pyridazinyl, pyrazinyl, pyrimidinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, purrocolinyl, benzotriazinyl, thienopyridinyl, thienopyrimidinyl, pyrazolopyrimidinyl, imidazopyridinyl, benzothiazolyl, benzofuryl, benzothiophenyl, indolyl, quinolinyl, isoquinolinyl, isothiazolyl, pyrazolyl, indazolyl, pteridinyl, imidazolyl, triazolyl, tetraazolyl, oxazolyl, isoxazolyl, thiadiazolyl, pyrrolyl, thiazolyl, furanyl, thienyl, etc. The respective substituents of the above aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above such as aryl, aryl sulfide, aryl alkyl. Accordingly, the term “arylalkyl” is meant to onclude those groups in which an aryl is attached to an alkyl group that is attached to the rest of the molecule (e.g. benzyl, phenethyl, pyridylmethyl and the like).

In some embodiments, the above terms (e.g., “alkyl,” “aryl” and “heteroaryl”) will include both substituted and unsubstituted forms of the indicated group. Preferred substituents for each type of group are provided below. For brevity, the terms aryl and heteroaryl will refer to the substituted or unsubstituted forms provided below, while the term “alkyl” and related aliphatic groups refer to the unsubstituted forms unless substituted is indicated.

Substituent for the alkyl group (including those groups usually referred to as alkylene, alkenyl, alkynyl, and cycloalkyl) may be a variety of groups selected from the group consisting of: -halogen, —OR′, —NR′R″, —SR′, —SiR′R″R″′, —OC(O)R′, —C(O)R′, —CO₂R′, —CONR′R″, —OC(O)NR′R″, —NR″ C(O)R′, —NR′—C(O)NR″R″′, —NR″ C(O)₂R′, —S(O)R′, —S(O)₂R′, —S(O)₂NR′R″, —NR'S(O)₂R″, —CN and —NO, in a number ranging from zero to (2 m′+1), wherein m′ is the total number of carbon atoms in the groups. R′, R″, and R″′ each independently refer hydrogen, unsubstituted C₁₋₈ alkyl, unsubstituted heteroalkyl, unsubstituted aryl, aryl substituted with 1-3 halogen, unsubstituted C₁₋₈ alkyl, C₁₋₈ alkoxy or C₁₋₈ thioalkoxy, or unsubstituted aryl-C₁₋₄ alkyl group. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. The term “acyl”, used alone or as part of another group, refers to a group wherein two substituents on the carbon that is closest to the point of attachment for the radical is replaced with the substituent=O (e.g., —C(O)CH₃, —C(O)CH₂CH₂OR′, etc.).

Similarly, the substituents for aryl and heteroaryl are diverse and are usually selected from: -halogen, —OR′, —OC(O)R′, —NR′R″, —SR′, —R′, —CN, —NO₂, —CO₂R′, —CONR′R″, —C(O)R′, —OC(O)NR′R″, —NR″ C(O)R′, —NR″ C(O)₂R′, —NR′—C(O)NR″R″′, —S(O)R′, —S(O)₂R′, —S(O)₂NR′R″, —NR'S(O)₂R″, —N₃, perfluoro(C₁-C₄)alkoxyl and perfluoro(C₁-C₄)alkyl, in a number from zero to the total number of open valences on the aromatic ring system; wherein R′, R″ and R″′ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl and unsubstituted aryloxy-C₁₋₄ alkyl. Other suitable substituents include each of the above aryl substituents attached to a ring atom by an alkylene chain of 1-4 carbon atoms.

As used herein, the term “heteroatom” is intended to include oxygen (O), nitrogen (N), sulfur (S), and silicon (Si).

As used herein, “halogen” refers to F, Cl, Br, and I. Preferably, the halogen atoms are selected from F, Cl, and Br.

For the compounds provided herein, a bond from a substituent (usually an R group) to the center of an aromatic ring (e.g., benzene, pyridine, etc.) will be understood to mean a bond providing a connection at any available vertices of the aromatic ring. In some embodiments, the depiction will also include a connection at a ring which is fused to the aromatic ring. For example, a bond drawn to the center of the benzene portion of an indole will represent a bond connected to any available vertex of the six-membered or five-membered ring part of the indole.

Unless otherwise stated, all compounds present in the present invention are intended to include all possible optical isomers, such as single chiral compounds or mixtures of various chiral compounds (i.e., racemes). Among all compounds of the present invention, each chiral carbon atom may optionally be of the R or S configuration, or a mixture of the R and S configurations. Preferably, as used herein, unless otherwise stated, when a single bond in the structure of a compound is represented by [symbol] , the compound includes a compound of a single configuration wherein said bond is of S configuration or R configuration, or a mixture of the S configuration and the R configuration, such as a raceme.

### Active Ingredient

As used herein, the term “compound of the invention” refers to a compound according to the first aspect of the invention. This term also includes various crystalline forms, pharmaceutically acceptable salts, hydrates, or solvates of the compound according to the first aspect of the invention.

As used herein, the term “pharmaceutically acceptable” ingredient refers to a substance that is suitable for use in humans/or animals without excessive adverse side effects reactions (e.g., toxicity, stimulation, and allergy), that is, a substance with a reasonable benefit/risk ratio.

As used herein, the term “therapeutically effective dose” refers to any following amount of a drug: when the amount of the drug is used alone or in combination with another therapeutic agent, the amount is able to promote regression of disease which is indicated by a reduction in the severity of disease symptoms, an increase in the frequency and duration of asymptomatic period of disease, or prevention of impairment or disability resulting from the disease. The therapeutically effective dose of the drug of the present invention also includes “prophylactic effective dose”, the “prophylactic effective dose” is any following amount of the drug: when the amount of the drug is administered alone or in combination with another therapeutic agent to a subject having a risk of developing the disease or suffering a recurrence of the disease, the amount is able to inhibit the occurrence or recurrence of the disease.

The term “pharmaceutically acceptable salt” is intended to include salts of the active compounds which are prepared with a relatively nontoxic acid or base, depending on the specific substituent on the compound described herein. When a compound of the present invention contains a relatively acidic functional group, a base addition salt can be obtained by contacting a neutral form of such compound with a sufficient amount of the desired base (either neat or in a suitable inert solvent). Examples of salts derived from pharmaceutically acceptable inorganic bases include aluminum, ammonium, calcium, copper, iron, ferrous iron, lithium, magnesium, manganese, manganous, potassium, sodium, zinc, etc. Salts derived from pharmaceutically acceptable organic bases include salts of primary, secondary, and tertiary amines, including substituted amines, cyclic amines, naturally-occurring amines, etc., such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hypamine, isopropylamine, Lysine, methylglucosamine, morpholine, piperazine, piperidine, polyamine resin, procaine, purines, theobromine, triethylamine, trimethylamine, tripropylamine, tromethamine and so on. When a compound of the present invention contains a relatively basic functional group, an acid addition salt can be obtained by contacting a neutral form of such compound with a sufficient amount of the desired acid (either neat or in a suitable inert solvent). Examples of pharmaceutically acceptable acid addition salts include those derived from inorganic acids, such as hydrochloric acid, hydrobromic acid, nitric acid, carbonic acid, monohydrogencarbonic acid, phosphoric acid, monohydrogenphosphoric acid, dihydrogenphosphoric acid, sulphuric acid, monohydrogensulphuric acid, hydroiodic acid, or phosphorous acid, and the like; and salts derived from relatively non-toxic organic acids such as acetic acid, propionic acid, isobutyric acid, malonic acid, benzoic acid, succinic acid, suberic acid, fumaric acid, mandelic acid, phthalic acid, benzenesulfonic acid, p-toluene sulfonic acid, citric acid, tartaric acid, mesylate, and the like. Also included are salts of amino acids, such as arginine salts, etc., and salts of organic acids, such as glucuronic acid or galactunoric acid, etc. (see, for example, Berge, S. M. et al., Pharmaceutical Salts, Journal of Pharmaceutical Science, 1977, 66, 1-19). Certain compounds of the present invention contain both basic and acidic functional groups, thereby enabling the conversion of the compound into a base addition or acid addition salt.

The neutral form of the compound can be regenerated by contacting the salt with a base or acid and separating the parent compound in a conventional manner. The parent form of the compound differs from the various salt forms in some physical properties, such as solubility in polar solvents, but in addition to the above, those salts are equivalent to the parent form compound for the purposes of the present invention.

In addition to the salt form, the present invention further provides compounds in the prodrug form. Prodrugs of the compounds described herein are those compounds that readily undergo chemical changes under physiological conditions to provide the compounds of the present invention. Alternatively, the prodrug can be converted into the compound of the invention by chemical or biochemical methods in an ex vivo environment. For example, the prodrug can be slowly converted to the compound of the invention when placed in a transdermal patch reservoir containing a suitable enzyme or chemical reagent.

Certain compounds of the present invention may exist in non-solvated forms as well as solvated forms (i.e., a solvate), including hydrated forms (i.e., a hydrate). The solvated form is usually equivalent to the non-solvated form and should be included within the scope of the present invention. Certain compounds of the present invention may exist in polymorphous or amorphous forms. Generally, all physical forms are equivalent for the application considered in the present invention and should be included within the scope of the present invention.

Certain compounds of the present invention possess asymmetric carbon atoms (optical centers) or double bonds; the racemes, diastereomers, geometric isomers, regional isomers, and individual isomers (e.g., separated enantiomers) are all should be included within the scope of the present invention. When compounds provided herein have defined stereochemistry (indicated as R or S, or indicated by dashed or wedged bonds), it will be understood by one skilled in the art that those compounds are substantially free of other isomers (e.g., at least 80%, 90%, 95%, 98%, 99% and at most 100% free of other isomers).

The compounds of the present invention may also contain unnatural proportions of atomic isotopes at one or more isotopic atoms constituting such compounds. The unnatural proportions of an isotope may be defined as an amount from the naturally found amount of the atom in question to 100% of the atom. For example, a compound may incorporate a radioactive isotope, such as tritium (³H), iodine-125 (¹²⁵I), or carbon-14 (¹⁴C), or a non-radioactive isotope, such as deuterium (²H) or carbon-13 (¹³C). Such isotope variants may provide additional uses in addition to those described in this application. For example, isotopic variants of the compounds of the invention may have additional uses, including but not limited to as diagnostic and/or imaging reagents, or as cytotoxic/radiotoxic therapeutic agents. Alternatively, isotopic variants of the compounds of the present invention may have altered pharmacokinetic and pharmacodynamic profiles, thereby contributing to increased safety, tolerability, or efficacy during treatment. All isotopic variants of the compound of the invention, whether radioactive or not, should be included within the scope of the invention.

### Pharmaceutical Composition and Method of Administration

Because the compounds of the present invention have excellent inhibitory activity against BCL9/β-catenin protein-protein interaction (BCL9/β-catenin PPI), the compounds of the present invention and various polymorphs, pharmaceutically acceptable inorganic or organic salts, hydrates or solvates thereof, and pharmaceutical compositions containing the compound of the present invention as main active ingredient can be used for treating, preventing, and alleviating diseases associated with BCL9/β-catenin protein-protein interaction. According to the prior art, the compounds of the present invention can be used to treat the following diseases: cancer, tumor, etc., for example, familial adenomatous polyposis (FAP), eye cancer, rectal cancer, colon cancer, colorectal cancer, cervical cancer, prostate cancer, breast cancer, bladder cancer, oral cancer, benign and malignant tumors, stomach cancer, liver cancer, pancreatic cancer, lung cancer, corpus uteri, ovarian cancer, prostate cancer, testicular cancer, kidney cancer, brain/CNS cancer, laryngeal cancer, multiple myeloma, skin melanoma, acute lymphoblastic leukemia, acute myeloid leukemia, Ewing's sarcoma, Kaposi's sarcoma, basal cell and squamous cell carcinoma, small cell lung cancer, choriocarcinoma, rhabdomyosarcoma, angiosarcoma, hemangioendothelioma, Wilm's tumor, neuroblastoma, oral/pharyngeal cancer, esophageal cancer, laryngeal cancer, lymphoma, neurofibromatosis, tuberous sclerosis, hemangioma, gastric cancer, ovarian cancer, hepatocellular carcinoma, lymphatic vessels, and the like.

In addition, the compounds of the present invention also have excellent ability to treat fibrosis; therefore, the compounds of the present invention and various polymorphs, pharmaceutically acceptable inorganic or organic salts, hydrates or solvates thereof, and pharmaceutical compositions containing the compound of the invention as main active ingredient can be used in the treatment, prevention, and alleviation of fibrosis and various diseases associated with fibrosis. Fibrosis can occur in a variety of organs, and the main pathological changes are the increase of fibrous connective tissue and the decrease of parenchymal cells in organ tissues. Continuous progression may lead to structural damage and hypofunction, and even failure of organs, which seriously threatens human health and life.

Examples of fibrosis and related diseases thereof are as follows:

| Major organs | Typical diseases and syndromes |
|---|---|
| 1 Lung | Diseases with known causes: inorganic dust occupational diseases (silicosis, |
| | asbestosis, coal lung, etc.); Organic dust and hypersensitivity pneumonitis |
| | (farmer's lung, air conditioner humidifier lung, pigeon breeder's lung, bagasse |
| | pneumoconiosis, etc.); Diseases related to drugs/treatment (antibiotics, |
| | non-steroidal anti-inflammatory agents, cardiovascular drugs, antineoplastic |
| | drugs, oral hypoglycemic drugs, oxygen, morphine, etc.); Infectious diseases |
| | (pulmonary tuberculosis, viral pneumonia, pneumocystis infection, etc.) |
| | secondary lung diseases (left heart failure, congenital heart disease, adult |
| | respiratory distress syndrome, chronic cardiac insufficiency, transplant |
| | rejection related lung diseases, etc.); Diseases with unknown etiology: |
| | primary pulmonary diseases (idiopathic interstitial pneumonia, bronchiolitis |
| | obliterans with organizing pneumonia, pulmonary lymphangioleiomyoma, |
| | etc.); Collagen-related pulmonary diseases (systemic lupus erythematosus, |
| | rheumatoid arthritis, progressive systemic sclerosis, polymyositis, |
| | dermatomyositis, mixed connective tissue disease, etc.); Alveolar filling |
| | diseases (diffuse alveolar hemorrhage syndrome, pulmonary alveolar |
| | proteinosis, eosinophilic pneumonia, pulmonary vasculitis, lymphocytic |
| | interstitial pneumonia, necrotizing nodular granuloma, familial pulmonary |
| | fibrosis, etc.) |
| 2 | Ischemic heart disease (replacement and interstitial fibrosis after myocardial |
| Cardiovascular | infarction); Hypertensive heart disease; Inflammatory cardiomyopathy (viral |
| system | myocarditis); Metabolic cardiomyopathy (hemochromatosis cardiomyopathy, |
| | amyloid cardiomyopathy, glycogen accumulation cardiomyopathy, diabetic |
| | cardiomyopathy, etc.); Keshan disease; Dilated cardiomyopathy; Hypertrophic |
| | cardiomyopathy, restrictive cardiomyopathy; Arrhythmogenic right |
| | ventricular cardiomyopathy |
| 3 Liver | Viral cirrhosis (hepatitis B, C, and D viral hepatitis); Liver cirrhosis caused by |
| | schistosomiasis; Alcoholic cirrhosis; Biliary cirrhosis (primary biliary |
| | cirrhosis, secondary gallstones, periportal inflammation); Metabolic cirrhosis |
| | (hepatolenticular degeneration, hemochromatosis); Toxic liver cirrhosis |
| | (organophosphorus poisoning, carbon tetrachloride poisoning, hepatotoxic |
| | drugs such as isoniazid, tetracycline, chlorpromazine poisoning, etc.); |
| | Dystrophic cirrhosis; Cardiogenic cirrhosis (chronic congestive heart failure) |
| 4 Pancreas | Acute pancreatitis; Pancreatic duct obstruction; Chronic alcoholism; |
| | Dysfunction of sphincter of Oddi; Pancreatic ischemia, etc. |
| 5 Kidneys | Vascular (high blood pressure); Immune (glomerulonephritis, systemic lupus |
| | erythematosus, scleroderma, renal transplant rejection) infectious |
| | (pyelonephritis, kidney stones) metabolic (hyperlipidemia, diabetes mellitus, |
| | hyperuricuria, hypercalciuria) and so on |
| 6 Spleen | Fibroproliferative disease of the spleen |
| 7 Eyes | After eye trauma and surgery, fibroplasia of the eye membrane of the diabetic |
| | optic mesh |
| 8. Nervous | Scar formation after spinal cord injury and stroke, Alzheimer's disease |
| system | |
| 9 Bone | Idiopathic and drug-induced myelofibrosis, polycythemia vera, chronic |
| marrow | myeloid leukemia, Hodgkin's disease |

The pharmaceutical composition of the invention comprises a compound of the invention or a pharmaceutically acceptable salt thereof in a safe and effective amount dosage range and pharmaceutically acceptable excipients or carriers. Wherein “safe and effective amount” means an amount of the compound which is sufficient to significantly improve the condition without causing serious side effects. Typically, the pharmaceutical composition contains 1-2000 mg of the compound of the invention per dose or, more preferably, 10-500 mg of the compound of the invention per dose. Preferably, the “one dose” is a capsule or tablet.

“Pharmaceutically acceptable carrier” means one or more compatible solid or liquid fillers or gel substances that are suitable for human use and that must be of sufficient purity and sufficiently low toxicity. “Compatibility” used herein refers to the components of the composition can be admixed with the compounds of the invention and each other without significantly reducing the efficacy of the compound. Some examples of pharmaceutical acceptable carriers are cellulose and its derivatives (e.g., sodium carboxymethyl cellulose, sodium ethyl cellulose, cellulose acetate, etc.), gelatin, talc, solid lubricants (e.g., stearic acid, magnesium stearate), calcium sulfate, vegetable oils (e.g., soybean oil, sesame oil, peanut oil, olive oil, etc.), polyols (e.g., propylene glycol, glycerol, mannitol, sorbitol, etc.), emulsifiers (such as Tween®), wetting agents (such as sodium dodecyl sulfate), colorants, flavoring agents, stabilizers, antioxidants, preservatives, pyrogen-free water, etc.

There is no particular limitation on the mode of administration of the compound or pharmaceutical composition of the invention, and representative modes of administration include but are not limited to oral, intratumoral, rectal, parenteral (intravenous, intramuscular, or subcutaneous), and topical administration.

Solid dosage forms for oral administration include capsules, tablets, pills, powders, and granules. In these solid dosage forms, the active compound is mixed with at least one conventional inert excipient (or carrier), such as sodium citrate or dicalcium phosphate, or with the following ingredients: (a) fillers or enhancers, for example, starch, lactose, sucrose, glucose, mannitol, and silicic acid; (b) binders, such as hydroxymethylcellulose, alginate, gelatin, polyvinylpyrrolidone, sucrose and gum Arabic; (c) moisturizers, for example, glycerin; (d) disintegrants such as agar, calcium carbonate, potato starch or cassava starch, alginic acid, certain complex silates, and sodium carbonate; (e) dissolution-retarding agents, such as paraffin; (f) absorption accelerators, for example, quaternary amine compounds; (g) wetting agents such as cetyl alcohol and glyceryl monostearate; (h) adsorbents, for example, kaolin; and (i) lubricants, for example, talc, calcium stearate, magnesium stearate, solid polyethylene glycol, sodium dodecyl sulfate, or mixtures thereof. In capsules, tablets, and pills, the dosage form may also comprise a buffer.

Solid dosage forms such as tablets, sugar pills, capsules, pills, and granules can be prepared using coating and shell materials such as casings and other materials well known in the art. They may contain an opaque agent, and the release of the active compound or compound in the composition may be released in a delayed manner in a portion of the digestive tract. Examples of the embedding components that can be employed are polymeric substances and waxes. When necessary, the active compound can also form microcapsules with one or more of the above excipients.

Liquid dosage forms for oral administration include pharmaceutically acceptable emulsions, solutions, suspensions, syrups, or tinctures. In addition to the active compound, the liquid dosage form may contain inert diluents conventionally used in the art, such as water or other solvents, solubilizers, and emulsifiers, e.g., ethanol, isopropanol, ethyl carbonate, ethyl acetate, propylene glycol, 1,3-butanediol, dimethylformamide, and oil, in particular, cottonseed oil, peanut oil, corn embryo oil, olive oil, castor oil and sesame oil, or mixtures of these substances, etc.

In addition to these inert diluents, the composition may also contain auxiliaries such as wetting agents, emulsifiers and suspending agents, sweeteners, corrigents, and fragrances.

In addition to the active compound, the suspension may contain suspending agents, for example, ethoxylated isooctadecanol, polyoxyethylene sorbitol and dehydrated sorbitol ester, microcrystalline cellulose, aluminum methanolol and agar or mixtures of these substances, and the like.

The composition for parenteral injection may contain a physiologically acceptable sterile aqueous or anhydrous solution, dispersion, suspension, or emulsion, and a sterile powder which can be re-dissolved into sterile injectable solution or dispersion. Suitable aqueous and non-aqueous carriers, diluents, solvents, or excipients include water, ethanol, polyols, and suitable mixtures thereof.

Dosage forms of the compounds of the invention for topical administration include ointments, dispersions, patches, sprays, and inhalants. The active ingredients are mixed under sterile conditions with a physiologically acceptable carriers and any preservatives, buffers, or propellants that may be required when necessary.

The compounds of the present invention may be administered alone or in combination with other pharmaceutically acceptable compounds.

In some embodiments, the pharmaceutical composition containing the compound of the invention may further comprise at least one additional agent. In some embodiments, the at least one additional agent is selected from one or more of a checkpoint inhibitor, an EGFR inhibitor, a VEGF inhibitor, a VEGFR inhibitor, and an anticancer drug.

In some embodiments, the pharmaceutical composition described herein may include a checkpoint inhibitor. In one embodiment, the checkpoint inhibitor is an anti-PD-1 antibody, anti-PD-L1 antibody, or anti-CTLA4 antibody. In one embodiment, the checkpoint inhibitor targets and stimulates checkpoint molecules such as, for example, CD27, CD40, OX40, GITR, or CD138. In yet another embodiment, the checkpoint inhibitor targets and stimulates checkpoint molecules, e.g., A2AR, B7-H3, B7-H4, B and T lymphocyte attenuator (BTLA), indoleamine 2,3-dioxygenase (IDO), killer cell immunoglobulin-like receptor (KIR), lymphocyte activating gene-3 (LAG3), T cell immunoglobulin and mucin domain protein 3 (TIM-3), VISTA (C10 or f54) or T cell activation V domain Ig inhibitors.

In some embodiments, the pharmaceutical composition described herein includes an EGFR inhibitor. In one embodiment, the EGFR inhibitor is erlotinib, gefitinib, lapatinib, panitumumab, vandetanib, or cetuximab.

In some embodiments, the pharmaceutical composition described herein may include a VEGF or VEGFR inhibitor. In one embodiment, the VEGF or VEGFR inhibitor is pazopanib, Avastin, sorafenib, sunitinib, axitinib, ponatinib, canregor, vandetanib, cabozantinib, ramucirumab, lenvatinib, or aflibercept.

In some embodiments, the pharmaceutical composition described herein includes an anticancer drug. Anticancer agents may be selected from cyclophosphamide, methotrexate, 5-fluorouracil (5-FU), doxorubicin, mustine, vincristine, methylbenzyl hydrazine, Prednisolone, dacarbazine, bleomycin, etoposide, cisplatin, epirubicin, capecitabine, leucovorin, actinomycin, all-trans retinoic acid, azacitidine, azathioprine, bortezomib, carboplatin, chlorambucil, cytarabine, Daunomycin, Eurotaxol, doxifluridine, fluorouracil, gemcitabine, hydroxyurea, idarubicin, imatinib, Irinotecan, mechlorethamine, mercaptopurine, mitoxantrone, paclitaxel, pemetrexed, teniposide, thioguanine, Topotecan, valrubicin, vinblastine, vindesine, vinorelbine, and oxaliplatin.

When the pharmaceutical composition is used, a safe and effective amount of the compound of the invention is applied to a mammal (such as human) in need of treatment, wherein the dose at the time of administration is the dose considered pharmaceutically effective, and for a person weighed 60 kg, the daily dose is usually 1 to 2000 mg, preferably 20 to 500 mg. Of course, the specific dose should also consider factors such as the route of administration, the health status of the patient and other factors, which are within the skill range of skilled physicians.

### BCL-9, β-Catenin, and Wnt Signaling

Aberrant activation of Wnt signaling is implicated in a variety of cancers, as tumors can depend on Wnt signaling for growth and survival. Up to 90% of all cases of sporadic colorectal cancer are associated with constitutive activation of Wnt signaling.

β-catenin is a protein that can engage in protein-protein interactions that stimulate Wnt signaling, resulting in changes in transcriptional activation that may contribute to tumor growth and development. β-catenin is normally phosphorylated and targeted for degradation by the axin complex. If there is stimulation of the Wnt signaling pathway, unphosphorylated β-catenin accumulates and binds to lymphoid enhancer factor/T-cell factor (LEF/TCF) and is translocated into the nucleus to stimulate transcription of Wnt target genes. Wnt target genes include c-myc and CD44, which are up-regulated genes in tumor models. BCL9 is a protein required for efficient β-catenin-mediated transcription in mammalian cells.

“Canonical” Wnt/β-catenin signaling is a pathway activated through the binding of Wnt ligands to the Frizzled family of cell surface receptors, which then regulate the expression and intracellular localization of β-catenin. In the absence of Wnt ligands, β-catenin is phosphorylated and ubiquitinated within a destruction complex composed of adenomatous polyposis coli (APC), glycogen synthase kinase-3 (GSK-3), casein kinase-1 (CK1), and axin, and is targeted for degradation in a proteasomal dependent manner. In the presence of Wnt ligands, β-catenin ubiquitination within the complex is inhibited, which results in saturation of phosphorylated β-catenin, which is then stabilized and translocated to the nucleus. There, phosphorylated β-catenin engages nuclear T-cell factor (TCF) transcription factors such as Lymphoid Enhancer Factor/3 (LEF/TCF) to induce the expression of genes that promote cell proliferation, migration, and survival, including c-Myc and Cyclin D.

Several molecules, including BCL9 and its homologue B-cell lymphoma 9-like (B9L), have been shown to be co-activators of Wnt/β-catenin transcription. Formation of a complex consisting of TCF, β-catenin, and BCL9 (or B9L) enhances β-catenin dependent Wnt transcriptional activity. In normal cells, this transcriptional pathway is turned off when Wnt ligands uncouple from their receptors. However, various loss-of-function mutations in APC and axin, as well as activating mutations in β-catenin itself, enable β-catenin to escape the destruction complex and accumulate in the nucleus. Such inappropriate persistence of β-catenin promotes oncogenesis in a wide range of common human epithelial cancers, including hepatocellular carcinoma, breast cancer, colorectal cancer, and hematologic malignancies such as multiple myeloma. In addition, active β-catenin signaling leads to T-cell rejection, particularly CD8+ T cell rejection, which results in therapy resistance and shorter patient survival. Thus, blocking Wnt signaling by targeting 3-cat may provide a powerful therapeutic approach for CRC, thereby potentially preventing tumorigenesis and metastasis.

Similar to other transcription factors, the development of selective nontoxic β-catenin inhibitors and and their clinical translation have proven to be a considerable challenge, as β-catenin interacts with most of its protein partners through the same binding surface. Therefore, Wnt pathway inhibitors targeting this common-binding surface have shown significant adverse effects in animal and clinical trials. Only a few drugs targeting β-catenin exist in clinical trials, including PRI-724 (Eisai Pharmaceuticals; Phase II), LGK974 (Novartis; Phase I), and OMP-54F28 and OMP-18R⁵ (OncoMed/Bayer; Phase I). In addition, disruption of the LEF/TCF interaction by small molecules and peptide inhibitors of β-cat may have serious side effects, including severe bone marrow dysplasia, anemia, and overall wasting in treated mice—a possible result of disruption of homeostatic Wnt signaling in normal normal hematopoietic and intestinal stem cells. Such therapeutic limitations may derive from disruption of beta-catenin-TCF and beta-catenin-E-cadherin interactions, which can affect epithelial tissue integrity. In addition, biologic agents targeting Frizzled receptors (OMP-54F28 and OMP-18R5) have shown significant bone marrow toxicity during clinical trials. Wnt ligands are essential for Wnt/ρ-cat activation, but APC and β-catenin mutations in cancer cells can induce downstream transcription in the absence of Wnt ligand activation, so blocking Wnt secretion fails to suppress endogenous oncogenic Wnt activity caused by the transcription of downstream genes induced by APC and β-catenin mutations. LGK974 only targets a small patient population, as indicated by certain biomarkers. PRI-724, a small-molecule inhibitor, is in phase II trials using daily infusions, but more than once-weekly intravenous (IV) doses exhibit undesirable properties and are untenable for clinical development.

Traditionally, the Wnt signaling pathway comprises three different types of signal transduction: a canonical Wnt signaling pathway where Wnt regulates various transcriptional target genes in a β-catenin dependent manner; a non-canonical Wnt signal transduction pathway mainly involved in planar cell polarity, where Wnt can act independently of β-catenin; and a non-canonical Wnt/calcium pathway that regulate intracellular calcium levels. In the present application, “canonical Wnt signaling” is interchangefully referred to as “canonical Wnt/β-catenin signaling” or “Wnt signaling”. As described herein, canonical Wnt/β-catenin signaling may refer to the component of the pathway that controls the amount of β-catenin in a patient or sample by modulating the stability of β-catenin. In some embodiments, canonical Wnt/β-catenin signaling includes a pathway component that transcriptionally modulates one or more genes such as c-myc, ccnd1, cd44, LGR5, VEGFA, AXIN2, and LEF1. In some embodiments, canonical Wnt/β-catenin signaling includes pathway components that are modulated through the interaction between β-catenin and BCL9. In some embodiments, canonical Wnt/β-catenin signaling comprises one or more genes that are transcriptionally controlled through the interaction between β-catenin and BCL9. The one or more genes controlled by the interaction between β-catenin and BCL9 may comprise c-myc, ccnd1, cd44, LGR5, VEGFA, AXIN2, and LEF1. In some embodiments, canonical Wnt/β-catenin signaling comprises one or more proteins, the transcriptional expression of which are modulated by the interaction between β-catenin and BCL9. These components may comprise, for example, c-Myc, Cyclin D1, CD44, LGR5, VEGFA, AXIN2, and LEF1.

### Methods of Use

In some embodiments, administration of the compound of the present invention to a subject inhibits Wnt signaling in the subject. In some embodiments, administration of the compound of the present invention inhibits the binding of BCL9 to β-catenin. In some embodiments, administration of the compound of the present invention canonical Wnt/β-catenin signaling. In some embodiments, administration of the compound of the present invention treats a disease in the subject.

In some embodiments, the compound of the present invention is capable of inhibiting BCL9 binding to β-catenin in vitro and/or in vivo. In some embodiments, the compound of the present invention has one or more improved effects. Such one or more effects may be selected from one or more of: (1) inhibition of BCL9 binding to β-catenin; (2) inhibition of canonical Wnt signaling; (3) reducing the survival of regulatory T cells; (4) decreasing the expression of VEGF in tumor cells; (5) increasing CD4+ and CD8+ T cells infiltrating into the tumor; (6) increasing the infiltration of T helper 17 (Th17) cells into the tumor; (7) reducing intratumoral dendritic cells; (8) having a half-life (T½) greater than at least 2 hours when administered to a subject; (9) inducing tumor microenvironment favoring an immune response; and (10) inhibiting tumor growth, cancer stem cell proliferation and/or tumor metastasis.

In some embodiments, the compounds of the present invention exhibit favorable biological functions in some or each of the classes listed above, for example, potencies in various biochemical and cell bioassays, including cell-based Wnt and/or β-catenin transcription assays.

### Binding of BCL9 to β-Catenin

Pygopus (Pygo) and Legless (Lgs) were discovered in Drosophila as novel components of Wnt signaling that are essential for armadillo-mediated transcription during normal development. Pygo and BCL9/Legless transduce Wnt signaling by promoting the transcriptional activity of β-catenin/Armadillo in normal and malignant cells. The ability of compounds to inhibit the binding of BCL9 to β-catenin can be evaluated in various assays in the art. In some embodiments, the ability of the compound of the present invention to inhibit the binding of BCL9 to β-catenin can be evaluated using a homogeneous time-resolved fluorescence (HTRF) binding assay. In this assay, the compound/small molecule conjugated to a marker that can recognize another marker attached to the target protein (i.e., β-catenin). When the compound/small molecule is binded to the target protein and therefore the two markers are in proximity, a signal is generated and can be read quantitatively to calculate the binding affinity of the compound/small molecule. In some embodiments, the binding affinity of the compound/small molecule in this assay is compared to that of the control to detect improved binding affinity as compared to that of the control.

In some embodiments, the ability of the compounds of the present invention to inhibit the binding of BCL9 to β-catenin can be evaluated in an amplified luminescence proximity homogeneous analysis (ALPHA). In this assay, the compound is conjugated to a donor bead, and its target protein (i.e., β-catenin) is attached to an acceptor bead. When two beads come in close proximity due to the binding of the compound to the target protein, a signal is generated and the binding affinity of the compound can be quantitatively calculated. In some embodiments, the binding affinity of the compound in this assay is compared to that of a vehicle or control to detect improved binding affinity as compared to that of the vehicle or control.

In various embodiments, the ability of the compound of the present invention to inhibit the binding of BCL9 to β-catenin can be evaluated in a Wnt transcription assay. In some embodiments, the Wnt transcription assay is a cell-based assay. In some embodiments, the cell-based Wnt transcription assay is a β-lactamase (bla) reporter assay. Various cell lines, transformed cell lines, or primary cells derived from a healthy subject or a subject suffering from a disease can be used in this assay. A cell lineknown to rely on canonical Wnt/β-catenin signaling for its survival may also be used. In some embodiments, CellSensor™ LEF/TCF-bla HCT-116 cells and Cignal Wnt reporter are used in this reporter assay. These cells contain a β-lactamase (BLA) reporter gene under the control of a β-lactamase/LEF/TCF response element that is stably integrated into HCT-116 cells. Because the cells constitutively express β-lactamase, adding a compound that inhibits binding of BCL9 to β-catenin in this assay will reduce production of β-lactamase. Thus, the efficiency of the compound in inhibiting Wnt transcription can be quantitavely calculated in this assay.

In some embodiments, the ability of the compound of the present invention to inhibit the binding of BLC9 to β-catenin can be evaluated in a cell viability assay. In some embodiments, the cell viability assay is a CellTiterGlo luminescence assay, wherein cell viability is quantitatively measured. Various cell lines, transformed cell lines, or primary cells derived from a healthy subject or subject suffering from a disease can be used in this assay.

### Canonical Wnt/β-Catenin Signaling

In certain embodiments, the ability of the compounds of the present invention to inhibit canonical Wnt/β-catenin signaling can be evaluated in various in vitro and/or in vivo assays. In some embodiments, the effect of the compound of the present invention on canonical Wnt/β-catenin signaling is evaluated in cell-based Wnt transcription assays, such as β-lactamase (bla) reporter assays. The β-lactamase (bla) reporter assay measures the strength of canonical Wnt/β-catenin signaling by its ability to control β-catenin/LEF/TCF response elements and can therefore be used to assess whether the test agent can attenuate or increase the strength of control of its transcriptional targeting by canonical Wnt/β-catenin signaling transcription.

The ability of the compounds of the present invention to inhibit canonical Wnt/β-catenin signaling can also be evaluated by measuring the gene expression and/or protein expression of target genes that are transcriptionally controlled by canonical Wnt/β-catenin signaling. The expression of target genes can be evaluated in transcribed cells in contact with the compounds of the present invention or in subjects administered with these compounds. The target genes include, for example, CMYC, CCND1, CD44, LGR5, VEGFA, AXIN2, and LEF1. The expression level of one or more target genes associated with canonical Wnt/β-catenin signaling can be analyzed using methods known in the art, such as cell staining, flow cytometry, immunoblotting, and/or real-time quantitative PCR (rt-qPCR) analysis.

### Regulatory T Cells Survival

Various markers such as CD4, FOXP3, and CD25 are known to be expressed on regulatory T cells. The ability of the compounds of the present invention to reduce the survival of regulatory T cells can be evaluated by counting the total number of regulatory T cells present in the blood and/or in specific tissues such as tumors. For example, a sample obtained from a subject contacted with a compound of the present invention may be stained with an antibody that detects a marker associated with regulatory T cells. The sample can also be processed and labeled with antibody that detect such markers and analyzed by flow cytometry. The gene and/or protein expression of such markers can be measured in the samples and analyzed by, for example, immunoblotting and/or rt-qPCR.

### VEGF Expression in Tumors

Various assays can be used to measure gene expression and/or protein expression of VEGF in tumor samples. For example, after contacting the subject to the compound, tumor cells can be collected and stained with an anti-VEGF antibody to detect VEGF protein. The cells can also be analyzed by, for example, rt-qPCR to determine the gene expression of VEGF. Other assays indicating changes in VEGF expression may be used. For example, a tumor sample from a subject contacted with a compound of the present invention may be analyzed to detect various angiogenic markers controlled by VEGF. In some embodiments, the compounds of the present invention reduce VEGF expression more effectively than a vehicle or control.

CD4+ and/or CD8+ T Cells Infiltrate into the Tumor

The infiltration of CD4+ T cells and/or CD8+ T cells into the tumor can be assessed by counting the total number of CD4+ and/or CD8+ T cells present in the tumor or a sample (for example, a biopsy) from the tumor. Various markers, for example, CD4 and CD45, are known to be expressed on CD4+ T cells (also known as helper T cells),. Various markers, for example, CD8 and CD45, are known to be expressed on CD8+ T cells (also known as cytotoxic T cells). The ability of a compound to increase the infiltration of CD4+ and/or CD8+ T cells into a tumor can be evaluated in vivo by administering the compound to a subject having tumors. Tumor samples can be collected from the subject and stained with antibodies that detect markers associated with CD4+/CD8+ T cells. The samples can also be processed and labeled with, for example, antibodies that detect such markers, and analyzed by, for example, flow cytometry. The gene and/or protein expression of such markers may also be measured in the sample and analyzed by, for example, immunoblotting and/or rt-qPCR.

T Helper 17 Cells Infiltration into the Tumor

In some embodiments, the compound of the present invention is capable of increasing T-helper 17 cell infiltration into a tumor when administered to a tumor-bearing subject. The infiltration of T-helper 17 cells into a tumor can be evaluated by counting the total number of T-helper 17 cells present in the tumor. Various markers, e.g., IL-17, are known to be expressed on T helper 17 cells. The ability of a compound to increase the infiltration of T helper 17 cells into tumors can be evaluated in vivo by administering the compound to a subject having a tumor. A tumor sample can be collected from the subject and stained with, for example, antibodies that detect markers associated with T-helper 17 cells. The samples can also be processed and labeled with antibodies that detect such markers and analyzed by flow cytometry. Gene and/or protein expression of such markers can also be measured in the samples and analyzed by, for example, immunoblotting and/or rt-qPCR. The sample can be analyzed to detect the amount of IL-17 present in the sample.

### Dendritic Cells in Tumors

In some embodiments, when administered to a tumor-bearing subject, the compound of the present invention is capable to modulate dendritic cells present in the tumor. The number of dendritic cells present in the tumor can be assessed, for example, by staining the tumor with antibodies that recognize one or more markers associated with the dendritic cells. Various markers, for example, CD11c, are known to be expressed on dendritic cells. The ability of a compound to reduce dendritic cells in a tumor can be assessed in vivo by administering the compound to a subject. Tumor samples can be collected from the subject and stained with antibodies that detect markers associated with dendritic cells. The sample may also be processed and labeled, for example, with antibodies that detect such markers, and analyzed, for example, by flow cytometry. Gene and/or protein expression of such markers is analyzed by, for example, immunoblotting and/or rt-qPCR.

### Biomarkers

The present disclosure also encompass methods for measuring at least one biomarker for monitoring the therapeutic efficacy of a compound or pharmaceutical composition of the present invention or for selecting a subject to be treated with such compound or pharmaceutical composition. In some embodiments, the biomarker is one or more of BCL9, CD44, Axin2, cMyc, LGR5, VEGFA, Sox2, Oct4, Nanog, and/or active β-catenin. Active β-catenin, as used herein, refers to the nonphosphorylated form of β-catenin.

Various known methods can be used to measure the gene expression level and/or protein level of such biomarkers. For example, a sample from a subject treated with the compound or pharmaceutical composition can be obtained, such as a biopsy of a tumour, blood, plasma, serum, urine, amniotic fluid, synovial fluid, endothelial cells, leukocytes, monocytes, other cells, organs, tissues, bone marrow, lymph nodes or spleen. In some embodiments, the sample is a tumor biopsy in a subject. The sample obtained from the subject may be stained with one or more antibodies or other detection reagents that detect such biomarkers. The sample may also or alternatively be processed to detect the presence of nucleic acids (such as mRNA) encoding the biomarker by, for example, rt-qPCR methods.

In some embodiments, a reduced gene expression level and/or protein level of BCL9, CD44, Axin2, cMyc, LGR5, VEGFA, Sox2, Oct4, Nanog, and/or active β-catenin indicates the therapeutic efficacy of a compound or pharmaceutical composition described herein. The expression level of such biomarker may be measured, for example, after 1 day, 2 days, 3 days, 4 days, 5 days, one week, or two week of administration of the compound or pharmaceutical composition, or any time period in between. In some embodiments, a method is disclosed comprising measuring the level of one or more biomarkers after one or more rounds of use of a compound or pharmaceutical composition of the invention. In some embodiments, the method further comprises continued administration of the compound or pharmaceutical composition if the level of the biomarker decreases. In some embodiments, the methods further include administering an increased dose of a compound or pharmaceutical composition of the invention if the biomarker level does not decrease, or increasing the frequency of subsequent administration. In some embodiments, treatment is stopped if the biomarker level does not decrease after the initial administration. In various embodiments, the marker levels are also measured before the first use of the compound or pharmaceutical composition of the invention and compared with the levels after one or more rounds of administration, wherein therapeutic efficacy and continued treatment steps are determined based on the change in a biomarker level from one or more levels prior to administration.

In some embodiments, an increased gene expression level and/or protein level of BCL9, CD44, Axin2, cMyc, LGR5, VEGFA, Sox2, Oct4, Nanog, and/or active β-catenin indicates that, in comparison with a subject without increased gene expression level and/or protein levels the subject will benefit from treatment with a compound or pharmaceutical composition according to the invention. In some embodiments, treatment methods are disclosed, including selection of patients with increased biomarker levels and administration of a compound or pharmaceutical composition of the invention.

In certain embodiments, a subject with increased gene and/or protein expression level of BCL9, CD44, Axin2, cMyc, LGR5, VEGFA, Sox2, Oct4, Nanog, and/or active β-catenin is selected for treatment with a compound or pharmaceutical composition of the invention. In some embodiments, after obtaining a tumor sample from the subject and identifying increased gene and/or protein expressions of BCL9, CD44, Axin2, cMyc, LGR5, VEGFA, Sox2, Oct4, Nanog, and/or active β-catenin, the subject having a tumor is selected for treatment. In some embodiments, after obtaining a tumor sample from a subject and identifying elevated gene and/or protein expression of BCL9, the subject having a tumor is selected for treatment. In some embodiments, after obtaining a tumor sample from a subject and identifying the elevated gene and/or protein expression of CD44, the subject having a tumor is selected for treatment. In some embodiments, after obtaining a tumor sample from a subject and identifying elevated gene and/or protein expression of active β-catenin, the subject having a tumor is selected for treatment.

### Half-Lives in Receptors

In some embodiments, the compound of the invention has one or more improved pharmacokinetic parameters as compared to a vehicle or control. Such pharmacokinetic parameters may comprise, for example, maximum observed concentration (Cmax), time to reach the maximum concentration (Tmax), terminal half-life (T½), total body clearance (CL), volume of distribution (Vz), area under the curve from time of administration to last measurable concentration (AUC0-t), area under the curve from time of administration extrapolated to infinity (AUC0-inf), and bioavailability.

The methods used for assessing pharmacokinetics of agents are known in the art. For example, a blood sample from a subject administered with a compound described herein may be obtained at 5 minutes, 1, 2, 4, 6, 8, 12, and 24 hours after administration. The concentration of the compound in the blood sample can be analyzed by various analytical tools, for example, LC/MS. Based on the concentration of the compound at each time point, the pharmacokinetic parameters are calculated. As used herein, the term “maximum observed concentration (Cₘₐₓ)” refers to the maximum serum concentration of a compound reaches after administration. Related to the concept of Cₘₐₓ, the time to reach the maximum concentration (Tₘₐₓ) is the time that a compound takes to reach the maximum serum concentration. The terms “terminal half-life (T_{1/2})” and “half-life (T_{1/2})” are used interchangeably and refer to the time that a compound takes to lose half of its serum concentration. Total body clearance (CL) indicates the amount of blood completely cleared of a compound per unit time. The term “volume of distribution (V_{z})” refers to a theoretically calculated volume that needs to contain the total amount of compound administered to a subject at the same concentration observed in blood. The term “bioavailability” refers to the extent and rate at which a drug is absorbed into a biological system or becomes available at the physiologically active site. Bioavailability may be a function of several of the properties previously described, including stability, solubility, immunogenicity, and pharmacokinetics, and may be evaluated using methods known to those skilled in the art.

The pharmacokinetic parameters of the compound can be evaluated in mammals, including, for example, mice, rats, or humans. The parameters can also be evaluated using various administration routes, such as intravenous, intraperitoneal, subcutaneous, and intramuscular administration routes. In some embodiments, pharmacokinetic parameters of the compounds of the invention are evaluated in mice. In some embodiments, pharmacokinetic parameters of the compounds described herein are evaluated in mice administered subcutaneously with the compounds. In some embodiments, pharmacokinetic parameters of the compounds of the invention are evaluated in humans. In some embodiments, pharmacokinetic parameters of the compounds of the invention are evaluated in the human after subcutaneous administration.

### Tumor Microenvironment Favoring Immune Response

In various embodiments, the compound of the invention induces a tumor microenvironment favoring an immune response. In various embodiments, the compounds of the invention induce a tumor microenvironment that is more favorable to an immune response than the vehicle or the control.

A variety of parameters can be used to evaluate the tumor microenvironment. For example, an increased ratio between cytotoxic T cells and regulatory T cells in and/or around the tumor tissue may indicate that the tumor microenvironment favors an immune response. Reduced numbers of dendritic cells and/or regulatory T cells in and/or around the tumor tissue may also indicate that the tumor microenvironment is favorable for immune responses. Other parameters include an increase in circulating T cells in the peripheral blood and an increase in the ratio of T helper 17 cells to regulatory T cells in and/or around the tumor tissue. These parameters may indicate that the tumor microenvironment favors immune responses.

In some embodiments, the compounds of the invention may increase the ratio of the amount of cytotoxic T cells to the amount of regulatory T cells in the tumor microenvironment. In some embodiments, the ratio change caused by the compound is greater than the ratio change caused by the vehicle or the control.

Tumor growth, tumor stem cell proliferation, and/or tumor metastasis.

Since Wnt signaling is a regulator of tumor growth, the therapeutic efficacy of compounds affecting BCL9 binding to β-catenin can be evaluated in animal models.

The in vivo efficacy of a compound of the present inventions may be assessed in models of human cancers using, e.g., BALB/c nude mice, since xenografts of human cancer cells will grow into tumors in these mice. For example, subcutaneously inoculation of Colo320DM tumor cells, a commercially available cell line derived from human colon cancer tissue, can be used to form a tumor in BALB/c nude mice. Additional in vivo models may also be utilized to evaluate the in vivo efficacy of the compound disclosed herein. For example, human DLD-1 colon cancer cells can be implanted into nude mice to assess tumor growth. A CT26 syngeneic mouse model of colon cancer can also be used, as such models allow assessment of tumor growth in the context of an intact immune system. Other cancer cell types, for example, B16 melanoma, 4T1 breast cancer, human renal cancer, and Lewis lung cancer cells may also be used in these known animal models to evaluate the in vivo efficacy of the compounds disclosed herein.

By administering a compound of the present invention to one or more animal models, the effect of the compound in reducing tumor growth in vivo can be evaluated. Based on data from animals treated with a stable BCL9 peptide, the ability of the peptide to inhibit Wnt signaling can be evaluated by, for example, staining tissue samples with markers of Wnt signaling. These downstream markers of Wnt signaling include, for example, Axin2 and CD44.

Orthotopic mouse models can be used to evaluate the effects of the compounds described here on tumor metastasis. For example, an orthotropic animal model could be injected with cells carrying a luciferase construct and then administered with its indicated treatment. The presence of injected cells can be detected by administering a luciferin substrate to each treated animal. The intensity of the bioluminescent signal can be measured quantitatively and used as an indicator of cell growth.

In some embodiments, the effect of the compounds of the invention on the proliferation of cancer stem cells can be evaluated by measuring various biomarkers of cancer stem cells. For example, the expression level of CD44 and/or LGR5 may indicate the amount of cancer stem cells present in the sample. Tumor samples can be collected from a subject and stained with antibodies that detect markers associated with cancer stem cells. The sample can also be processed and labeled, for example, with antibodies that detect such markers, and analyzed, for example, by flow cytometry. Gene and/or protein expression of such markers can be detected and analyzed by, for example, immunoblotting and/or rt-qPCR.

Disorders with Abnormal Wnt/β-Catenin Signaling

Aberrant Wnt/β-catenin signaling is associated with the malignant transformation of normal cells into cancer cells. Activation of Wnt signaling and β-catenin nuclear localization has been linked to tumor phenotypes in multiple models.

The present disclosure encompasses compositions for use and methods of using compounds disclosed herein to inhibit the binding of BCL9 to 3-catenin in a subject by administering the compound or a pharmaceutical composition comprising the compound to the subject. The present disclosure also encompasses inhibition of canonical Wnt/β-catenin signaling in a subject by administration of a compound or pharmaceutical composition disclosed herein. The present disclosure further encompasses methods of treating a disease in a subject by administering a compound or pharmaceutical composition of the invention to the subject. Such diseases may be cancer or other neoplastic disorders associated with aberrant canonical Wnt/β-catenin signaling.

In some embodiments, the disease, disorder, or condition may be a disease which could benefit from inhibition of canonical Wnt/β-catenin signaling. In some embodiments, such disease, disorder or condition may be cancer. In some embodiments, the cancer is a cancer with high expression of BCL9 and/or β-catenin. In some embodiments, the cancer is a cancer where BCL9 and β-catenin are co-localized in the nucleus of the cancer cell. In some embodiments, the cancer is selected from Familial adenomatous polyposis (FAP), ocular cancer, rectal cancer, colon cancer, colorectal cancer, cervical cancer, prostate cancer, breast cancer, bladder cancer, oral cancer, benign and malignant tumors, stomach cancer, liver cancer, pancreatic cancer, lung cancer, corpus uteri, ovarian cancer, prostate cancer, testicular cancer, kidney cancer, brain/CNS cancer, laryngeal cancer, multiple myeloma, skin melanoma, acute lymphoblastic leukemia, acute myeloid leukemia, Ewing's sarcoma, Kaposi's sarcoma, basal cell and squamous cell carcinoma, small cell lung cancer, choriocarcinoma, rhabdomyosarcoma, angiosarcoma, hemangioendothelioma, Wilm's tumor, neuroblastoma, oral/pharyngeal cancer, esophageal cancer, laryngeal cancer, lymphoma, neurofibromatosis, tuberous sclerosis complex, hemangioma, gastric cancer, ovarian cancer, hepatocellular carcinoma, and lymphangiogenesis. In some embodiments, the cancer is colorectal cancer. In some embodiments, the cancer is gastric cancer. In some embodiments, the cancer is ovarian cancer. In some embodiments, the cancer is hepatocellular carcinoma. In some embodiments, the cancer is breast cancer. In some embodiments, the cancer is prostate cancer. In some embodiments, the cancer is a cutaneous melanoma. In some embodiments, the cancer is lung cancer.

In some embodiments, any compound or variant in the compound or variant disclosed herein or a pharmaceutical composition comprising such compound may be used to treat a disease, such as cancer, listed above.

Treatment and the measured treatment parameters may be evaluated after administration of the compound or pharmaceutical composition alone or in combination with one or more additional therapeutic agents (e.g., as a single bolus or in separate sequential administration). Additional agents may be any of additional therapeutic agents mentioned herein or known to one skilled in the art. Depending on the regimen chosen, the compound or the pharmaceutical composition comprising the compound and/or the additional agent may be administered one or multiple times.

The present invention also encompasses compounds or pharmaceutical compositions disclosed herein for use in the treatment of a disease in the subject. In some embodiments, the disease may benefit from inhibition of canonical Wnt/β-catenin signaling. In some embodiments, the disease is cancer.

The present disclosure further encompasses the use of a compound or pharmaceutical composition disclosed herein in the manufacture of a drug for the treatment of a disease in the subject. In some embodiments, the disease may benefit from inhibition of canonical Wnt/β-catenin signaling. In some embodiments, the disease is cancer.

In another embodiment, the disease being treated is a disease other than cancer. In certain embodiments, The disease is a bone density defect, an ocular vascular defect, familial exudative vitreoretinopathy, early coronary disease, Alzheimer's disease, autosomal dominant oligodontia, retinal angiogenesis, osteogenesis imperfecta, Tetra-Amelia syndrome, Mullerian-duct regression and andvirilization, SERKAL syndrome, type II diabetes mellitus, Fuhrmannsyndrome, odonto-onycho-dermal dysplasia, obesity, split hand/foot malformation, caudal duplication, tooth agenesis, skeletal dysplasia, focal dermal hypoplasia, autosomal recessive scleroderma, neural tube defects or sclerosteosis and Van Buchem disease.

### Combination Therapy

In certain embodiments, the compound or pharmaceutical composition disclosed herein is administered together with at least one additional agent. That is, a compound of the present disclosure and additional agents may be administered continuously or simultaneously to patients in separate dosage forms as described herein. In some embodiments, said at least one additional agent selected from a checkpoint inhibitor, an EGFR inhibitor, a VEGF inhibitor, a VEGFR inhibitor, an anticancer drug (e.g., any of the additional therapeutic agents described herein). The stapled peptide, and additional agents may be administered in a therapeutically effective amount.

In certain embodiments, the subject administered with a compound or pharmaceutical composition disclosed herein is also treated with radiotherapy and/or chemotherapy before, after, or at the same time as administration of the compound or pharmaceutical composition.

### Kits.

The present invention also includes a pharmaceutical kit for use, for example, in the treatment of a disorder, disease, and condition described herein, said pharmaceutical kit comprising one or more containers containing a pharmaceutical composition comprising a therapeutically effective amount of a compound of the present invention. If desired, such kit may also comprise various conventional pharmaceutical kit components, e.g., one or more container having one or more pharmaceutically acceptable carriers, additional containers, etc. The kit may also comprises an instruction manual, either as an insert or as a label, indicating the amount of the component to be administered, a guide for administration, and/or a guide for mixing the components.

Kits for performing the methods described herein are also disclosed herein. In various embodiments, kits are provided for manufacturing a compound of the invention. In some embodiments, the kit includes a compound capable of undergoing a reaction to form one or more hydrocarbon linked groups. In some embodiments, the kit includes a metal catalyst for performing a metal-mediated closed-ring metadecomposition.

In some embodiments, the kit includes an agent for detecting the gene and/or protein expression of BCL9, CD44, Axin2, cMyc, LGR5, VEGFA, Sox2, Oct4, Nanog, and/or active β-catenin.

The present invention was further described below in combination with specific embodiments. It should be understood that these examples are intended only used to illustrate the invention and not to limit the scope of the invention. The experimental methods without specific conditions in the following examples generally follow the conventional conditions or the conditions suggested by the manufacturer. Unless otherwise stated, percentages and parts are percentages and parts by weight.

### PREPARATION EXAMPLES

### General Synthetic Method

The compounds of the present invention can be prepared, isolated, or obtained by any method obvious to one skilled in the art. The compounds of the present invention can also be prepared according to exemplary preparation schemes, such as methods in examples, provided below. Reaction conditions, steps, and reactants not provided in the exemplary preparation scheme are obvious and known to one skilled in the art. As used herein, symbols and usage used in these processes, schemes, and examples have meanings that are well known to those skilled in the art, whether particular abbreviations are specifically defined or not. Specifically, but not limited to, the following abbreviations may be used in examples and throughout the specification: r.t. (room temperature); g (gram); mg (milligram); mL (milliliter); μL (microliter); mM (millimole); μM (micromoles); MHz (Hertz); MHz (megahertz); mmol (millimole); hr (hour); min (minute); MS) (mass spectrum); ESI (electrospray ionization); TLC (thin layer chromatography); HPLC (high performance liquid chromatography); BOC (t-butyloxycarbonyl); tBu (tert-butyl); HATU (2-(7-aza-benzotriazole)-N,N,N′,N′-tetramethylurea hexafluorophosphate); TFA (trifluoroacetic acid); Pd₂(dba)₃ (tri(dibenzylacetone)dipalladium); DIPEA (N,N-diisopropylethylamine).

For example, some compounds of the present invention can be prepared by the following scheme:

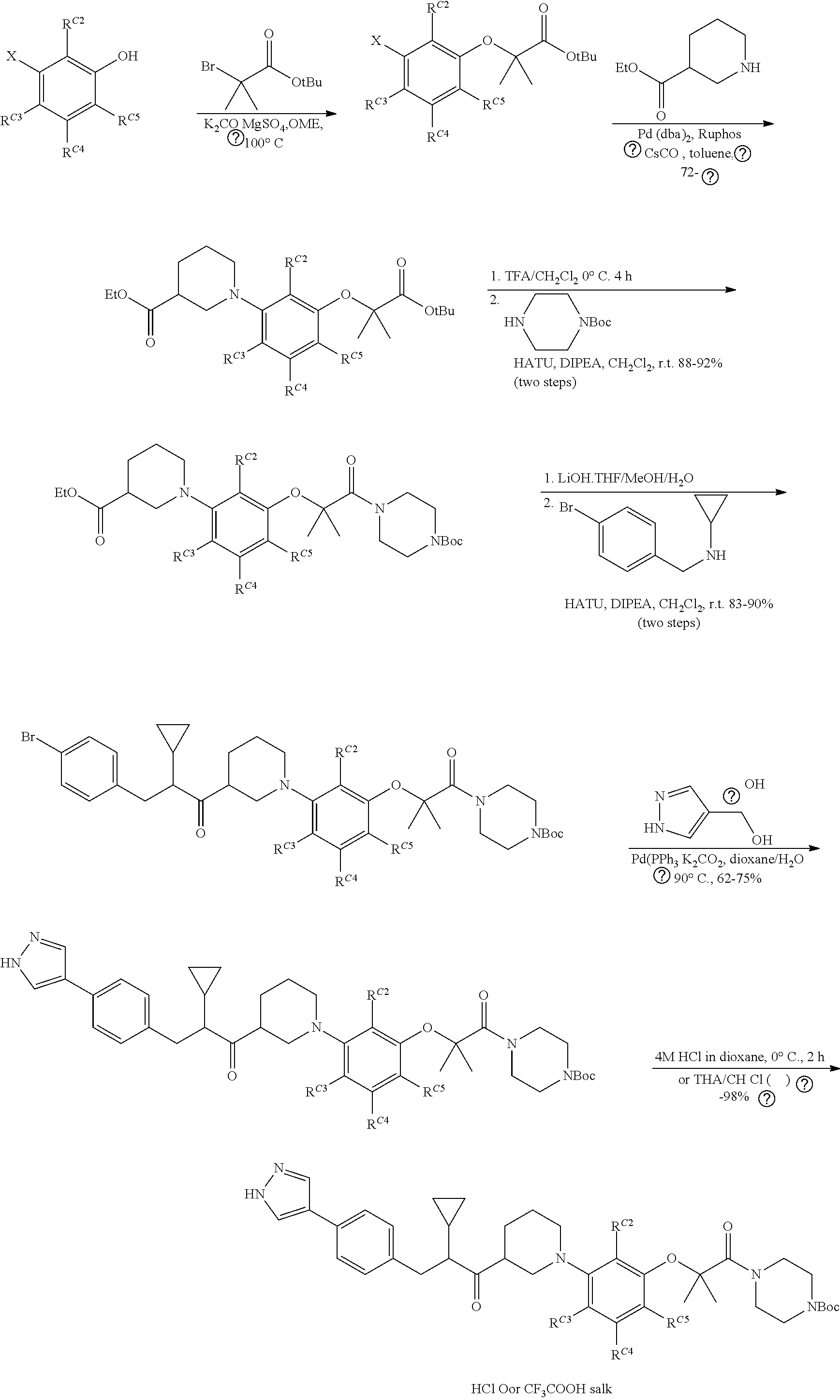

wherein, R^{C2}, R^{C3}, R^{C4} and R^{C5} are as defined above; X is a suitable leaving group.

### Preparation Example 1

### Example 1.1 Preparation of Compound I-1

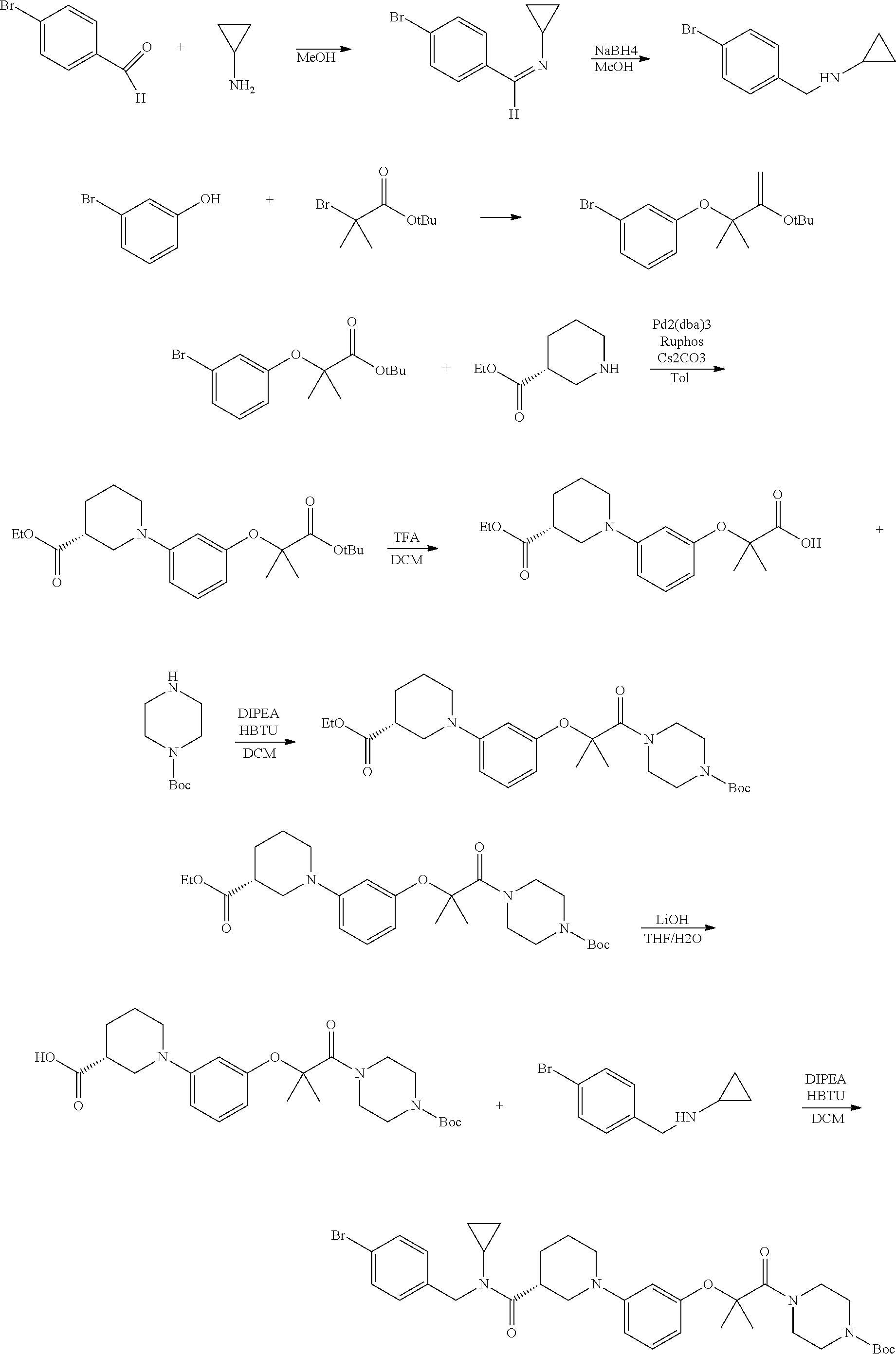

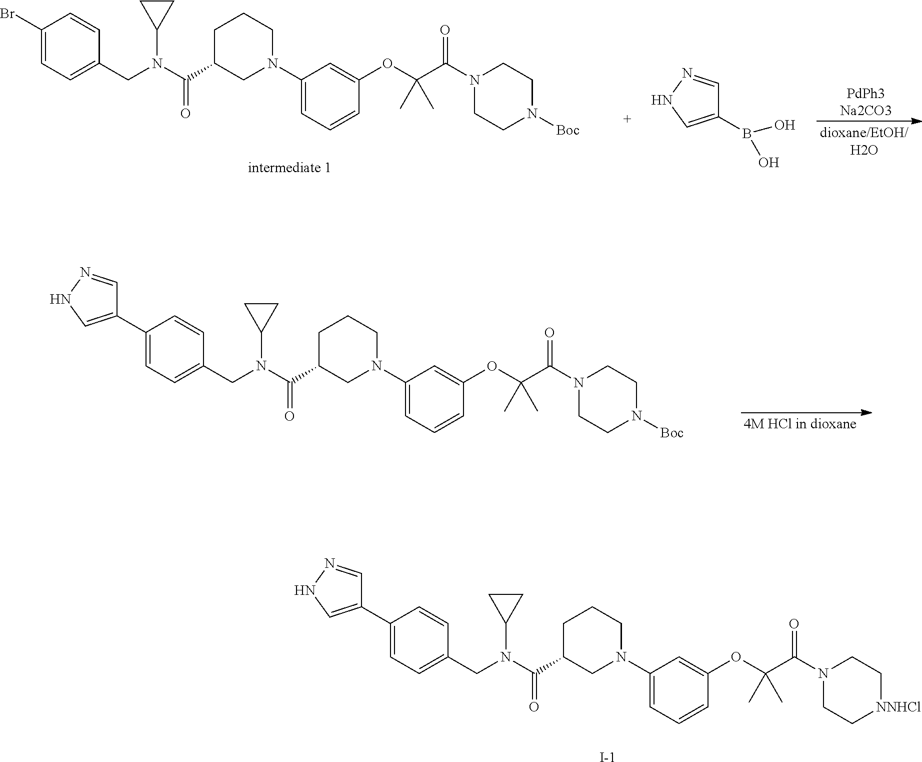

Compound I-1 was synthesized by the synthetic route as shown above.

### Example 1.2: Synthesis of Compound I-2

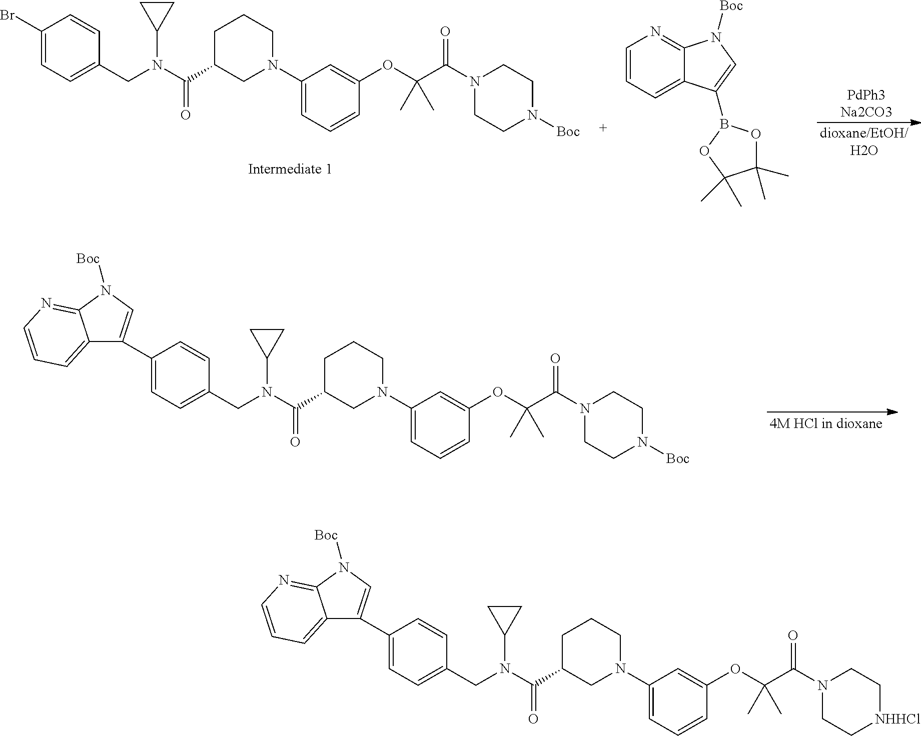

Compound I-2 was synthesized by the synthetic route as shown above.

### Example 1.3: Compounds C37-012 to C37-016 can be Synthesized According to the Following Process

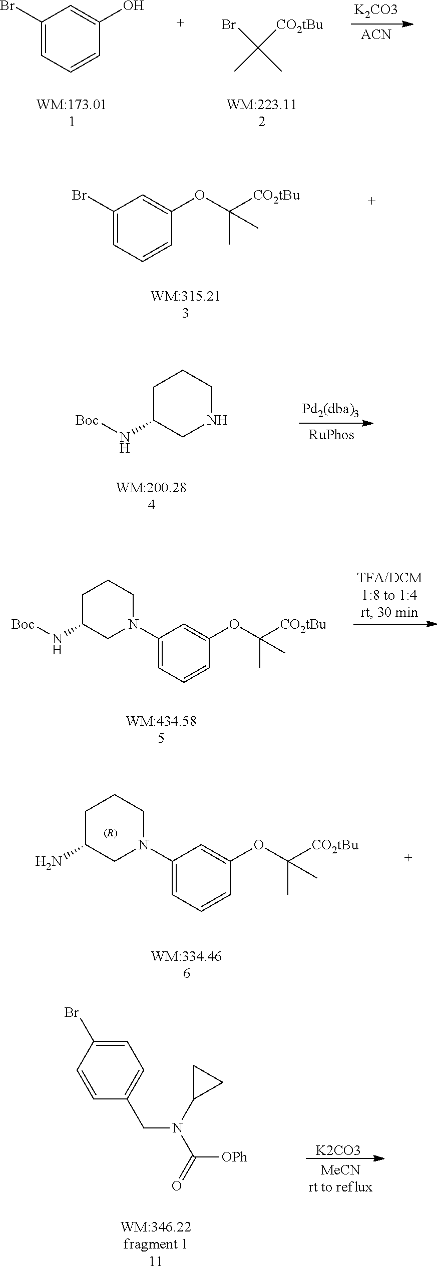

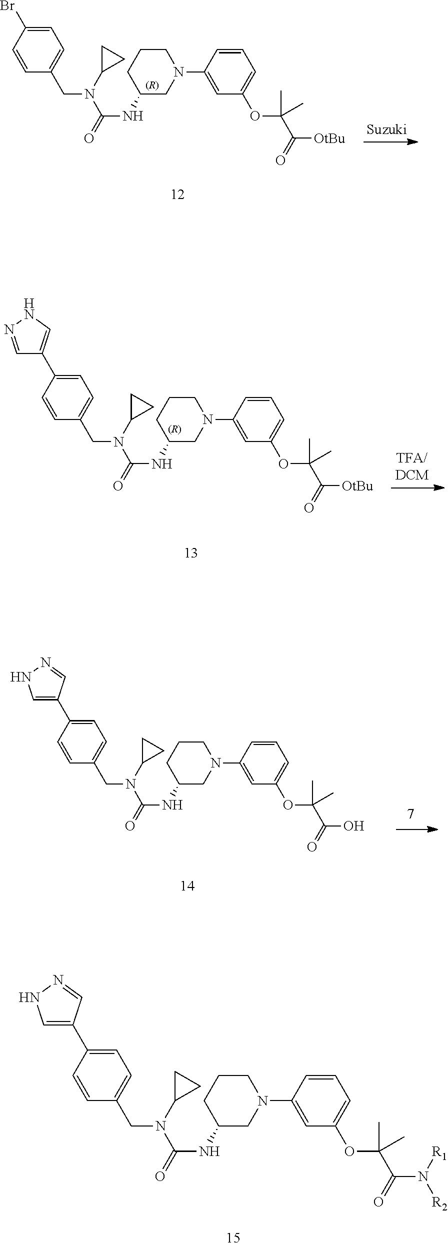

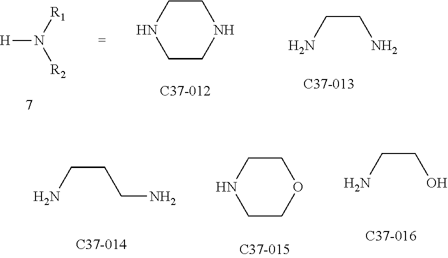

### Example 1.4: Compounds C37-018 to C37-022 can be Synthesized According to the Following Process

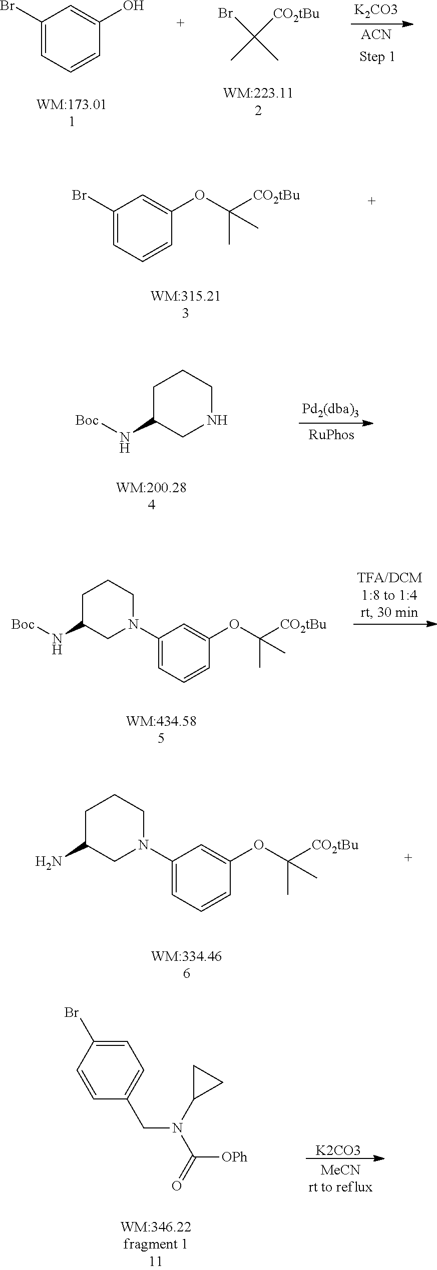

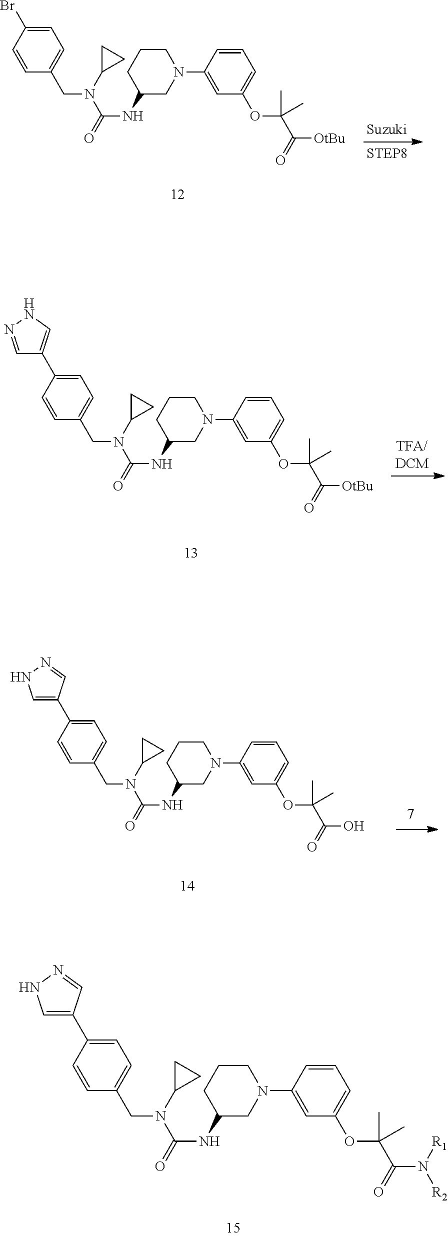

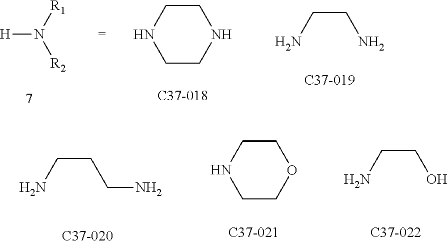

### Example 1.5: Compounds C37-032 to C37-033 can be Synthesized According to the Following Process

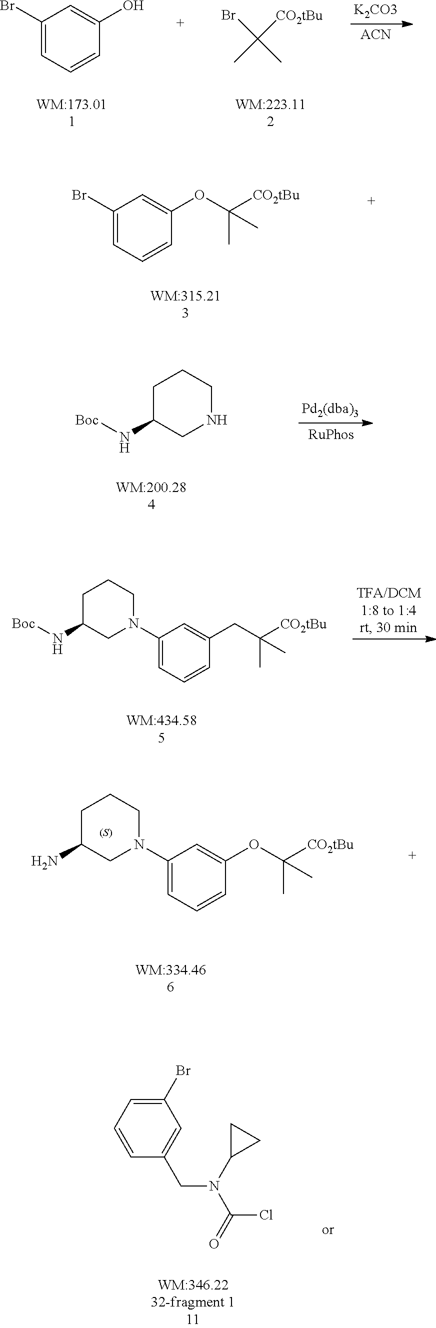

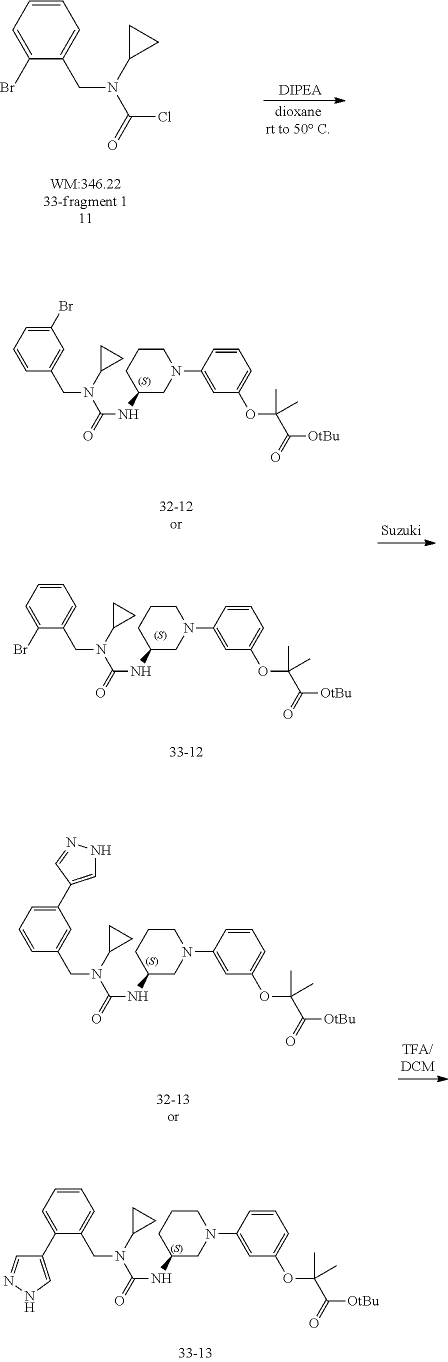

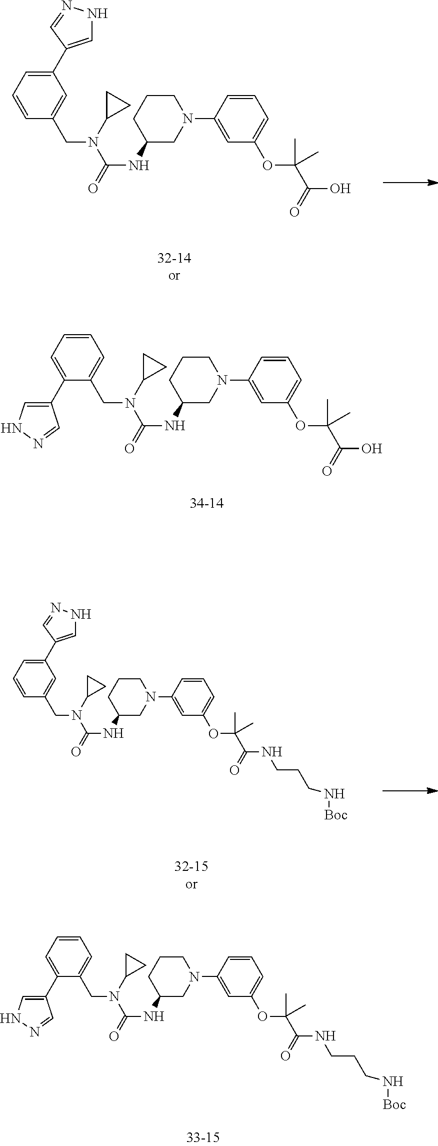 [SMILES: CC(C)(Oc1cccc(N2CCC[C@H](NC(=O)N(Cc3cccc(-c4cn[nH]c4)c3)C3CC3)C2)c1)C(=O)O ; CC(C)(Oc1cccc(N2CCC[C@H](NC(=O)N(Cc3ccccc3-c3cn[nH]c3)C3CC3)C2)c1)C(=O)O ; CC(C)(C)OC(=O)NCCCNC(=O)C(C)(C)Oc1cccc(N2CCC[C@H](NC(=O)N(Cc3cccc(-c4cn[nH]c4)c3)C3CC3)C2)c1 ; CC(C)(C)OC(=O)NCCCNC(=O)C(C)(C)Oc1cccc(N2CCC[C@H](NC(=O)N(Cc3ccccc3-c3cn[nH]c3)C3CC3)C2)c1]

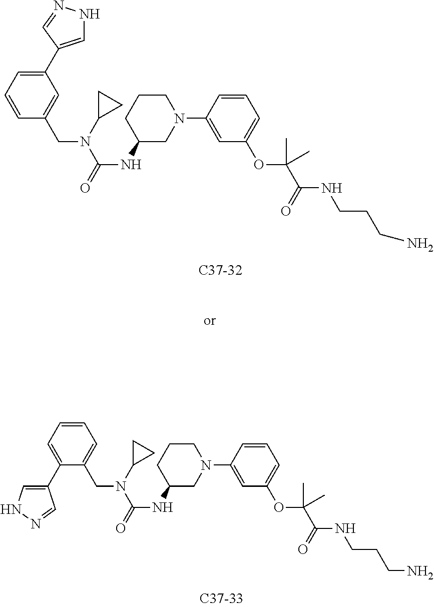 [SMILES: CC(C)(Oc1cccc(N2CCC[C@H](NC(=O)N(Cc3cccc(-c4cn[nH]c4)c3)C3CC3)C2)c1)C(=O)NCCCN ; CC(C)(Oc1cccc(N2CCC[C@H](NC(=O)N(Cc3ccccc3-c3cn[nH]c3)C3CC3)C2)c1)C(=O)NCCCN]

### Example 1.6: Compound C37-035 can be Synthesized According to the Following Process

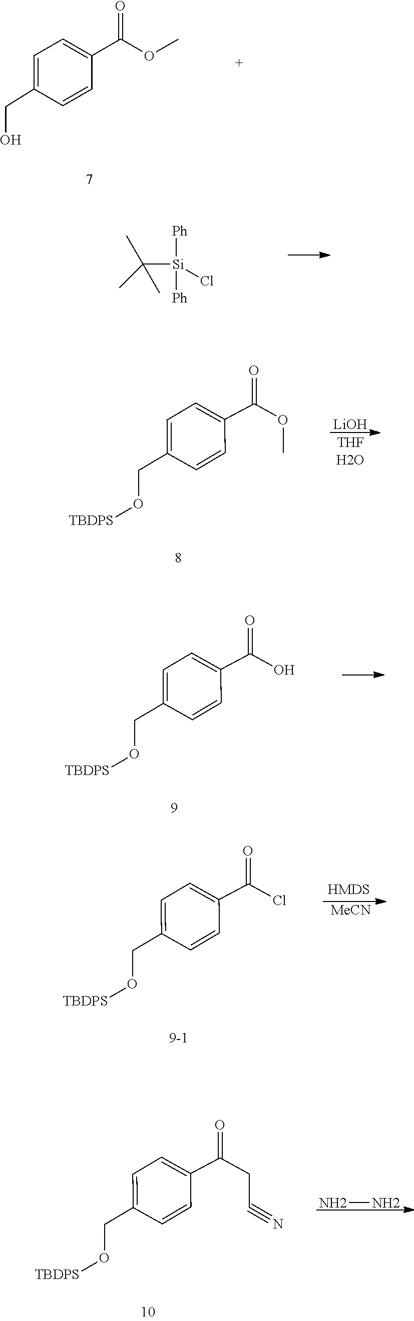

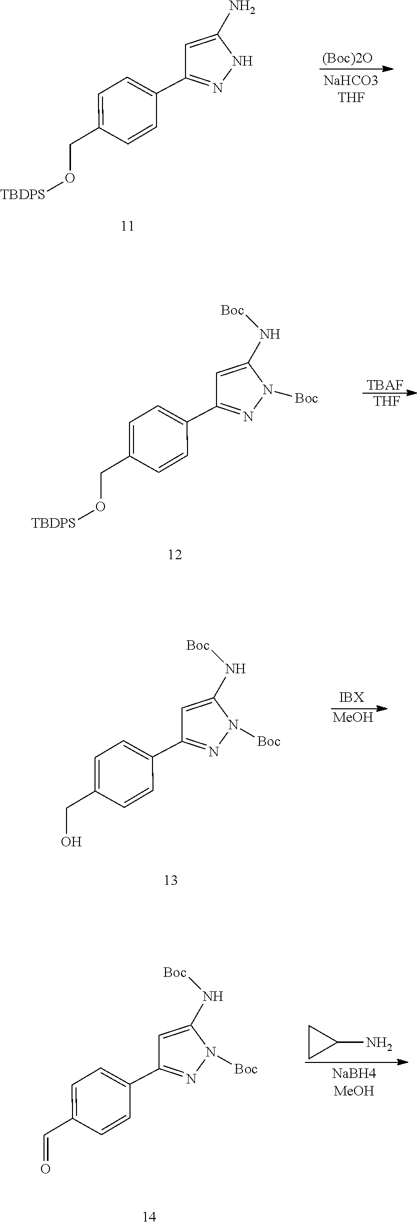

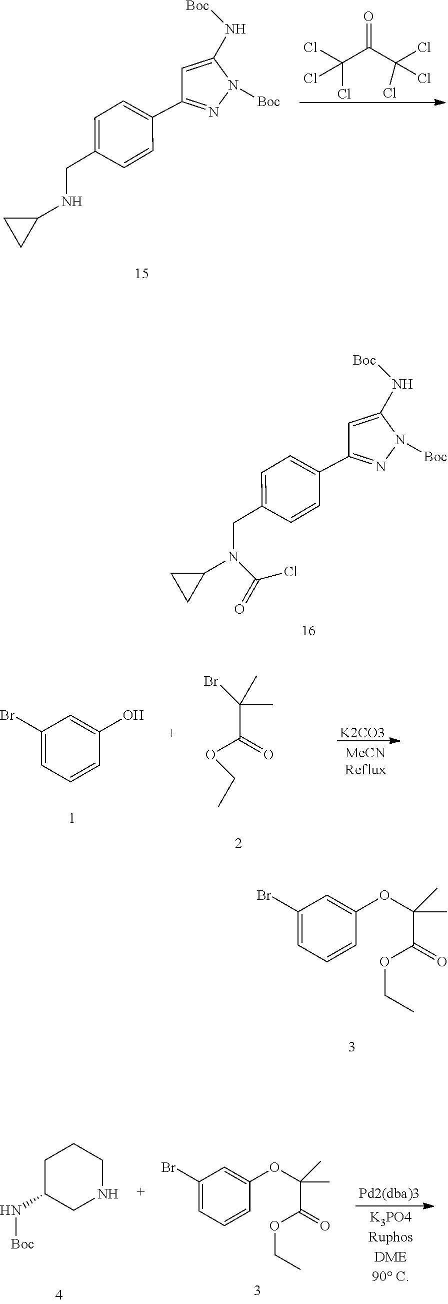

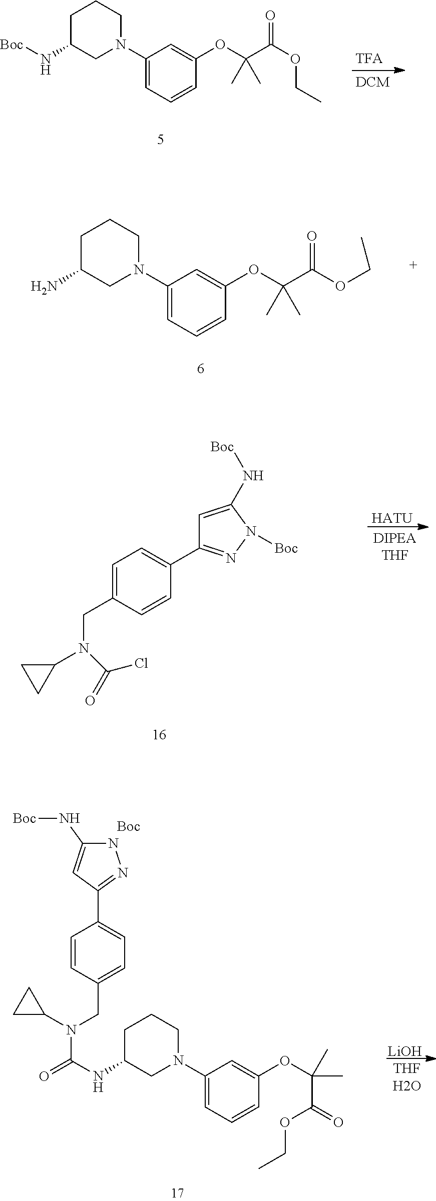

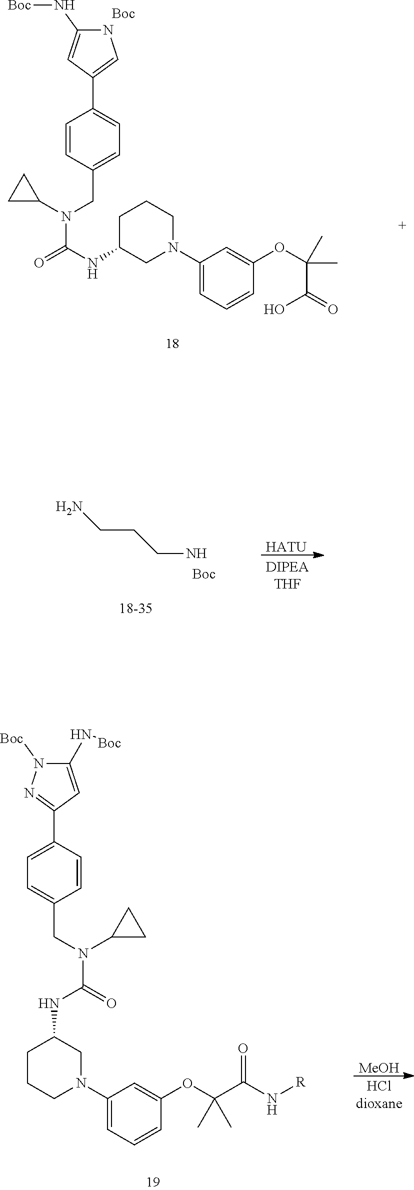

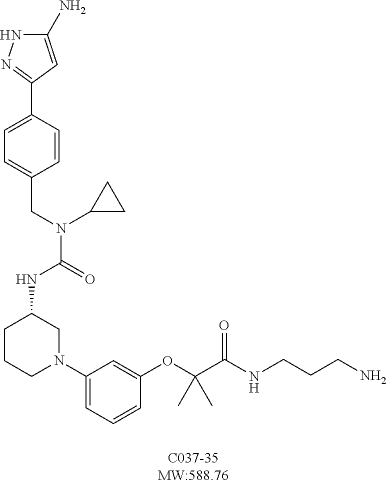

### Example 1.7: Compound C37-036 can be Synthesized According to the Following Process

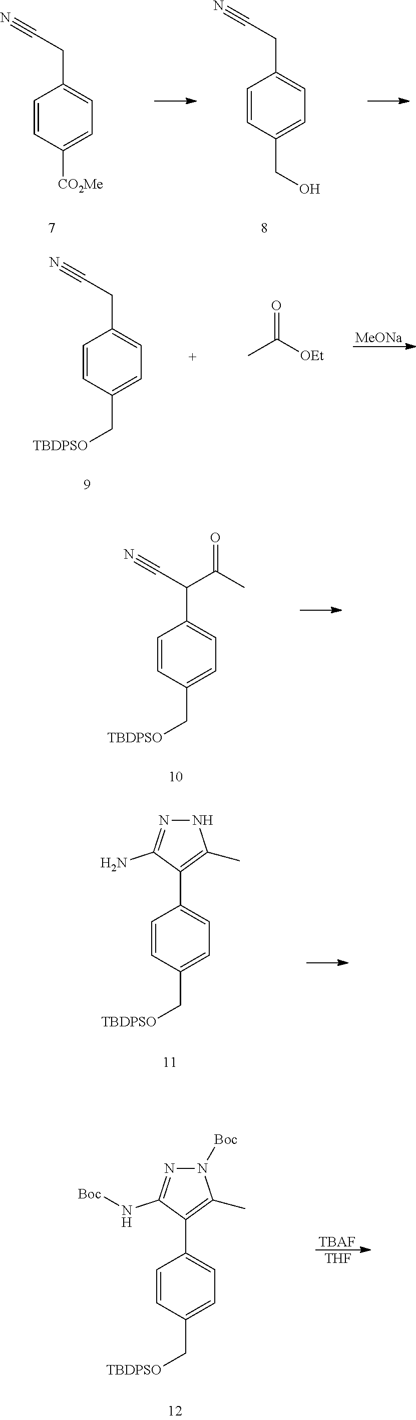

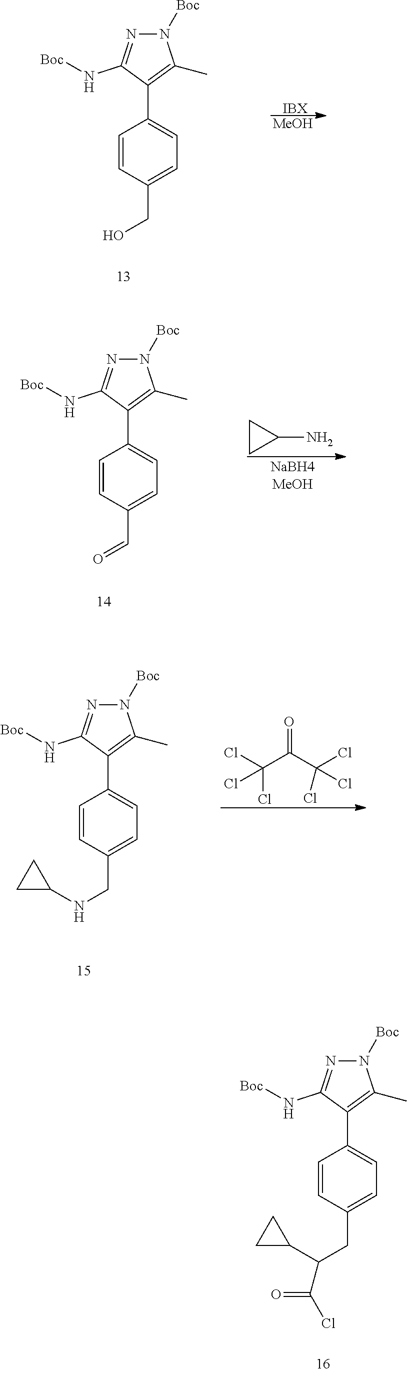

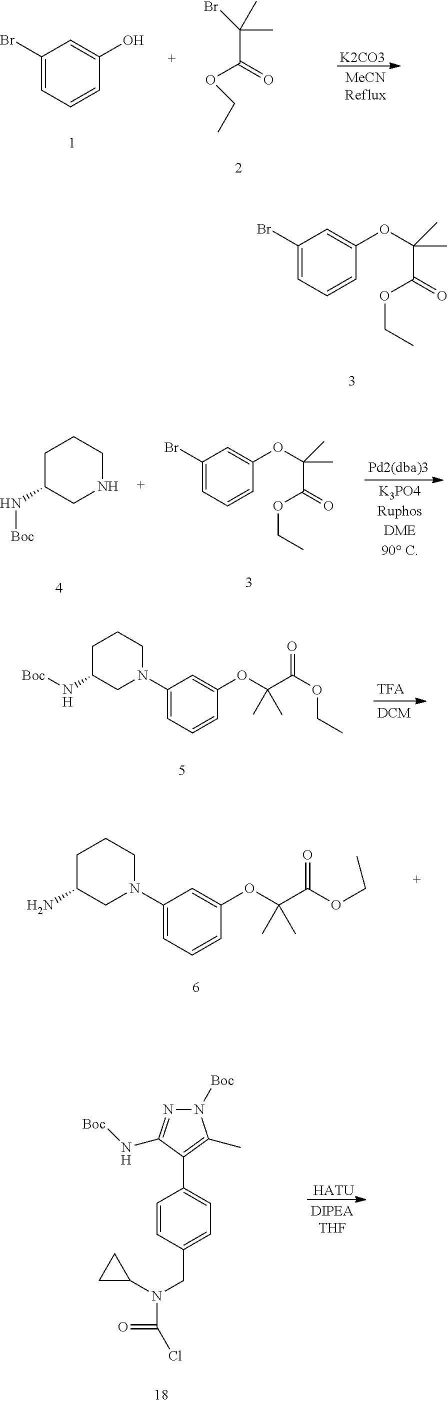

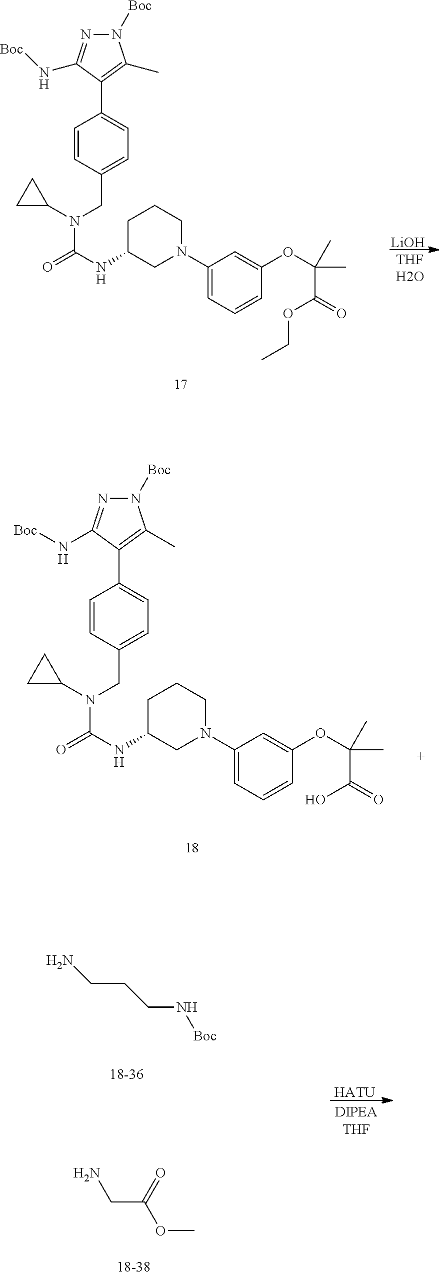

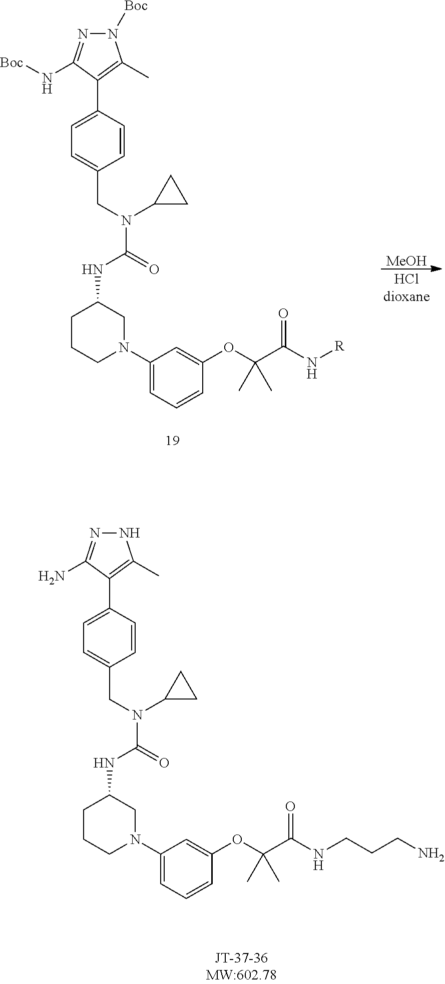

### Example 1.8: Compounds C37-043 to C37-044 can be Synthesized According to the Following Process

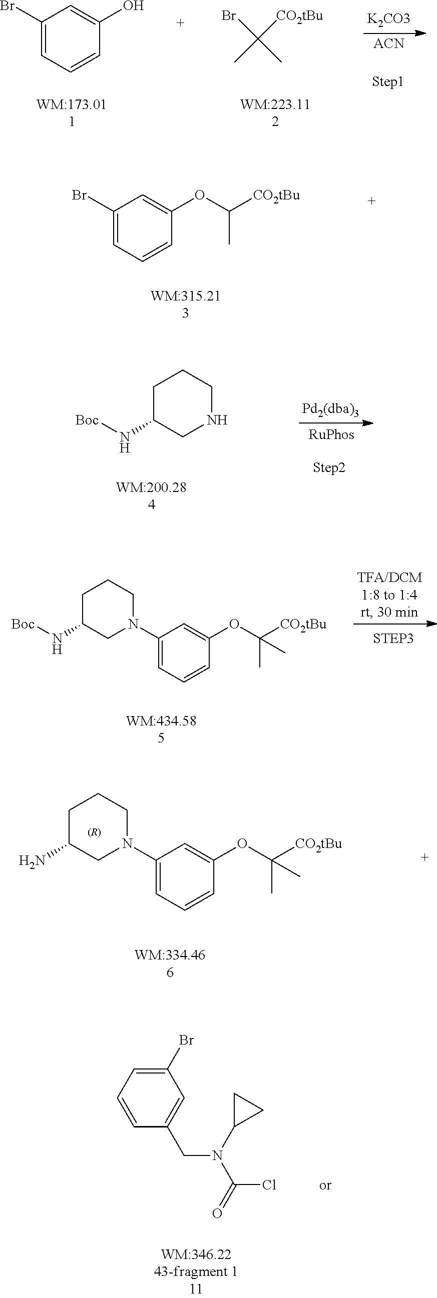

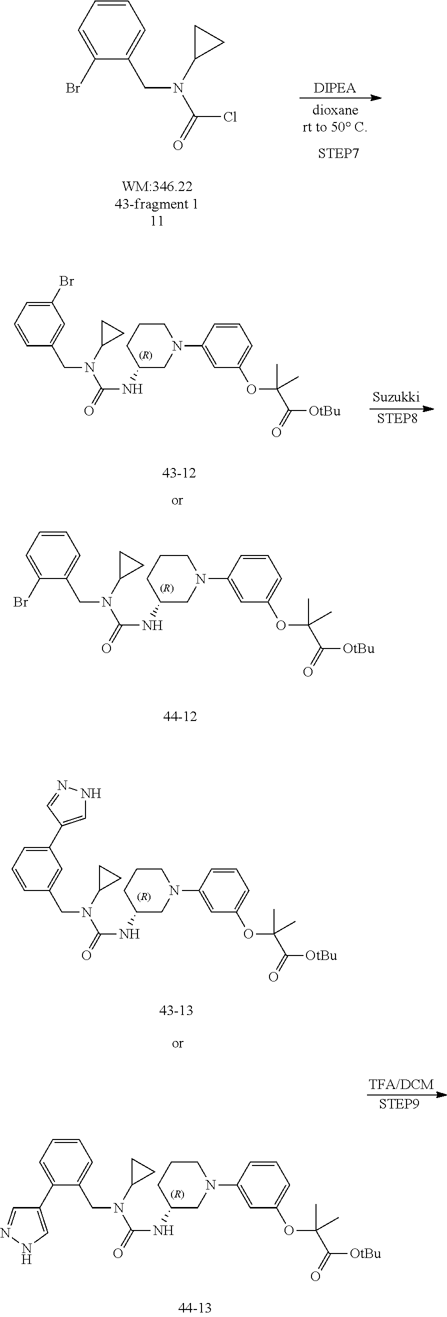

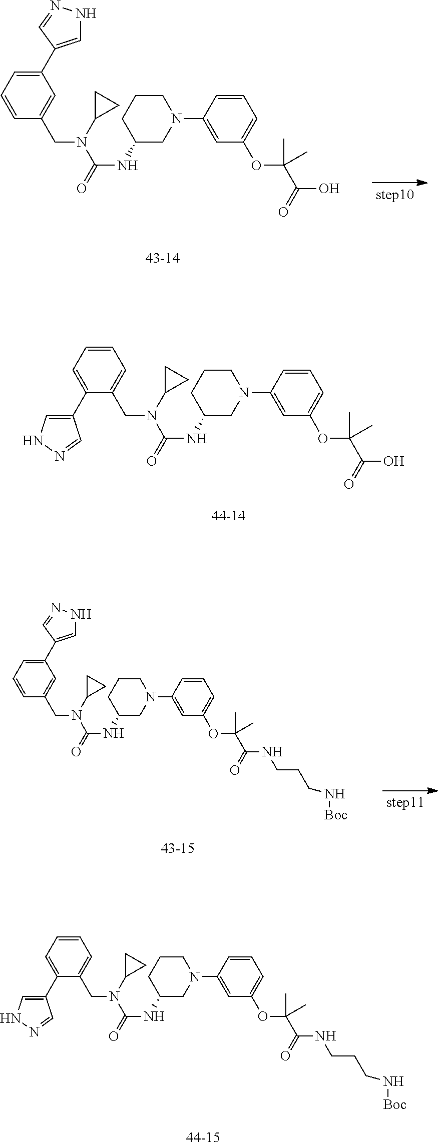 [SMILES: CC(C)(Oc1cccc(N2CCC[C@@H](NC(=O)N(Cc3cccc(-c4cn[nH]c4)c3)C3CC3)C2)c1)C(=O)O ; CC(C)(Oc1cccc(N2CCC[C@@H](NC(=O)N(Cc3ccccc3-c3cn[nH]c3)C3CC3)C2)c1)C(=O)O ; CC(C)(C)OC(=O)NCCCNC(=O)C(C)(C)Oc1cccc(N2CCC[C@@H](NC(=O)N(Cc3cccc(-c4cn[nH]c4)c3)C3CC3)C2)c1 ; CC(C)(C)OC(=O)NCCCNC(=O)C(C)(C)Oc1cccc(N2CCC[C@@H](NC(=O)N(Cc3ccccc3-c3cn[nH]c3)C3CC3)C2)c1]

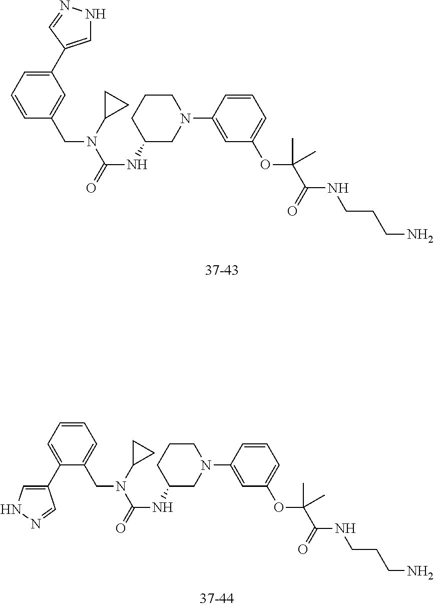 [SMILES: CC(C)(Oc1cccc(N2CCC[C@@H](NC(=O)N(Cc3cccc(-c4cn[nH]c4)c3)C3CC3)C2)c1)C(=O)NCCCN ; CC(C)(Oc1cccc(N2CCC[C@@H](NC(=O)N(Cc3ccccc3-c3cn[nH]c3)C3CC3)C2)c1)C(=O)NCCCN]

### Example 1.8: Compound C37-045 can be Synthesized According to the Following Process

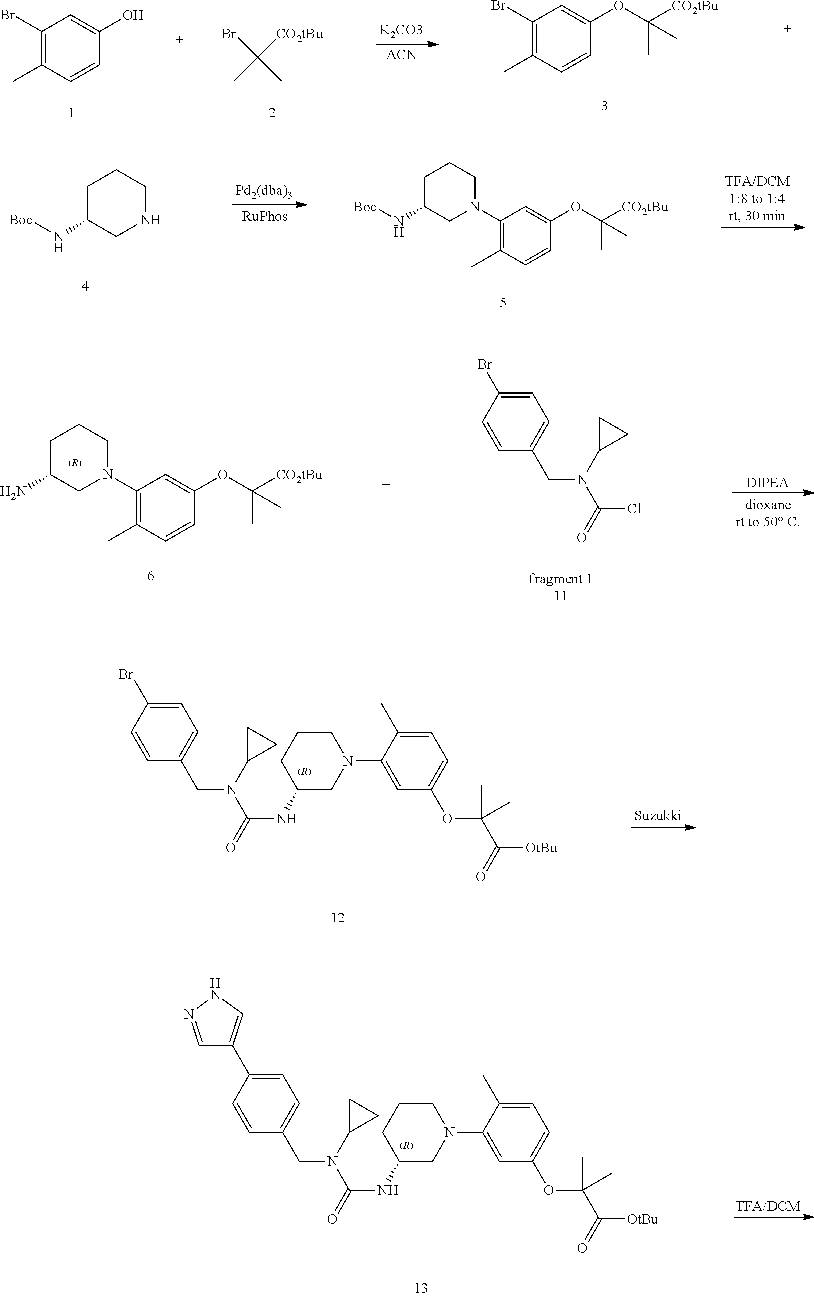

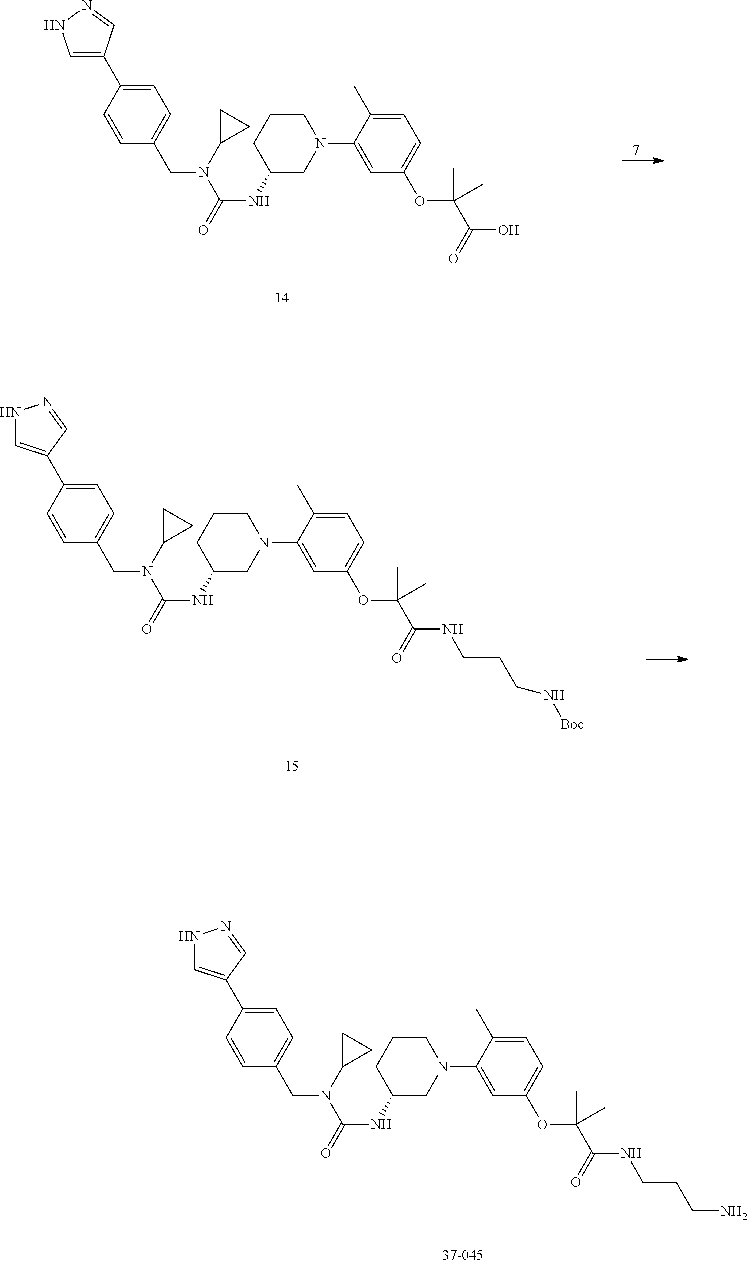 [SMILES: Cc1ccc(OC(C)(C)C(=O)O)cc1N1CCC[C@@H](NC(=O)N(Cc2ccc(-c3cn[nH]c3)cc2)C2CC2)C1 ; Cc1ccc(OC(C)(C)C(=O)NCCCNC(=O)OC(C)(C)C)cc1N1CCC[C@@H](NC(=O)N(Cc2ccc(-c3cn[nH]c3)cc2)C2CC2)C1 ; Cc1ccc(OC(C)(C)C(=O)NCCCN)cc1N1CCC[C@@H](NC(=O)N(Cc2ccc(-c3cn[nH]c3)cc2)C2CC2)C1]

### Example 1.7: Compound C37-046 can be Synthesized According to the Following Process

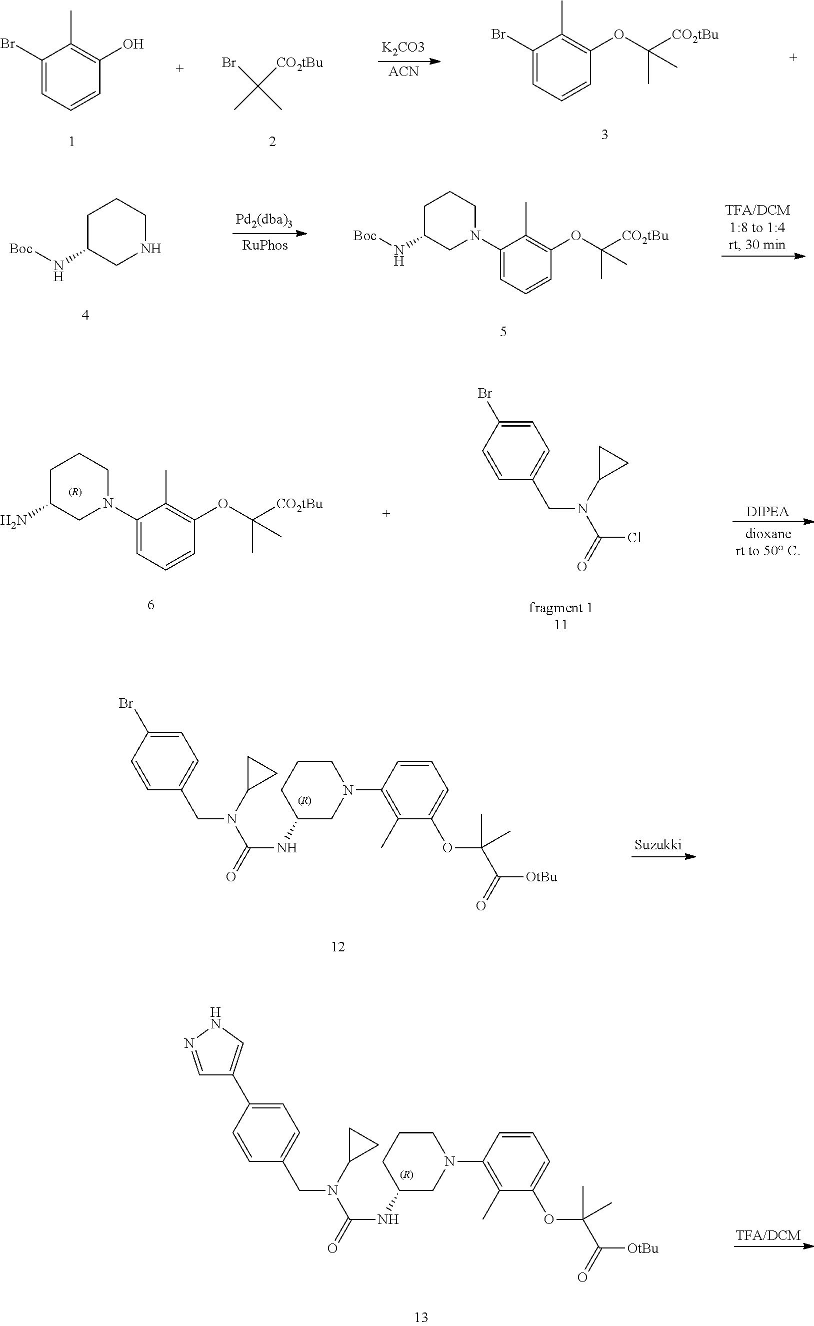

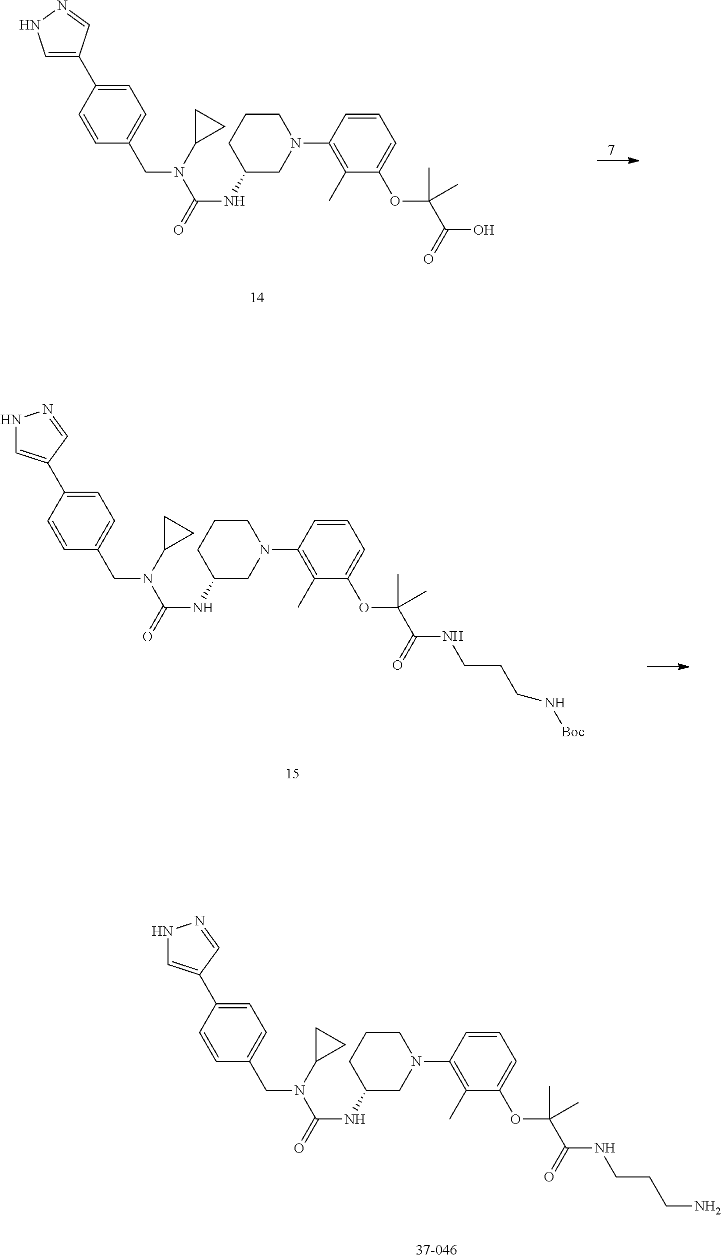 [SMILES: Cc1c(OC(C)(C)C(=O)O)cccc1N1CCC[C@@H](NC(=O)N(Cc2ccc(-c3cn[nH]c3)cc2)C2CC2)C1 ; Cc1c(OC(C)(C)C(=O)NCCCNC(=O)OC(C)(C)C)cccc1N1CCC[C@@H](NC(=O)N(Cc2ccc(-c3cn[nH]c3)cc2)C2CC2)C1 ; Cc1c(OC(C)(C)C(=O)NCCCN)cccc1N1CCC[C@@H](NC(=O)N(Cc2ccc(-c3cn[nH]c3)cc2)C2CC2)C1]

### Preparation Example 2

### Example 2.1

i. Compounds 1 to 6 were synthesized as described in Scheme 1.

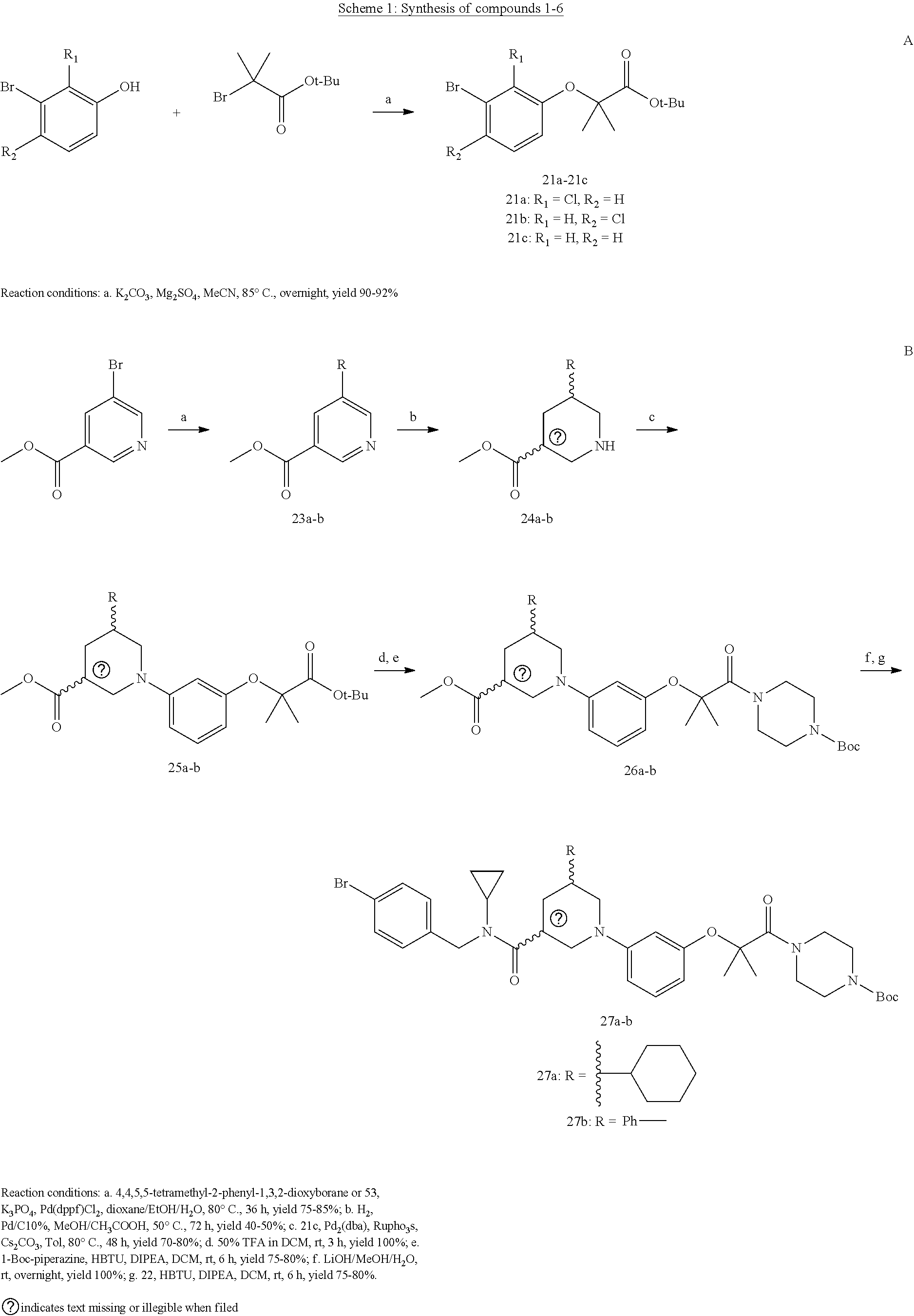

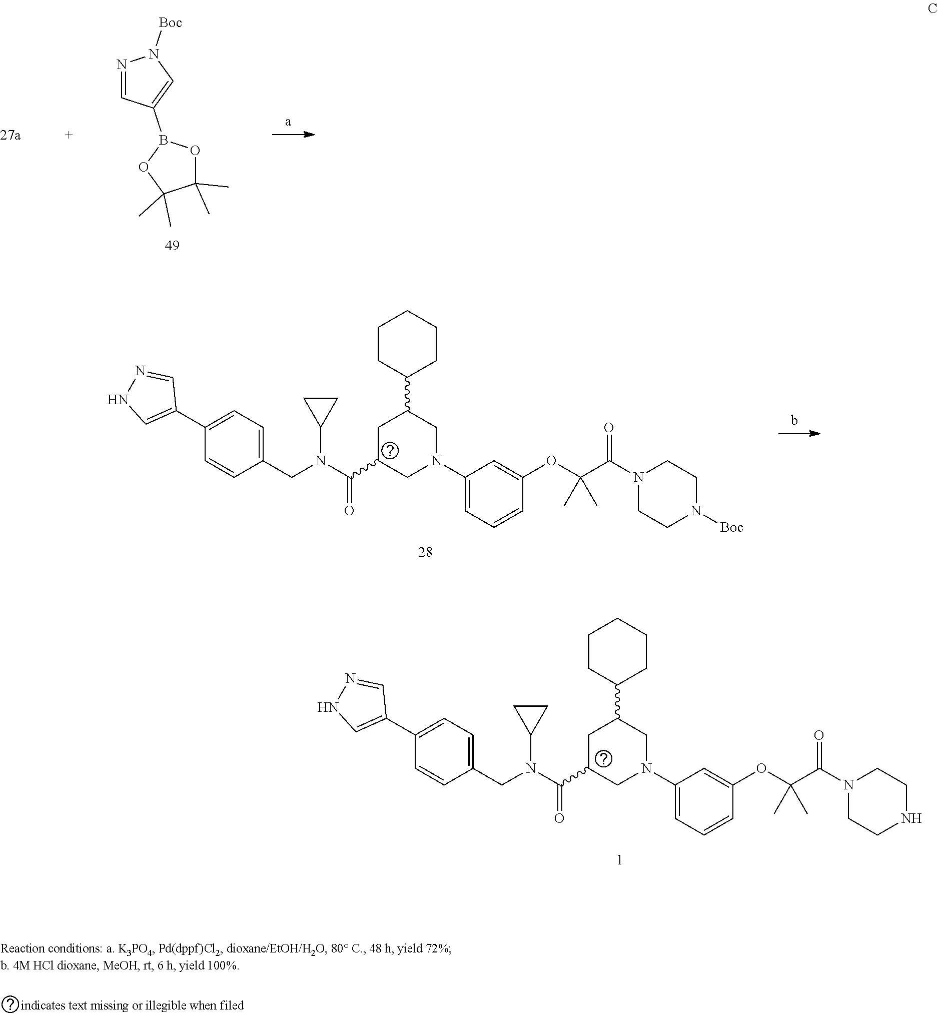 [SMILES: CC(C)(C)OC(=O)n1cc(B2OC(C)(C)C(C)(C)O2)cn1 ; CC(C)(C)OC(=O)N1CCN(C(=O)C(C)(C)Oc2cccc(N3CC(C(=O)N(Cc4ccc(-c5cn[nH]c5)cc4)C4CC4)CC(C4CCCCC4)C3)c2)CC1 ; CC(C)(Oc1cccc(N2CC(C(=O)N(Cc3ccc(-c4cn[nH]c4)cc3)C3CC3)CC(C3CCCCC3)C2)c1)C(=O)N1CCNCC1]

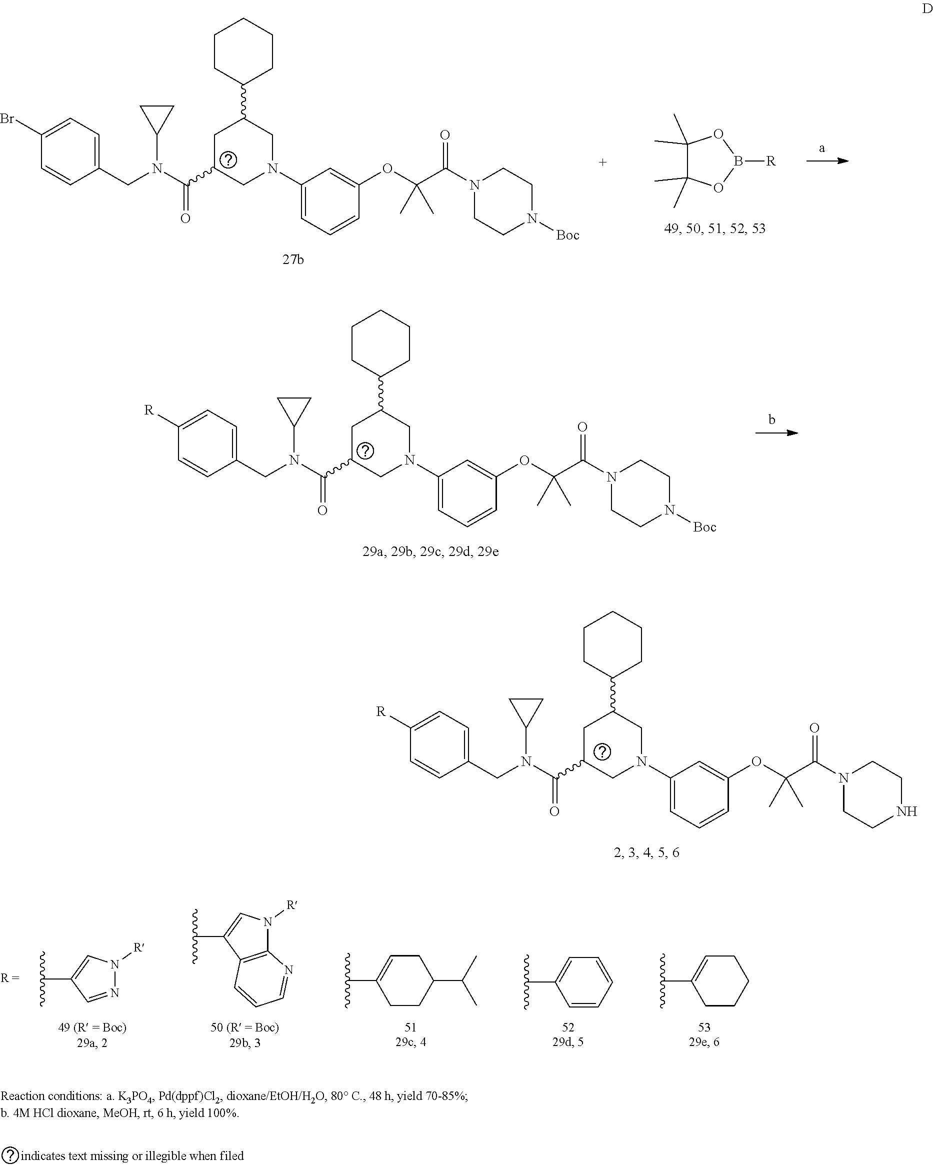

ii. Specific steps are as follows:

### General Steps for Synthesis of Intermediate 21a-c

3-A solutions of 3-bromophenol (1.00 g, 4.48 mmol), t-butyl 2-bromo-2-methylpropionate (1.55 g, 8.96 mmol), K₂CO₃ (2.47 g, 17.9 mmol) and MgSO₄ (0.54 g, 4.48 mmol) in MeCN was stirred at 85° C. overnight. MeCN was removed under reduced pressure. The residue was added with water and ethyl acetate. The obtained organic layer was washed with brine, dried over Na₂SO₄, and concentrated under vacuum. The residue was purified by column chromatography to yield the target compound 21a (1.27 g, 90% yield). The synthetic procedures of 21b and 21c were the same as that of 21a.

### General Steps for Synthesis of Intermediates 22 and 34

A solution of 4-bromobenzaldehyde (10 g, 54 mmol) and cyclopropylamine (18.50 g, 324 mmol) in methanol (100 mL) was stirred overnight at room temperature under N₂ protection. NaBH₄ (4.10 g, 108 mmol) was then added to the reaction mixture in batches at 0° C. The mixture was stirred at 0° C. for 1 hour. The reaction was quenched by adding saturated NH₄Cl, the solvent was removed under reduced pressure, then ethyl acetate and water were added. The organic layer was collected, washed with brine, dried over anhydrous NaS₂O₄, and concentrated under reduced pressure, and the residue was purified by column chromatography to give product 22 (9.78 g, 80% yield). The synthetic procedures of 34 was the same as that of 22.

### General Steps for Synthesis of Intermediate 23a-b

To a solution of bromobenzene derivative (3.00 g, 13.8 mmol) in dioxane/ethanol/water (25/10/5 mL) was added with boric acid pinacol ester (1.2 mmol), Pd(dppf)Cl₂ (1.01 g, 1.40 mmol), and K₃PO₄ (8.83 g, 41.67 mmol). The reaction mixture was heated to 80° C. under argon gas and stirred overnight. The reaction mixture was cooled to room temperature, concentrated under reduced pressure, and then redissolved with ethyl acetate, the solution was washed with water and brine, and dried over NaS₂O₄. The resulting solution was concentrated under vacuum. The residue was purified by column chromatography to obtain the target compound 23a (2.28 g, 77.21% yield). The synthetic procedure of 23b was the same as that of 23a.

### General Steps for Synthesis of Intermediate 24a-b

Pd/C (0.2 g, 10% by weight) was added to the solution of pyridine derivative (2 g, 9.38 mmol) in methanol/acetic acid (20/20 ml). The reaction mixture was heated to 50° C. for 72 h under a 2PSI hydrogen balloon. The reaction mixture was cooled to room temperature and concentrated under reduced pressure, then redissolved with water and ethyl acetate, and the pH of the solution was adjusted to 9-10 with saturated sodium carbonate. The organic layer was collected, washed with brine, dried over anhydrous NaS₂O₄, and concentrated under reduced pressure to obtain the target compound (1.02 g, 50.01% yield) which was directly used in the next step. The synthesis procedure for 24a was the same as that for 24b.

### General Steps for Synthesis of Intermediate 25a-b

A solution of amine (1.02 g, 1 eq), bromobenzene derivative (1.72 g, 1.2 eq), Pd₂(dba)₃ (0.1 eq), RuPhos (0.2 eq) and Cs₂CO₃ (5.94 g, 4 eq) in toluene (50 mL) was heated to 80° C. under N₂ and stirred overnight. The reaction mixture was cooled to room temperature. The solids were removed, the filtrate was concentrated under reduced pressure and purified by column chromatography to give the target compound 25a (1.65 g, 80% yield). The synthesis procedure for 25b was the same as that for 25a.

### General Steps for Synthesis of Intermediate 26a-b

TFA (20 mL) was added dropwise to a solution of tert-butyl ester (1.65 g) in CH₂Cl₂ (20 mL) at 0° C. The reaction mixture was stirred at 20° C. for 6 hours. After completion, the solvent was evaporated under reduced pressure. TFA was removed by adding CH₂Cl₂ for 3 times to give the desired product (1.44 g). To a mixture of carboxylic acid (1.44 g, 1 eq), amine (0.81 g, 1.2 eq) and HBTU (1.37 g, 2 eq) in CH₂Cl₂ (40 mL) was added dropwise with N,N-diisopropylethylamine (DIPEA) (1.87 g, 4 eq). The reaction mixture was warmed to room temperature and stirred overnight. After the reaction was complete, more CH2Cl2 was added and the organic phase was washed with 1M HCl, saturated NaHCO₃ and brine, dried over anhydrous Na₂SO₄, and concentrated under vacuum. The target compound 26a (1.64 g, 80% yield) was purified by column chromatography. The synthetic procedure of 26b was the same as that of 26a.

### General Steps for Synthesis of Intermediate 27a-b

To a solution of ethyl ester (1.0 g, 1 eq) in THF (16 mL) and methanol (4 mL) was added with LiOH (0.096 g, 2 eq) in H₂O (4 mL). The mixture was stirred for 3 hours at room temperature. After the reaction was complete, the solvent was removed under reduced pressure and the residue was redissolved in H₂O and acidified with 1M HCl. Ethyl acetate was added, the organic phase was washed with brine, dried over anhydrous Na₂SO₄, and concentrated under reduced pressure to obtain the target compound, which was directly used in the next step. To a mixture of carboxylic acid (0.97 g, 1 eq), amine (0.48 g, 1.2 eq) and HBTU (1.33 g, 2 eq) in CH₂Cl₂ (40 mL) was added dripwise with N,N-diisopropylethylamine (DIPEA) (0.91 g, 4 eq). The reaction mixture was warmed to room temperature and stirred overnight. After the reaction was complete, more CH₂Cl₂ was added and the organic phase was washed with 1M HCl, saturated NaHCO₃ and brine, dried over anhydrous Na₂SO₄ and concentrated under vacuum. The target compound 27a (1.01 g, 75.67% yield) was purified by column chromatography. The synthetic procedure of 27b was the same as that of 27a.

### General Steps for Synthesis of Intermediate 28

To a solution of bromobenzene derivative (0.50 g, 1 eq) in dioxane/ethanol/water (25/10/5 mL) was added with boric acid pinacol ester (0.23 g, 1.2 eq), Pd(dppf)Cl₂ (0.047 g, 0.1 eq) and K₃PO₄ (0.41 g, 3 eq). The reaction mixture was heated to 80° C. under argon gas and stirred overnight. The reaction mixture was cooled to room temperature, concentrated under reduced pressure, then redissolved in ethyl acetate, washed with water and brine, and dried over Na₂SO₄. The resulting solution was concentrated under a vacuum. The residue was purified by column chromatography to yield the target compound 28 (0.35 g, 72% yield).

### General Steps for the Synthesis of Compound 1

To a solution of Boc-protected amine (0.35 g, 1 eq) in 10 mL of methanol was added with 4M dioxane hydrochloride (2 mL) at room temperature. The reaction mixture was stirred at 20° C. for 6 hours. The resulting solution was concentrated under a vacuum. The residue was dissolved in MeOH/H₂O (2/6 ml), frozen at −40° C. for 4 h, and lyophilized for 12 h to obtain the target compound 1 (0.3 g, 100% yield).

### General Steps for Synthesis of Intermediate 29a-e

To a solution of bromobenzene derivative (0.50 g, 1 eq) in dioxane/ethanol/water (25/10/5 mL) was added the boric acid pinacol ester (0.23 g, 1.2 eq), Pd(dppf)Cl₂ (0.048 g, 0.1 eq) and K₃PO₄ (0.44 g, 3 eq). The reaction mixture was heated to 80° C. under argon gas and stirred overnight. The reaction mixture was cooled to room temperature, concentrated under reduced pressure, then redissolved in ethyl acetate, washed with water and brine, and dried over NaS₂O₄. The resulting solution was concentrated under a vacuum. The residue was purified by column chromatography to yield the target compound 28 (0.38 g, 73% yield). The synthetic procedures of 29b, 29c, 29d and 29e were the same as that of 29a.

### General Steps for the Synthesis of Compounds 2-6

To a solution of Boc-protected amine (0.38 g, 1 eq) in 10 mL methanol was added with 4M dioxane hydrochloride (2 mL) at room temperature. The reaction mixture was stirred at 20° C. for 6 hours. The resulting solution was concentrated under vacuum. The residue was dissolved in MeOH/H₂O (2/6 ml), frozen at −40° C. for 4 h, and lyophilized for 12 h to obtain the target compound 2 (0.3 g, 10000 yield). The synthesis procedures for 3, 4, 5, and 6 were the same as that for compound 2.

### Example 2.2

i. Compounds 7-14 were synthesized as described in Scheme 2.

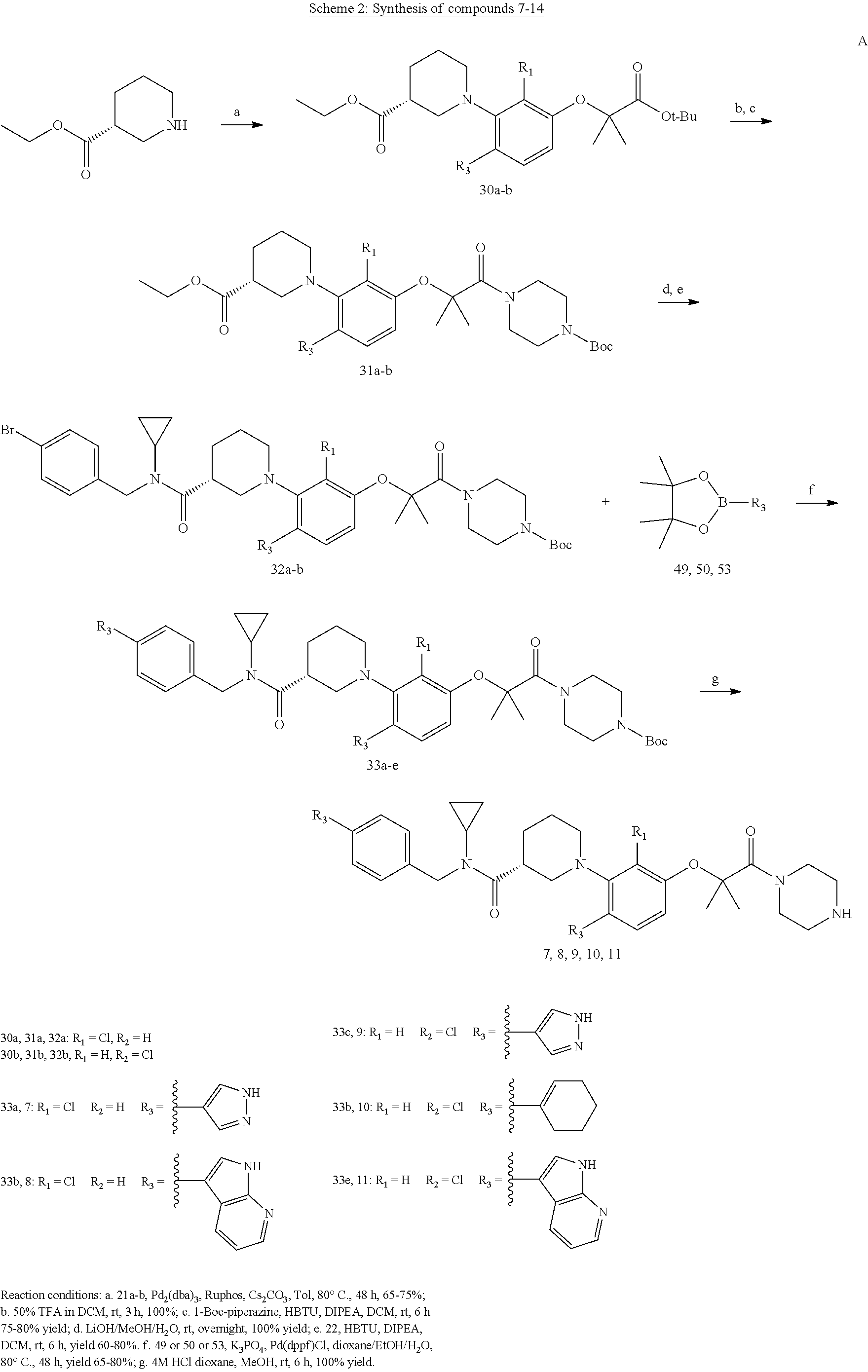

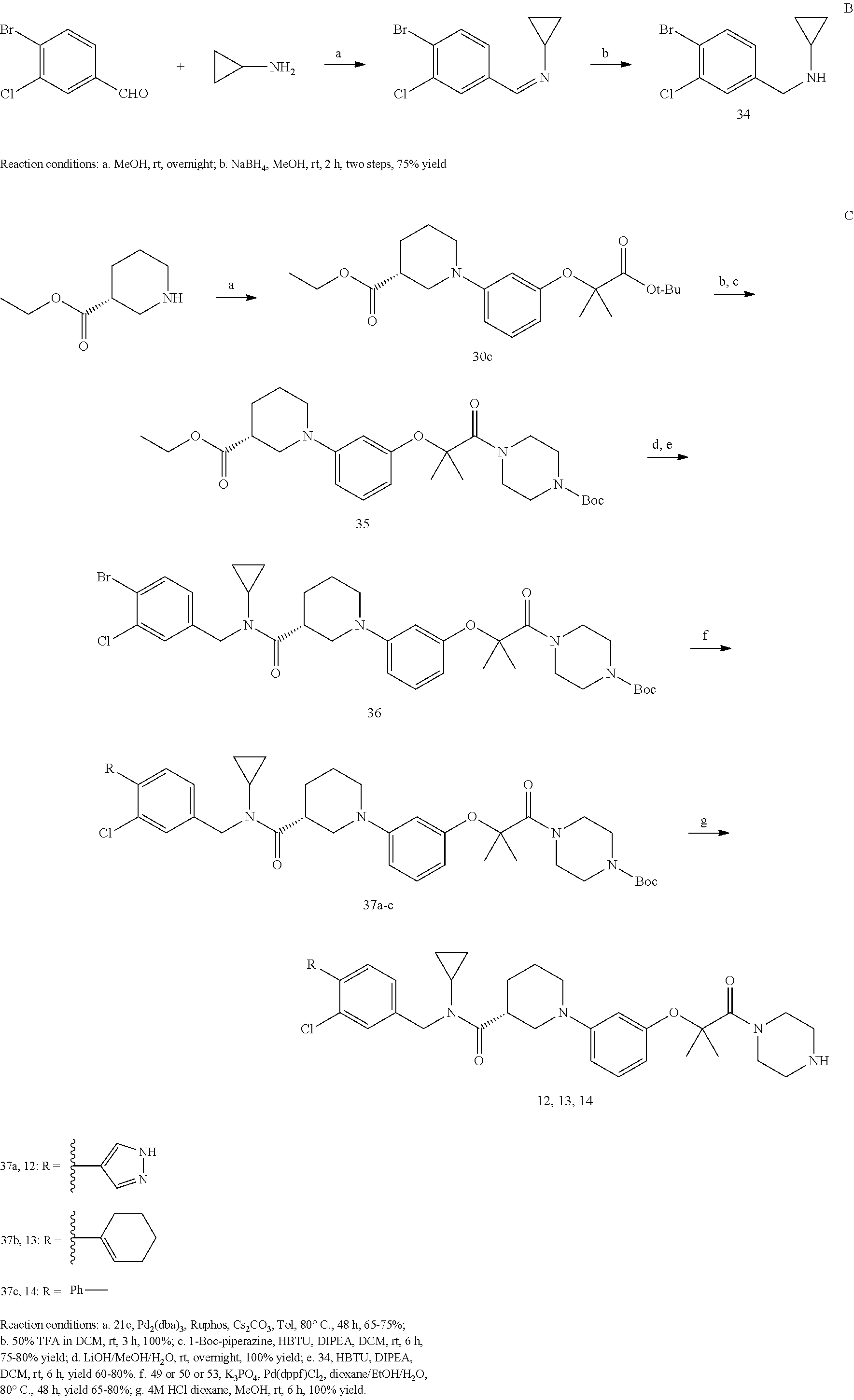

ii. Specific steps are as follows:

### General Steps for Synthesis of Intermediate 30a-c

A solution of amine (0.27 g, 1.2 eq), bromobenzene derivative (0.5 g, 1 eq), Pd₂(dba)₃ (0.13 g, 0.1 eq), RuPhos (0.133 g, 0.2 eq) and Cs₂CO₃ (5.94 g, 4 eq) in toluene (30 mL) was heated to 80° C. under N2 and stirred overnight. The reaction mixture was cooled to room temperature. The solids were removed, the filtrate was concentrated under reduced pressure and purified by column chromatography to give the target compound 30a (0.45 g, 80% yield). The synthesis procedures for 30b and 30c were the same as those for 30a.

### General Steps for Synthesis of Intermediates 31a-b and 35

TFA (20 mL) was added dropwise to a solution of tert-butyl ester (0.45 g) in CH₂Cl₂ (20 mL) at 0° C. The reaction mixture was stirred at 20° C. for 6 hours. After completion, solvent evaporated under reduced pressure. TFA was removed by adding CH₂Cl₂ for 3 times to give the desired product (0.39 g). N,N-diisopropylethylamine (DIPEA) (0.54 g, 4 eq) was added dropwise to a mixture of carboxylic acid (0.39 g, 1 eq), amine (0.24 g, 1.2 eq), and HBTU (0.8 g, 2 eq) in CH₂Cl₂ (40 mL). The reaction mixture was warmed to room temperature and stirred overnight. After the reaction was complete, more CH₂Cl₂ was added, the organic phase was washed with 1M HCl, saturated NaHCO₃, and brine, dried over anhydrous Na₂SO₄, and concentrated under vacuum. The target compound 31a (0.42 g, 64% yield) was purified by column chromatography. The synthetic procedures of 31b and 35 were the same as that of 31a.

### General Steps for Synthesis of Intermediates 32a-b and 36

To a solution of ethyl ester (0.42 g, 1 eq) in THF (16 mL) and methanol (4 mL) was added with LiOH (0.037 g, 2 eq) in H₂O (4 mL). The mixture was stirred for 3 hours at room temperature. After the reaction was complete, the solvent was removed under reduced pressure and the residue is redissolved in H₂O and acidified with 1M HCl. Ethyl acetate was added, the organic phase was washed with brine, dried over anhydrous Na₂SO₄ and concentrated under reduced pressure to obtain the target compound, which was directly used in the next step. N,N-diisopropylethylamine (DIPEA) (0.41 g, 4 equivalents) was added dropwise to the mixture of carboxylic acid (0.41 g, 1 eq), amine (0.21 g, 1.2 eq), and HBTU (0.59 g, 2 eq) in CH₂Cl₂ (40 mL). The reaction mixture was warmed to room temperature and stirred overnight. After the reaction was complete, more CH₂Cl₂ was added, the organic phase was washed with 1M HCl, saturated NaHCO₃, and brine, dried over anhydrous Na₂SO₄, and concentrated under vacuum. The target compound 32a (0.43 g, 77% yield) was purified by column chromatography. The synthetic procedures of 32b and 36 were the same as that of 32a.

### General Steps for Synthesis of Intermediate 33a-e

To a solution of bromobenzene derivative (0.20 g, 1 eq) in dioxane/ethanol/water (25/10/5 mL) was added with boric acid pinacol ester (0.10 g, 1.2 eq), Pd(dppf)Cl₂ (0.021 g, 0.1 eq) and K₃PO₄ (0.177 g, 3 eq). The reaction mixture was heated to 80° C. under argon gas and stirred overnight. The reaction mixture was cooled to room temperature, concentrated under reduced pressure, then redissolved in ethyl acetate, washed with water and brine, and dried over NaS₂O₄. The resulting solution was concentrated under vacuum. The residue was purified by column chromatography to yield the target compound 33a (0.12 g, 63% yield). The synthetic procedures of 33b, 33c, 33d, and 33e were the same as that of 33a.

### General Steps for the Synthesis of Compounds 7-11

4M dioxane hydrochloride (2 mL) was added to a solution of Boc-protected amine (0.12 g, 1 eq) in 10 mL methanol at room temperature. The reaction mixture was stirred at 20° C. for 6 hours. The resulting solution was concentrated under vacuum. The residue was dissolved in MeOH/H₂O (2/6 ml), frozen at −40° C. for 4 h, and lyophilized for 12 h to obtain the target compound 7 (0.1 g, 100% yield). The synthesis procedures for 8, 9, 10, and 11 was the same as for compound 7.

### General Steps for Synthesis of Intermediate 37a-c

To a solution of brominobenzene derivative (0.10 g, 1 eq) in dioxane/ethanol/water (10/4/2 mL) was added with the boric acid pinacol ester (0.05 g, 1.2 eq), Pd(dppf)Cl₂ (0.011 g, 0.1 eq) and K₃PO₄ (0.09 g, 3 eq). The reaction mixture was heated to 80° C. under argon gas and stirred overnight. The reaction mixture was cooled to room temperature, concentrated under reduced pressure, then redissolved in ethyl acetate, washed with water and brine, and dried over Na₂SO₄. The resulting solution was concentrated under vacuum. The residue was purified by column chromatography to yield the target compound 37a (0.063 g, 65% yield). The synthesis procedures for 37b and 37c were the same as that for 37a.

### General Steps for the Synthesis of Compounds 12-14

4M dioxane hydrochloride (2 mL) was added to a solution of Boc-protected amines (0.063 g, 1 eq) in 10 mL of methanol at room temperature. The reaction mixture was stirred at 20° C. for 6 hours. The resulting solution was concentrated under vacuum. The residue was dissolved in MeOH/H₂O (2/6 ml), frozen at −40° C. for 4 h, and then lyophilized for 12 h to obtain the target compound 12 (0.054 g, 100% yield). The synthesis procedure for 13 and 14 was the same as that for compound 12.

### Example 2.3

i. Compounds 15-16 were synthesized as described in Scheme 3.

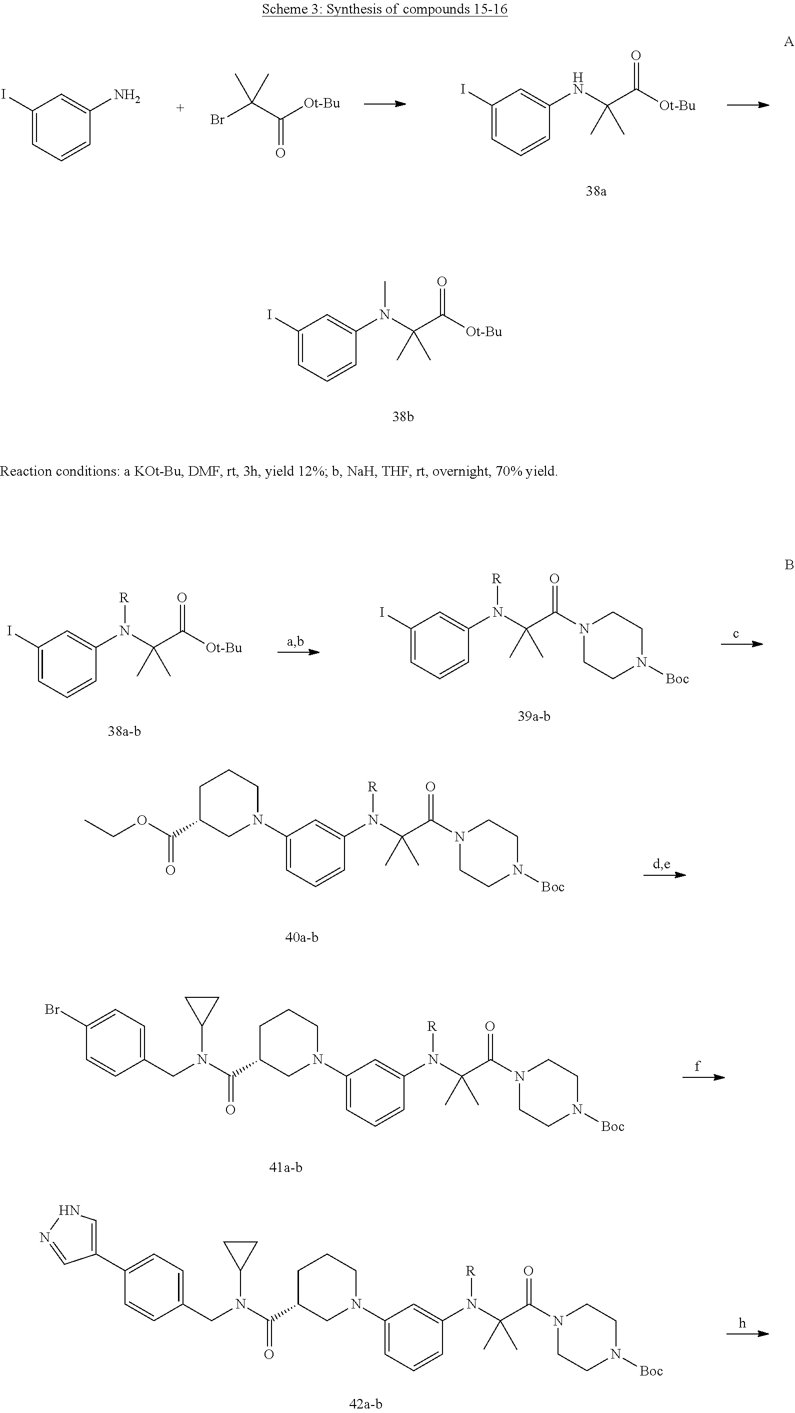

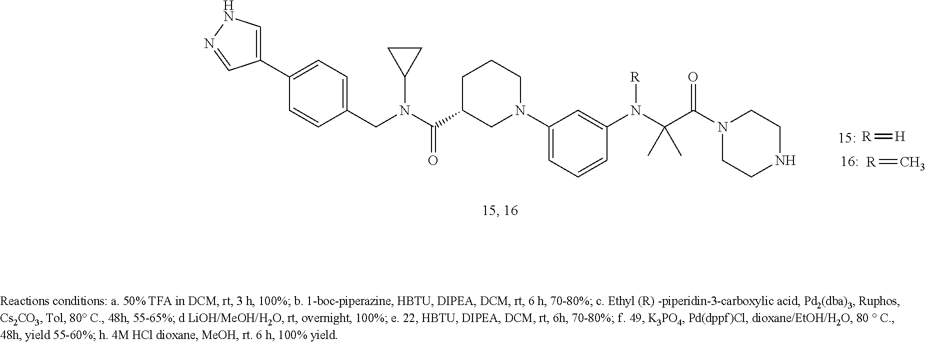

ii. Specific steps are as follows:

### General Steps for Synthesis of Intermediate 38a

A solution of 3-iodoaniline (5 g, 1 eq), tert-butyl 2-bromo-2-methylpropionate (15.25 g, 3 eq), KOtBu (6.65 g, 3 eq) in DMF was stirred at 0° C. for 3 hours. Water and ethyl acetate were added. The obtained organic layer was washed with brine, dried over Na₂SO₄, and concentrated under vacuum. The residue was purified by column chromatography to yield the target compound 38a (1.2 g, 15% yield).

### General Steps for Synthesis of Intermediate 38b

A solution of tert-butyl 2-((3-iodophenyl)amino)-2-methylpropionate (0.6 g, 1 eq), 60% NaH (0.66 g, 1.5 eq) in THF was stirred at 0° C. for 1 hour. CH₃I (1.2 eq) was added dropwise to the solution of tert-butyl ester in THF (30 mL). The reaction mixture was stirred at 20° C. for 6 hours. Water (10 mL) was added. The resulting solution was concentrated under vacuum, then re-dissolved in ethyl acetate, washed with water and brine, and dried over Na₂SO₄. The resulting solution was concentrated under vacuum. The residue was purified by column chromatography to yield the target compound 38b (0.43 g, 70% yield).

### General Steps for Synthesis of Intermediate 39a-b

TFA (20 mL) was added dropwise to a solution of tert-butyl ester (0.43 g) in CH₂Cl₂ (20 mL) at 0° C. The reaction mixture was stirred at 20° C. for 6 hours. After completion, the solvent was evaporated under reduced pressure. TFA was removed by adding CH₂Cl₂ for 3 times to give the desired product (0.37 g). N,N-diisopropylethylamine (DIPEA) (0.54 g, 4 eq) was added dropwise to the mixture of carboxylic acid (0.37 g, 1 eq), amine (0.24 g, 1.2 eq), and HBTU (0.8 g, 2 eq) in CH₂Cl₂ (40 mL). The reaction mixture was warmed to room temperature and stirred overnight. After the reaction was complete, more CH₂Cl₂ was added, the organic phase was washed with 1M HCl, saturated NaHCO₃, and brine, dried over anhydrous Na₂SO₄, and concentrated under vacuum. The target compound 39a (0.56 g, 74% yield) was purified by column chromatography. The synthetic procedure of 39b was the same as that of 39a.

### General Steps for Synthesis of Intermediate 40a-b

A solution of amine (0.22 g, 1.2 eq), bromobenzene derivative (0.56 g, 1 eq), Pd₂(dba) (0.11 g, 0.1 eq), RuPhos (0.11 g, 0.2 eq) and Cs₂CO₃ (1.54 g, 4 eq) in toluene (50 mL) were heated to 80° C. under N₂ and stirred overnight. The reaction mixture was cooled to room temperature. The solids were removed, the filtrate was concentrated under reduced pressure and purified by column chromatography to give the target compound 40a (0.45 g, 80% yield). The synthetic procedure of 40b was the same as that of 40a.

### General Steps for Synthesis of Intermediate 41a-b

To a solution of ethyl ester (0.36 g, 1 eq) in THF (16 mL) and methanol (4 mL) was added with LiOH (0.034 g, 2 eq) in H₂O (4 mL). The mixture was stirred for 3 hours at room temperature. After the reaction is complete, the solvent was removed under reduced pressure and the residue was redissolved in H₂O and acidified with 1M HCl. Ethyl acetate was added, the organic phase was washed with brine, dried over anhydrous Na₂SO₄, and concentrated under reduced pressure to obtain the target compound, which was directly used in the next step. N,N-diisopropylethylamine (DIPEA) (0.37 g, 4 eq) was added dropwise to the mixture of carboxylic acid (0.34 g, 1 eq), amine (0.19 g, 1.2 eq), and HBTU (0.53 g, 2 eq) in CH₂Cl₂ (40 mL). The reaction mixture was stirred overnight at room temperature. After the reaction was complete, more CH₂Cl₂ was added, the organic phase was washed with 1M HCl, saturated NaHCO₃, and brine, dried over anhydrous Na₂SO₄, and concentrated under vacuum. The target compound 41a (0.34 g, 70% yield) was purified by column chromatography. The synthetic procedure of 41b was the same as that of 41a.

### General Steps for Synthesis of Intermediate 42a-b

To a solution of bromobenzene derivative (0.34 g, 1 eq) in dioxane/ethanol/water (25/10/5 mL) was added with boric acid pinacol ester (0.17 g, 1.2 eq), Pd(dppf)Cl₂ (0.036 g, 0.1 eq), and K₃PO₄ (0.32 g, 3 eq) were added. The reaction mixture was heated to 80° C. under argon gas and stirred overnight. The reaction mixture was cooled to room temperature, concentrated under reduced pressure, then redissolved in ethyl acetate, washed with water and brine, and dried over NaS₂O₄. The resulting solution was concentrated under vacuum. The residue was purified by column chromatography to yield the target compound 42a (0.35 g, 72% yield). The synthetic procedure of 42b was the same as that of 42a.

### General Steps for the Synthesis of Compounds 15-16

4M dioxane hydrochloride (2 mL) was added to a solution of Boc-protected amine (0.1 g, 1 eq) in 10 mL of methanol at room temperature. The reaction mixture was stirred at 20° C. for 6 hours. The resulting solution was concentrated under vacuum. The residue was dissolved in MeOH/H₂O (2/6 ml), frozen at −40° C. for 4 h, and then lyophilized for 12 h to obtain the target compound 15 (0.085 g, 10000 yield). The synthesis procedure for 16 was the same as that for compound 15.

### Example 2.4

i. Compounds 17-20 were Synthesized as Described in Scheme 1.

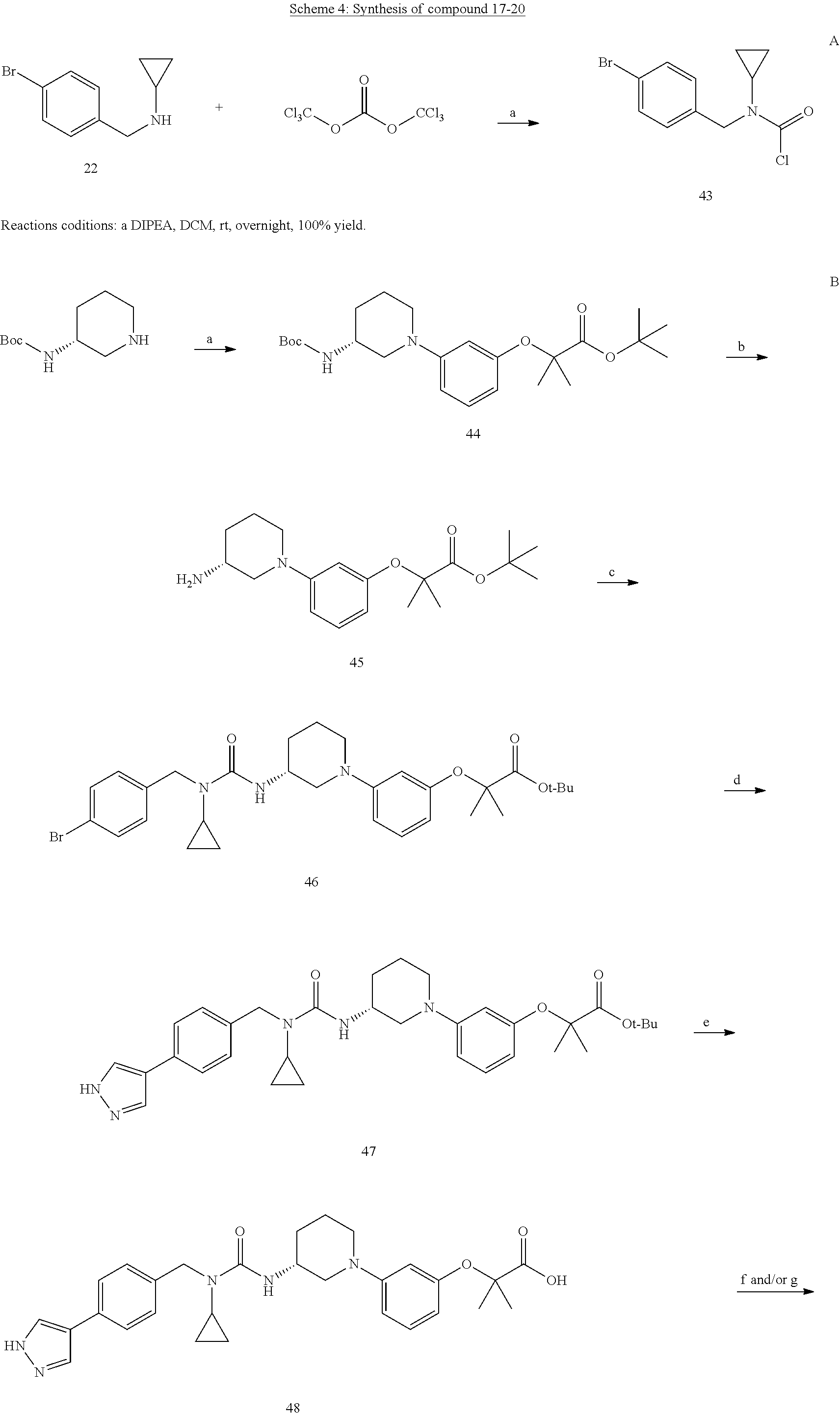

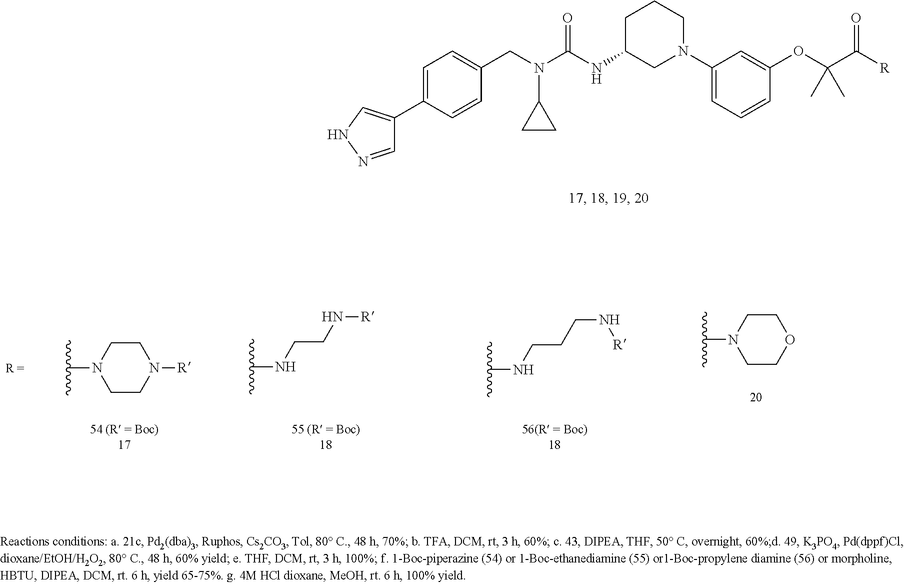

ii. Specific Steps are as Follows:

### General Steps for Synthesis of Intermediate 43

At 0° C., triphosgene (6.5 g, 2 eq) was added dropwise to a solution of N-[(4-bromophenyl)methyl]cyclopropylamine (2.5 g, 1 eq) in THF (50 mL). The reaction mixture was stirred at 20° C. overnight. After completion, the solvent was evaporated under reduced pressure and directly used in the next step.

### General Steps for Synthesis of Intermediate 44

A solutions of amine (1.52 g, 1.2 eq), bromobenzene derivative (2.0 g, 1 eq), Pd₂(dba)₃ (0.58 g, 0.1 eq), RuPhos (0.59 g, 0.2 eq) and Cs₂CO₃ (8.25 g), 4 eq) in toluene (80 mL) was heated under N₂ to 80° C. and stirred overnight. The reaction mixture was cooled to room temperature. The solids were removed, the filtrate was concentrated under reduced pressure and purified by column chromatography to give the target compound 44 (1.9 g, 75% yield).

### General Steps for Synthesis of Intermediate 45

TFA (2 mL) was added dropwise to a solution of tert-butyl ester (1.50 g) in CH₂Cl₂ (20 m1) at 0° C. The reaction mixture was stirred at 20° C. for 4 hours. After completion, the solvent was evaporated under reduced pressure, then the residue was re-dissolved in CH₂Cl₂, washed with 1M NaOH and brine, and dried over Na₂SO₄. The resulting solution was concentrated under vacuum and used directly in the next step.

### General Steps for Synthesis of Intermediate 46

N,N-diisopropylethylamine (DIPEA) (0.37 g), 4 eq) was added dropwise to a mixture of amine (0.87 g, 1 eq) and (4-bromobenzyl)(cyclopropyl)carbamoyl chloride (0.90 g, 1.2 eq) in THF (40 mL). The reaction mixture was warmed to 50° C. and stirred overnight. After completion, the solvent was evaporated under reduced pressure, then the residue was redissolved in CH₂Cl₂, washed with water and brine, and dried over Na₂SO₄. The resulting solution was concentrated under vacuum. The residue was purified by column chromatography to yield the target compound 46 (0.91 g, 60% yield).

### General Steps for Synthesis of Intermediate 47

To a solution of bromobenzene derivative (0.45 g, 1 eq) in dioxane/ethanol/water (25/10/5 mL) was added the boric acid pinacol ester (0.26 g, 1.2 eq), Pd(dppf)Cl₂ (0.056 g, 0.1 eq) and K₃PO₄ (0.49 g, 3 eq). The reaction mixture was heated to 80° C. under argon gas and stirred overnight. The reaction mixture was cooled to room temperature, concentrated under reduced pressure, then the residue was redissolved in ethyl acetate, washed with water and brine, and dried over NaS₂O₄. The resulting solution was concentrated under vacuum. The residue was purified by column chromatography to yield the target compound 47 (0.53 g, 60% yield).

### General Steps for Synthesis of Intermediate 48

TFA (20 mL) was added dropwise to a solution of tert-butyl ester (0.53 g) in CH₂Cl₂ (20 mL) at 0° C. The reaction mixture was stirred at 20° C. for 6 hours. After completion, the solvent was evaporated under reduced pressure. TFA was removed by adding CH₂Cl₂ for 3 times to give the desired product (0.46 g).

### General Steps for Synthesis of Intermediates 54, 55, 56, and 20

N,N-diisopropyl ethylamine (DIPEA) (0.1 g, 4 equivalents) was added dropwise to a mixture of carboxylic acid (0.1 g, 1 eq), amine (0.043 g, 1.2 eq) and HATU (0.15 g, 2 eq) in THF (20 mL). The reaction mixture was warmed to room temperature and stirred overnight. After the reaction was complete, the solvent was evaporated under reduced pressure, then redissolved with CH₂Cl₂, washed with water and brine, and dried with Na₂SO₄. The resulting solution was concentrated under vacuum. The target compound 54 (0.09 g, 70% yield) was purified by column chromatography. The synthetic procedures of 55, 56, and 20 were the same as that of 54.

### General Steps for the Synthesis of Compounds 17, 18, and 19

4M dioxane hydrochloride (2 mL) was added to a solution of Boc-protected amines (0.09 g, 1 eq) in 10 mL of methanol at room temperature. The reaction mixture was stirred at 20° C. for 6 hours. The resulting solution was concentrated under vacuum. The residue was dissolved in MeOH/H₂O (2/6 ml), frozen at −40° C. for 4 h, and then lyophilized for 12 h to obtain the target compound 17 (0.077 g, 1000 yield). The synthesis procedure for 18 and 19 was the same as that for compound 17.

Characterization data for the intermediates and compounds 1=−20 synthesized in Examples 3.1-3.4 are as follows:

Unless otherwise stated, mass spectrometry data for each intermediate and compounds 1-20 were determined by ESI LCMS UV determination performed as follows, PLC (om, 4.6 mm×150 mm) on XBridge C18 column, Gradient water/acetonitrile+0.10% formic acid (0-100% acetonitrile, 10 min).

The purity of Compounds 1-20 were all more than 95% (column is Shimadu C18 (5 μm, 4.6 mm×250 mm), gradient water/acetonitrile=30:70 or 5:95. (20 min)).

i. Characterization data for each intermediate are as follows:

tert-butyl 2-(3-bromo-2-chlorophenoxy)-2-methylpropanoate (21a).
¹H NMR (400 MHz, Chloroform-d) δ 7.35 (dd, J = 8.0, 1.3 Hz, 1H), 7.22 (dd, J = 8.0, 7.0 Hz, 1H),
7.05 (dd, J = 7.0, 1.2 Hz, 1H), 1.59 (s, 6H), 1.43 (s, 9H). ESI-MS calculated for C₁₄H₁₈BrClO₃[M]⁺:
350.01, Observed: 350.29
tert-butyl 2-(3-bromo-4-chlorophenoxy)-2-methylpropanoate(21b).
¹H NMR (400 MHz, Chloroform-d) δ 7.33-7.28 (m, 2H), 6.88 (dd, J = 8.1, 2.2 Hz, 1H), 1.56 (s,
6H), 1.43 (s, 9H). ESI-MS calculated for C₁₄H₁₈BrClO₃[M]⁺: 350.01, Observed: 350.21
tert-butyl 2-(3-bromophenoxy)-2-methylpropanoate (21c)
¹H NMR (400 MHz, Chloroform-d) δ 7.30 (ddd, J = 8.0, 2.2, 1.3 Hz, 1H), 7.25 (dd, J = 8.1, 7.0
Hz, 1H), 7.07 (t, J = 2.2 Hz, 1H), 6.88 (ddd, J = 7.0, 2.3, 1.4 Hz, 1H), 1.56 (s, 6H), 1.43 (s,
9H). ESI-MS calculated for C₁₄H₁₉BrO₃[M]⁺: 315.21, Observed: 315.25
N-[(4-bromophenyl)methyl]cyclopropanamine. (22)
¹H NMR (400 MHz, Chloroform-d)δ 7.47-7.41 (m, 2H), 7.21 (dt, J = 7.9, 1.1 Hz, 2H), 4.12 (dt, J =
6.2, 3.9 Hz, 1H), 3.93 (ddt, J = 13.9, 4.0, 1.1 Hz, 1H), 3.86 (ddt, J = 13.9, 3.8, 0.9 Hz, 1H), 2.52
(dp, J = 6.2, 4.3 Hz, 1H), 0.65 (tddd, J = 11.7, 5.7, 3.2, 1.5 Hz, 4H). ESI-MS calculated for
C₁₀H₁₂BrN[M + H]⁺: 227.23, Observed: 227.12
N-[(4-bromo-3-chlorophenyl)methyl]cyclopropanamine (34).
¹H NMR (400 MHz, Chloroform-d)δ 7.48 (d, J = 7.9 Hz, 1H), 7.36 (dd, J = 2.2, 1.1 Hz, 1H), 7.17
(ddt, J = 8.0, 2.2, 1.0 Hz, 1H), 4.22 (dt, J = 6.4, 3.9 Hz, 1H), 3.95 (ddt, J = 13.7, 4.0, 1.0 Hz, 1H),
3.89 (ddt, J = 13.6, 3.8, 0.9 Hz, 1H), 2.52 (dp, J = 6.2, 4.3 Hz, 1H), 0.65 (tddd, J = 11.7, 5.7, 3.2,
1.5 Hz, 4H). ESI-MS calculated for C₁₀H₁₁BrClN[M + H]⁺: 261.56, Observed: 261.42
methyl 5-cyclohexylpyridine-3-carboxylate (23a).
¹H NMR (400 MHz, Chloroform-d) δ 9.05 (t, J = 1.7 Hz, 1H), 8.63-8.23 (m, 1H), 8.00 (dd, J =
2.1, 1.4 Hz, 1H), 3.90 (s, 3H), 2.86 (qd, J = 6.3, 5.5 Hz, 1H), 1.96-1.73 (m, 2H), 1.77-
1.56 (m, 5H), 1.57-1.20 (m, 5H). ESI-MS calculated for C₁₃H₁₇NO₂[M]⁺: 219.27, Observed:
219.28
methyl 5-phenylpyridine-3-carboxylate (23b).
¹H NMR (400 MHz, Chloroform-d) δ 9.09 (t, J = 1.7 Hz, 1H), 8.83 (t, J = 1.7 Hz, 1H), 8.64
(t, J = 1.6 Hz, 1H), 7.86-7.35 (m, 2H), 7.55-7.32 (m, 2H), 7.41-6.89 (m, 1H), 3.90 (s, 2H).
ESI-MS calculated for C₁₃H₁₁NO₂[M + H]⁺: 214.57, Observed: 214.44
methyl
1-(3-{[1-(tert-butoxy)-2-methyl-1-oxopropan-2-yl]oxy}phenyl)-5-cyclohexylpiperidine-3-
carboxylate (24a).
¹H NMR (400 MHz, Chloroform-d) δ 7.67-6.87 (m, 1H), 6.65 (ddd, J = 7.3, 2.1, 1.3 Hz,
1H), 6.62-6.23 (m, 2H), 3.96-3.63 (m, 2H), 3.64 (s, 2H), 3.47-3.32 (m, 1H), 3.34-3.14
(m, 1H), 2.75 (tt, J = 7.7, 4.9 Hz, 1H), 2.30-1.97 (m, 1H), 1.97-1.76 (m, 3H), 1.74-1.49
(m, 5H), 1.56-1.20 (m, 17H), 1.32-1.08 (m, 2H). ESI-MS calculated for C₂₁H₄₁NO₅[M + H]⁺:
461.57, Observed: 461.63
methyl
1-(3-{[1-(tert-butoxy)-2-methyl-1-oxopropan-2-yl]oxy}phenyl)-5-phenylpiperidine-3-
carboxylate (24b).
¹H NMR (400 MHz, Chloroform-d) δ 7.62-6.88 (m, 5H), 6.65 (ddd, J = 7.3, 2.1, 1.3 Hz,
1H), 6.56-6.21 (m, 2H), 3.92 (ddd, J = 37.4, 12.4, 4.9 Hz, 2H), 3.72 (ddd, J = 12.5, 4.9, 2.1
Hz, 2H), 3.64 (s, 2H), 3.31-3.16 (m, 1H), 2.99 (tt, J = 7.5, 5.1 Hz, 1H), 2.07 (dt, J = 12.5,
7.4 Hz, 1H), 1.92 (dt, J = 12.4, 7.4 Hz, 1H), 1.56 (d, J = 25.1 Hz, 5H), 1.43 (s, 7H). ESI-MS
calculated for C₂₇H₃₅NO₅[M + H]⁺: 454.57, Observed: 454.58
tert-butyl
4-(2-{3-[3-cyclohexyl-5-(methoxycarbonyl)piperidin-1-yl]phenoxy}-2-methylpropanoyl)piperazine-
1-carboxylate (25a)..
¹H NMR (400 MHz, Chloroform-d) δ 7.44-6.98 (m, 1H), 6.87-6.56 (m, 1H), 6.42 (ddd, J =
5.8, 2.3, 1.4 Hz, 2H), 3.93-3.65 (m, 4H), 3.72-3.44 (m, 5H), 3.54 (ddd, J = 28.0, 6.0, 3.2
Hz, 4H), 3.43-2.97 (m, 2H), 5.78-0.55 (m, 0H), 2.75 (tt, J = 7.7, 4.9 Hz, 1H), 2.18-1.99
(m, 1H), 1.99-1.76 (m, 3H), 1.55 (s, 3H), 1.48 (d, J = 22.0 Hz, 15H), 1.77-1.17 (m, 6H),
1.33-1.18 (m, 2H). ESI-MS calculated for C₃₂H₄₉N₃O₆[M + H]⁺: 573.57, Observed: 573.76
tert-butyl
4-(2-{3-[3-(methoxycarbonyl)-5-phenylpiperidin-1-yl]phenoxy}-2-methylpropanoyl)piperazine-
1-carboxylate (25b).
¹H NMR (400 MHz, Chloroform-d) δ 7.33-7.18 (m, 5H), 7.16-7.01 (m, 1H), 6.72-6.55
(m, 1H), 6.56-6.38 (m, 2H), 3.96 (dd, J = 12.3, 4.8 Hz, 1H), 3.89 (dd, J = 12.4, 5.0 Hz, 1H),
3.77-3.64 (m, 4H), 3.67-3.60 (m, 5H), 3.54 (ddd, J = 28.0, 6.0, 3.2 Hz, 4H), 3.26 (ddt, J =
7.8, 7.0, 4.7 Hz, 1H), 2.99 (tt, J = 7.5, 5.1 Hz, 1H), 2.00 (ddt, J = 75.6, 12.4, 7.4 Hz, 2H), 1.52
(d, J = 24.9 Hz, 5H), 1.45 (s, 7H). ESI-MS calculated for C₃₂H₄₃N₃O₆[M]⁺:
566.57, Observed: 566.71
tert-butyl
4-{2-[3-(3-{[(4-bromophenyl)methyl](cyclopropyl)carbamoyl}-5-cyclohexylpiperidin-1-
yl)phenoxy]-2-methylpropanoyl}piperazine-1-carboxylate (26a).
¹H NMR (400 MHz, Chloroform-d) δ 7.63-7.41 (m, 2H), 7.23 (dt, J = 8.3, 1.1 Hz, 2H), 7.19-
7.08 (m, 1H), 6.80-6.63 (m, 1H), 6.42 (ddd, J = 5.8, 2.2, 1.4 Hz, 2H), 4.57 (dt, J = 12.6,
1.1 Hz, 1H), 4.24 (dt, J = 12.4, 0.9 Hz, 1H), 3.97-3.64 (m, 3H), 3.60 (ddd, J = 29.5, 6.0, 3.2
Hz, 4H), 3.63-3.48 (m, 2H), 5.13-0.40 (m, 0H), 3.41 (dd, J = 12.4, 4.5 Hz, 1H), 3.37-
2.99 (m, 2H), 2.56 (tt, J = 7.9, 5.1 Hz, 1H), 2.02-1.70 (m, 4H), 1.58-1.44 (m, 17H), 1.51-
1.37 (m, 5H), 2.37-0.92 (m, 1H), 1.39-1.32 (m, 2H), 1.30-1.17 (m, 2H), 0.78-0.50 (m,
4H). ESI-MS calculated for C₄₁H₅₇BrN₄O₅[M + H]⁺: 766.84, Observed: 766.83
tert-butyl
4-{2-[3-(3-{[(4-bromophenyl)methyl](cyclopropyl)carbamoyl}-5-phenylpiperidin-1-yl)phenoxy]-
2-methylpropanoyl}piperazine-1-carboxylate (26b).
¹H NMR (400 MHz, Chloroform-d) δ 7.67-7.40 (m, 2H), 7.83-6.76 (m, 0H), 7.28-7.19
(m, 7H), 7.14 (d, J = 7.3 Hz, 1H), 6.66 (ddd, J = 7.3, 2.2, 1.1 Hz, 1H), 6.56-6.37 (m, 2H),
4.57 (dt, J = 12.6, 1.1 Hz, 1H), 4.24 (dt, J = 12.4, 0.9 Hz, 1H), 3.96 (dd, J = 12.3, 4.8 Hz, 1H),
3.80 (dd, J = 12.4, 5.0 Hz, 1H), 3.89-3.71 (m, 2H), 3.71 (d, J = 0.7 Hz, 1H), 3.70-3.64 (m,
1H), 3.60 (ddd, J = 29.5, 6.0, 3.2 Hz, 4H), 3.51 (dd, J = 6.0, 3.3 Hz, 2H), 3.32 (p, J = 5.7 Hz,
1H), 3.24 (tt, J = 7.7, 4.7 Hz, 1H), 2.91 (tt, J = 7.5, 4.9 Hz, 1H), 2.14 (d, J = 15.0 Hz, 0H),
2.39-2.07 (m, 1H), 1.89 (dt, J = 12.3, 7.5 Hz, 1H), 1.55 (s, 2H), 1.48 (d, J = 22.0 Hz, 9H),
0.81-0.19 (m, 4H). ESI-MS calculated for C₄₁H₅₁BrN₄O₅[M + H]⁺: 759.94, Observed: 759.79
tert-butyl
4-[2-(3-{3-cyclohexyl-5-[cyclopropyl({[4-(1H-pyrazol-4-yl)phenyl]methyl})carbamoyl]piperidin-
1-yl}phenoxy)-2-methylpropanoyl]piperazine-1-carboxylate (28).
¹H NMR (400 MHz, Chloroform-d) δ 8.41 (d, J = 1.6 Hz, 1H), 7.97 (dd, J = 3.5, 1.6 Hz, 1H),
7.57-7.37 (m, 4H), 7.19-7.11 (m, 1H), 6.95-6.59 (m, 1H), 6.42 (ddd, J = 5.8, 2.2, 1.4 Hz,
2H), 4.41 (ddt, J = 165.4, 12.6, 0.9 Hz, 2H), 3.89-3.67 (m, 3H), 3.70-3.58 (m, 3H), 3.71-
3.37 (m, 2H), 3.51 (dd, J = 6.0, 3.3 Hz, 2H), 3.41 (dd, J = 12.4, 4.5 Hz, 1H), 3.34-3.12 (m,
2H), 2.56 (tt, J = 7.9, 5.1 Hz, 1H), 2.07-1.69 (m, 3H), 1.89-1.83 (m, 2H), 1.65-1.45 (m,
17H), 1.54-1.38 (m, 5H), 1.28 (s, 0H), 0.66 (dtdd, J = 10.3, 8.6, 7.2, 3.2 Hz, 5H). ESI-MS
calculated for C₄₄H₆₀N₆O₅[M + H]⁺: 755.54, Observed: 755.56
tert-butyl
4-[2-(3-{3-[cyclopropyl({[4-(1H-pyrazol-4-yl)phenyl]methyl})carbamoyl]-5-phenylpiperidin-
1-yl}phenoxy)-2-methylpropanoyl]piperazine-1-carboxylate (29a).
¹H NMR (400 MHz, Chloroform-d) δ 8.41 (d, J = 1.6 Hz, 1H), 7.97 (dd, J = 3.5, 1.6 Hz, 1H),
7.73-7.36 (m, 4H), 7.30-7.19 (m, 5H), 7.28-7.08 (m, 1H), 6.66 (ddd, J = 7.3, 2.2, 1.1 Hz,
1H), 6.72-6.43 (m, 2H), 4.41 (ddt, J = 165.2, 12.6, 0.9 Hz, 2H), 3.96 (dd, J = 12.3, 4.8 Hz,
1H), 3.80 (dd, J = 12.4, 5.0 Hz, 1H), 3.77-3.69 (m, 3H), 4.88-0.72 (m, 1H), 3.64-3.52
(m, 3H), 3.57-3.47 (m, 3H), 3.32 (p, J = 5.7 Hz, 1H), 3.28-3.10 (m, 1H), 2.91 (tt, J = 7.5,
4.9 Hz, 1H), 2.01 (ddt, J = 119.2, 12.3, 7.5 Hz, 2H), 1.55 (s, 2H), 1.48 (d, J = 22.0 Hz, 9H),
0.80-0.52 (m, 5H). ESI-MS calculated for C₄₄H₅₄N₆O₅[M]⁺: 747.84, Observed: 747.95
tert-butyl
4-{2-[3-(3-{cyclopropyl[(4-{1H-pyrrolo[2,3-b]pyridin-3-yl}phenyl)methyl]carbamoyl}-5-
phenylpiperidin-1-yl)phenoxy]-2-methylpropanoyl}piperazine-1-carboxylate (29b).
¹H NMR (400 MHz, Chloroform-d) δ 9.81 (d, J = 7.0 Hz, 1H), 8.69 (dd, J = 3.8, 2.2 Hz, 1H),
8.42 (dd, J = 7.3, 2.2 Hz, 1H), 7.64-7.49 (m, 2H), 7.40 (dt, J = 8.4, 1.0 Hz, 2H), 7.30-7.19
(m, 8H), 7.15 (t, J = 7.3 Hz, 1H), 6.66 (ddd, J = 7.3, 2.2, 1.1 Hz, 1H), 6.51-6.19 (m, 2H),
4.41 (ddt, J = 165.4, 12.6, 0.9 Hz, 2H), 3.96 (dd, J = 12.3, 4.8 Hz, 1H), 3.81 (d, J = 4.9 Hz,
0H), 3.79-3.72 (m, 2H), 4.63-2.96 (m, 3H), 3.68 (s, 0H), 3.60 (ddd, J = 29.5, 6.0, 3.2 Hz,
4H), 3.51 (dd, J = 6.0, 3.3 Hz, 2H), 3.32 (p, J = 5.7 Hz, 1H), 3.27-3.13 (m, 1H), 2.91 (tt, J =
7.5, 4.9 Hz, 1H), 2.01 (ddt, J = 119.2, 12.3, 7.5 Hz, 2H), 1.55 (s, 2H), 1.48 (d, J = 22.0 Hz,
9H), 0.66 (dtdd, J = 10.3, 8.6, 7.2, 3.2 Hz, 4H). ESI-MS calculated for C₄₈H₅₆N₆O₅[M + H]⁺:
797.34, Observed: 797.31
tert-butyl
4-[2-(3-{3-[cyclopropyl({4-[4-(propan-2-yl)cyclohex-1-en-1-yl]phenyl}methyl)carbamoyl]-5-
phenylpiperidin-1-yl}phenoxy)-2-methylpropanoyl]piperazine-1-carboxylate (29c).
¹H NMR (400 MHz, Chloroform-d) δ 7.63-7.28 (m, 1H), 7.35-7.02 (m, 2H), 7.25-7.18
(m, 6H), 7.15 (t, J = 7.3 Hz, 1H), 6.66 (ddd, J = 7.3, 2.2, 1.1 Hz, 1H), 6.56-6.36 (m, 2H),
5.98 (tt, J = 5.9, 1.1 Hz, 1H), 4.41 (ddt, J = 165.4, 12.6, 1.0 Hz, 2H), 3.96 (dd, J = 12.3, 4.8
Hz, 1H), 3.80 (dd, J = 12.4, 5.0 Hz, 1H), 3.73-3.65 (m, 4H), 4.47-3.02 (m, 1H), 3.71-
3.57 (m, 3H), 3.51 (dd, J = 6.0, 3.3 Hz, 2H), 3.32 (p, J = 5.7 Hz, 1H), 3.26-3.17 (m, 1H),
2.98-2.81 (m, 1H), 2.77-2.51 (m, 2H), 2.24-1.92 (m, 3H), 1.89 (dt, J = 12.3, 7.5 Hz, 1H),
1.55 (s, 3H), 1.45 (s, 7H), 1.00-0.80 (m, 6H), 0.66 (dddd, J = 8.8, 5.5, 3.1, 1.7 Hz, 4H).
ESI-MS calculated for C₅₀H₆₆N₄O₅[M + H]⁺: 803.94, Observed: 803.91
tert-butyl
4-(2-{3-[3-{cyclopropyl[(4-phenylphenyl)methyl]carbamoyl}-5-phenylpiperidin-1-yl]phenoxy}-
2-methylpropanoyl)piperazine-1-carboxylate (29d).
¹H NMR (400 MHz, Chloroform-d) δ 7.59 (dd, J = 8.2, 1.4 Hz, 2H), 8.06-6.18 (m, 1H), 7.53
(d, J = 1.2 Hz, 1H), 7.49-7.44 (m, 1H), 7.44-7.37 (m, 2H), 7.27 (ddq, J = 13.6, 7.8, 1.2 Hz,
4H), 7.29-7.16 (m, 3H), 7.15 (t, J = 7.3 Hz, 1H), 6.66 (ddd, J = 7.3, 2.2, 1.1 Hz, 1H), 6.55-
6.34 (m, 2H), 4.41 (ddt, J = 165.4, 12.6, 0.9 Hz, 2H), 3.96 (dd, J = 12.3, 4.8 Hz, 1H), 3.84-
3.72 (m, 2H), 4.14-2.88 (m, 2H), 3.70-3.62 (m, 3H), 3.54 (ddd, J = 28.0, 6.0, 3.2 Hz, 4H),
3.39-3.21 (m, 1H), 3.26-3.18 (m, 1H), 2.91 (tt, J = 7.5, 4.9 Hz, 1H), 2.01 (ddt, J = 119.2,
12.3, 7.5 Hz, 2H), 1.52 (d, J = 24.9 Hz, 5H), 1.45 (s, 7H), 0.93-0.53 (m, 4H). ESI-MS
calculated for C₄₇H₅₆N₄O₅[M]⁺: 756.23, Observed: 756.12
tert-butyl
4-(2-{3-[-3-({[4-(cyclohex-1-en-1-yl)phenyl]methyl}(cyclopropyl)carbamoyl)-5-phenylpiperidin-
1-yl]phenoxy}-2-methylpropanoyl)piperazine-1-carboxylate (29e).
¹H NMR (400 MHz, Chloroform-d) δ 7.49-7.21 (m, 8H), 7.15 (t, J = 7.3 Hz, 1H), 6.66 (ddd,
J = 7.3, 2.2, 1.1 Hz, 1H), 6.53-6.40 (m, 2H), 6.11 (tt, J = 4.6, 1.0 Hz, 1H), 4.59-4.21 (m,
2H), 3.97 (d, J = 4.8 Hz, 0H), 3.80 (dd, J = 12.4, 5.0 Hz, 1H), 4.15-3.34 (m, 1H), 3.81-
3.66 (m, 4H), 3.69-3.57 (m, 4H), 3.51 (dd, J = 6.0, 3.3 Hz, 2H), 3.32 (p, J = 5.7 Hz, 1H),
3.30-3.21 (m, 1H), 2.91 (tt, J = 7.5, 4.9 Hz, 1H), 2.73 (tq, J = 5.8, 1.1 Hz, 2H), 2.30-2.06
(m, 2H), 2.21-1.81 (m, 2H), 1.52 (d, J = 24.9 Hz, 5H), 1.45 (s, 7H), 0.93-0.53 (m, 4H).
ESI-MS calculated for C₄₇H₆₀N₄O₅[M]⁺: 760.43, Observed: 760.56
ethyl
(3R)-1-(3-{[1-(tert-butoxy)-2-methyl-1-oxopropan-2-yl]oxy}-2-chlorophenyl)piperidine-3-
carboxylate (30a).
¹H NMR (400 MHz, Chloroform-d) δ 7.14 (t, J = 7.3 Hz, 1H), 6.83 (dd, J = 7.3, 1.1 Hz, 1H),
6.76 (dd, J = 7.4, 1.2 Hz, 1H), 4.12 (q, J = 6.3 Hz, 2H), 3.85 (dd, J = 12.5, 3.8 Hz, 1H), 3.60
(dd, J = 12.5, 6.4 Hz, 1H), 3.57-3.44 (m, 2H), 2.63 (dddd, J = 8.0, 6.6, 5.2, 3.8 Hz, 1H),
1.96-1.86 (m, 1H), 1.78 (dqd, J = 8.3, 5.6, 4.1 Hz, 2H), 1.72-1.62 (m, 1H), 1.55 (s, 3H),
1.43 (s, 9H), 1.24 (t, J = 6.4 Hz, 3H).. ESI-MS calculated for
C₂₃H₃₅ClNO₅[M + H]+: 426.14, Observed: 425.95
ethyl
(3R)-1-(5-{[1-(tert-butoxy)-2-methyl-1-oxopropan-2-yl]oxy}-2-chlorophenyl)piperidine-3-
carboxylate (30b).
¹H NMR (400 MHz, Chloroform-d) δ 7.14 (d, J = 8.7 Hz, 1H), 6.79 (dd, J = 8.6, 2.4 Hz, 1H),
6.42 (d, J = 2.5 Hz, 1H), 4.12 (q, J = 6.3 Hz, 2H), 3.83 (dd, J = 12.4, 3.8 Hz, 1H), 3.59 (dd, J =
12.4, 6.5 Hz, 1H), 3.55-3.45 (m, 2H), 2.63 (dddd, J = 8.0, 6.5, 5.2, 3.7 Hz, 1H), 1.96-
1.86 (m, 1H), 1.84-1.72 (m, 2H), 1.72-1.62 (m, 1H), 1.54 (s, 3H), 1.43 (s, 9H), 1.24 (t, J =
6.4 Hz, 3H).. ESI-MS calculated for C₂₃H₃₅ClNO₅[M + H]⁺: 427.05, Observed: 426.95
ethyl
(3R)-1-(3-{[1-(tert-butoxy)-2-methyl-1-oxopropan-2-yl]oxy}phenyl)piperidine-3-carboxylate
(30c).
¹H NMR (400 MHz, Chloroform-d) δ 7.04 (t, J = 8.2 Hz, 1H), 6.54 (dd, J = 8.1, 2.3 Hz, 1H),
6.45 (t, J = 2.4 Hz, 1H), 6.31-6.21 (m, 1H), 4.12 (q, J = 7.1 Hz, 2H), 3.67 (ddt, J = 12.4, 3.5, 1.5
Hz, 1H), 3.42 (ddd, J = 12.3, 4.9, 3.1 Hz, 1H), 2.95 (dd, J = 12.4, 9.9 Hz, 1H), 2.80-2.68 (m,
1H), 2.60 (tt, J = 10.0, 3.9 Hz, 1H), 2.05-1.93 (m, 1H), 1.74 (th, J = 9.2, 3.1 Hz, 1H), 1.69-
1.59 (m, 2H), 1.53 (s, 6H), 1.41 (s, 9H), 1.24 (t, J = 7.2 Hz, 3H). ESI-MS calculated for
C₂₃H₃₆NO₅[M]⁺: 392.45, Observed: 392.51
tert-butyl
4-(2-{2-chloro-3-[(3R)-3-(ethoxycarbonyl)piperidin-1-yl]phenoxy}-2-methylpropanoyl)piperazine-
1-carboxylate (31a).
¹H NMR (400 MHz, Chloroform-d) δ 7.05 (t, J = 8.2 Hz, 1H), 6.55 (dd, J = 8.0, 2.3 Hz, 1H),
6.41 (t, J = 2.3 Hz, 1H), 6.27 (ddd, J = 8.2, 2.5, 0.7 Hz, 1H), 4.15 (q, J-7.1 Hz, 2H), 3.81 (t,
J = 5.2 Hz, 2H), 3.67 (ddt, J = 12.4, 3.5, 1.5 Hz, 1H), 3.59 (t, J = 5.2 Hz, 2H), 3.50-3.39 (m,
1H), 3.33 (q, J = 5.5 Hz, 2H), 3.07 (t, J = 5.2 Hz, 2H), 2.97 (dd, J = 12.4, 9.9 Hz, 1H), 2.81-
2.70 (m, 1H), 2.62 (tt, J = 10.0, 3.9 Hz, 1H), 2.07-1.92 (m, 1H), 1.78 (qq, J = 4.9, 3.2, 2.2 Hz,
1H), 1.62 (s, 8H), 1.41 (s, 9H), 1.26 (t, J = 7.1 Hz, 3H). ESI-MS calculated for
C₂₇H₄₀ClN₃O₆[M + H]⁺: 540.45, Observed: 540.48
tert-butyl
4-(2-{4-chloro-3-[(3R)-3-(ethoxycarbonyl)piperidin-1-yl]phenoxy}-2-methylpropanoyl)piperazine-
1-carboxylate (31b).
¹HNMR (400 MHz, Chloroform-d) δ 7.65 30-7.61 (m, 1H), 7.45-7.38 (m, 2H), 4.16 (q, J =
7.2 Hz, 2H), 3.67 (ddt, J = 12.7, 3.6, 1.5Hz, 1H), 3.43 (d, J = 12.4 Hz, 1H), 3.01 (dd, J = 12.5,
9.9 Hz, 1H), 2.81 (t, J = 11.5 Hz, 1H), 2.62 (s, 1H), 2.02 (d, J = 9.2 Hz, 1H), 1.82-1.73 (m,
1H), 1.72-1.63 (m, 2H), 1.56 (d, J = 1.5 Hz, 6H), 1.45 (s, 9H), 1.28 (t, J = 7.1 Hz, 3H).
ESI-MS calculated for C₂₇H₄₀ClN₃O₆[M]⁺: 539.45, Observed: 539.08
tert-butyl
4-(2-{3-[(3R)-3-(ethoxycarbonyl)piperidin-1-yl]phenoxy}-2-methylpropanoyl)piperazine-
1-carboxylate (35). ¹H NMR (400 MHz, Chloroform-d) δ 10 7.18 (d, J = 8.7 Hz, 1H), 6.61-
6.50 (m, 2H), 4.19-4.11 (m, 2H), 3.60 (ddt, J = 12.3, 3.1, I. 5 Hz, 1H), 3.42-3.32 (m, 1H),
2.95 (dd, J = 12.3, 9.9 Hz, 1H), 2.80-2.70 (m, 1H), 2.61 (dq, J = 9.9, 5.3, 4.4 Hz, 1H), 2.01
(dt, J = 9.8, 5.3 Hz, 1H), 1.78 (dtt, J = 11.3, 7.8, 3.1 Hz, 1H), 1.68-1.61 (m, 2H), 1.57 (d, J =
1.9 Hz, 6H), 1.46 (d, J = 1.4 Hz, 9H), 1.27 (td, J = 7.1, 1.3 Hz, 3H). ESI-MS calculated for
C₂₇H₄₀ClN₃O₆[M + H]⁺: 504.65, Observed: 504.64
tert-butyl
4-(2-{3-[(3R)-3-{[(4-bromophenyl)methyl](cyclopropyl)carbamoyl}piperidin-1-yl]-2-
chlorophenoxy}-2-methylpropanoyl)piperazine-1-carboxylate (32a). ¹H NMR(400 MHz,
Chloroform-d) δ 7.40-7.33 (m, 2H), 7.10-7.00 (m, 2H), 6.43 (t, J = 2.0 Hz, 1H), 6.21 (dt, J =
7.2, 2.2 Hz, 2H), 4.58 (d, J = 14.7 Hz, 1H), 4.39 (d, J = 14.7 Hz, 1H), 3.71 (d, J = 5.4 Hz,
2H), 3.60-3.44 (m, 4H), 3.30 (dq, J = 25.6, 6.5, 5.5 Hz, 3H), 3.05 (t, J = 4.8 Hz, 2H), 2.94
(dd, J = 12.7, 11.0 Hz, 1H), 2.72 (s, 1H), 2.56 (tt, J = 6.9, 4.0 Hz, 1H), 1.87 (dd, J = 12.2, 3.7
Hz, 1H), 1.77-1.60 (m, 3H), 1.56 (d, J = 2.0 Hz, 6H), 1.37 (s, 9H), 0.81-0.75 (m, 4H).
ESI-MS calculated for C₃₆H₄₇BrClN₃O₅[M]⁺: 718.45, Observed: 718.13
tert-butyl
4-(2-{3-[(3R)-3-{[(4-bromophenyl)methyl](cyclopropyl)carbamoyl}piperidin-1-yl]-4-
chlorophenoxy}-2-methylpropanoyl)piperazine-1-carboxylate (32b). ¹H NMR (400 MHz,
Chloroform-d) δ 7.45-7.39 (m, 2H), 7.17 (d, J = 8.4 Hz, 1H), 7.11 (d, J = 8.0 20 Hz, 2H), 6.43
(d, J = 8.1 Hz, 2H), 4.58 (d, J = 14.8 Hz, 1H), 4.50 (d, J = 14.8 Hz, 1H), 3.78 (s, 2H), 3.53 (td,
J = 14.3, 13.8, 7.3 Hz, 4H), 3.37 (t, J = 10.9 Hz, 1H), 3.29 (s, 2H), 3.09 (d, J = 13.3 Hz, 2H),
2.95-2.82 (m, 1H), 2.74-2.57 (m, 2H), 1.91 (d, J = 11.5 Hz, 1H), 1.83 (s, 1H), 1.66 (d, J = 3.4
Hz, 8H), 1.42 (d, J = 1.4 Hz, 9H), 0.92-0.76 (m, 4H). ESI-MS calculated for
C₃₆H₄₇BrClN₃O₅[M]⁺: 718.25, Observed: 718.13
tert-butyl
4-(2-{2-chloro-3-[(3R)-3-[cyclopropyl({[4-(1H-pyrazol-4-yl)phenyl]methyl})carbamoyl]piperidin-
1-yl]phenoxy}-2-methylpropanoyl)piperazine-1-carboxylate (33a).
¹H NMR (400 MHz, Chloroform-d) δ 7.81 (s, 2H), 7.46-7.40 (m, 2H), 7.24-7.19 (m, 2H),
6.50 (t, J = 1.9 Hz, 1H), 6.26 (d, J = 1.9 Hz, 2H), 4.71 (d, J = 14.7 Hz, 1H), 4.50 (d, J = 14.7 Hz,
1H), 3.76 (d, J = 6.0 Hz, 2H), 3.66-3.55 (m, 4H), 3.40 (ddt, J = 25 11.2, 7.2, 3.5 Hz, 1H), 3.33
(s, 2H), 3.10 (d, J = 5.5 Hz, 2H), 3.02 (dd, J = 12.7, 11.0 Hz, 1H), 2.78 (td, J = 12.4, 2.5 Hz,
1H), 2.64 (tt, J = 6.8, 4.2 Hz, 1H), 1.95 (d, J = 11.8 Hz, 1H), 1.84-1.66 (m, 3H), 1.61 (s, 6H),
1.42 (s, 9H), 0.87-0.82 (m, 4H). ESI-MS calculated for C₃₉H₅₀ClN₅O₅[M]⁺: 705.35,
Observed: 705.30
tert-butyl
4-(2-{2-chloro-3-[(3R)-3-{cyclopropyl[(4-{1H-pyrrolo[2,3-b]pyridin-3-yl}phenyl)meth-
yl]carbamoyl}piperidin-1-yl]phenoxy}-2-methylpropanoyl)piperazine-1-carboxylate (33b).
¹H NMR (400 MHz, Chloroform-d) δ 9.81 (d, J = 7.0 Hz, 1H), 8.69 (dd, J = 3.8, 2.2 Hz, 1H),
8.42 (dd, J = 7.3, 2.2 Hz, 1H), 7.57-7.46 (m, 2H), 7.40 (dt, J = 8.4, 1.0 Hz, 2H), 7.28 (dd, J =
7.2, 4.1 Hz, 2H), 7.15 (t, J = 7.3 Hz, 1H), 6.66 (ddd, J = 7.3, 2.2, 1.1 Hz, 1H), 6.53-6.44
(m, 2H), 6.43 (s, 0H), 4.68-4.22 (m, 2H), 3.81-3.71 (m, 3H), 3.71 (s, 1H), 3.63-3.53 (m,
3H), 3.59-3.48 (m, 3H), 3.40 (s, 0H), 3.32 (p, J = 5.7 Hz, 1H), 2.71 (dddd, J = 7.7, 6.2, 5.0,
3.5 Hz, 1H), 6.09--1.16 (m, 4H), 2.06-1.63 (m, 1H), 1.85-1.73 (m, 2H), 1.89-1.50 (m,
3H), 1.48 (d, J = 22.0 Hz, 10H), 0.66 (dddd, J = 8.8, 5.5, 3.1, 1.7 Hz, 4H). ESI-MS calculated
for C₄₃H₅₂ClN₅O₅[M]⁺: 754.35, Observed: 754.36
tert-butyl
4-(2-{4-chloro-3-[(3R)-3-[cyclopropyl({[4-(1H-pyrazol-4-yl)phenyl]methyl})carbamoyl]piper-
idin-1-yl]phenoxy}-2-methylpropanoyl)piperazine-1-carboxylate (33c).
¹H NMR (400 MHz, Chloroform-d) δ 7.82 (s, 2H), 7.45 (d, J = 7.8 Hz, 2H), 7.24 (d, J = 7.8
Hz, 2H), 7.18 (d, J = 8.6 Hz, 1H), 6.51-6.37 (m, 2H), 4.67 (d, J = 14.6 Hz, 1H), 4.54 (d, J =
14.7 Hz, 1H), 3.79 (s, 2H), 3.63-3.47 (m, 4H), 3.46-3.36 (m, 1H), 3.29 (s, 2H), 3.18-3.03
(m, 2H), 2.95 (t, J = 11.6 Hz, 1H), 2.78-2.63 (m, 2H), 1.94 (d, J = 12.2 Hz, 1H), 1.89 10-1.81
(m, 1H), 1.67 (s, 8H), 1.42 (d, J = 1.5 Hz, 9H), 0.90-0.82 (m, 4H). ESI-MS calculated for
C₃₉H₅₀ClN₅O₅[M]⁺: 705.45, Observed: 705.30
tert-butyl
4-(2-{4-chloro-3-[(3R)-3-{cyclopropyl[(4-{1H-pyrrolo[2,3-b]pyridin-3-yl}phenyl)methyl]car-
bamoyl}piperidin-1-yl]phenoxy}-2-methylpropanoyl)piperazine-1-carboxylate (33d).
¹H NMR (400 MHz, Chloroform-d) δ 9.81 (d, J = 7.0 Hz, 1H), 8.69 (dd, J = 3.8, 2.2 Hz, 1H),
8.42 (dd, J = 7.3, 2.2 Hz, 1H), 7.40 (dt, J = 8.4, 1.0 Hz, 2H), 7.28 (dd, J = 7.2, 4.1 Hz, 2H),
7.14 (d, J = 8.7 Hz, 1H), 6.80-6.74 (m, 1H), 6.41 (d, J = 2.4 Hz, 1H), 4.60-4.14 (m, 2H),
3.84-3.65 (m, 3H), 3.57 (dd, J = 6.0, 3.2 Hz, 2H), 3.31 (q, J = 5.7 Hz, 1H), 2.68 (dddd, J =
7.7, 6.2, 4.9, 3.4 Hz, 1H), 1.81 (s, 0H), 1.81-1.73 (m, 2H), 1.67-1.35 (m, 13H), 0.66-0.45
(m, 2H). ESI-MS calculated for C₄₃H₅₄ClN₅O₅[M + H]⁺: 755.43, Observed: 755.36
tert-butyl
4-(2-{4-chloro-3-[(3R)-3-({[4-(cyclohex-1-en-1-yl)phenyl]methyl}(cyclopropyl)carbamoyl)piper-
idin-1-yl]phenoxy}-2-methylpropanoyl)piperazine-1-carboxylate (33e).
¹H NMR (400 MHz, Chloroform-d) δ 7.45-7.17 (m, 2H), 7.15 (t, J = 7.3 Hz, 1H), 7.03 (ddt,
J = 8.8, 2.2, 1.0 Hz, 1H), 6.68-6.65 (m, 1H), 6.56-6.38 (m, 2H), 6.06 (tt, J = 4.6, 1.0 Hz,
1H), 3.76-3.67 (m, 3H), 3.63 (dd, J = 6.0, 3.2 Hz, 2H), 3.56 (d, J = 3.2 Hz, 1H), 4.05-3.08
(m, 2H), 3.52-3.43 (m, 4H), 3.40 (dd, J = 5.5, 4.3 Hz, 2H), 2.89-2.77 (m, 2H), 2.79-2.66
(m, 4H), 2.61 (dddd, J = 7.7, 6.0, 4.7, 3.1 Hz, 1H), 2.09 (tdt, J = 6.4, 4.6, 1.1 Hz, 2H), 1.99-
1.83 (m, 1H), 1.97-1.64 (m, 7H), 5.76-0.33 (m, 2H), 1.56-1.26 (m, 13H), 0.84--0.12
(m, 5H). ESI-MS calculated for C₄₂H₅₆ClN₃O₅[M]⁺: 719.38, Observed: 719.36
tert-butyl
4-(2-{3-[(3R)-3-{[(4-bromo-3-chlorophenyl)methyl](cyclopropyl)carbamoyl}piperidin-1-yl]
phenoxy}-2-methylpropanoyl)piperazine-1-carboxylate (36).
¹H NMR (400 MHz, Chloroform-d) δ 7.48 (d, J = 8.0 Hz, 1H), 7.30-6.91 (m, 3H), 6.66
(ddd, J = 7.3, 2.2, 1.1 Hz, 1H), 6.56-6.29 (m, 2H), 4.42-3.67 (m, 3H), 3.60 (ddd, J = 29.5,
6.0, 3.2 Hz, 4H), 3.56-3.38 (m, 3H), 3.44-3.07 (m, 2H), 2.88-2.59 (m, 3H), 2.62 (dddd, J =
7.7, 6.0, 4.7, 3.1 Hz, 1H), 2.18-1.08 (m, 0H), 1.94-1.55 (m, 4H), 1.74-1.25 (m, 13H),
0.60-0.09 (m, 4H). ESI-MS calculated for C₃₆H₄₇BrClN₃O₅[M]⁺: 718.2, Observed: 718.13
tert-butyl
4-(2-{3-[(3R)-3-({[3-chloro-4-(1H-pyrazol-4-yl)phenyl]methyl}(cyclopropyl)carbamoyl)piper-
idin-1-yl]phenoxy}-2-methylpropanoyl)piperazine-1-carboxylate (37a).
¹H NMR (400 MHz, Chloroform-d) δ 7.75 (s, 2H), 7.45 (d, J = 1.8 Hz, 1H), 7.30 (dd, J = 7.9,
1.8 Hz, 1H), 7.06 (t, J = 7.7 Hz, 2H), 6.62-6.49 (m, 1H), 6.49-6.37 (m, 1H), 20 6.27 (dd, J =
8.1, 2.3 Hz, 1H), 4.77 (d, J = 15.6 Hz, 1H), 4.65 (d, J = 15.6 Hz, 1H), 3.85-3.47 (m, 7H), 3.40-
3.21 (m, 2H), 3.14-2.95 (m, 3H), 2.81-2.64 (m, 2H), 2.05-1.92 (m, 1H), 1.85-1.70 (m,
3H), 1.62 (d, J = 2.0 Hz, 6H), 1.40 (s, 9H), 0.92-0.74 (m, 4H). ESI-MS calculated for
C₃₉H₅₀ClN₅O₅[M]⁺: 705.32, Observed: 705.30
tert-butyl
4-(2-{3-[(3R)-3-({[3-chloro-4-(cyclohex-1-en-1-yl)phenyl]methyl}(cyclopropyl)carbamoyl)piperidin-
1-yl]phenoxy}-2-methylpropanoyl)piperazine-1-carboxylate (37b)
¹H NMR (400 MHz, Chloroform-d) δ 7.46-7.15 (m, 2H), 7.15 (t, J = 7.3 Hz, 1H), 7.03 (ddt,
J = 8.8, 2.2, 1.0 Hz, 1H), 6.68-6.65 (m, 1H), 6.56-6.38 (m, 2H), 6.06 (tt, J = 4.6, 1.0 Hz,
1H), 3.76-3.67 (m, 3H), 3.63 (dd, J = 6.0, 3.2 Hz, 2H), 3.56 (d, J = 3.2 Hz, 1H), 4.05-3.08
(m, 2H), 3.52-3.43 (m, 4H), 3.40 (dd, J = 5.6, 4.3 Hz, 2H), 2.89-2.77 (m, 2H), 2.79-2.66
(m, 4H), 2.61 (dddd, J = 7.7, 6.0, 4.7, 3.1 Hz, 1H), 2.09 (tdt, J = 6.4, 4.6, 1.1 Hz, 2H), 1.99-
1.83 (m, 1H), 1.97-1.64 (m, 7H), 5.76-0.33 (m, 2H), 1.56-1.26 (m, 13H), 0.84-−0.12
(m, 5H). ESI-MS calculated for C₄₂H₅₈ClN₃O₅[M]⁺: 719.32, Observed: 719.36
tert-butyl
4-(2-{3-[(3R)-3-{[(3-chloro-4-phenylphenyl)methyl](cyclopropyl)carbamoyl}piperidin-1-yl]
phenoxy}-2-methylpropanoyl)piperazine-1-carboxylate (37c).
¹H NMR (400 MHz, Chloroform-d) δ 7.60 (d, J = 8.7 Hz, 1H), 7.54-7.41 (m, 1H), 7.49-
7.38 (m, 3H), 7.42-7.35 (m, 1H), 7.26 (dt, J = 2.1, 1.0 Hz, 1H), 7.93-6.12 (m, 0H), 7.21-
6.99 (m, 2H), 6.66 (ddd, J = 7.3, 2.2, 1.1 Hz, 1H), 6.49 (ddd, J = 7.5, 2.3, 1.2 Hz, 1H), 6.44 (t,
J = 2.2 Hz, 1H), 3.78-3.64 (m, 3H), 3.60 (ddd, J = 29.5, 6.0, 3.2 Hz, 4H), 3.55-3.39 (m,
3H), 3.41 (td, J = 5.5, 4.2 Hz, 2H), 2.97-2.62 (m, 3H), 3.08-2.62 (m, 0H), 2.61 (dddd, J =
7.7, 6.0, 4.7, 3.1 Hz, 1H), 4.23-0.08 (m, 0H), 1.92 (dddd, J = 11.9, 8.9, 6.2, 4.6 Hz, 1H),
1.87-1.71 (m, 3H), 1.64 (dddd, J = 12.0, 8.8, 7.4, 6.2 Hz, 1H), 1.68-1.18 (m, 12H), 0.58-
0.28 (m, 4H). ESI-MS calculated for C₄₂H₅₂ClN₃O₅[M]⁺: 715.32, Observed: 715.33
tert-butyl 2-[(3-iodophenyl)amino]-2-methylpropanoate (38a)
¹H NMR (400 MHz, Chloroform-d) δ 7.36 (ddd, J = 7.3, 2.3, 1.3 Hz, 1H), 7.19-6.90 (m,
3H), 6.56 (ddd, J = 6.8, 2.2, 1.2 Hz, 1H), 5.66 (s, 1H), 1.55 (s, 6H), 1.42 (s, 9H). ESI-MS
calculated for C₁₄H₂₀INO₂[M]⁺: 361.32, Observed: 361.22
tert-butyl 2-[(3-iodophenyl)(methyl)amino]-2-methylpropanoate (38b).
¹H NMR (400 MHz, Chloroform-d) δ 7.58 (ddd, J = 7.7, 2.2, 1.2 Hz, 1H), 7.17 (t, J = 2.2 Hz,
1H), 7.04 (dd, J = 7.6, 6.3 Hz, 1H), 6.63 (ddd, J = 6.4, 2.2, 1.1 Hz, 1H), 3.00 (s, 2H), 1.46 (s,
5H), 1.42 (s, 7H). ESI-MS calculated for C₁₅H₂₀INO₂[M]⁺: 375.72, Observed: 375.35
tert-butyl 4-{2-[(3-iodophenyl)amino]-2-methylpropanoyl}piperazine-1-carboxylate (39a).
¹H NMR (400 MHz, Chloroform-d) δ 7.58 (ddd, J = 7.7, 2.2, 1.2 Hz, 1H), 7.18 (t, J = 2.3 Hz,
1H), 7.09-7.03 (m, 1H), 6.67 (ddd, J = 6.4, 2.3, 1.2 Hz, 1H), 3.71 (dd, J = 6.1, 3.1 Hz, 2H),
3.65-3.53 (m, 4H), 3.52 (dd, J = 6.0, 3.1 Hz, 2H), 3.03 (s, 2H), 1.45 (s, 7H), 1.39 (s, 5H).
ESI-MS calculated for C₁₉H₂₈IN₃O₃[M]⁺: 473.92, Observed: 473.75
tert-butyl 4-{2-[(3-iodophenyl)(methyl)amino]-2-methylpropanoyl}piperazine-1-carboxylate
(39b).
¹H NMR (400 MHz, Chloroform-d) δ 7.62 (ddd, J = 7.7, 2.5, 1.3 Hz, 1H), 7.28 (t, J = 2.5 Hz,
1H), 7.09-7.02 (m, 1H), 6.76 (ddd, J = 6.4, 2.3, 1.2 Hz, 1H), 3.81 (dd, J = 6.1, 3.1 Hz, 2H),
3.65-3.52 (m, 4H), 3.52 (dd, J = 6.0, 3.1 Hz, 2H), 3.03 (s, 2H), 1.45 (s, 7H), 1.39 (s,
5H). ESI-MS calculated for C₂₀H₃₀IN₃O₃[M + H]⁺: 488.52, Observed: 488.38
tert-butyl
4-[2-({3-[(3R)-3-(ethoxycarbonyl)piperidin-1-yl]phenyl}(methyl)amino)-2-methylpropanoyl]
piperazine-1-carboxylate (40a).
¹H NMR (400 MHz, Chloroform-d) δ 10 7.18 (d, J = 8.7 Hz, 1H), 6.61-6.50 (m, 2H),
4.19-4.11 (m, 2H), 3.60 (ddt, J = 12.3, 3.1, I. 5 Hz, 1H), 3.42-3.32 (m, 1H), 2.95 (dd, J = 12.3,
9.9 Hz, 1H), 2.80-2.70 (m, 1H), 2.61 (dq, J = 9.9, 5.3, 4.4 Hz, 1H), 2.01 (dt, J = 9.8, 5.3 Hz,
1H), 1.78 (dtt, J = 11.3, 7.8, 3.1 Hz, 1H), 1.68-1.61 (m, 2H), 1.57 (d, J = 1.9 Hz, 6H), 1.46 (d,
J = 1.4 Hz, 9H), 1.27 (td, J = 7.1, 1.3 Hz, 3H). ESI-MS calculated for C₂₇H₄₂IN₄O₅[M]⁺:
502.72, Observed: 502.65.
tert-butyl
4-[2-({3-[(3R)-3-(ethoxycarbonyl)piperidin-1-yl]phenyl}(methyl)amino)-2-methylpropanoyl]
piperazine-1-carboxylate (40b)
¹H NMR (400 MHz, Chloroform-d) δ 10 7.35 (d, J = 8.7 Hz, 1H), 6.62-6.50 (m, 2H),
4.32-4.15 (m, 2H), 3.60 (ddt, J = 12.5, 3.1, I. 5 Hz, 1H), 3.42-3.34 (m, 1H), 2.95 (dd, J = 12.3,
9.9 Hz, 1H), 2.83-2.73 (m, 1H), 2.61 (dq, J = 9.9, 5.3, 4.4 Hz, 1H), 2.01 (dt, J = 9.8, 5.3 Hz,
1H), 1.78 (dtt, J = 11.3, 7.8, 3.1 Hz, 1H), 1.68-1.61 (m, 2H), 1.58 (d, J = 1.9 Hz, 6H), 1.56 (d,
J = 1.3 Hz, 9H), 1.27 (td, J = 7.1, 1.2 Hz, 3H). ESI-MS calculated for C₂₈H₄₄N₄O₅[M]⁺:
516.92, Observed: 516.68
tert-butyl
4-[2-({3-[(3R)-3-{[(4-bromophenyl)methyl](cyclopropyl)carbamoyl}piperidin-1-yl]phenyl}amino)-
2-methylpropanoyl]piperazine-1-carboxylate (41a).
¹H NMR (400 MHz, Chloroform-d) δ 7.56-7.30 (m, 2H), 7.22 (dt, J = 7.8, 1.0 Hz, 2H), 7.02-
6.88 (m, 1H), 6.43 (dddd, J = 17.9, 7.6, 2.2, 1.1 Hz, 2H), 6.28 (t, J = 2.2 Hz, 1H), 3.82-
3.67 (m, 3H), 3.59 (dd, J = 6.0, 3.1 Hz, 2H), 3.60-3.48 (m, 3H), 3.56-3.51 (m, 3H), 3.41
(ddd, J = 6.6, 5.6, 4.0 Hz, 2H), 2.87 (dt, J = 9.0, 6.3 Hz, 1H), 2.83-2.63 (m, 2H), 2.59 (dddd,
J = 7.6, 6.0, 4.7, 3.2 Hz, 1H), 1.45 (d, J = 18.9 Hz, 10H), 1.37 (s, 2H), 0.71-0.21 (m, 4H).
ESI-MS calculated for C35H₄₈BrN₅O₄[M + H]⁺: 681.75, Observed: 681.72
tert-butyl
4-[2-({3-[(3R)-3-{[(4-bromophenyl)methyl](cyclopropyl)carbamoyl}piperidin-1-
yl]phenyl}(methyl)amino)-2-methylpropanoyl]piperazine-1-carboxylate (41b).
¹H NMR (400 MHz, Chloroform-d) δ 7.52-7.33 (m, 2H), 7.22 (dt, J = 7.9, 1.0 Hz, 2H), 7.02-
6.89 (m, 1H), 6.43 (dddd, J = 17.9, 7.5, 2.2, 1.1 Hz, 2H), 6.28 (t, J = 2.2 Hz, 1H), 3.82-
3.67 (m, 3H), 3.59 (dd, J = 6.0, 3.1 Hz, 2H), 3.60-3.47 (m, 3H), 3.56-3.51 (m, 3H), 3.41
(ddd, J = 6.6, 5.6, 4.0 Hz, 2H), 2.87 (dt, J = 9.0, 6.3 Hz, 1H), 2.83-2.63 (m, 2H), 2.59 (dddd,
J = 7.6, 6.0, 4.7, 3.2 Hz, 1H), 1.44 (d, J = 18.9 Hz, 10H), 1.37 (s, 2H), 0.71-0.18 (m,
4H). ESI-MS calculated for C₃₆H₅₀BrN₅O₄[M + H]+: 698.75, Observed: 698.73
tert-butyl
4-[2-({3-[(3R)-3-[cyclopropyl({[4-(1H-pyrazol-4-yl)phenyl]methyl})carbamoyl]piperidin-1-
yl]phenyl}amino)-2-methylpropanoyl]piperazine-1-carboxylate (42a).
¹H NMR (400 MHz, Chloroform-d) δ 8.41 (d, J = 1.6 Hz, 1H), 7.97 (dd, J = 3.5, 1.6 Hz, 1H),
7.60-7.32 (m, 3H), 7.37 (dt, J = 8.5, 1.0 Hz, 3H), 7.09 (t, J = 7.2 Hz, 1H), 6.57 (ddd, J = 7.1,
2.2, 1.2 Hz, 1H), 6.45 (ddd, J = 7.1, 2.3, 1.2 Hz, 1H), 6.10 (t, J = 2.2 Hz, 1H), 5.68 (s, 1H),
3.95-3.65 (m, 4H), 3.66-3.50 (m, 5H), 3.53-3.43 (m, 4H), 3.41 (ddd, J = 6.6, 5.6, 4.0 Hz,
2H), 2.97-2.75 (m, 1H), 2.80-2.63 (m, 3H), 2.59 (dddd, J = 7.6, 6.0, 4.7, 3.2 Hz, 1H), 1.92
(dddd, J = 12.1, 9.1, 6.3, 4.7 Hz, 1H), 1.89-1.58 (m, 6H), 1.52 (s, 3H), 1.46 (d, J = 8.6 Hz,
13H), 0.57-−0.02 (m, 6H). ESI-MS calculated for C₃₈H₅₁N₇O₄[M + H]⁺: 670.85, Observed: 670.87
tert-butyl
4-[2-({3-[(3R)-3-[cyclopropyl({[4-(1H-pyrazol-4-yl)phenyl]methyl})carbamoyl]piperidin-1-
yl]phenyl}(methyl)amino)-2-methylpropanoyl]piperazine-1-carboxylate (42b).
¹H NMR (400 MHz, Chloroform-d) δ 8.41 (d, J = 1.6 Hz, 1H), 7.97 (dd, J = 3.5, 1.6 Hz, 1H),
7.60-7.37 (m, 2H), 7.37 (dt, J = 8.5, 1.0 Hz, 2H), 7.02 (dd, J = 7.6, 6.6 Hz, 1H), 6.43 (dddd,
J = 17.9, 7.5, 2.2, 1.1 Hz, 2H), 6.28 (t, J = 2.2 Hz, 1H), 3.97-3.66 (m, 3H), 3.70-3.52 (m,
4H), 3.56-3.44 (m, 4H), 3.41 (ddd, J = 6.6, 5.6, 4.0 Hz, 2H), 4.10-3.25 (m, 0H), 3.03 (s,
2H), 2.87 (dt, J = 9.0, 6.3 Hz, 1H), 2.79-2.62 (m, 2H), 2.59 (s, 0H), 4.29-0.12 (m, 1H),
2.09-1.77 (m, 1H), 1.90-1.74 (m, 3H), 1.81-1.71 (m, 1H), 2.10-0.57 (m, 1H), 1.69-
1.54 (m, 1H), 1.44 (d, J = 18.9 Hz, 10H), 1.37 (s, 2H), 0.64-0.19 (m, 4H). ESI-MS
calculated for C₃₉H₅₃N₇O₄[M + H]⁺: 684.95, Observed: 684.90
N-[(4-bromophenyl)methyl]-N-cyclopropylcarbamoyl chloride (43).
¹H NMR (400 MHz, Chloroform-d) δ 8.00-7.28 (m, 2H), 7.23 (dt, J = 8.2, 1.0 Hz, 2H), 4.96-
4.09 (m, 2H), 3.30 (p, J = 6.0 Hz, 1H), 1.28-0.66 (m, 2H), 0.65-0.10 (m, 2H). ESI-MS
calculated for C₁₁H₁₁BrClNO[M + H]⁺: 288.56, Observed: 288.53
tert-butyl
2-{3-[(3R)-3-{[(tert-butoxy)carbonyl]amino}piperidin-1-yl]phenoxy}-2-methylpropanoate
(44).
¹H NMR (400 MHz, Chloroform-d) δ 7.15 (t, J = 7.3 Hz, 1H), 6.65 (ddd, J = 7.3, 2.2, 1.1 Hz,
1H), 6.49 (ddd, J = 7.3, 2.2, 1.1 Hz, 1H), 6.44 (t, J = 2.2 Hz, 1H), 5.38 (d, J = 8.6 Hz, 1H),
3.88 (dtdd, J = 9.9, 6.6, 3.6, 2.0 Hz, 1H), 3.54 (dd, J = 12.5, 2.0 Hz, 1H), 3.48-3.36 (m, 2H),
3.27 (ddd, J = 12.4, 6.0, 3.3 Hz, 1H), 1.98 (dddd, J = 12.4, 9.0, 6. 1, 3.6 Hz, 1H), 1.92-1.58
(m, 3H), 1.56 (d, J = 25.1 Hz, 5H), 1.42 (d, J = 8.8 Hz, 18H). ESI-MS calculated for
C₂₄H₃₈N₂O₅[M]⁺: 434.56, Observed: 434.58
tert-butyl
2-{3-[(3R)-3-({[(4-bromophenyl)methyl](cyclopropyl)carbamoyl}amino)piperidin-1-yl]phenoxy}-
2-methylpropanoate (46).
¹H NMR (400 MHz, Chloroform-d) δ 7.61-7.23 (m, 2H), 7.23 (dt, J = 8.3, 1.0 Hz, 2H), 7.15
(t, J = 7.3 Hz, 1H), 6.65 (ddd, J = 7.3, 2.2, 1.1 Hz, 1H), 6.56-6.33 (m, 2H), 6.44 (s, 0H),
5.64 (d, J = 9.2 Hz, 1H), 4.37 (t, J = 1.0 Hz, 2H), 1.97 (dddd, J = 12.3, 8.8, 6.1, 3.7 Hz, 1H),
1.94-1.71 (m, 2H), 1.56 (d, J = 25.1 Hz, 5H), 1.43 (s, 7H), 0.86-0.47 (m, 5H). ESI-MS
calculated for C₃₀H₄₀BrN₃O₄[M]⁺: 587.55, Observed: 587.57
tert-butyl
2-{3-[(3R)-3-{[cyclopropyl({[4-(1H-pyrazol-4-yl)phenyl]methyl})carbamoyl]amino}piperidin-
1-yl]phenoxy}-2-methylpropanoate (47).
¹H NMR (400 MHz, Chloroform-d) δ 8.41 (d, J = 1.6 Hz, 1H), 7.97 (dd, J = 3.5, 1.6 Hz, 1H),
7.42 (s, 4H), 7.15 (t, J = 7.3 Hz, 1H), 6.65 (ddd, J = 7.3, 2.2, 1.1 Hz, 1H), 6.49 (ddd, J = 7.3,
2.2, 1.1 Hz, 1H), 6.44 (t, J = 2.2 Hz, 1H), 5.64 (d, J = 9.2 Hz, 1H), 4.37 (s, 2H), 4.07-3.70
(m, 1H), 3.54 (dd, J = 12.5, 2.2 Hz, 1H), 3.50-3.34 (m, 2H), 3.27 (ddd, J = 12.4, 6.0, 3.3 Hz,
1H), 3.19 (p, J = 5.6 Hz, 1H), 1.97 (dddd, J = 12.3, 8.8, 6.1, 3.7 Hz, 1H), 1.93-1.65 (m, 2H),
1.56 (d, J = 25.1 Hz, 5H), 0.92-0.33 (m, 5H). ESI-MS calculated for C₃₃H₄₃N₅O₄[M + H]⁺:
575.70, Observed: 575.74
tert-butyl
4-(2-{3-[(3R)-3-{[cyclopropyl({[4-(1H-pyrazol-4-yl)phenyl]methyl})carbamoyl]amino}piperidin-
1-yl]phenoxy}-2-methylpropanoyl)piperazine-1-carboxylate (54).
¹H NMR (400 MHz, Chloroform-d) δ 8.41 (d, J = 1.6 Hz, 1H), 7.97 (dd, J = 3.5, 1.6 Hz, 1H),
7.15 (t, J = 7.3 Hz, 1H), 6.49 (ddd, J = 7.3, 2.2, 1.1 Hz, 1H), 6.44 (d, J = 2.2 Hz, 1H), 5.64 (d,
J = 9.2 Hz, 1H), 4.37 (s, 2H), 4.07-3.77 (m, 1H), 3.72 (dd, J = 6.0, 3.2 Hz, 2H), 3.65-3.57
(m, 3H), 3.64-3.33 (m, 4H), 3.43-3.26 (m, 3H), 4.77-2.55 (m, 0H), 3.19 (p, J = 5.6 Hz,
1H), 1.97 (dddd, J = 12.3, 8.8, 6.1, 3.7 Hz, 1H), 1.88-1.63 (m, 3H), 1.52 (d, J = 24.9 Hz,
5H), 1.45 (s, 7H), 0.86-0.51 (m, 5H). ESI-MS calculated for C₃₈H₅₁N₇O₅[M + H]⁺: 687.65,
Observed: 687.87
tert-butyl
N-[2-(2-{3-[(3R)-3-{[cyclopropyl({[4-(1H-pyrazol-4-yl)phenyl]methyl})carbamoyl]amino}piper-
idin-1-yl]phenoxy}-2-methylpropanamido)ethyl]carbamate(55).
¹H NMR (400 MHz, Chloroform-d) δ 8.41 (d, J = 1.6 Hz, 1H), 7.97 (dd, J = 3.5, 1.6 Hz, 1H),
7.46 (t, J = 4.6 Hz, 1H), 7.42 (s, 4H), 7.15 (t, J = 7.3 Hz, 1H), 6.66 (dd, J = 2.3, 1.2 Hz, 0H),
6.71-6.34 (m, 2H), 6.49 (ddd, J = 7.3, 2.2, 1.1 Hz, 1H), 5.64 (d, J = 9.2 Hz, 1H), 5.32 (t, J =
5.1 Hz, 1H), 4.37 (s, 2H), 4.00-3.78 (m, 1H), 3.51-3.33 (m, 4H), 3.32-3.24 (m, 3H), 3.18
(d, J = 5.6 Hz, 1H), 5.85-0.48 (m, 2H), 2.22-1.83 (m, 1H), 1.87-1.57 (m, 2H), 1.55-
1.26 (m, 12H), 0.90-0.46 (m, 5H). ESI-MS calculated for C₃₆H₄₉N₇O₅[M + H]⁺: 661.69,
Observed: 661.83
tert-butyl (R)-(3-(2-(3-(3-(3-(4-(1H-pyrazol-4-yl) benzyl)-3-cyclopropylureido)
piperidin-1-yl) phenoxy)-2-methylpropanamido) propyl) carbamate (56)
¹H NMR (500 MHz, Chloroform-d) δ 8.41 (d, J = 1.6 Hz, 1H), 7.97 (dd, J = 3.5, 1.6 Hz, 1H),
7.50 (t, J = 4.6 Hz, 1H), 7.15 (t, J = 7.3 Hz, 1H), 6.66 (ddd, J = 7.3, 2.2, 1.1 Hz, 1H), 6.49
(ddd, J = 7.3, 2.2, 1.1 Hz, 1H), 6.44 (t, J = 2.2 Hz, 1H), 5.64 (d, J = 9.2 Hz, 1H), 5.11 (t, J =
5.1 Hz, 1H), 4.37 (s, 2H), 3.90-3.81 (m, 1H), 3.54 (dd, J = 12.5, 2.2 Hz, 1H), 3.45-3.34
(m, 2H), 3.27 (ddd, J = 12.4, 6.0, 3.3 Hz, 1H), 3.23-3.17 (m, 2H), 3.17-3.15 (m, 1H), 3.15-
3.12 (m, 2H), 1.97 (dddd, J = 12.3, 8.8, 6.1, 3.7 Hz, 1H), 1.84 (dddd, J = 14.8, 12.1, 6.0, 3.4
Hz, 1H), 1.76-1.74 (m, 1H), 1.74-1.67 (m, 2H), 1.67-1.59 (m, 1H), 1.47 (s, 3H), 1.42 (s,
9H), 0.75-0.58 (m, 4H). ESI-MS calculated for C₃₇H₅₁N₇O₅[M + H]⁺: 674.69, Observed: 674.86

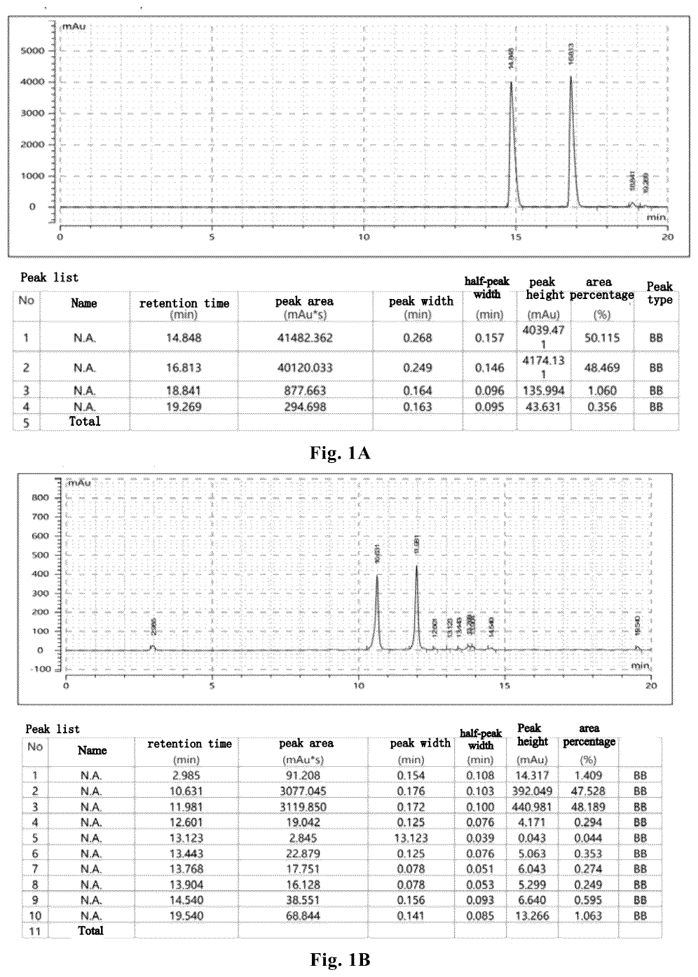

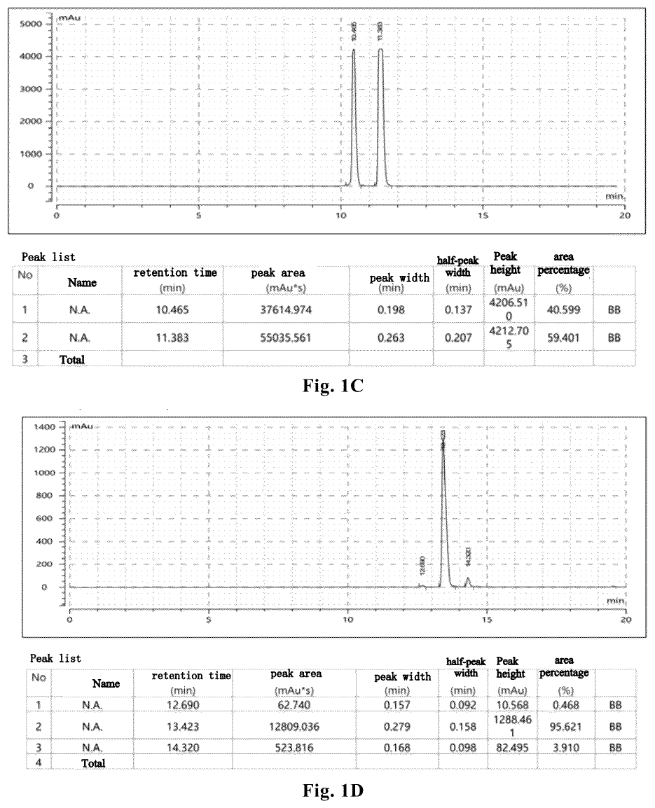

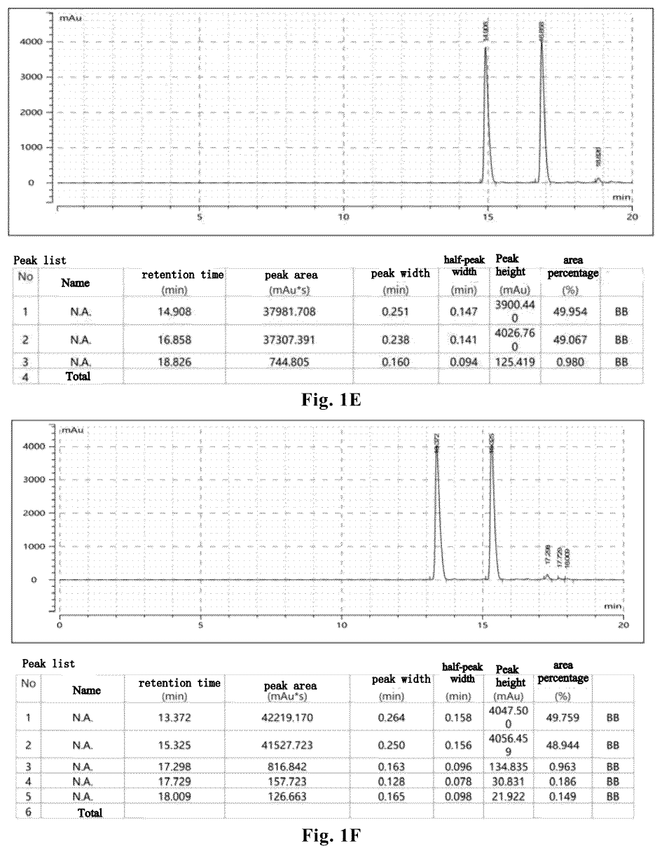

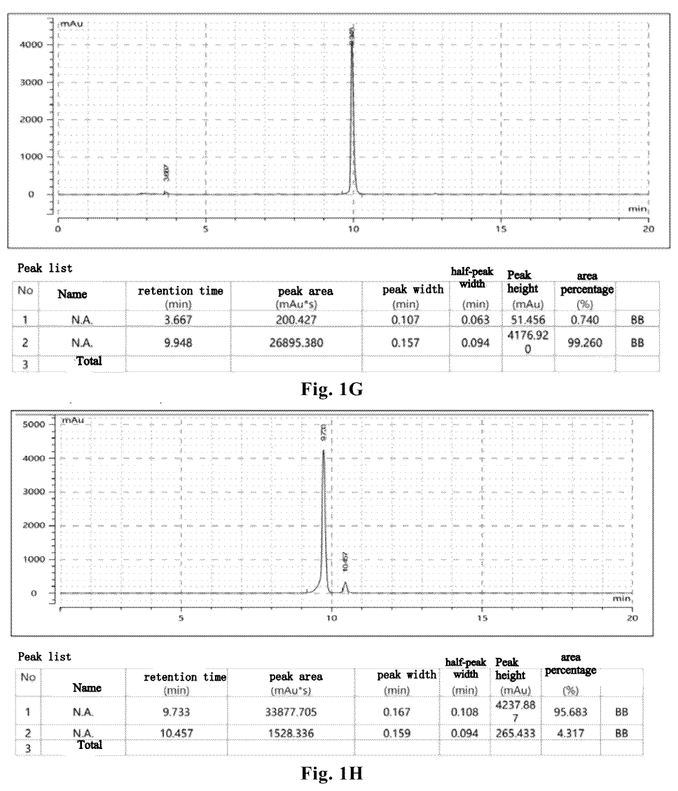

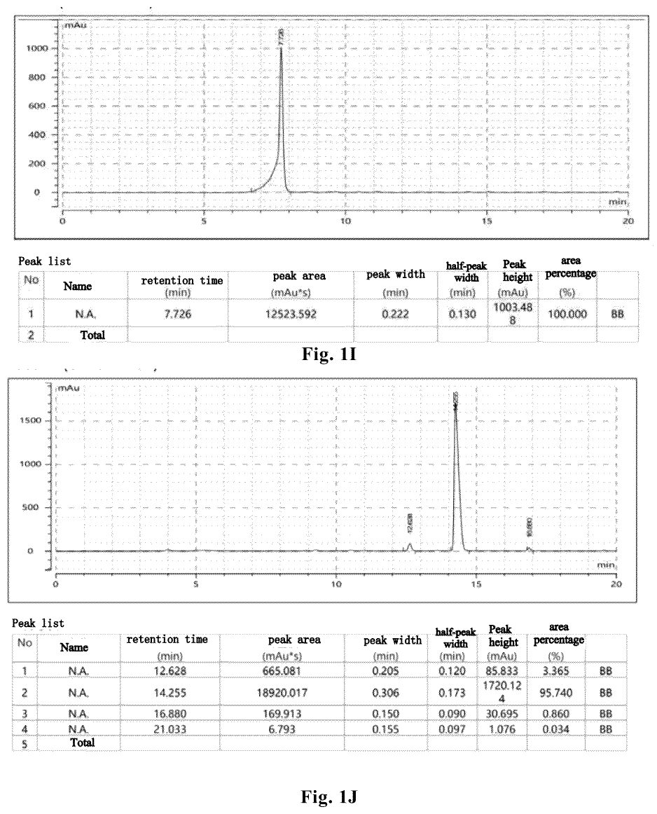

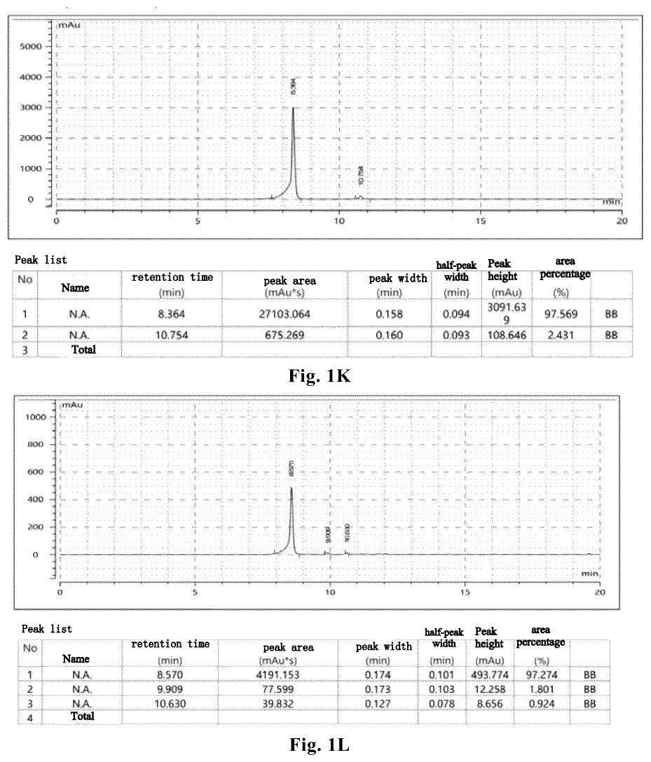

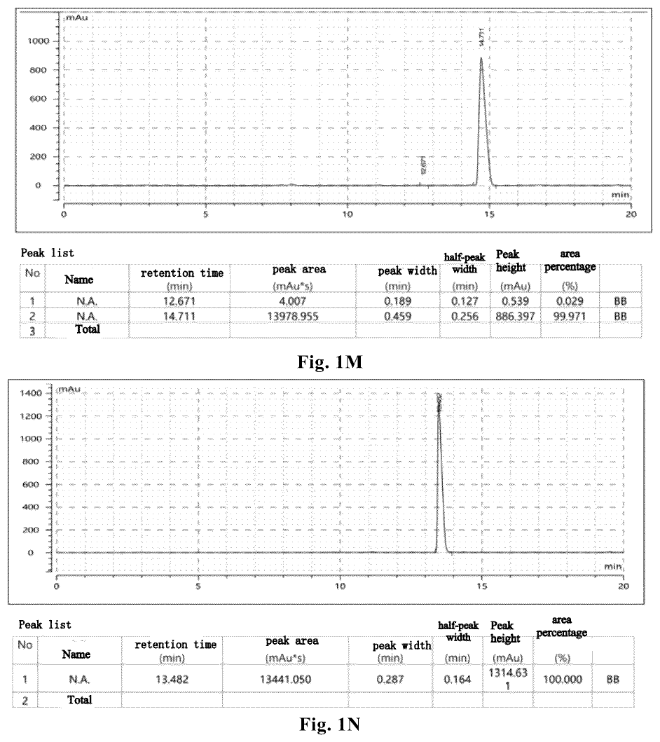

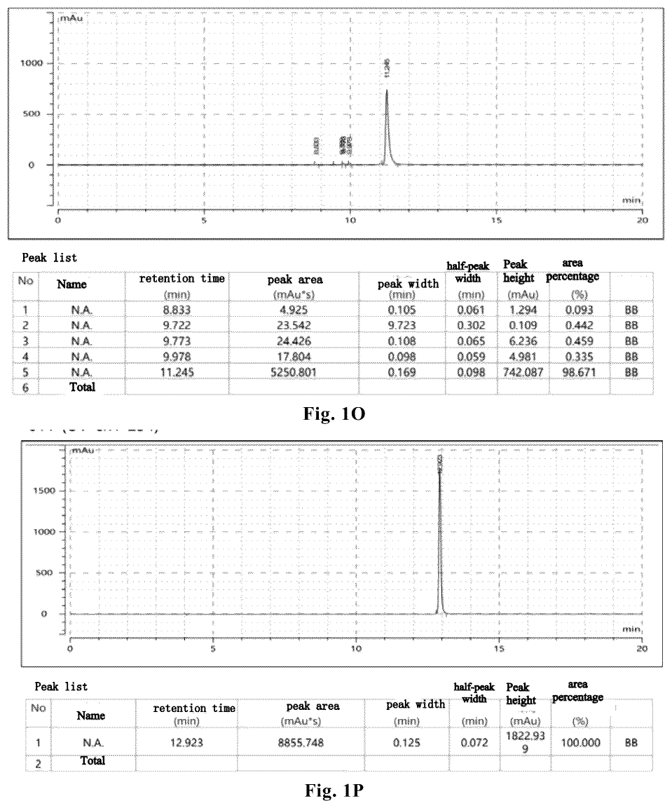

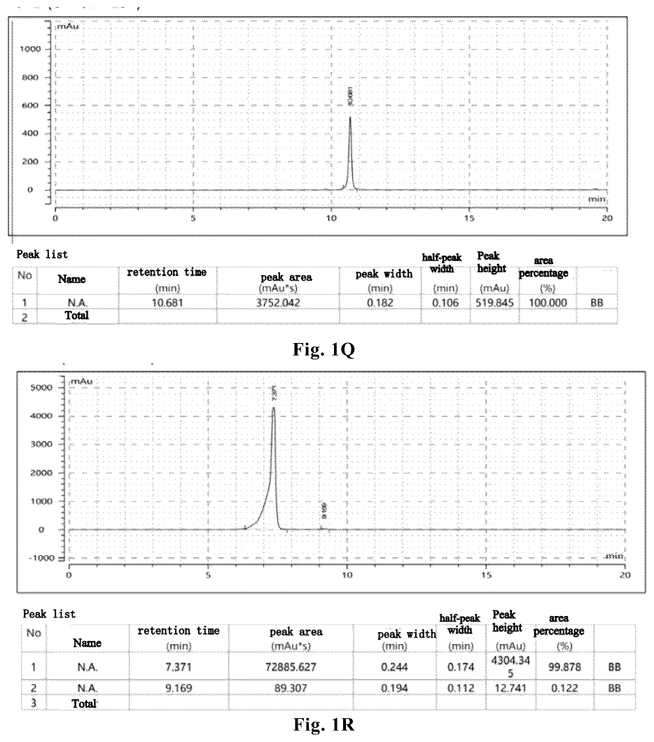

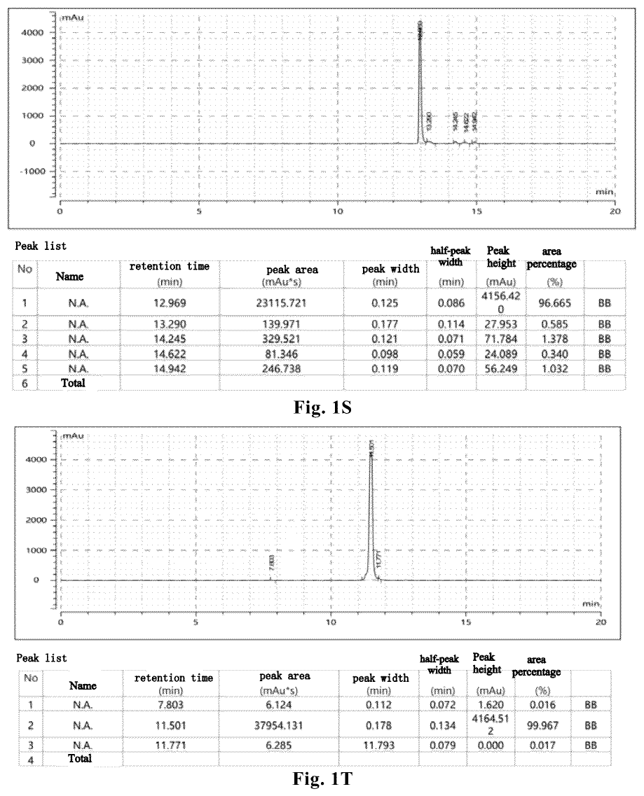

ii. The characterization results of compounds 1-20 are as follows:

| | |
|---|---|
| Compound 1 Free base form of A-049 | 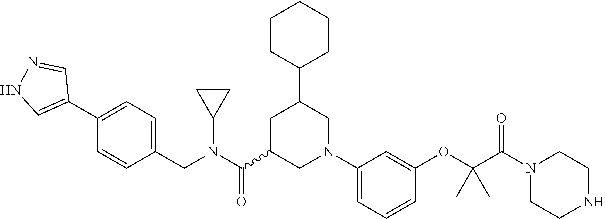 [SMILES: CC(C)(Oc1cccc(N2CC(C(=O)N(Cc3ccc(-c4cn[nH]c4)cc3)C3CC3)CC(C3CCCCC3)C2)c1)C(=O)N1CCNCC1] |
| | (Racemic) Purity: 98.584% (see FIG. 1A) |
| | 5-cyclohexyl-N-cyclopropyl-1-(3-{[2-methyl-1-oxo-1-(piperazin-1-yl)propan-2-yl] |
| | oxy}phenyl)-N-{[4-(1H-pyrazol-4-yl)phenyl]methyl}piperidine-3-carboxamide |
| | (1). ¹H NMR (400 MHz, Methanol-d₄) δ 8.55 (d, J = 12.9 Hz, 2H), 7.69 (t, J = 9.2 |
| | Hz, 2H), 7.57 (q, J = 8.3 Hz, 1H), 7.43 (dd, J = 19.0, 7.9 Hz, 3H), 7.26 (s, 1H), |
| | 7.06 (d, J = 8.1 Hz, 1H), 5.15 (d, J = 14.6 Hz, 2H), 4.41-4.27 (m, 1H), 3.97 (d, |
| | J = 13.8 Hz, 2H), 3.77-3.50 (m, 6H), 3.37 (s, 1H), 3.20 (s, 17H), 2.87 (s, 1H), |
| | 2.33-2.11 (m, 2H), 1.98 (s, 1H), 1.73-1.66 (m, 6H), 0.98 (s, 4H). |
| | ESI-MS calculated for C₃₉H₅₂N₆O₃[M + H]⁺: 653.88, Observed: 653.43 |
| Compound 2 Free base form of A-052 | 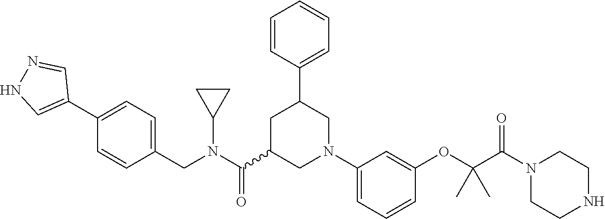 [SMILES: CC(C)(Oc1cccc(N2CC(C(=O)N(Cc3ccc(-c4cn[nH]c4)cc3)C3CC3)CC(c3ccccc3)C2)c1)C(=O)N1CCNCC1] |
| | (Racemic) Purity: 95.717% (see FIG. 1B) |
| | N-cyclopropyl-1-(3-{[2-methyl-1-oxo-1-(piperazin-1-yl)propan-2-yl]oxy}phenyl)- |
| | 5-phenyl-N-{[4-(1H-pyrazol-4-yl)phenyl]methyl}piperidine-3-carboxamide (2). |
| | ¹H NMR (400 MHz, DMSO-d₆) δ 8.73 (s, 1H), 8.01 (s, 2H), 7.49 (dd, J = 21.7, |
| | 7.8 Hz, 4H), 7.31 (t, J = 7.5 Hz, 2H), 7.21 (d, J = 7.4 Hz, 1H), 7.19-7.13 (m, |
| | 3H), 6.69 (d, J = 7.9 Hz, 1H), 6.45 (s, 1H), 6.25 (dd, J = 8.1, 2.3 Hz, 1H), 4.56 (d, |
| | J = 15.0 Hz, 1H), 3.95 (s, 4H), 3.77-3.66 (m, 4H), 3.60-3.47 (m, 3H), 3.36 (d, |
| | J = 8.6 Hz, 1H), 3.16 (ddd, J = 25.0, 11.8, 6.7 Hz, 3H), 3.00 (s, 2H), 2.76 (s, 1H), |
| | 2.45 (d, J = 5.3 Hz, 1H), 2.10 (d, J = 13.9 Hz, 1H), 2.04-1.99 (m, 1H), 1.56 (d, |
| | J = 2.5 Hz, 6H), 0.68-0.55 (m, 2H), 0.39 (d, J = 34.3 Hz, 2H). |
| | ESI-MS calculated for C₃₉H₄₆N₆O₃[M + H]⁺: 648.37, Observed: 648.08 |
| Compound 3 | 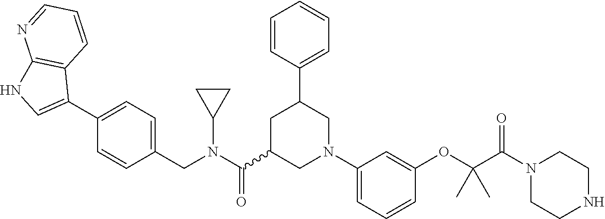 [SMILES: CC(C)(Oc1cccc(N2CC(C(=O)N(Cc3ccc(-c4c[nH]c5ncccc45)cc3)C3CC3)CC(c3ccccc3)C2)c1)C(=O)N1CCNCC1] |
| | (Racemic) Purity: 99.999%(see FIG. 1C) |
| | N-cyclopropyl-1-(3-{[2-methyl-1-oxo-1-(piperazin-1-yl)propan-2-yl]oxy}phenyl)- |
| | 5-phenyl-N-[(4-{1H-pyrrolo[2,3-b]pyridin-3-yl}phenyl)methyl]piperidine-3- |
| | carboxamide (3). ¹H NMR (400 MHz, Methanol-d₄) δ 8.46 (ddd, J = 12.7, 7.9, 1.3 |
| | Hz, 1H), 8.31 (ddd, J = 4.7, 2.8, 1.5 Hz, 1H), 7.78-7.64 (m, 3H), 7.49 (d, J = 7.6 |
| | Hz, 1H), 7.41-7.35 (m, 4H), 7.35-7.23 (m, 3H), 7.20 (q, J = 8.1 Hz, 1H), |
| | 6.77-6.65 (m, 1H), 6.57-6.30 (m, 2H), 4.81-4.60 (m, 2H), 4.15 (s, 1H), 3.91- |
| | 3.73 (m, 4H), 3.65 (d, J = 12.4 Hz, 1H), 3.14-2.99 (m, 3H), 2.97-2.78 (m, 3H), |
| | 2.25-2.14 (m, 2H), 2.12-1.99 (m, 1H), 1.66 (s, 6H), 1.01-0.72 (m, 4H). |
| | ESI-MS calculated for C₄₃H₄₈N₆O₃[M]⁺: 696.38, Observed: 696.51 |
| Compound 4 | 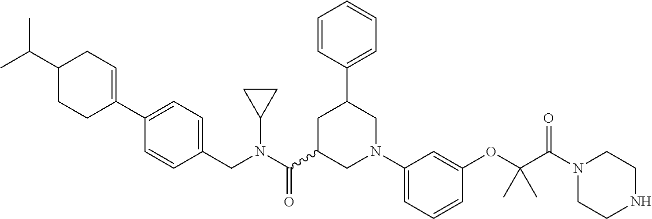 [SMILES: CC(C)C1CC=C(c2ccc(CN(C(=O)C3CC(c4ccccc4)CN(c4cccc(OC(C)(C)C(=O)N5CCNCC5)c4)C3)C3CC3)cc2)CC1] |
| | (Racemic) Purity: 95.621% (see FIG. 1D) |
| | N-cyclopropyl-1-(3-{[2-methyl-1-oxo-1-(piperazin-1-yl)propan-2-yl]oxy}phenyl)- |
| | 5-phenyl-N-({4-[4-(propan-2-yl)cyclohex-1-en-1-yl]phenyl}methyl)piperidine- |
| | 3-carboxamide (4). |
| | ¹H NMR (400 MHz, Chloroform-d) δ 7.45 (d, J = 4.3 Hz, 4H), 7.44-7.35 (m, |
| | 2H), 7.35-7.26 (m, 3H), 7.18 (t, J = 8.1 Hz, 2H), 6.75 (dd, J = 8.2, 2.2 Hz, 1H), |
| | 6.62 (t, J = 2.4 Hz, 1H), 6.42 (dd, J = 8.1, 2.3 Hz, 1H), 4.77 (d, J = 14.9 Hz, 1H), |
| | 4.49 (d, J = 14.9 Hz, 1H), 4.15 (s, 1H), 3.91 (s, 1H), 3.69 (d, J = 12.1 Hz, 3H), |
| | 3.13 (t, J = 11.6 Hz, 1H), 3.02 (s, 2H), 2.93-2.82 (m, 2H), 2.74 (tt, J = 6.9, 4.0 |
| | Hz, 2H), 2.66-2.61 (m, 2H), 2.04 (d, J = 12.4 Hz, 1H), 1.77 (td, J = 11.9, 4.8 |
| | Hz, 1H), 1.65 (s, 6H), 0.92 (dtdd, J = 29.9, 15.7, 7.5, 4.5 Hz, 5H), 0.09 (s, 1H). |
| | ESI-MS calculated for C₄₅H₅₈N₄O₃[M]⁺: 702.45, Observed: 702.48 |
| Compound 5 | 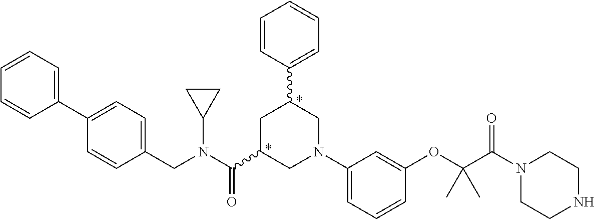 [SMILES: CC(C)(Oc1cccc(N2CC(C(=O)N(Cc3ccc(-c4ccccc4)cc3)C3CC3)CC(c3ccccc3)C2)c1)C(=O)N1CCNCC1] |
| | (Racemic) Purity: 99.020% (see FIG. 1D) |
| | N-cyclopropyl-1-(3-{[2-methyl-1-oxo-1-(piperazin-1-yl)propan-2-yl]oxy}phenyl)- |
| | 5-phenyl-N-[(4-phenylphenyl)methyl]piperidine-3-carboxamide (5). ¹H NMR |
| | (400 MHz, Chloroform-d) δ 7.57-7.50 (m, 3H), 7.41 (t, J = 7.5 Hz, 2H), 7.35- |
| | 7.29 (m, 4H), 7.26 (d, J = 3.7 Hz, 5H), 7.19 (t, J = 8.1 Hz, 2H), 6.82 (d, J = 8.2 |
| | Hz, 1H), 6.73 (s, 1H), 6.50-6.43 (m, 1H), 4.83 (d, J = 14.7 Hz, 1H), 4.45 (d, J = |
| | 14.6 Hz, 1H), 4.10 (s, 1H), 3.91 (s, 1H), 3.86 (s, 2H), 3.75 (d, J = 8.4 Hz, 2H), |
| | 3.32 (s, 1H), 3.22 (s, 1H), 2.97 (d, J = 11.6 Hz, 3H), 2.74-2.67 (m, 1H), 2.64 (s, |
| | 2H), 2.20 (d, J = 12.1 Hz, 1H), 2.10 (t, J = 12.2 Hz, 1H), 1.62 (s, 6H), 1.01-0.83 |
| | (m, 4H). |
| | ESI-MS calculated for C₄₂H₄₈N₄O₃[M]⁺: 656.37, Observed: 656.59 |
| Compound 6 | 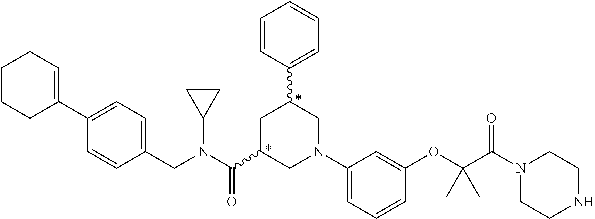 [SMILES: CC(C)(Oc1cccc(N2CC(C(=O)N(Cc3ccc(C4=CCCCC4)cc3)C3CC3)CC(c3ccccc3)C2)c1)C(=O)N1CCNCC1] |
| | (Racemic) Purity: 98.703% (see FIG. 1F) |
| | N-{[4-(cyclohex-1-en-1-yl)phenyl]methyl}-N-cyclopropyl-1-(3-{[2-methyl-1- |
| | oxo-1-(piperazin-1-yl)propan-2-yl]oxy}phenyl)-5-phenylpiperidine-3-carboxamide |
| | (6). |
| | ¹H NMR (400 MHz, Chloroform-d) δ 7.55 (dd, J = 10.6, 7.6 Hz, 4H), 7.43 (t, J = |
| | 7.5 Hz, 3H), 7.35 (q, J = 7.2 Hz, 3H), 7.30 (s, 1H), 7.28 (s, 4H), 7.26-7.18 (m, |
| | 2H), 6.88-6.82 (m, 1H), 6.76 (s, 1H), 6.54 (d, J = 7.7 Hz, 1H), 4.84 (d, J = 14.4 |
| | Hz, 1H), 4.42 (d, J = 14.3 Hz, 1H), 4.11 (s, 1H), 3.88 (s, 1H), 3.81-3.75 (m, |
| | 2H), 3.51 (s, 1H), 3.39 (d, J = 11.3 Hz, 1H), 3.33 (s, 1H), 3.01 (s, 1H), 2.50 (s, |
| | 1H), 2.16 (s, 1H), 1.63 (d, J = 6.9 Hz, 6H), 0.70 (d, J = 36.7 Hz, 5H). |
| | ESI-MS calculated for C₄₂H₅₂N₆O₃[M]⁺: 660.40, Observed: 660.67 |
| Compound 7 | 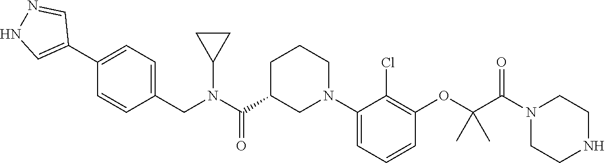 [SMILES: CC(C)(Oc1cccc(N2CCC[C@@H](C(=O)N(Cc3ccc(-c4cn[nH]c4)cc3)C3CC3)C2)c1Cl)C(=O)N1CCNCC1] |
| | Purity: 98.260% (see FIG. 1G) |
| | (3R)-1-(2-chloro-3-{[2-methyl-1-oxo-1-(piperazin-1-yl)propan-2-yl]oxy}phenyl)- |
| | N-cyclopropyl-N-{[4-(1H-pyrazol-4-yl)phenyl]methyl}piperidine-3-carboxamide |
| | (7) |
| | ¹H NMR (400 MHz, DMSO-d₆) δ 8.71 (s, 2H), 8.02 (s, 1H), 7.54 (d, J = 8.0 Hz, |
| | 2H), 7.23 (t, J = 8.2 Hz, 1H), 7.15 (d, J = 8.0 Hz, 2H), 6.88 (d, J = 8.1 Hz, 1H), |
| | 6.55 (d, J = 8.3 Hz, 1H), 4.58 (d, J = 14.9 Hz, 1H), 4.42 (s, 1H), 3.95 (s, 3H), |
| | 3.72 (s, 3H), 3.54 (t, J = 10.8 Hz, 2H), 3.33 (d, J = 11.0 Hz, 1H), 3.24 (d, J = 11.1 |
| | Hz, 1H), 2.84 (s, 1H), 2.71 (q, J = 10.8, 10.4 Hz, 4H), 1.99 (dt, J = 15.8, 8.9 Hz, |
| | 2H), 1.84 (d, J = 12.4 Hz, 1H), 1.71 (d, J = 12.5 Hz, 1H), 1.57 (d, J = 3.3 Hz, |
| | 6H), 0.93-0.75 (m, 4H). |
| | ESI-MS calculated for C33H41ClN6O3 [M]+: 605.30, Observed: 605.33 |
| Compound 8 | 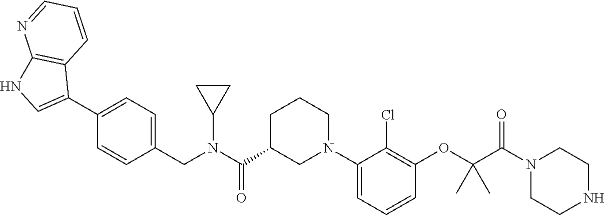 [SMILES: CC(C)(Oc1cccc(N2CCC[C@@H](C(=O)N(Cc3ccc(-c4c[nH]c5ncccc45)cc3)C3CC3)C2)c1Cl)C(=O)N1CCNCC1] |
| | Purity: 95.683% (see FIG. 1H) |
| | (3R)-1-(2-chloro-3-{[2-methyl-1-oxo-1-(piperazin-1-yl)propan-2-yl]oxy}phenyl)- |
| | N-cyclopropyl-N-[(4-{1H-pyrrolo[2,3-b]pyridin-3-yl}phenyl)methyl]piperidine- |
| | 3-carboxamide.(8) |
| | ¹H NMR (400 MHz, Methanol-d₄) δ 8.77-8.69 (m, 1H), 8.41 (dd, J = 5.5, 1.3 |
| | Hz, 1H), 7.86 (d, J = 3.6 Hz, 1H), 7.77-7.66 (m, 2H), 7.49 (dd, J = 8.0, 5.4 Hz, |
| | 1H), 7.37 (d, J = 8.0 Hz, 2H), 7.23 (t, J = 8.2 Hz, 1H), 6.94 (d, J = 8.2 Hz, 1H), |
| | 6.66 (d, J = 8.3 Hz, 1H), 4.82 (d, J = 15.0 Hz, 1H), 4.60 (d, J = 15.0 Hz, 1H), |
| | 4.17 (s, 1H), 3.89 (s, 1H), 3.83-3.73 (m, 1H), 3.46 (d, J = 11.6 Hz, 1H), 3.37 (s, |
| | 1H), 2.85 (s, 2H), 2.08 (d, J = 13.0 Hz, 1H), 1.93 (s, 1H), 1.68 (s, 6H), 1.07- |
| | 0.88 (m, 4H). |
| | ESI-MS calculated for C37H43ClN6O3 [M]+: 655.24, Observed: 654.90 |
| Compound 9 | 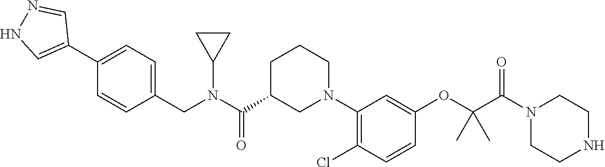 [SMILES: CC(C)(Oc1ccc(Cl)c(N2CCC[C@@H](C(=O)N(Cc3ccc(-c4cn[nH]c4)cc3)C3CC3)C2)c1)C(=O)N1CCNCC1] |
| | Purity: 100.000% (see FIG. 1I) |
| | (3R)-1-(2-chloro-3-{[2-methyl-1-oxo-1-(piperazin-1-yl)propan-2-yl]oxy}phenyl)- |
| | N-cyclopropyl-N-{[4-(1H-pyrazol-4-yl)phenyl]methyl}piperidine-3-carboxamide. |
| | (9) |
| | ¹H NMR (400 MHz, DMSO-d₆) δ 8.78 (s, 1H), 8.03 (s, 2H), 7.55 (d, J = 7.9 Hz, |
| | 2H), 7.17 (d, J = 8.0 Hz, 2H), 6.98 (d, J = 8.5 Hz, 2H), 6.67 (d, J = 8.5 Hz, 1H), |
| | 4.57 (s, 1H), 4.45 (d, J = 14.9 Hz, 1H), 3.97 (s, 1H), 3.74-3.68 (m, 1H), 3.41 (d, |
| | J = 14.2 Hz, 2H), 3.02 (s, 1H), 2.84-2.72 (m, 2H), 2.68 (d, J = 12.2 Hz, 1H), |
| | 2.63 (q, J = 5.5 Hz, 1H), 2.02-1.94 (m, 1H), 1.82 (d, J = 12.7 Hz, 1H), 1.68 (d, |
| | J = 12.8 Hz, 1H), 1.53 (d, J = 8.8 Hz, 6H), 0.83 (dp, J = 10.3, 5.9, 4.3 Hz, 4H). |
| | ESI-MS calculated for C₃₃H₄₁ClN₆O₃ [M]⁺: 605.30, Observed: 605.33 |
| Compound 10 | 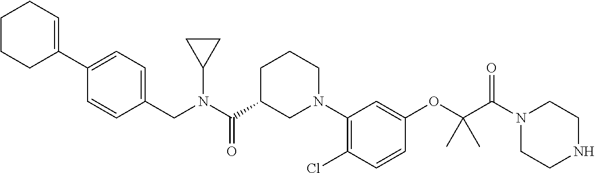 [SMILES: CC(C)(Oc1ccc(Cl)c(N2CCC[C@@H](C(=O)N(Cc3ccc(C4=CCCCC4)cc3)C3CC3)C2)c1)C(=O)N1CCNCC1] |
| | Purity: 95.740% (see FIG. 1J) |
| | (3R)-1-(2-chloro-5-{[2-methyl-1-oxo-1-(piperazin-1-yl)propan-2-yl]oxy}phenyl)- |
| | N-{[4-(cyclohex-1-en-1-yl)phenyl]methyl}-N-cyclopropylpiperidine-3- |
| | carboxamide (10) |
| | ¹H NMR (400 MHz, Methanol-d₄) δ 7.46 (s, 1H), 7.37 (d, J = 8.1 Hz, 2H), 7.22 |
| | (d, J = 8.0 Hz, 2H), 6.95 (s, 1H), 6.13 (td, J = 4.0, 2.0 Hz, 1H), 4.51 (d, J = 14.8 |
| | Hz, 1H), 3.98 (s, 1H), 3.66 (s, 1H), 2.86 (s, 1H), 2.45-2.37 (m, 2H), 2.23 (tt, J = |
| | 5.7, 3.1 Hz, 3H), 2.13 (s, 1H), 2.05 (d, J = 6.0 Hz, 1H), 1.86-1.80 (m, 2H), 1.78 |
| | (d, J = 12.4 Hz, 3H), 1.74-1.60 (m, 7H), 1.04 (d, J = 6.8 Hz, 1H), 0.97 (d, J = |
| | 6.6 Hz, 2H), 0.96-0.88 (m, 2H). |
| | ESI-MS calculated for C36H47ClN4O3 [M]+: 618.33, Observed: 618.64 |
| Compound 11 | 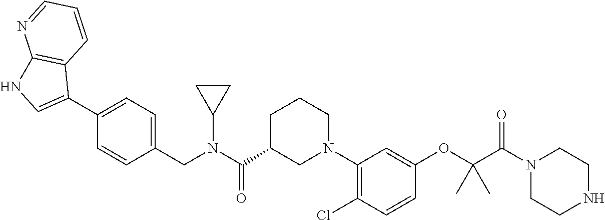 [SMILES: CC(C)(Oc1ccc(Cl)c(N2CCC[C@@H](C(=O)N(Cc3ccc(-c4c[nH]c5ncccc45)cc3)C3CC3)C2)c1)C(=O)N1CCNCC1] |
| | Purity: 97.569% (see FIG. 1K) |
| | (3R)-1-(2-chloro-5-{[2-methyl-1-oxo-1-(piperazin-1-yl)propan-2-yl]oxy}phenyl)- |
| | N-cyclopropyl-N-[(4-{1H-pyrrolo[2,3-b]pyridin-3-yl}phenyl)methyl]piperidine- |
| | 3-carboxamide.(11) |
| | ¹H NMR (400 MHz, DMSO-d₆) δ 11.98 (s, 1H), 8.79 (s, 1H), 8.34-8.28 (m, |
| | 2H), 7.87 (t, J = 2.8 Hz, 1H), 7.68 (d, J = 7.9 Hz, 2H), 7.24 (dd, J = 8.3, 2.2 Hz, |
| | 2H), 7.18 (dd, J = 7.9, 4.8 Hz, 1H), 6.89 (d, J = 8.1 Hz, 1H), 6.55 (d, J = 8.3 Hz, |
| | 1H), 4.64 (d, J = 15.1 Hz, 1H), 4.49 (d, J = 15.1 Hz, 1H), 3.95 (s, 1H), 3.72 (s, |
| | 1H), 3.56 (s, 1H), 3.39-3.32 (m, 1H), 3.25 (d, J = 11.2 Hz, 1H), 2.85 (s, 1H), |
| | 2.80-2.60 (m, 4H), 2.00 (d, J = 12.3 Hz, 1H), 1.85 (d, J = 12.8 Hz, 1H), 1.60 (s, |
| | 2H), 1.56 (d, J = 2.4 Hz, 6H), 0.92-0.82 (m, 4H). |
| | ESI-MS calculated for C₃₇H₄₃ClN₆O₃ [M]⁺: 654.24, Observed: 654.48 |
| Compound 12 | 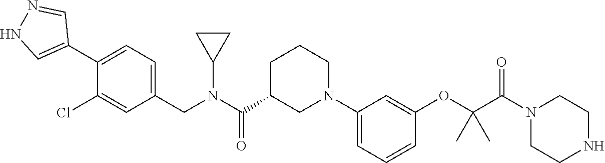 [SMILES: CC(C)(Oc1cccc(N2CCC[C@@H](C(=O)N(Cc3ccc(-c4cn[nH]c4)c(Cl)c3)C3CC3)C2)c1)C(=O)N1CCNCC1] |
| | Purity: 97.274% (see FIG. 1L) |
| | (3R)-N-{[3-chloro-4-(1H-pyrazol-4-yl)phenyl]methyl}-N-cyclopropyl-1-(3-{[2- |
| | methyl-1-oxo-1-(piperazin-1-yl)propan-2-yl]oxy}phenyl)piperidine-3-carboxamide. |
| | (12)¹H NMR (400 MHz, DMSO-d₆) δ 9.51 (s, 1H), 8.03 (s, 2H), 7.56 (d, J = |
| | 8.0 Hz, 2H), 7.44-7.30 (m, 3H), 7.16 (dd, J = 8.1, 1.7 Hz, 2H), 6.65 (s, 1H), |
| | 4.65 (d, J = 15.2 Hz, 1H), 4.42 (s, 1H), 3.75 (s, 2H), 3.70-3.61 (m, 2H), 3.57 (d, |
| | J = 11.2 Hz, 2H), 3.39 (s, 3H), 2.99 (s, 1H), 2.78 (ddd, J = 20.9, 10.5, 4.6 Hz, |
| | 4H), 2.03 (d, J = 13.1 Hz, 2H), 1.89 (d, J = 13.0 Hz, 1H), 1.68 (d, J = 12.1 Hz, |
| | 1H), 1.57 (s, 6H), 0.84 (dt, J = 15.1, 5.0 Hz, 4H). |
| | ESI-MS calculated for C₃₃H₄₁ClN₆O₃ [M]⁺: 605.18, Observed: 605.33 |
| Compound 13 | 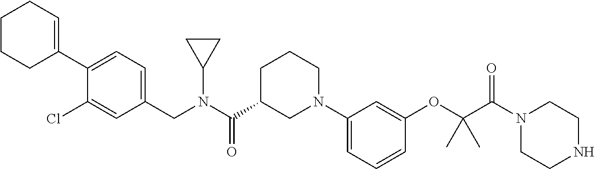 [SMILES: CC(C)(Oc1cccc(N2CCC[C@@H](C(=O)N(Cc3ccc(C4=CCCCC4)c(Cl)c3)C3CC3)C2)c1)C(=O)N1CCNCC1] |
| | Purity: 99.971% (see FIG. 1M) |
| | (3R)-N-{[3-chloro-4-(cyclohex-1-en-1-yl)phenyl]methyl}-N-cyclopropyl-1-(3-{[2- |
| | methyl-1-oxo-1-(piperazin-1-yl)propan-2-yl]oxy}phenyl)piperidine-3-carboxamide |
| | (13).¹H NMR (400 MHz, Chloroform-d) δ 7.22-7.15 (m, 2H), 7.12 (d, J = |
| | 7.8 Hz, 1H), 7.04 (dd, J = 7.8, 1.7 Hz, 1H), 6.73 (dd, J = 8.3, 2.2 Hz, 1H), 6.60 (t, |
| | J = 2.3 Hz, 1H), 6.41 (dd, J = 8.2, 2.3 Hz, 1H), 5.67 (tt, J = 3.7, 1.7 Hz, 1H), 4.71 |
| | (d, J = 14.9 Hz, 1H), 4.41 (d, J = 14.9 Hz, 1H), 4.16 (s, 1H), 3.92 (s, 1H), 3.69 (s, |
| | 1H), 3.63 (d, J = 15.3 Hz, 2H), 3.10 (t, J = 11.2 Hz, 1H), 3.03 (s, 1H), 2.91-2.80 |
| | (m, 1H), 2.68 (s, 1H), 2.64 (s, 1H), 2.33-2.22 (m, 4H), 2.22-2.15 (m, 4H), 2.00 |
| | (s, 1H), 1.90 (s, 1H), 1.82-1.66 (m, 7H), 0.94 (dt, J = 24.1, 6.3 Hz, 2H), 0.84 (q, |
| | J = 8.9, 6.6 Hz, 2H). |
| | ESI-MS calculated for C₃₆H₄₇ClN₄O₃ [M]⁺: 618.33, Observed: 618.62 |
| Compound 14 | Purity: 100.000% (see FIG. 1N) |
| | (3R)-N-[(3-chloro-4-phenylphenyl)methyl]-N-cyclopropyl-1-(3-{[2-methyl-1-oxo- |
| | 1-(piperazin-1-yl)propan-2-yl]oxy}phenyl)piperidine-3-carboxamide (14). ¹H |
| | NMR (400 MHz, Chloroform-d) δ 7.45 (d, J = 1.1 Hz, 2H), 7.44-7.35 (m, 3H), |
| | 7.35-7.27 (m, 3H), 7.23 (t, J = 8.3 Hz, 1H), 7.16 (dd, J = 7.9, 1.8 Hz, 1H), 6.84 |
| | (dd, J = 8.2, 2.2 Hz, 1H), 6.75 (t, J = 2.3 Hz, 1H), 6.52 (dd, J = 8.2, 2.3 Hz, 1H), |
| | 4.78 (d, J = 14.9 Hz, 1H), 4.47 (d, J = 14.9 Hz, 1H), 4.12 (d, J = 6.7 Hz, 1H), |
| | 3.91 (s, 1H), 3.69 (d, J = 12.2 Hz, 2H), 3.23 (t, J = 11.5 Hz, 1H), 3.02 (s, 1H), |
| | 3.01-2.92 (m, 1H), 2.74 (tt, J = 6.9, 4.1 Hz, 2H), 2.63 (s, 2H), 2.06 (d, J = 13.2 |
| | Hz, 1H), 1.97 (s, 1H), 1.65 (s, 5H), 1.08-0.82 (m, 4H). |
| | ESI-MS calculated for C₃₆H₄₃ClN₄O₃ [M + H]⁺: 616.22, Observed: 616.31 |
| Compound 15 | 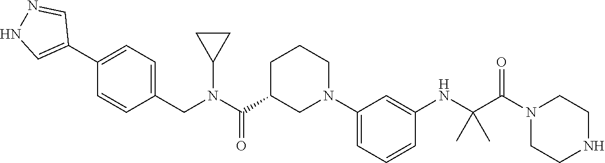 [SMILES: CC(C)(Nc1cccc(N2CCC[C@@H](C(=O)N(Cc3ccc(-c4cn[nH]c4)cc3)C3CC3)C2)c1)C(=O)N1CCNCC1] |
| | Purity: 98.671% (see FIG. 1O) |
| | (3R)-N-cyclopropyl-1-(3-{[2-methyl-1-oxo-1-(piperazin-1-yl)propan-2-yl]amino} |
| | phenyl)-N-{[4-(1H-pyrazol-4-yl)phenyl]methyl}piperidine-3-carboxamide |
| | (15). ¹H NMR (400 MHz, DMSO-d₆) δ 8.77 (s, 1H), 8.03 (s, 2H), 7.55 (d, J = 8.2 |
| | Hz, 2H), 7.30 (dp, J = 14.7, 8.2, 7.4 Hz, 1H), 7.18 (d, J = 8.0 Hz, 2H), 7.08 (t, J = |
| | 8.2 Hz, 1H), 6.55 (s, 1H), 6.34 (s, 1H), 6.19 (s, 1H), 4.64 (d, J = 15.0 Hz, 1H), |
| | 4.42 (d, J = 15.0 Hz, 1H), 4.07 (s, 1H), 3.69 (s, 1H), 3.60 (dd, J = 19.3, 12.1 Hz, |
| | 3H), 2.74 (q, J = 4.9, 4.4 Hz, 1H), 2.69 (s, 1H), 1.99 (d, J = 11.8 Hz, 1H), 1.84 (s, |
| | 1H), 1.63 (d, J = 13.2 Hz, 1H), 1.44 (s, 5H), 1.26 (dd, J = 11.4, 5.2 Hz, 1H), 0.90- |
| | 0.77 (m, 3H). |
| | ESI-MS calculated for C₃₃H₄₃N₇O₃ [M]⁺: 569.75, Observed: 569.71 |
| Compound 16 | 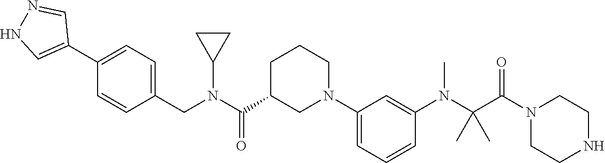 [SMILES: CN(c1cccc(N2CCC[C@@H](C(=O)N(Cc3ccc(-c4cn[nH]c4)cc3)C3CC3)C2)c1)C(C)(C)C(=O)N1CCNCC1] |
| | Purity: 100.00% (see FIG. 1P) |
| | (3R)-N-cyclopropyl-1-(3-{methyl[2-methyl-1-oxo-1-(piperazin-1-yl)propan-2-yl] |
| | amino}phenyl)-N-{[4-(1H-pyrazol-4-yl)phenyl]methyl}piperidine-3-carboxamid |
| | (16). ¹H NMR (400 MHz, Methanol-d₄) δ 7.96 (s, 2H), 7.57 (d, J = 7.9 Hz, 2H), |
| | 7.33 (dd, J = 30.0, 8.1 Hz, 4H), 6.81 (d, J = 16.8 Hz, 2H), 5.36 (t, J = 4.8 Hz, |
| | 1H), 4.53 (d, J = 14.9 Hz, 2H), 4.00 (s, 1H), 3.82 (s, 1H), 3.70 (t, J = 12.3 Hz, |
| | 3H), 3.02 (s, 3H), 2.98 (s, 1H), 2.84 (s, 1H), 2.21 (t, J = 7.5 Hz, 1H), 2.11 (s, 1H), |
| | 2.05 (d, J = 5.7 Hz, 2H), 1.61 (s, 1H), 1.56 (d, J = 2.6 Hz, 5H), 0.99-0.88 (m, |
| | 4H). |
| | ESI-MS calculated for C₃₄H₄₅N₇O₃ [M]⁺: 583.78, Observed: 583.75 |
| Compound 17 | 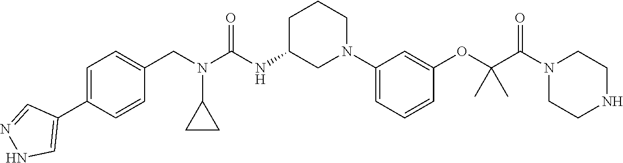 [SMILES: CC(C)(Oc1cccc(N2CCC[C@@H](NC(=O)N(Cc3ccc(-c4cn[nH]c4)cc3)C3CC3)C2)c1)C(=O)N1CCNCC1] |
| | Purity: 100.00% (see FIG. 1Q) |
| | 1-cyclopropyl-3-[(3R)-1-(3-{[2-methyl-1-oxo-1-(piperazin-1-yl)propan-2-yl]oxy} |
| | phenyl)piperidin-3-yl]-1-{[4-(1H-pyrazol-4-yl)phenyl]methyl}urea (17)¹H |
| | NMR (400 MHz, Methanol-d₄) δ 7.95 (s, 1H), 7.59-7.52 (m, 2H), 7.28 (d, J = |
| | 8.2 Hz, 2H), 7.15 (t, J = 8.2 Hz, 1H), 6.68 (dd, J = 8.2, 2.2 Hz, 1H), 6.48 (t, J = |
| | 2.3 Hz, 1H), 6.32 (dd, J = 7.8, 2.3 Hz, 1H), 5.36 (t, J = 4.9 Hz, 1H), 4.57 (s, 1H), |
| | 4.15 (s, 1H), 3.91 (dt, J = 8.4, 4.5 Hz, 1H), 3.85 (s, 1H), 3.55 (dd, J = 12.2, 3.5 |
| | Hz, 1H), 3.09 (s, 1H), 3.01 (t, J = 9.2 Hz, 1H), 2.93 (dd, J = 12.0, 8.1 Hz, 1H), |
| | 2.79 (s, 1H), 2.45 (tt, J = 6.9, 3.8 Hz, 1H), 2.25-2.17 (m, 1H), 2.05 (d, J = 6.1 |
| | Hz, 1H), 1.95 (d, J = 11.0 Hz, 1H), 1.85 (s, 1H), 1.65 (d, J = 3.5 Hz, 6H), 0.96- |
| | 0.88 (m, 1H), 0.88-0.78 (m, 2H), 0.76 (s, 1H). |
| | ESI-MS calculated for C₃₃H₄₃N₇O₃ [M + 2]⁺: 588.74, Observed: 588.97 |
| Compound 18 | 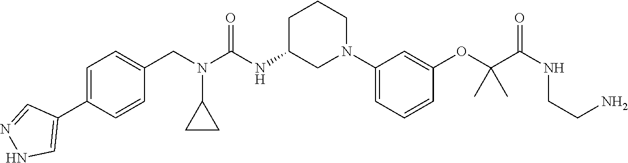 [SMILES: CC(C)(Oc1cccc(N2CCC[C@@H](NC(=O)N(Cc3ccc(-c4cn[nH]c4)cc3)C3CC3)C2)c1)C(=O)NCCN] |
| | Purity: 99.878% (see FIG. 1R) |
| | N-(2-aminoethyl)-2-{3-[(3R)-3-{[cyclopropyl({[4-(1H-pyrazol-4-yl)phenyl] |
| | methyl})carbamoyl]amino}piperidin-1-yl]phenoxy}-2-methylpropanamide |
| | (18). ¹H NMR (400 MHz, Methanol-d₄) δ 8.50 (t, J = 5.8 Hz, 1H), 7.95 (s, 2H), |
| | 7.58-7.51 (m, 2H), 7.30-7.24 (m, 2H), 7.17 (t, J = 8.2 Hz, 1H), 6.78 (dd, J = |
| | 7.9, 2.3 Hz, 1H), 6.65 (t, J = 2.3 Hz, 1H), 6.47 (dd, J = 7.8, 2.2 Hz, 1H), 4.56 (s, |
| | 2H), 3.98 (dt, J = 8.1, 4.2 Hz, 1H), 3.52 (p, J = 6.3 Hz, 3H), 3.13-2.97 (m, 4H), |
| | 2.44 (tt, J = 6.7, 3.8 Hz, 1H), 1.98-1.85 (m, 2H), 1.81-1.66 (m, 2H), 1.52 (d, |
| | J = 1.8 Hz, 6H), 1.32 (s, 1H), 0.87-0.70 (m, 4H). |
| | ESI-MS calculated for C₃₁H₄₁N₄O₃ [M]⁺: 559.74, Observed: 559.98 |
| Compound 19 | 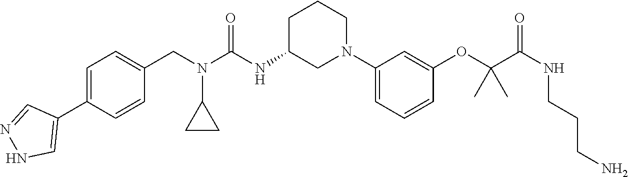 [SMILES: CC(C)(Oc1cccc(N2CCC[C@@H](NC(=O)N(Cc3ccc(-c4cn[nH]c4)cc3)C3CC3)C2)c1)C(=O)NCCCN] |
| | Purity: 96.665% (see FIG. 1S) |
| | N-(3-aminopropyl)-2-{3-[(3R)-3-{[cyclopropyl({[4-(1H-pyrazol-4-yl)phenyl] |
| | methyl})carbamoyl]amino}piperidin-1-yl]phenoxy}-2-methylpropanamide (19). |
| | ¹H NMR (400 MHz, Chloroform-d) δ 7.44-7.35 (m, 3H), 7.25 (d, J = 8.0 Hz, 4H), |
| | 7.12 (ddd, J = 14.0, 10.1, 5.8 Hz, 3H), 6.65 (d, J = 8.2 Hz, 1H), 6.55 (s, 1H), 6.43- |
| | 6.36 (m, 1H), 5.77 (d, J = 8.1 Hz, 1H), 4.69 (d, J = 15.2 Hz, 1H), 4.44 (d, J = |
| | 15.2 Hz, 1H), 4.13 (s, 1H), 3.27 (h, J = 11.9 Hz, 5H), 3.19-3.02 (m, 3H), 2.38 |
| | (s, 2H), 2.04 (d, J = 6.1 Hz, 1H), 1.81 (s, 2H), 1.68 (dd, J = 13.1, 6.4 Hz, 3H), |
| | 1.51 (d, J = 3.8 Hz, 6H), 1.38 (d, J = 14.9 Hz, 6H), 1.30 (d, J = 11.9 Hz, 16H), |
| | 0.77 (s, 4H), 0.09 (s, 80H). |
| | ESI-MS calculated for C₃₂H₄₃N₇O₃ [M]⁺: 573.74, Observed: 573.82 |
| Compound 20 | 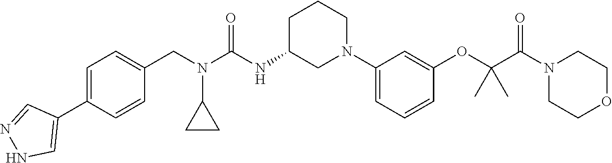 [SMILES: CC(C)(Oc1cccc(N2CCC[C@@H](NC(=O)N(Cc3ccc(-c4cn[nH]c4)cc3)C3CC3)C2)c1)C(=O)N1CCOCC1] |
| | Purity: 99.967 (see FIG. 1T) |
| | 1-cyclopropyl-3-[(3R)-1-(3-{[2-methyl-1-(morpholin-4-yl)-1-oxopropan-2-yl] |
| | oxy}phenyl)piperidin-3-yl]-1-{[4-(1H-pyrazol-4-yl)phenyl]methyl}urea (20). ¹H |
| | NMR (400 MHz, Methanol-d₄) δ 7.94 (s, 1H), 7.58-7.51 (m, 2H), 7.28 (d, J = |
| | 8.1 Hz, 2H), 7.12 (t, J = 8.2 Hz, 1H), 6.66 (dd, J = 8.1, 2.3 Hz, 1H), 6.47 (t, J = |
| | 2.4 Hz, 1H), 6.32 (dd, J = 8.1, 2.4 Hz, 1H), 6.20 (d, J = 8.1 Hz, 1H), 5.36 (dd, J = |
| | 5.6, 4.1 Hz, 1H), 4.56 (d, J = 2.7 Hz, 2H), 3.90 (s, 1H), 3.60 (s, 1H), 3.55 (s, 1H), |
| | 3.43 (dd, J = 12.0, 3.3 Hz, 1H), 3.26 (s, 2H), 3.21 (d, J = 6.0 Hz, 1H), 3.10 (s, |
| | 1H), 3.04 (dd, J = 12.2, 7.3 Hz, 2H), 2.47-2.39 (m, 1H), 2.21 (t, J = 7.6 Hz, 1H), |
| | 2.08-2.00 (m, 1H), 1.88 (s, 1H), 1.73 (s, 1H), 1.62 (d, J = 1.6 Hz, 4H), 0.96- |
| | 0.88 (m, 1H), 0.82 (dd, J = 6.8, 4.6 Hz, 2H), 0.78-0.72 (m, 2H). |
| | ESI-MS calculated for C₃₆H₄₂N₆O₄ [M + 2]⁺: 588.74, Observed: 588.97 |

### Test Examples

### I Test Methods

(1) FP (BCL-9 avidity):

Adding 200 μl FP buffer (25 mM HEPES, 100 mM NaCl, 0.01% Triton X-100, 0.1% BSA) and 1 μM beta-catenin to a black 96-well plate and adding C37 series compounds of different concentrations to the wells with a concentration gradient of 0.625, 1.25, 2.5, 5, and 10 mol/L, setting three repeat wells for each concentration condition. Incubating for 2 hours on a horizontal shaker. Then adding 2 nM FAP-Bcl9 tracer to each well, and after incubation for 2H on a horizontal shaker, measuring the absorbance. A positive control representing 100% inhibition contains the tracer only. A negative control representing 0% inhibition contains the tracer and β-catenin.

### (2) CCK8

1. HCT116 cell plating

Digesting, counting, and plating the cells in 96-well plates (flat bottom, transparent) with 10,000 cells/100 ul DMEM (10% FBS) per well;
2. Observing the state of cells on the next day, and start dosing after cell attachment is intact;
3. diluting 514 with DMEM (2% FBS) by gradient dilution with concentrations of 20 μM, 10 μM, 5 μM, 2.5 μM, 1.25 μM, 0.625 μM, 0.3125 μM, 0.15625 μM, 0 μM (equal volume of DMSO);
4. Adding 100 μl of the above different concentrations of 514 to each well, and making two repeat wells for each concentration gradient; leaving three blank wells (only adding DMEM 2% FBS culture medium without cells).
5. Culturing at 37° C. for 24 hours;
6. Adding 10 μl CCK-8 enhanced solution to each well: due to the small amount added of CCK-8 in each well, it may cause errors resulting from the reagent staining on the wall of the well. It is recommended to gently tap the culture plate after adding the reagent to help mixing;
7. Incubating in the incubator for 0.5-4 hours: the amount of Formazan formed is not the same for different cell types, in most cases, incubation for 1 hour is enough. If the color development is not enough, incubation can be continued to determine optimal conditions.
8. Measuring the absorbance at 450 nm and 600 nm (excluding the interference of the background color of the reagent and the absorbance value of the well plate itself);
9. Using OD450 nm-OD600 nm for the final absorbance, and calculating the inhibition rate:

$$Inhibition ratio = [ ( A c - As ) / ( Ac - A b ) ] \times 100 \%$$

As: absorbance of experimental wells (medium containing cells, CCK-8, drug to be tested);
Ac: absorbance of control wells (medium containing cells, CCK-8, and no drug to be tested);
Ab: absorbance of blank wells (medium without cells and drug to be tested, CCK-8).
   (3) qPCR
1. HCT116 cell plating

Cells were digesting, counting, and plating cells in 24-well plates (flat bottom, clear), with 3×10\hat{ }5 cells/500 ul DMEM (10% FBS) per well.
2. Observing the state of cells on the next day, and start dosing after cell attachment is intact

Diluting 514 with DMEM (2% FBS) by gradient dilution with concentrations of 20 μM, 10 μM, 5 μM, 2.5 μM, 1.25 μM, 0.625 μM, 0.3125 μM, 0.15625 μM, 0 μM (equal volume DMSO)

Adding 500 μl of the above different concentrations of 514 to each well, and setting a single well for each concentration gradient except three repeat wells for the 0 μM concentration (double wells for qPCR).
3. Incubating at 37° C. for 24 hours
4. Discarding the supernatant, washing once with PBS, and adding trizol at 500/well
5. RNA extraction (step omitted)
6. Reverse transcription (step omitted, according to the kit)
7. qPCR (step omitted, following the kit steps)

### Primer Human AXIN2

H-AXIN2-F
cggaaactgttgacagtggat
H-AXIN2-R
ggtgcaaagacatagccagaa
Human β-actin
H-beta-actin-F
TGGCACCCAGCACAATGAA
H-beta-actin-R
CTAAGTCATAGTCCGCCTAGAAGCA

8. calculation of inhibition rate

$$\begin{matrix}Calculating 2 \hat{} ( - ΔΔ CT ) for each concentration gradient . \\ Inhibition = ( 1 - experimental well / control well ) \times 100 \%\end{matrix}$$

(4) Transformation of Fibroblasts into Myofibroblasts (Anti-Fibrosis Test)
I. Main materials

| No | Material name | Brand | Item number |
|---|---|---|---|
| 1 | human cell line HFL1 | Kebai | |
| 2 | TGF - beta 1 | MCE | HY-P70543 |
| 3 | a-SMA | Shanghai Zhenke | |
| 4 | Collagen type I Assay | Nanjing Jiancheng | H142-1-2 |
| | Kit | | |
| 5 | Collagen type III Assay | Nanjing Jiancheng | H144-1-2 |
| | Kit | | |

HFL1 medium: F12K+10% FBS, adherent growth
II. Experimental Setup
1. Normal group (medium only), model group (induced by 20 ng/ml TGF-β1), test sample group (20 ng/ml TGF-β1+ compound with different final concentration)
2. 5 compounds, 3 concentration gradients, each gradient 2 repeat wells.
II. Experimental steps
1. Digesting and counting, 5e⁵ cells/well/2 ml, and seeded in 6-well plates.
2. After adherent, adding TGF-β1 at a final concentration of 20 ng/ml and stimulating for 48 hours. Adding compounds with different final concentrations (0, 5 uM, 20 uM).
3. Collecting the supernatant, and detecting the amount of col1 and col3 in the supernatant by kit. Lysing the cells with lysate solution, centrifuging, and collecting the supernatant.
4. Detection of a-SMA and other genes by qPCR
   ii. Test Results
(1) BCL-9 affinity results are shown in Tables 1.1, 1.2, and 1.3, and the test methods were as described above.

TABLE 1.1
BCL-9 affinity of compounds 1-6

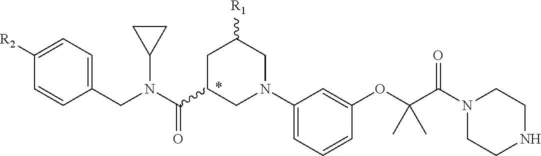

| | | | | BCL-9 avidity FP IC50 |
|---|---|---|---|---|
| Compounds | *Chirality | R₁ | R₂ | (μM) |
| 1 | Racemic | 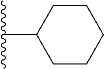 [SMILES: *C1CCCCC1] | 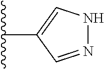 [SMILES: *c1cn[nH]c1] | >10 μM |
| 2 | Racemic | Ph- | 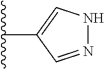 [SMILES: *c1cn[nH]c1] | >10 μM |
| 3 | Racemic | Ph- | 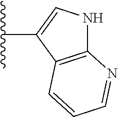 [SMILES: *c1c[nH]c2ncccc12] | 2.760 μM |
| 4 | Racemic | Ph- | 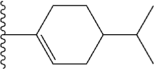 [SMILES: *C1=CCC(C(C)C)CC1] | |
| 5 | Racemic | Ph- | Ph- | 2.246 μM |
| 6 | Racemic | Ph- | 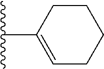 [SMILES: *C1=CCCCC1] | 7.753 μM |

TABLE 1.2
BCL-9 affinity of compounds 7-16

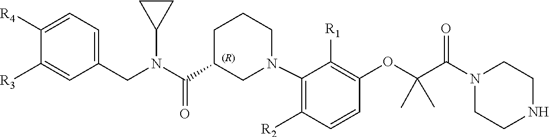

| | | | | | BCL-9 avidity FP IC₅₀ |
|---|---|---|---|---|---|
| | R₁ | R₂ | R₃ | R₄ | (μM) |
| Compound 7 | Cl | H | H | 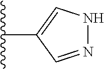 [SMILES: *c1cn[nH]c1] | 3.145 μM |
| Compound 8 | Cl | H | H | 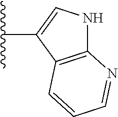 [SMILES: *c1c[nH]c2ncccc12] | >10 μM |
| Compound 9 | H | Cl | H | 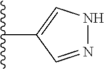 [SMILES: *c1cn[nH]c1] | >10 μM |
| Compound 10 | H | Cl | H | 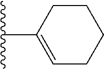 [SMILES: *C1=CCCCC1] | 2.7 μM |
| Compound 11 | H | Cl | H | 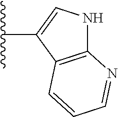 [SMILES: *c1c[nH]c2ncccc12] | 2.437 μM |
| Compound 12 | H | H | Cl | 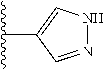 [SMILES: *c1cn[nH]c1] | >10 μM |
| Compound 13 | H | H | Cl | 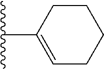 [SMILES: *C1=CCCCC1] | |
| Compound 14 | H | H | Cl | Ph- | |

TABLE 1.3
affinity of compounds 15, 16 as well as compounds 17-20 containing urea
structures

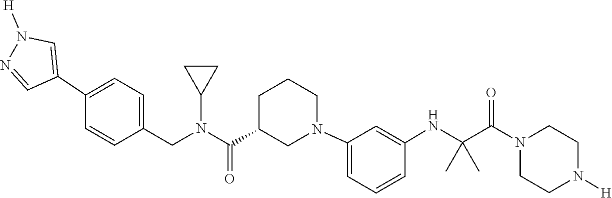 [SMILES: CC(C)(Nc1cccc(N2CCC[C@@H](C(=O)N(Cc3ccc(-c4cn[nH]c4)cc3)C3CC3)C2)c1)C(=O)N1CCNCC1]

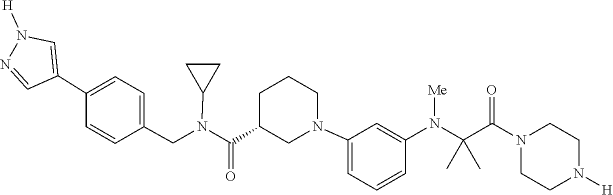 [SMILES: CN(c1cccc(N2CCC[C@@H](C(=O)N(Cc3ccc(-c4cn[nH]c4)cc3)C3CC3)C2)c1)C(C)(C)C(=O)N1CCNCC1]

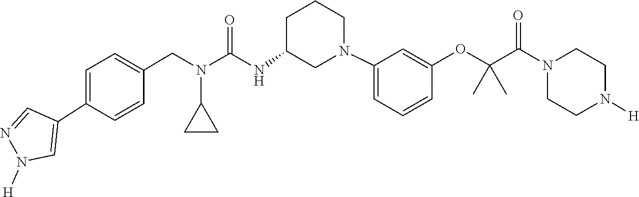 [SMILES: CC(C)(Oc1cccc(N2CCC[C@@H](NC(=O)N(Cc3ccc(-c4cn[nH]c4)cc3)C3CC3)C2)c1)C(=O)N1CCNCC1]

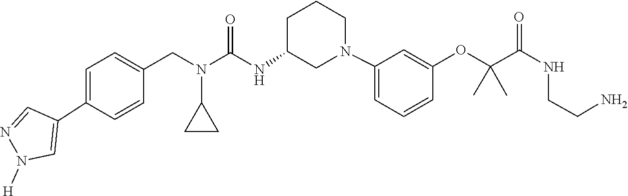 [SMILES: CC(C)(Oc1cccc(N2CCC[C@@H](NC(=O)N(Cc3ccc(-c4cn[nH]c4)cc3)C3CC3)C2)c1)C(=O)NCCN]

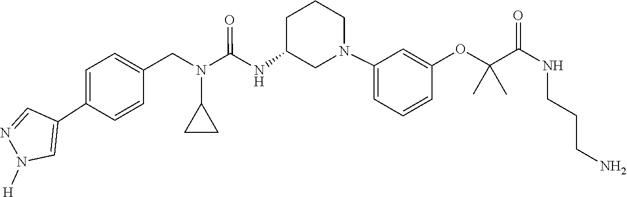 [SMILES: CC(C)(Oc1cccc(N2CCC[C@@H](NC(=O)N(Cc3ccc(-c4cn[nH]c4)cc3)C3CC3)C2)c1)C(=O)NCCCN]

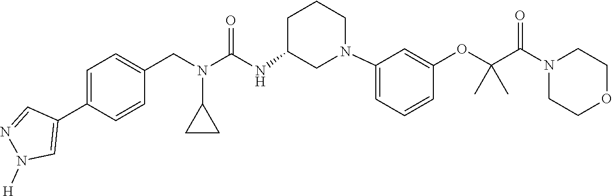 [SMILES: CC(C)(Oc1cccc(N2CCC[C@@H](NC(=O)N(Cc3ccc(-c4cn[nH]c4)cc3)C3CC3)C2)c1)C(=O)N1CCOCC1]

| Compounds | BCL-9 avidity FP IC50 (μM) |
|---|---|
| Compound 15 | 10~20 μM |
| Compound 16 | 10~20 μM |
| Compound 17 | 7.154 μM |
| Compound 18 | 10~20 μM |
| Compound 19 | 2.42 μM |
| Compound 20 | 10~20 μM |

(2) The results of FP (BCL-9 affinity), qPCR, and CCK8 tests are shown in Table 2, and the tests were performed as described above

TABLE 2

| Compound number | FP (μM) | | QPCR (μM) | | CCK8 (μM) | |
|---|---|---|---|---|---|---|
| Compound 1 | 10~50 | μM | 10~50 | μM | 10~50 | μM |
| Compound 2 | 10~50 | μM | 10~20 | μM | 10~50 | μM |
| Compound 3 | 2.760 | μM | 10~20 | μM | 10~20 | μM |
| Compound 5 | 6.203 | μM | 10~20 | μM | 10~20 | μM |
| Compound 6 | 7.753 | μM | 10~20 | μM | 10~20 | μM |
| Compound 4 | 10~50 | μM | 10~50 | μM | 10~50 | μM |
| Compound 12 | 10~20 | μM | 10~20 | μM | 10~50 | μM |
| Compound 13 | 10~50 | μM | 10~50 | μM | 10~50 | μM |
| Compound 14 | 10~50 | μM | 10~50 | μM | 10~50 | μM |
| Compound 7 | 3.145 | μM | 1.456 | μM | 1.053 | μM |
| Compound 8 | 10~50 | μM | 10~50 | μM | 10~20 | μM |
| Compound 9 | 10~20 | μM | 10~50 | μM | 10~50 | μM |
| Compound 10 | 2.7 | μM | 10~20 | μM | 10~20 | μM |
| Compound 11 | 2.437 | μM | 10~20 | μM | 10~20 | μM |
| Compound 15 | 10~20 | μM | 10~20 | μM | 2.419 | μM |
| Compound 16 | 10~20 | μM | 10~20 | μM | 2.792 | μM |
| Compound 17 | 7.154 | μM | 4.666 | μM | 1.836 | μM |
| Compound 18 | 10~20 | μM | 2.537 | μM | 3.455 | μM |
| Compound 19 | 2.426 | μM | 6.111 | μM | 3.259 | μM |

(3) The results of anti-fibrosis test are shown in Table 3

TABLE 3

| | | Human | Human | |
|---|---|---|---|---|
| | | COL1 | COL1 | |
| | | (collagen | (collagen | Human |
| IC50 | Compound structure | type I) | type III) | a-SMA |
| 37 | 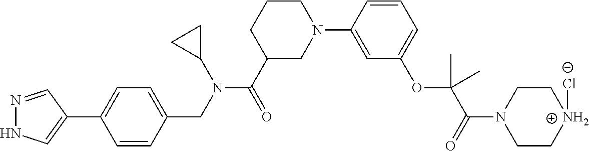 | 10~20 uM | 19.2 uM | 20.2 uM |
| Compound 7 | 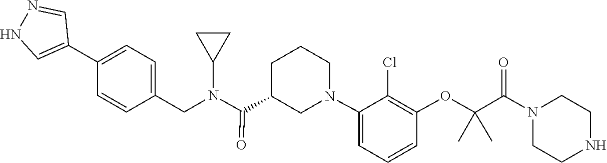 [SMILES: CC(C)(Oc1cccc(N2CCC[C@@H](C(=O)N(Cc3ccc(-c4cn[nH]c4)cc3)C3CC3)C2)c1Cl)C(=O)N1CCNCC1] | 10~20 uM | 10~20 uM | 20~30 uM |
| Compound 17 | 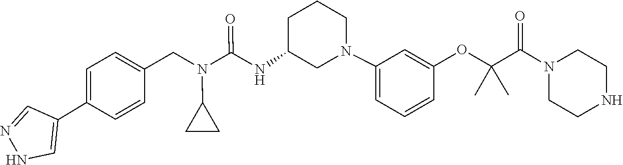 [SMILES: CC(C)(Oc1cccc(N2CCC[C@@H](NC(=O)N(Cc3ccc(-c4cn[nH]c4)cc3)C3CC3)C2)c1)C(=O)N1CCNCC1] | 10~20 uM | 20-30 uM | 5~10 uM |
| Compound 19 | 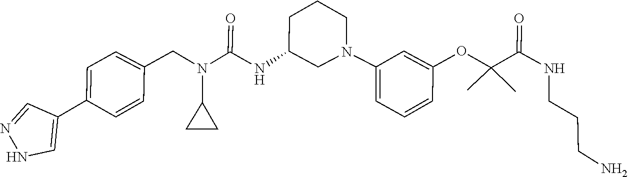 [SMILES: CC(C)(Oc1cccc(N2CCC[C@@H](NC(=O)N(Cc3ccc(-c4cn[nH]c4)cc3)C3CC3)C2)c1)C(=O)NCCCN] | 5.9 uM | 5-20 uM | 5~20 uM |
| 37 | 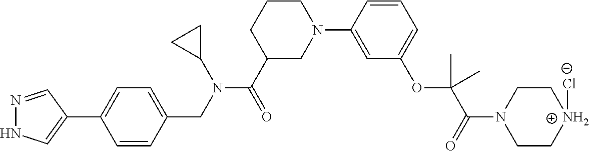 | 10~20 uM | 19.2 uM | 20.2 uM |
| Compound 7 | 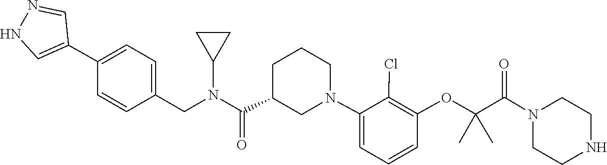 [SMILES: CC(C)(Oc1cccc(N2CCC[C@@H](C(=O)N(Cc3ccc(-c4cn[nH]c4)cc3)C3CC3)C2)c1Cl)C(=O)N1CCNCC1] | 10~20 uM | 10~20 uM | 20~30 uM |
| Compound 17 | 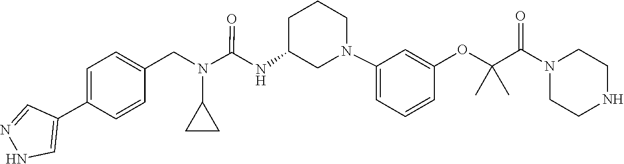 [SMILES: CC(C)(Oc1cccc(N2CCC[C@@H](NC(=O)N(Cc3ccc(-c4cn[nH]c4)cc3)C3CC3)C2)c1)C(=O)N1CCNCC1] | 10~20 uM | 20-30 uM | 5~10 uM |
| Compound 19 | 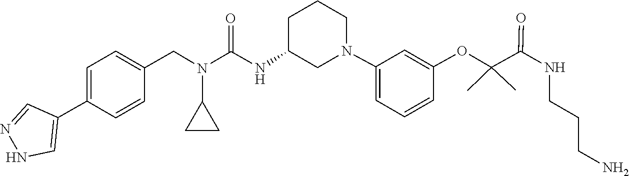 [SMILES: CC(C)(Oc1cccc(N2CCC[C@@H](NC(=O)N(Cc3ccc(-c4cn[nH]c4)cc3)C3CC3)C2)c1)C(=O)NCCCN] | 5.9 uM | 5-20 uM | 5~20 uM |

All documents mentioned in the present invention are cited as references in this application, just as each document is individually cited as a reference. In addition, it should be understood that, after reading the above teaching content of the present invention, those skilled in the art can make various changes or modifications to the present invention, and these equivalent forms also fall within the scope defined by the appended claims of the present application.

1. A compound or a pharmaceutically acceptable salt thereof, or an isomer, solvate, crystal form or a prodrug thereof, wherein the compound is of Formula I: 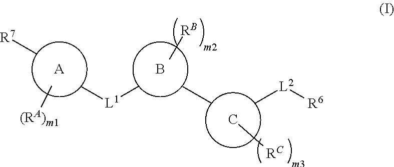
wherein,
R⁷ is an optionally substituted group selected from the group consisting of: optionally substituted C₁₋₆ alkyl, C₃₋₁₀ cycloalkyl, 4 to 10-membered heterocycloalkyl, C₃₋₁₀ cycloalkenyl, 4 to 10-membered heterocycloalkenyl, C₆₋₁₀ aryl, and 5 to 10-membered heteroaryl;
Ring A is an optionally substituted ring selected from the group consisting of: C₆₋₁₀aryl; 5 to 10 membered heteroaryl; C₆₋₁₀ aryl substituted with C₃₋₁₀ cycloalkyl, 4 to 10-membered heterocycloalkyl, C₃₋₁₀cycloalkenyl, 4 to 10-membered heterocycloalkenyl, C₆₋₁₀ aryl, or 5 to 10-membered heteroaryl; 5 to 10-membered heteroaryl substituted with C₃₋₁₀ cycloalkyl, 4 to 10-membered heterocycloalkyl, C₃₋₁₀cycloalkenyl, 4 to 10-membered heterocycloalkenyl, C₆₋₁₀ aryl, or 5 to 10-membered heteroaryl; C₆₋₁₀ aryl fused with C₃₋₁₀ cycloalkyl, 4 to 10-membered heterocycloalkyl, C₃₋₁₀cycloalkenyl, 4 to 10-membered heterocycloalkenyl, C₆₋₁₀ aryl, or 5 to 10-membered heteroaryl; and 5 to 10-membered heteroaryl group fused with C₃₋₁₀ cycloalkyl, 4 to 10-membered heterocycloalkyl, C₃₋₁₀ cycloalkenyl, 4 to 10-membered heterocycloalkenyl, C₆₋₁₀ aryl, or 5 to 10-membered heteroaryl;
m1=0, 1, 2, 3 or 4;
each R^{A} is independently R^{A1} or R^{s};
each R^{A1} is independently selected from the group consisting of: halogen, optionally substituted C₁₋₆ alkyl, optionally substituted C₁₋₆ haloalkyl, optionally substituted C₁₋₆ alkoxy, and optionally substituted C₁₋₆ alkylthio;
L¹ is a linker group of —(W¹)ₙ₁—;
each W¹ is independently selected from the group consisting of: —O—, —S—, C(O)—, —S(O), —S(O)₂, —N(R¹)—, —CH(R⁸)— and —C(R^{s})₂—;
subscript n1=1, 2, 3, 4, or 5;
each R¹ and R⁸ are independently selected from the group consisting of: H, optionally substituted C₁₋₆ alkyl, optionally substituted C₃₋₆ cycloalkyl, halogen, optionally substituted C₁₋₆ haloalkyl, optionally substituted C₁₋₆ alkoxy, optionally substituted C₁₋₆ haloalkyloxy (—O—C₁₋₆ haloalkyl), optionally substituted C₁₋₆alkyl-O—C₁₋₆alkylene, optionally substituted C₁₋₆haloalkyl —O—C₁₋₆alkylene, optionally substituted C₁₋₆haloalkyl-S—C₁₋₆alkylene, optionally substituted C₁₋₆ aminoalkyl, optionally substituted C₃₋₁₀cycloalkyl, optionally substituted 4-10-membered heterocycloalkyl, optionally substituted C₆₋₁₀ aryl, optionally substituted 5 to 10-membered heteroaryl, optionally substituted C₃₋₁₀cycloalkenyl, optionally substituted 4 to 10-membered heterocycloalkenyl, optionally substituted C₃₋₁₀ cycloalkyl-C₁₋₄ alkylene, optionally substituted 4 to 10-membered heterocycloalkyl-C₁₋₄alkylene, optionally substituted C₆₋₁₀aryl-C₁₋₄ alkylene, optionally substituted 5 to 10-membered heteroaryl-C₁₋₄alkylene, optionally substituted C₃₋₁₀cycloalkenyl-C₁₋₄alkylene, and optionally substituted 4 to 10-membered heterocycloalkenyl-C₁₋₄alkylene; or, R¹ or R⁸, together with the R^{s} on ring A, form an optionally substituted C₄₋₁₀cycloalkyl or 4 to 10-membered heterocycloalkyl;
Ring B is an optionally substituted ring selected from the group consisting of: C₃₋₁₂ cycloalkyl, and 4- to 12-membered heterocycloalkyl;
m2=0, 1, 2, 3 or 4;
each R^{B} is independently R^{B1} or R^{s};
each R^{B1} is independently selected from the group consisting of: halogen, hydroxyl, cyano, optionally substituted C₁₋₆alkyl, optionally substituted C₁₋₆alkoxy, optionally substituted C₁₋₆alkylthio, optionally substituted C₃₋₁₀cycloalkyl, optionally substituted 4 to 10-membered heterocycloalkyl, optionally substituted C₃₋₁₀cycloalkenyl, optionally substituted 4 to 10-membered heterocycloalkenyl, optionally substituted C₆₋₁₀ aryl, and optionally substituted 5 to 10-membered heteroaryl;
Ring C is an optionally substituted ring selected from the group consisting of: C₆₋₁₀ aryl, and 5 to 10-membered heteroaryl;
m3=0, 1, 2, 3 or 4;
each R^{C} is independently R^{C1} or R^{s};
each R^{C1} is independently selected from the group consisting of: halogen, optionally substituted C₁₋₆alkyl, optionally substituted C₁₋₆haloalkyl, hydroxyl and optionally substituted C₁₋₆alkoxy, and optionally substituted C₁₋₆haloalkoxy;
L² is a linker group of —(W²)ₙ₂—;
each W² is independently selected from the group consisting of: —O—, —S—, —C(O)—, —S(O), —S(O)₂, —N(R^{s})—, and —CR²R³—,
n2=1, 2, 3, 4, or 5;
R² and R³ are each independently selected from the group consisting of: H, optionally substituted C₁₋₄alkyl, halogen, cyano, optionally substituted C₁₋₆haloalkyl, optionally substituted C₁₋₆ alkyl-O—C₁₋₆alkylene, optionally substituted C₁₋₆haloalkyl-O—C₁₋₆alkylene, optionally substituted C₁₋₆haloalkyl-S—C₁₋₆alkylene, optionally substituted C₃₋₁₀cycloalkyl, optionally substituted 4 to 10-membered heterocycloalkyl, optionally substituted C₆₋₁₀aryl, optionally substituted 5 to 10-membered heteroaryl, optionally substituted C₃₋₁₀cycloalkenyl, optionally substituted 4 to 10-membered heterocycloalkenyl, optionally substituted C₃₋₁₀cycloalkyl-C₁₋₄alkylene, optionally substituted 4 to 10-membered heterocycloalkyl-C₁₋₄alkylene, optionally substituted C₆₋₁₀aryl-C₁₋₄alkylene, optionally substituted 5 to 10-membered heteroaryl-C₁₋₄alkylene, optionally substituted C₃₋₁₀cycloalkenyl-C₁₋₄alkylene, optionally substituted 4 to 10-membered heterocycloalkenyl-C₁₋₄alkylene; or, R² and R³, together with the carbon atoms to which they are attached to, form a group selected from the group consisting of: optionally substituted C₃₋₁₀cycloalkyl, optionally substituted 4 to 10-membered heterocycloalkyl, optionally substituted C₃₋₁₀cycloalkenyl, and optionally substituted 4 to 10-membered heterocycloalkenyl;
R⁶ is selected from the group consisting of: —OH, C₃₋₁₂cycloalkyl group, 4 to 10-membered heterocycloalkyl attached to the rest of the compound of Formula I via a carbon atom in the heterocycloalkyl, and —NR⁴R⁵;
R⁴ and R⁵ are independently selected from the group consisting of: H, optionally substituted C₁₋₆ alkyl, optionally substituted C₃₋₁₀ cycloalkyl, optionally substituted 4 to 8-membered heterocycloalkyl, optionally substitute C₆₋₁₀aryl, optionally substituted 5 to 10-membered heteroaryl, optionally substituted C₃₋₁₀cycloalkenyl, and optionally substituted 4 to 10-membered heterocycloalkenyl; or, R⁴ and R, together with the nitrogen atom to which they are connected to, form a ring selected from the group consisting of: optionally substituted 4 to 10-membered heterocycloalkyl, optionally substituted 4 to 10-membered heterocycloalkenyl, optionally substituted 4 to 10-membered heterocycloalkenyl, and optionally substituted 5 to 10-membered heteroaryl;
each R^{s} is independently H or optionally substituted C₁₋₄alkyl;
unless otherwise defined, said optionally substituted means unsubstituted or means that one or more hydrogen atoms in the group are substituted with a substituent chosen from the group—consisting of: D, halogen, C₁₋₆ alkyl, C₁₋₆ haloalkyl, C₁₋₆ hydroxyalkyl, C₂₋₆ alkenyl, C₂₋₆ alkynyl, —CN, —OR″, —NO₂″, —NR″R″″, —SR″, —OC(O)R″, —C(O)R″, —CO₂R″, —CONR″, —OC(O)NR″R″″, —NR″″C(O)R″, —NR″″—C(O)NR″R″″, —NR″″C(O)₂R″, —S(O)R″, —S(O)₂R″, —S(O)₂NR″R″″, —NR″″S(O)₂R″, C₃₋₁₀ cycloalkyl optionally substituted with one or more R″″″, 4 to 10-membered heterocycloalkyl optionally substituted with one or more R″″″, C₆₋₁₀ aryl optionally substituted with one or more R″″″, 5 to 10-membered heteroaryl optionally substituted with one or more R″″″, —C₁₋₄alkylene-C₃₋₁₀ cycloalkyl optionally substituted with one or more R″″″, —C₁₋₄ alkylene-4 to 10-membered heterocycloalkyl optionally substituted with one or more R″″″, —C₁₋₄ alkylene-C₆₋₁₀ aryl optionally substituted with one or more R″″″, and —C₁₋₄alkylene-5 to 10-membered heteroaryl optionally substituted with one or more R″″″;
each R″ is independently selected from the group consisting of: H, D, C₁₋₆ alkyl, C₁₋₆ haloalkyl, C₃₋₁₀cycloalkyl optionally substituted with one or more R″″″, 4 to 10 heterocycloalkyl optionally substituted with one or more R″″″, C₆₋₁₀ aryl optionally substituted with one or more R″″″, 5 to 10 heteroaryl optionally substituted with one or more R″″″, —C₁₋₄alkylene-C₃₋₁₀ cycloalkyl optionally substituted with one or more R″″″, —C₁₋₄ alkylene-4 to 10-membered heterocycloalkyl optionally substituted with one or more R″″″, —C₁₋₄alkylene-C₆₋₁₀ aryl optionally substituted with one or more R″″″, and —C₁₋₄alkylene-5 to 10-membered heteroaryl optionally substituted with one or more R″″″;
each R″″ is selected from the group consisting of: H, D, C₁₋₄alkyl, C₁₋₄haloalkyl, and C₃₋₄ cycloalkyl;
each R″″″ is independently selected from the group consisting of: D, halogen, hydroxyl, nitro, —CN, C₁₋₆alkyl, and C₁₋₆haloalkyl.

2. The compound of claim 1 or the pharmaceutically acceptable salt thereof, or the isomer, solvate, crystal form, wherein
R⁷ is an optionally substituted group selected from the group consisting of: optionally substituted C₁₋₆alkyl, C₃₋₁₀cycloalkyl, 4 to 10-membered heterocycloalkyl, C₆₋₁₀ aryl, and 5 to 10-membered heteroaryl; and
R⁴ and R⁵ are each independently selected from the group consisting of: optionally substituted C₁₋₆alkyl, optionally substituted C₃₋₁₀ cycloalkyl, optionally substituted 4 to 8-membered heterocycloalkyl, optionally substituted C₆₋₁₀ aryl, optionally substituted 5 to 10-membered heteroaryl, optionally substituted C₃₋₁₀cycloalkenyl, and optionally substituted 4 to 10-membered heterocycloalkenyl; or, R⁴ and R⁵, together with the nitrogen atom to which they are connected to, form a ring selected from the group consisting of: optionally substituted 4 to 10-membered heterocycloalkyl, optionally substituted 4 to 10-membered heterocycloalkenyl, and optionally substituted 5 to 10-membered heteroaryl.

3. The compound of claim 1 or the pharmaceutically acceptable salt thereof, or the isomer, solvate, crystal form or prodrug thereof, wherein,
R⁷ is optionally substituted C₃₋₁₀cycloalkenyl or optionally substituted 5-10 membered heteroaryl group;
Ring A is 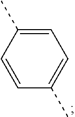
m1=0 or 1;
R^{A} is H or R^{A1}; and R^{A1} is selected from the group consisting of: halogen, optionally substituted C₁₋₆ haloalkyl, and optionally substituted C₁₋₆ alkoxy;
L¹ is-CH(R⁸)—N(R¹)—C(O)— or —CH(R⁸)—N(R¹)—C(O)—NH—, wherein the CH(R⁸) terminal is attached to Ring A; and wherein, R¹ is optionally substituted C₃₋₆ cycloalkyl, R⁸ is selected from the group consisting of: H, and optionally substituted C₁₋₆alkyl; 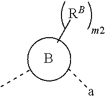
is 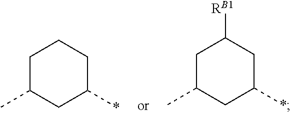
wherein * refers to the attachment to Ring C; and wherein R^{B1} is selected from the group consisting of: optionally substituted C₃₋₁₀ cycloalkyl, optionally substituted 4 to 10-membered heterocycloalkyl, optionally substituted C₆₋₁₀ aryl, and optionally substituted 5 to 10-membered heteroaryl;
Ring C is 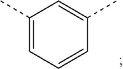
m3=0, 1 or 2;
R^{C} is H, C₁₋₄ alkyl or R^{C1}; and R^{C1} is selected from the group consisting of: halogen-, C₁₋₆ haloalkyl, and C₁₋₆ alkoxy;
L₂ is —W²—CR²R³—C(O)— and W² is selected from the group consisting of: —O—, —S—, —N(R^{s})—; wherein both R² and R³ are optionally substituted C₁₋₄ alkyl.

4. The compound of claim 1, wherein the compound is selected from the following compounds:

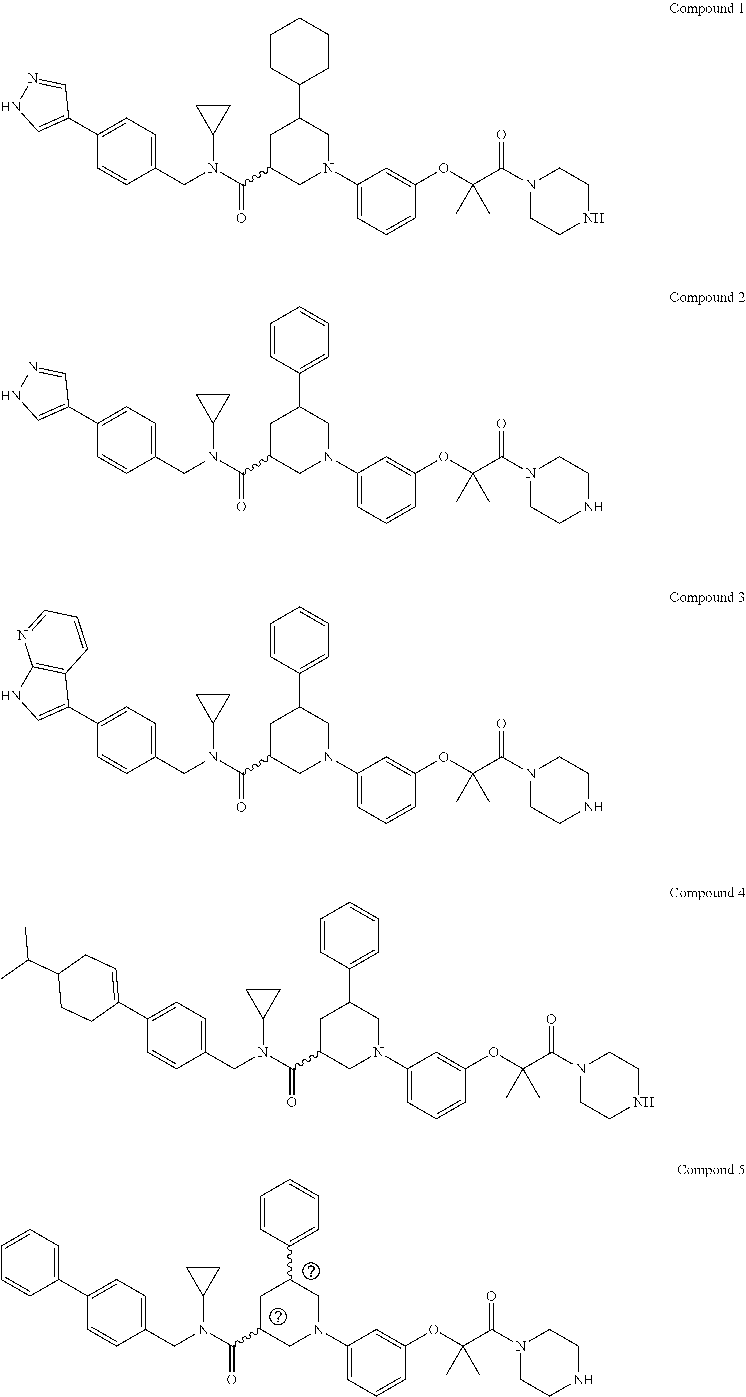 [SMILES: CC(C)(Oc1cccc(N2CC(C(=O)N(Cc3ccc(-c4cn[nH]c4)cc3)C3CC3)CC(C3CCCCC3)C2)c1)C(=O)N1CCNCC1 ; CC(C)(Oc1cccc(N2CC(C(=O)N(Cc3ccc(-c4cn[nH]c4)cc3)C3CC3)CC(c3ccccc3)C2)c1)C(=O)N1CCNCC1 ; CC(C)(Oc1cccc(N2CC(C(=O)N(Cc3ccc(-c4c[nH]c5ncccc45)cc3)C3CC3)CC(c3ccccc3)C2)c1)C(=O)N1CCNCC1 ; CC(C)C1CC=C(c2ccc(CN(C(=O)C3CC(c4ccccc4)CN(c4cccc(OC(C)(C)C(=O)N5CCNCC5)c4)C3)C3CC3)cc2)CC1 ; CC(C)(Oc1cccc(N2CC(C(=O)N(Cc3ccc(-c4ccccc4)cc3)C3CC3)CC(c3ccccc3)C2)c1)C(=O)N1CCNCC1]

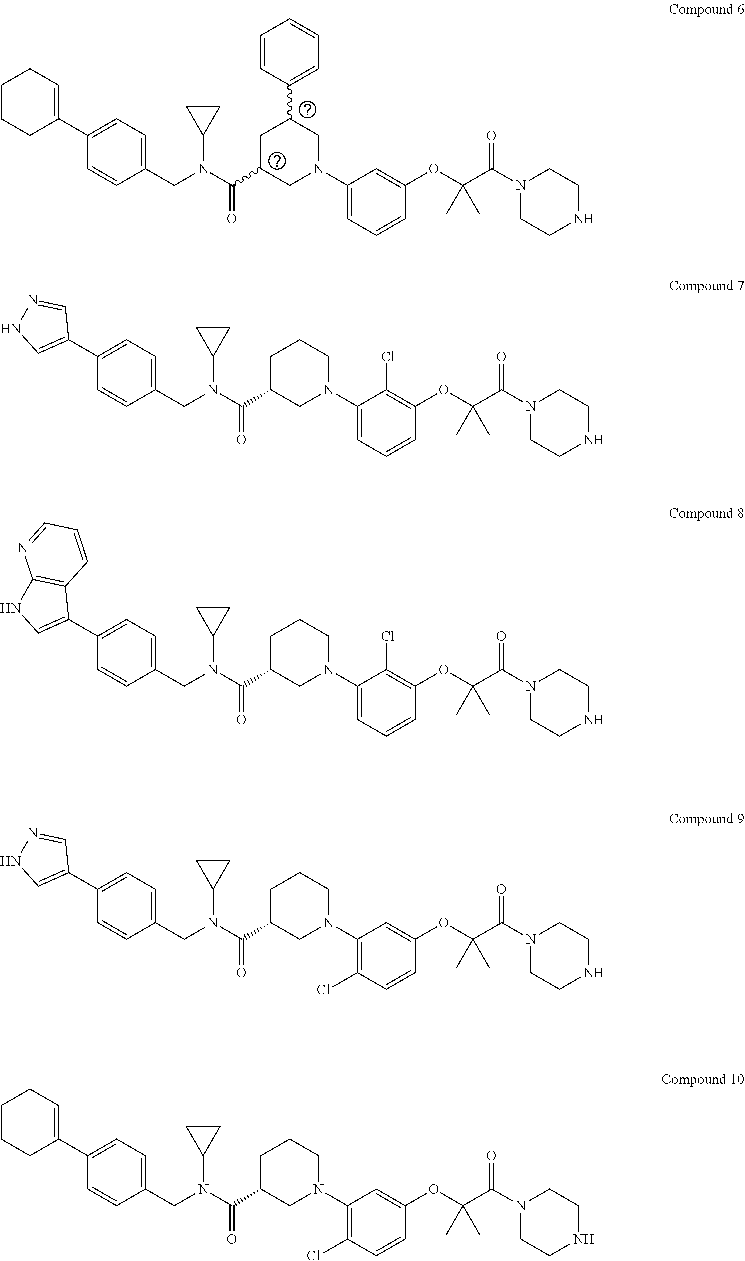 [SMILES: CC(C)(Oc1cccc(N2CC(C(=O)N(Cc3ccc(C4=CCCCC4)cc3)C3CC3)CC(c3ccccc3)C2)c1)C(=O)N1CCNCC1 ; CC(C)(Oc1cccc(N2CCC[C@@H](C(=O)N(Cc3ccc(-c4cn[nH]c4)cc3)C3CC3)C2)c1Cl)C(=O)N1CCNCC1 ; CC(C)(Oc1cccc(N2CCC[C@@H](C(=O)N(Cc3ccc(-c4c[nH]c5ncccc45)cc3)C3CC3)C2)c1Cl)C(=O)N1CCNCC1 ; CC(C)(Oc1ccc(Cl)c(N2CCC[C@@H](C(=O)N(Cc3ccc(-c4cn[nH]c4)cc3)C3CC3)C2)c1)C(=O)N1CCNCC1 ; CC(C)(Oc1ccc(Cl)c(N2CCC[C@@H](C(=O)N(Cc3ccc(C4=CCCCC4)cc3)C3CC3)C2)c1)C(=O)N1CCNCC1]

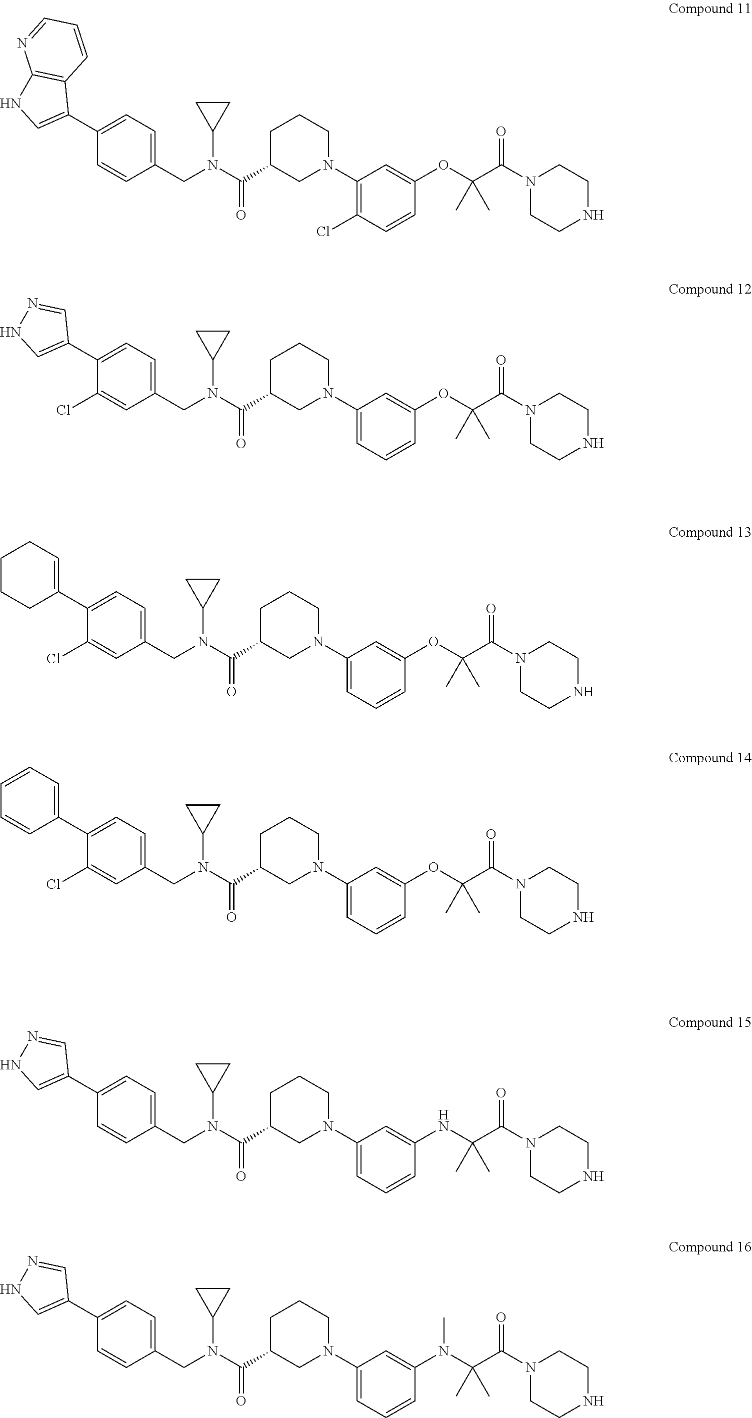

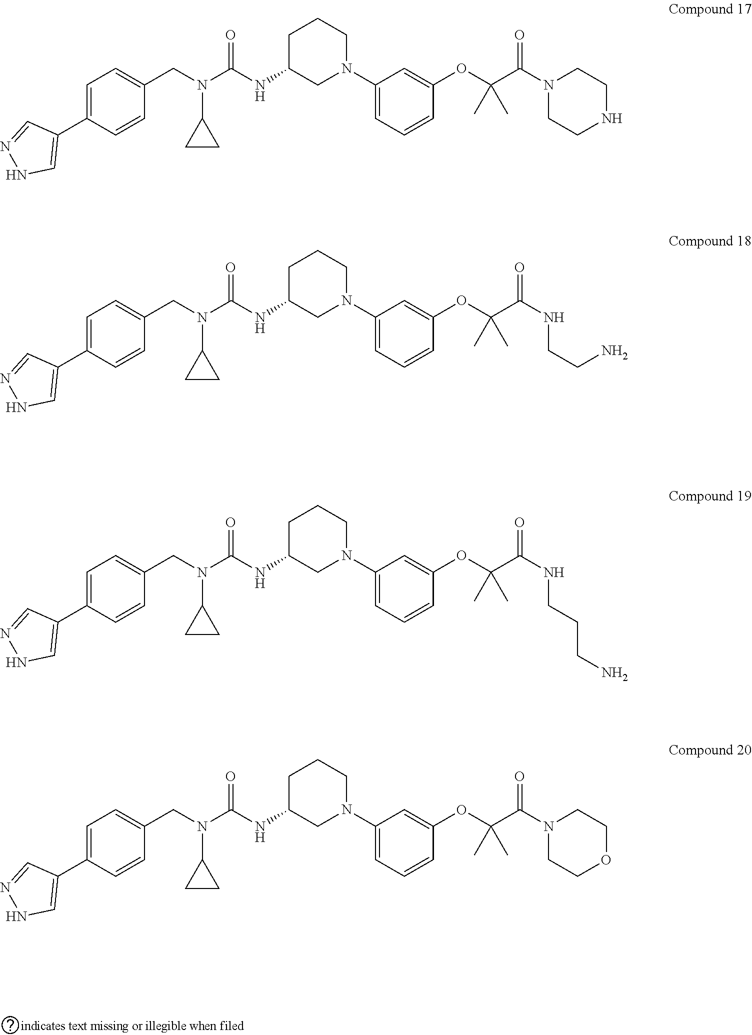 [SMILES: CC(C)(Oc1cccc(N2CCC[C@@H](NC(=O)N(Cc3ccc(-c4cn[nH]c4)cc3)C3CC3)C2)c1)C(=O)N1CCNCC1 ; CC(C)(Oc1cccc(N2CCC[C@@H](NC(=O)N(Cc3ccc(-c4cn[nH]c4)cc3)C3CC3)C2)c1)C(=O)NCCN ; CC(C)(Oc1cccc(N2CCC[C@@H](NC(=O)N(Cc3ccc(-c4cn[nH]c4)cc3)C3CC3)C2)c1)C(=O)NCCCN ; CC(C)(Oc1cccc(N2CCC[C@@H](NC(=O)N(Cc3ccc(-c4cn[nH]c4)cc3)C3CC3)C2)c1)C(=O)N1CCOCC1]

| Molecular | |
|---|---|
| numbering | Structural formula |
| C37-005 | 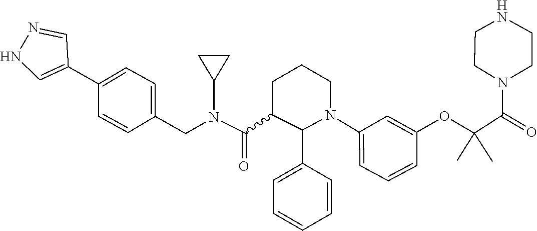 [SMILES: CC(C)(Oc1cccc(N2CCCC(C(=O)N(Cc3ccc(-c4cn[nH]c4)cc3)C3CC3)C2c2ccccc2)c1)C(=O)N1CCNCC1] |
| C37-015 | 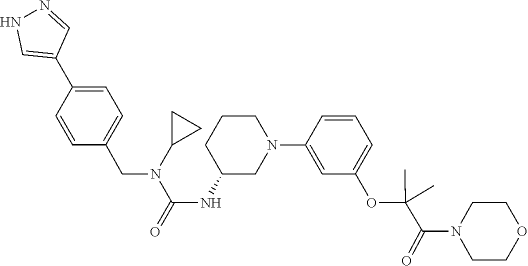 |
| C37-016 | 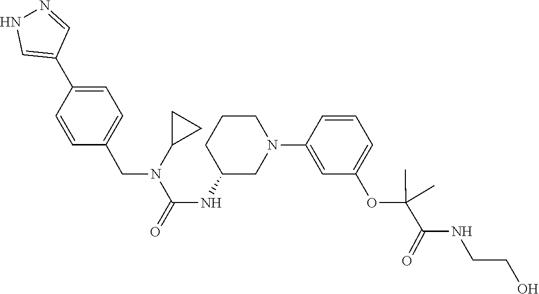 |
| C37-018 | 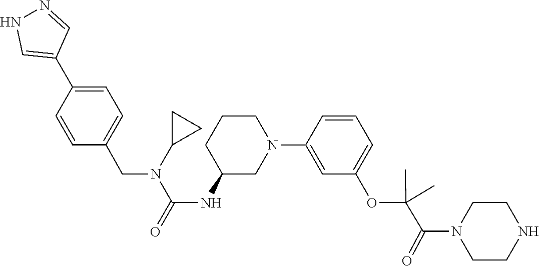 |
| C37-019 | 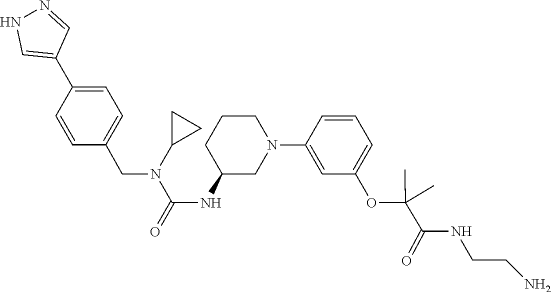 |
| C37-020 | 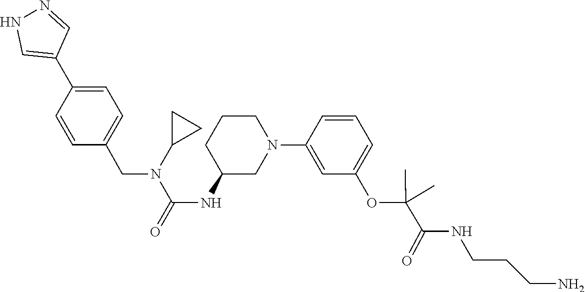 |
| C37-021 | 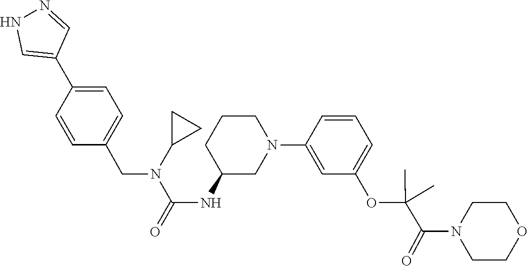 |
| C37-022 | 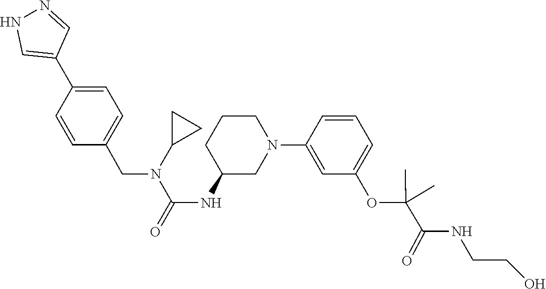 |
| C37-032 | 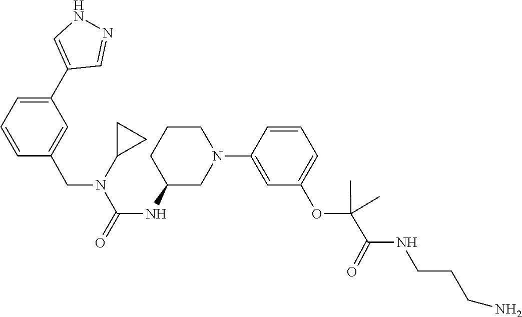 |
| C37-033 | 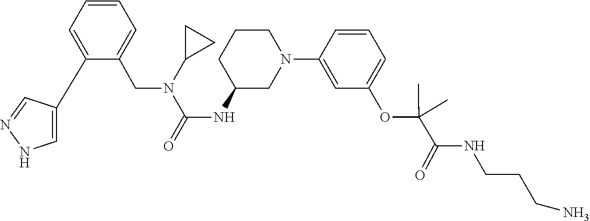 |
| C37-035 | 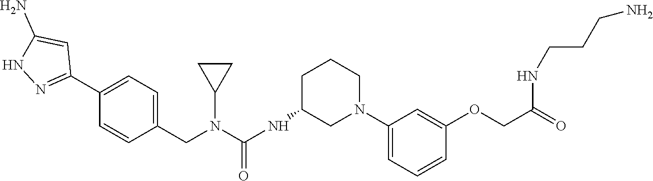 [SMILES: NCCCNC(=O)COc1cccc(N2CCC[C@@H](NC(=O)N(Cc3ccc(-c4cc(N)[nH]n4)cc3)C3CC3)C2)c1] |
| C37-036 | 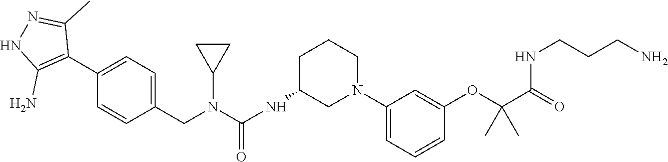 [SMILES: Cc1n[nH]c(N)c1-c1ccc(CN(C(=O)N[C@@H]2CCCN(c3cccc(OC(C)(C)C(=O)NCCCN)c3)C2)C2CC2)cc1] |
| C37-043 | 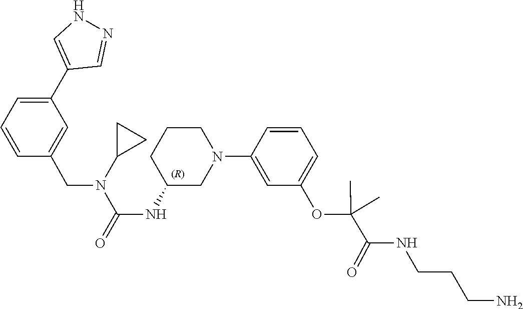 |
| C37-044 | 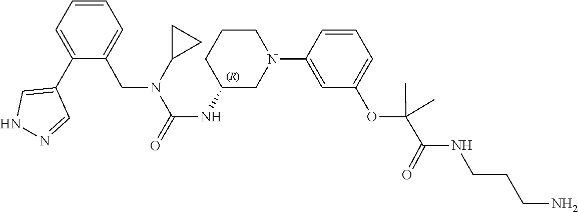 |
| C37-045 | 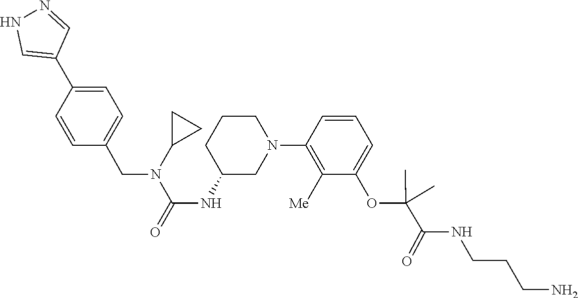 |
| C37-046 | 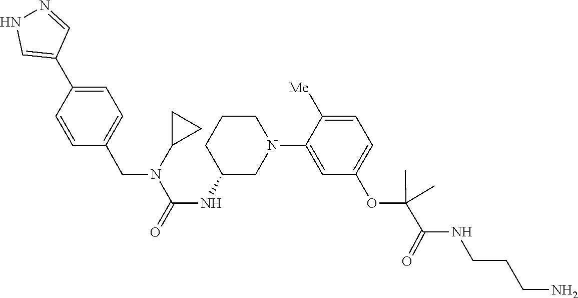 |

or a pharmaceutically acceptable salt thereof, or an isomer, solvate, crystal form or a prodrug thereof.

5. The compound of claim 1 or the pharmaceutically acceptable salt thereof, or the isomer, solvate, crystal form, or prodrug thereof, wherein the compound is of Formula III:

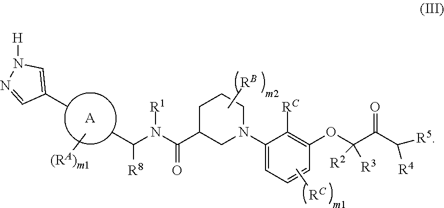

6. The compound of claim 1 or the pharmaceutically acceptable salt thereof, or the isomer, solvate, crystal form or prodrug thereof, wherein the compound is of Formula V, Formula Va or Formula Vb.

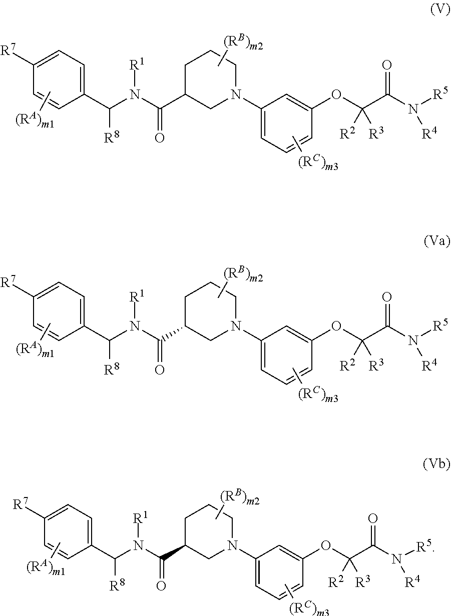

7. The compound of claim 1 or the pharmaceutically acceptable salt thereof, or the isomer, solvate, crystal form or prodrug thereof, wherein the compound is of Formula IV: 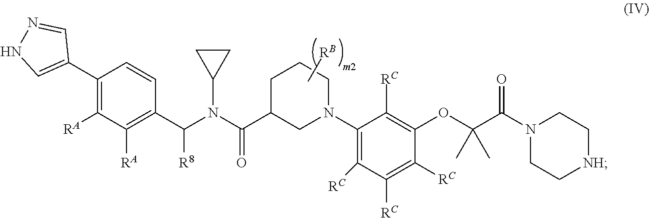
wherein, at least one of R^{A} is R^{A1}.

8. The compound of claim 1 or the pharmaceutically acceptable salt thereof, or the isomer, solvate, crystal form or prodrug thereof, wherein the compound of Formula IV-1 or Formula IV-2;

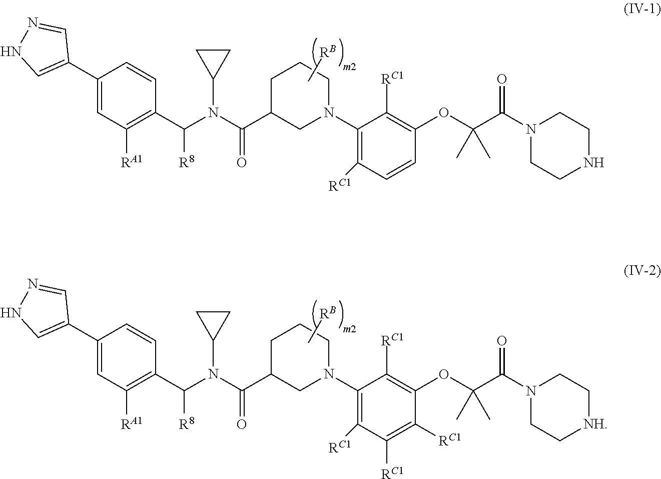

9. The compound of claim 1, wherein the compound is selected from Table A1, Table A2, Table A3, Table A4, Table A5, or Table A6, Table B and Table C:
TABLE A1

| | |
|---|---|
| A-022 | 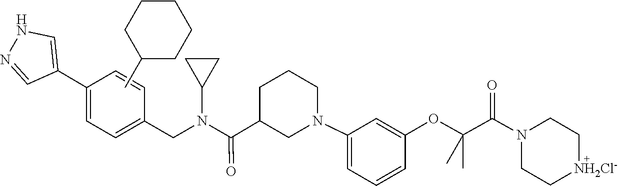 |
| A-023 | 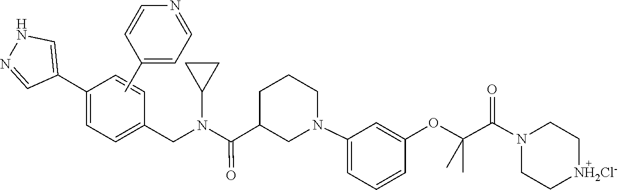 |
| A-024 | 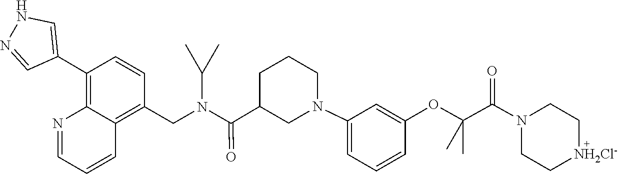 |
| A-025 | 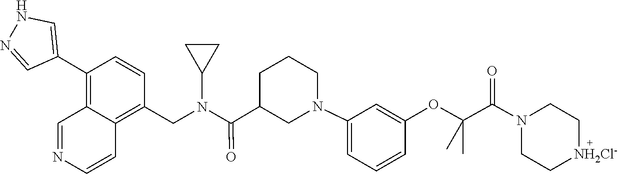 |
| A-026 | 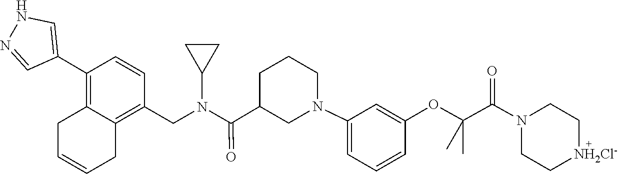 |
| A-027 | 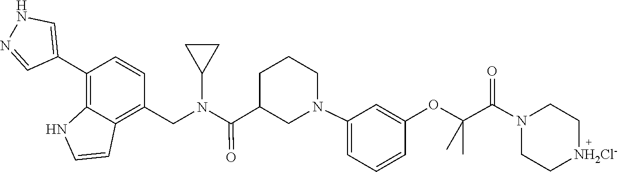 |

TABLE A2

| | |
|---|---|
| A-001 | 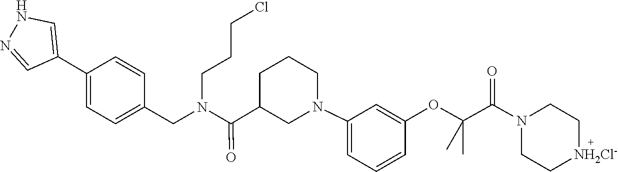 |
| A-002 | 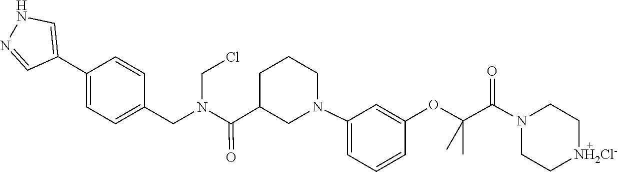 |
| A-003 | 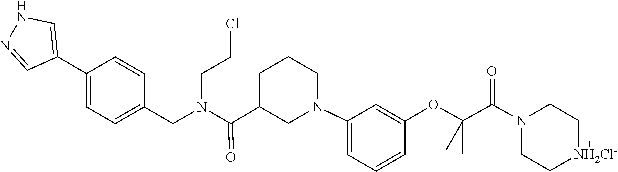 |
| A-004 | 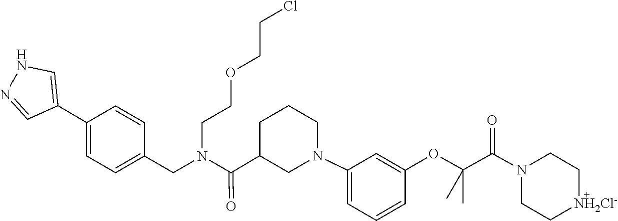 |
| A-005 | 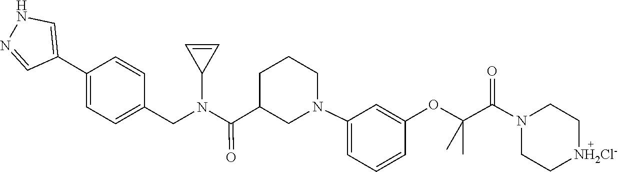 |
| A-006 | 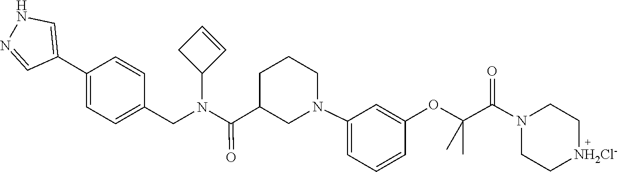 |
| A-007 | 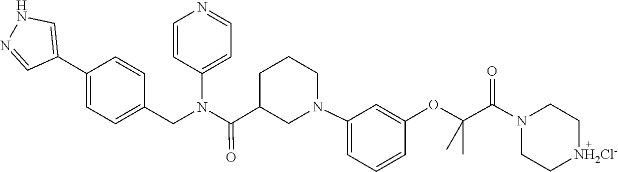 |
| A-008 | 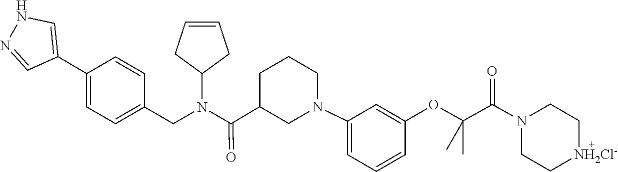 |
| A-009 | 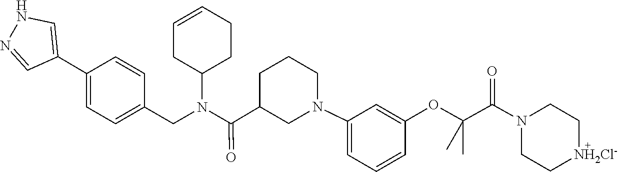 |

TABLE A3

| | |
|---|---|
| A-034 | 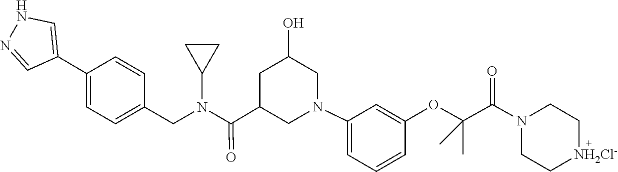 |
| A-035 | 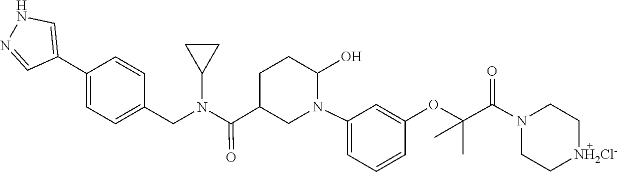 |
| A-036 | 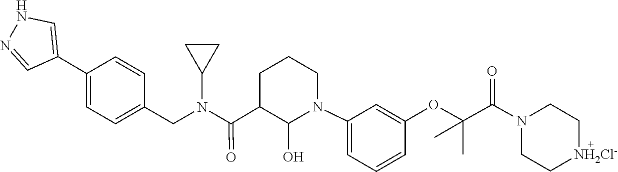 |
| A-037 | 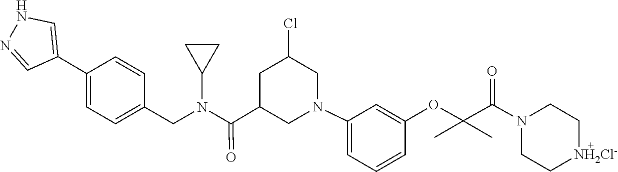 |
| A-038 | 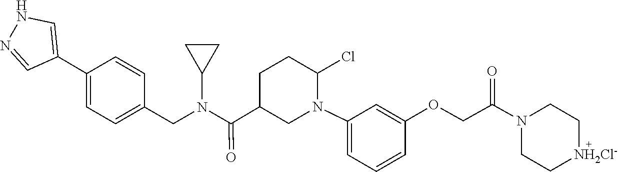 |
| A-039 | 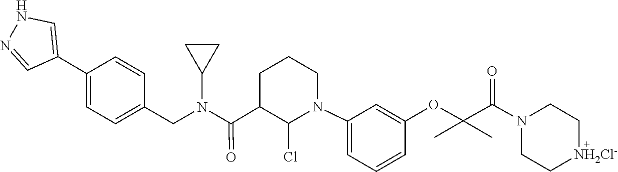 |
| A-040 | 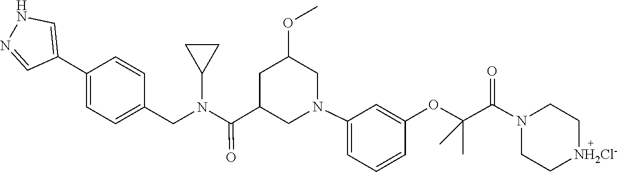 |
| A-041 | 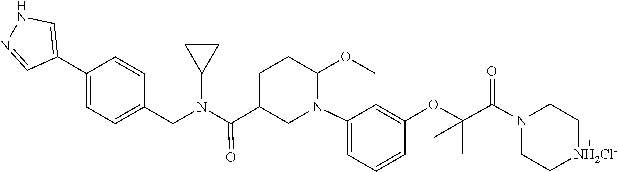 |
| A-042 | 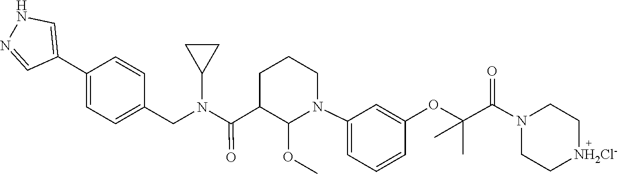 |
| A-043 | 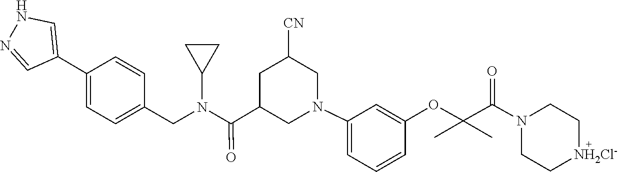 |
| A-044 | 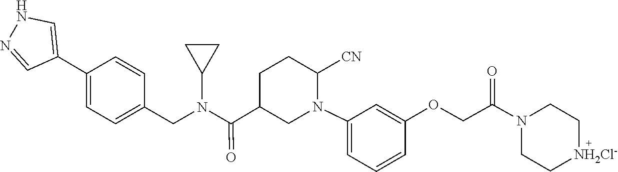 |
| A-045 | 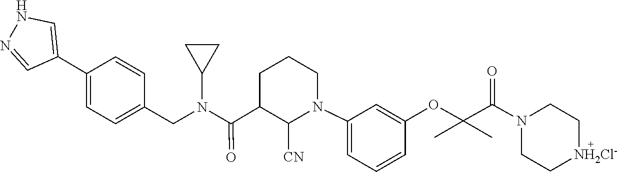 |
| A-046 | 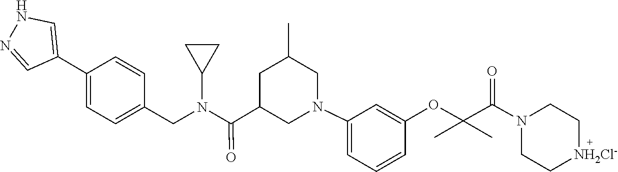 |
| A-047 | 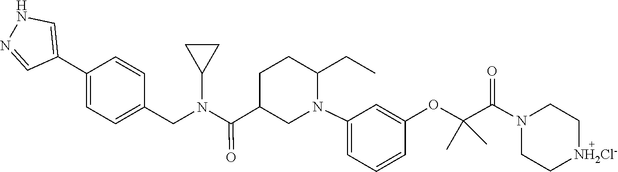 |
| A-048 | 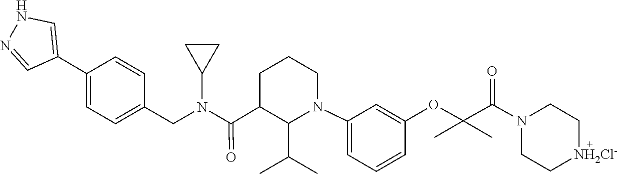 |
| A-049 | 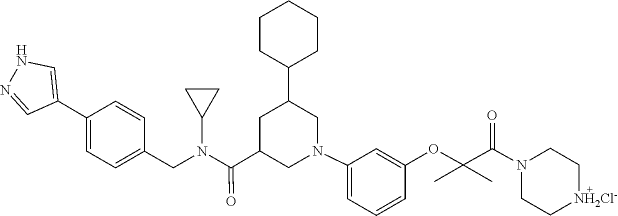 |
| A-050 | 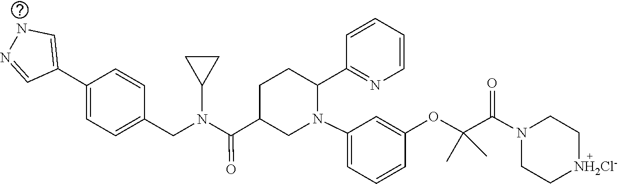 |
| A-051 | 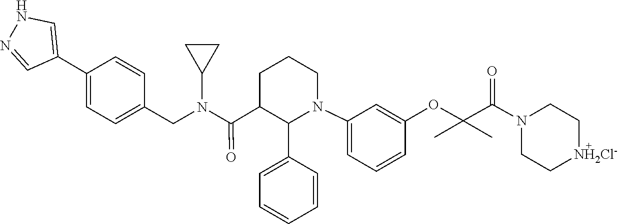 |
| A-052 | 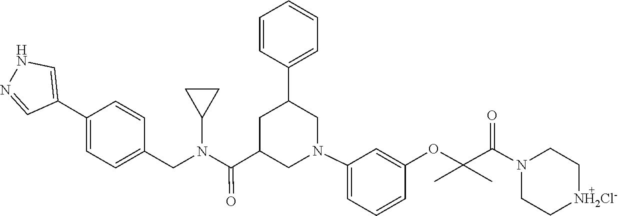 |
| A-053 | 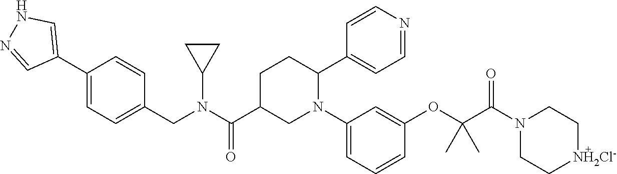 |
| A-054 | 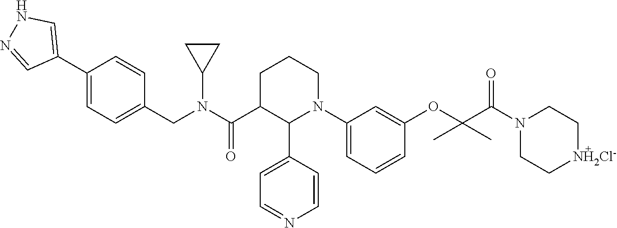 |

TABLE A4

| | |
|---|---|
| A-019 | 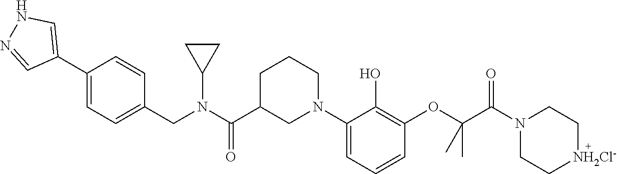 |
| A-020 | 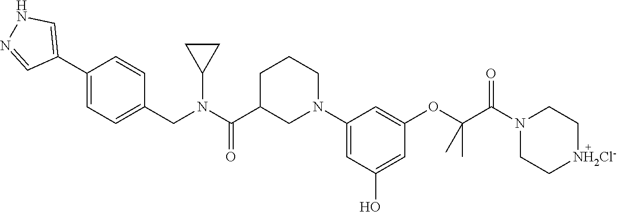 |
| A-021 | 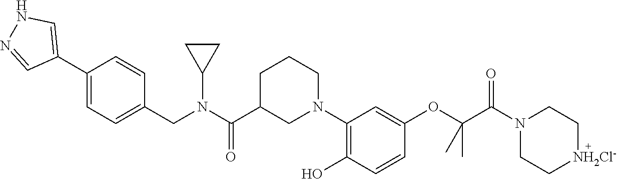 |

TABLE A5

| | |
|---|---|
| A-028 | 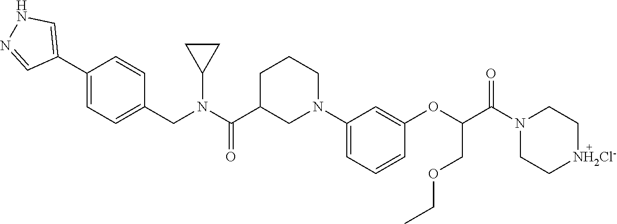 |
| A-029 | 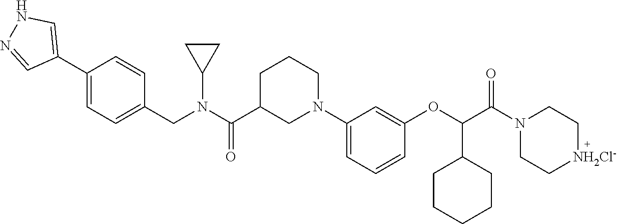 |
| A-030 | 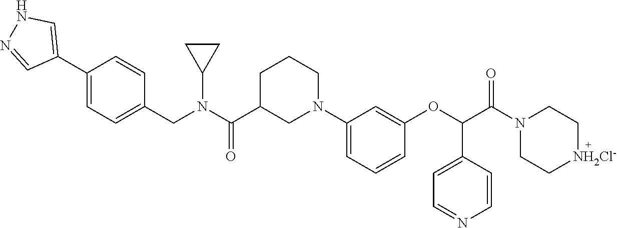 |
| A-031 | 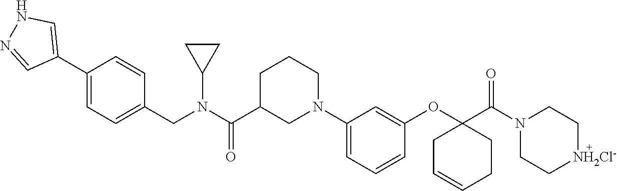 |
| A-032 | 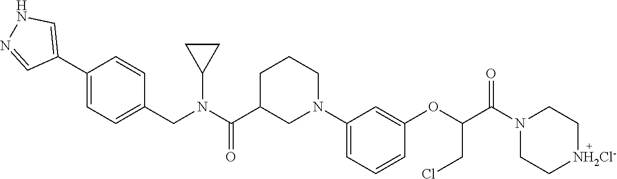 |
| A-033 | 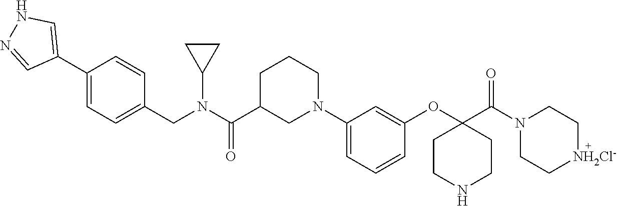 |

TABLE A6

| | |
|---|---|
| A-010 | 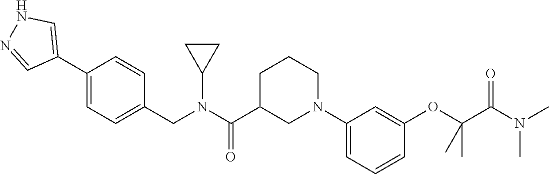 [SMILES: CN(C)C(=O)C(C)(C)Oc1cccc(N2CCCC(C(=O)N(Cc3ccc(-c4cn[nH]c4)cc3)C3CC3)C2)c1] |
| A-011 | 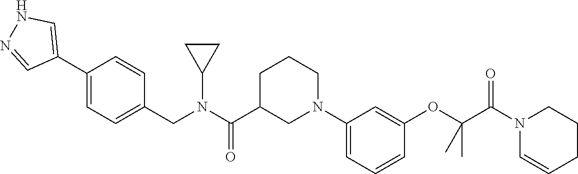 [SMILES: CC(C)(Oc1cccc(N2CCCC(C(=O)N(Cc3ccc(-c4cn[nH]c4)cc3)C3CC3)C2)c1)C(=O)N1C=CCCC1] |
| A-012 | 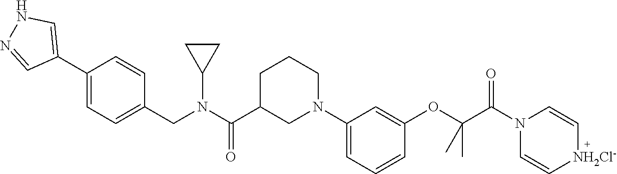 |
| A-013 | 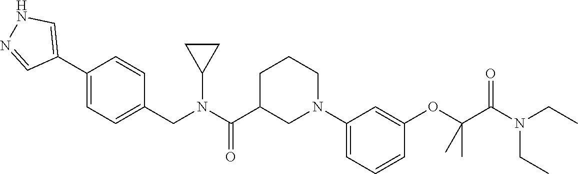 [SMILES: CCN(CC)C(=O)C(C)(C)Oc1cccc(N2CCCC(C(=O)N(Cc3ccc(-c4cn[nH]c4)cc3)C3CC3)C2)c1] |
| A-014 | 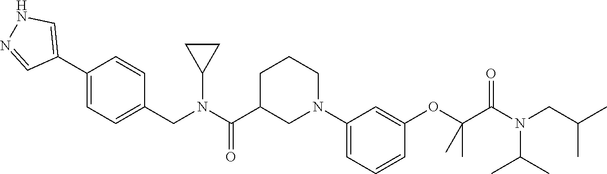 [SMILES: CC(C)CN(C(=O)C(C)(C)Oc1cccc(N2CCCC(C(=O)N(Cc3ccc(-c4cn[nH]c4)cc3)C3CC3)C2)c1)C(C)C] |
| A-015 | 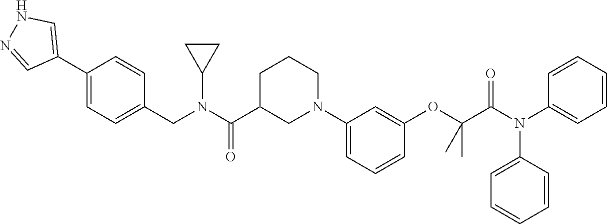 [SMILES: CC(C)(Oc1cccc(N2CCCC(C(=O)N(Cc3ccc(-c4cn[nH]c4)cc3)C3CC3)C2)c1)C(=O)N(c1ccccc1)c1ccccc1] |
| A-016 | 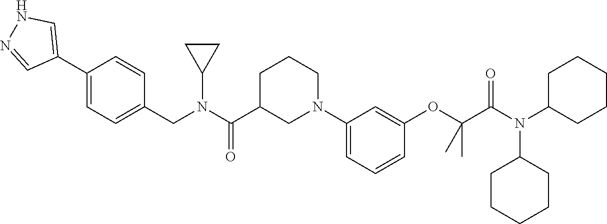 [SMILES: CC(C)(Oc1cccc(N2CCCC(C(=O)N(Cc3ccc(-c4cn[nH]c4)cc3)C3CC3)C2)c1)C(=O)N(C1CCCCC1)C1CCCCC1] |
| A-017 | 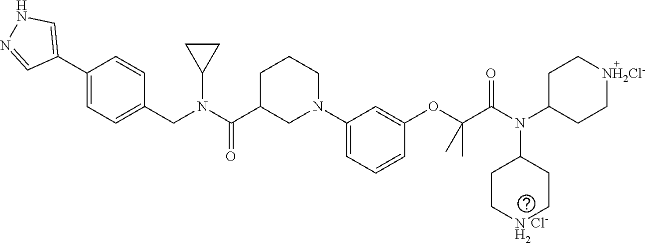 |
| A-018 | 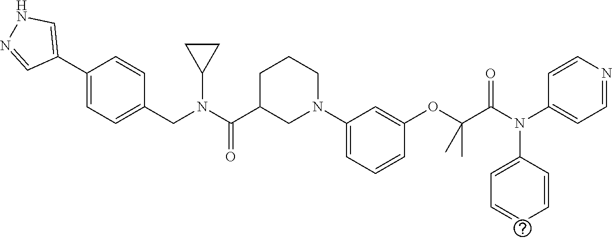 [SMILES: CC(C)(Oc1cccc(N2CCCC(C(=O)N(Cc3ccc(-c4cn[nH]c4)cc3)C3CC3)C2)c1)C(=O)N(c1ccccc1)c1ccncc1] |

TABLE B 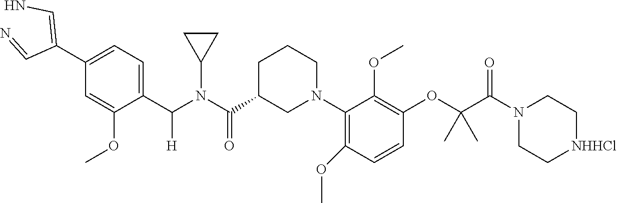 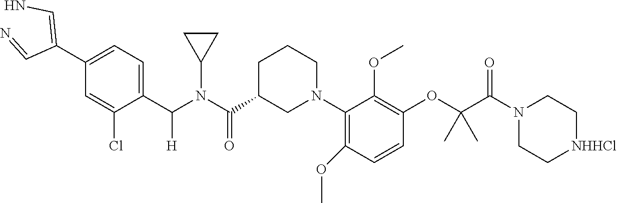 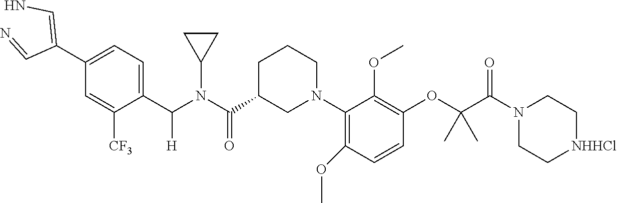 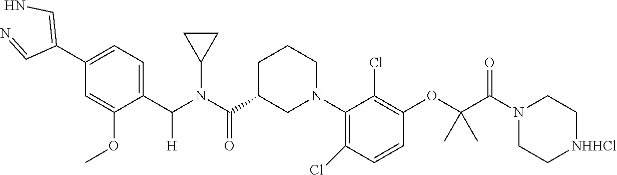 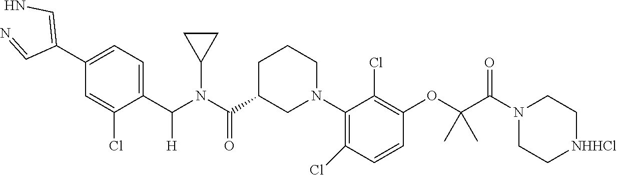 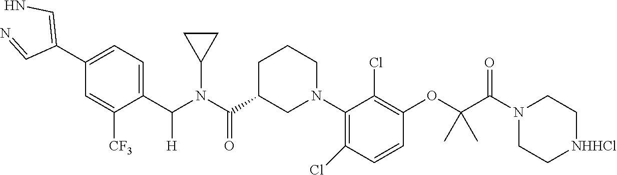 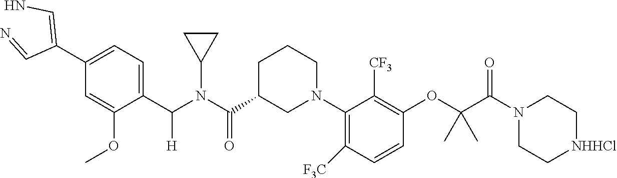 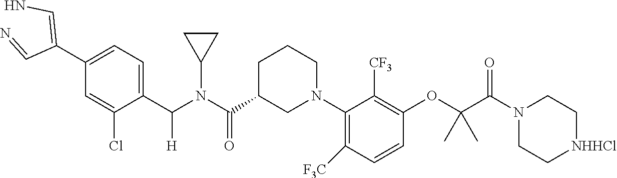 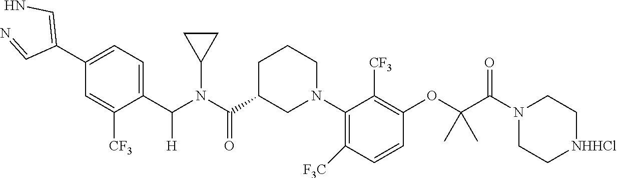 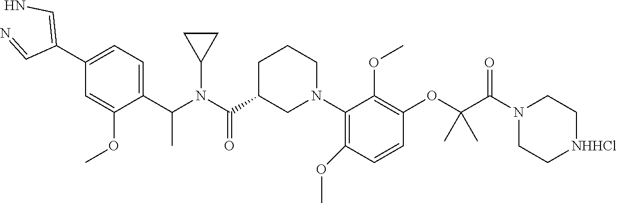 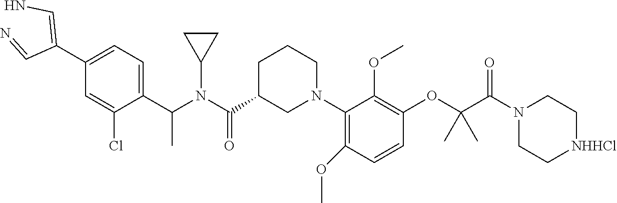 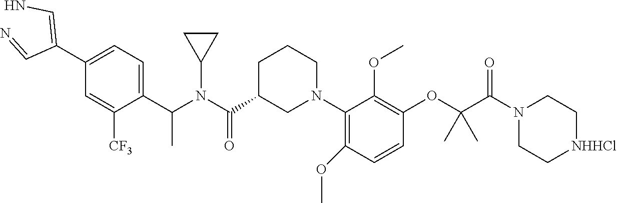 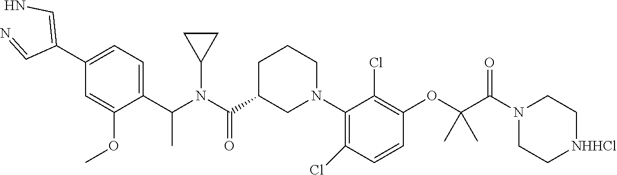 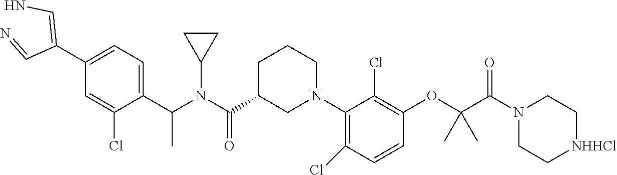 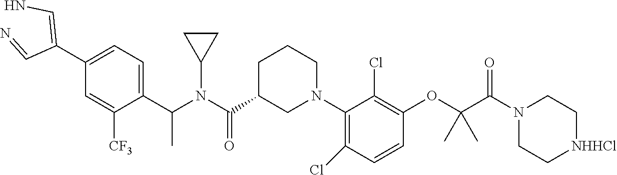 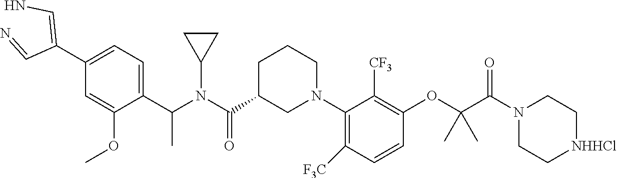 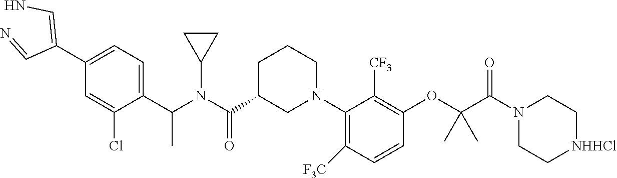 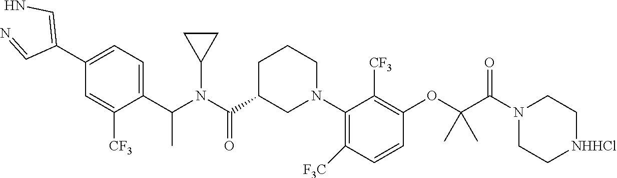 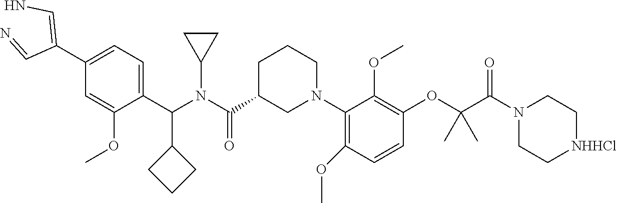 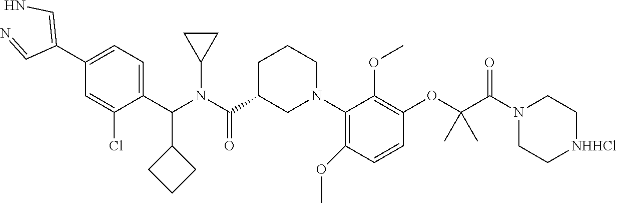 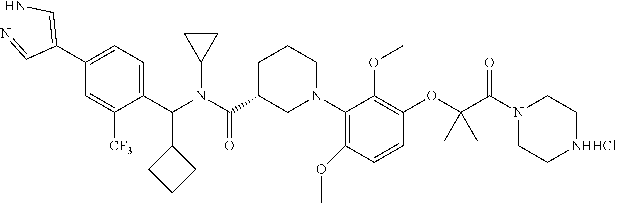 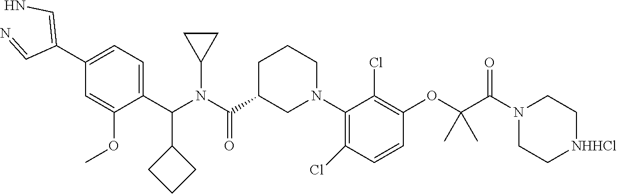 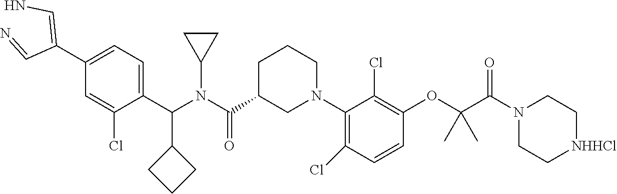 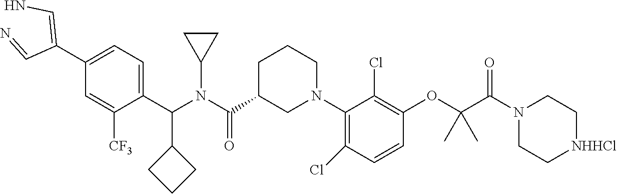 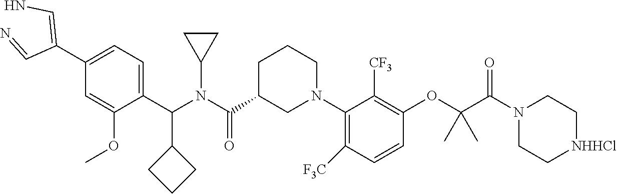 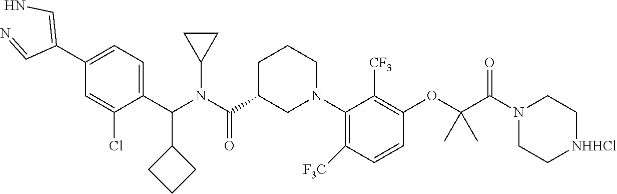 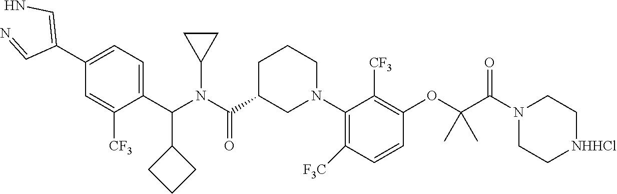 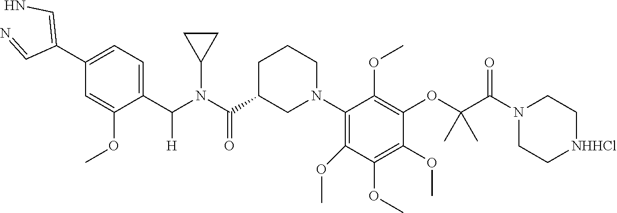 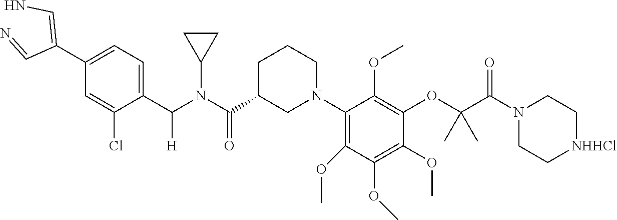 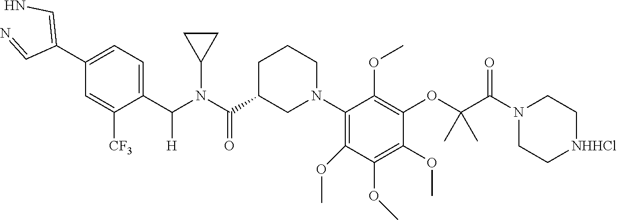 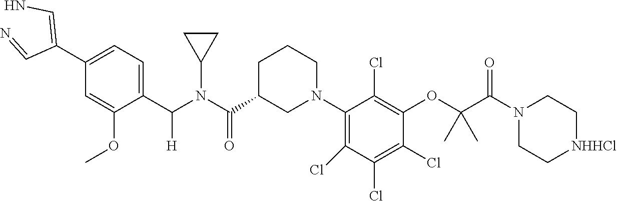 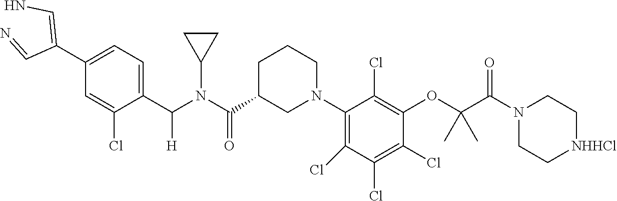 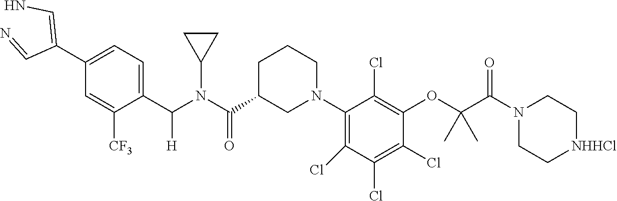 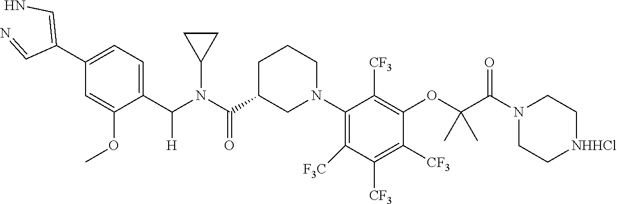 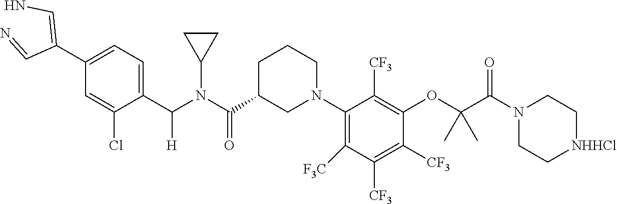 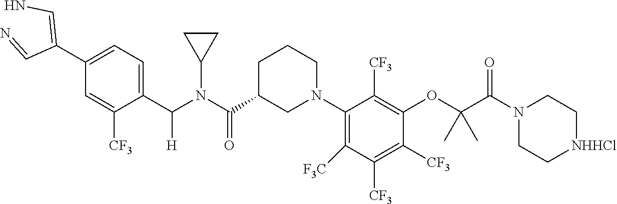 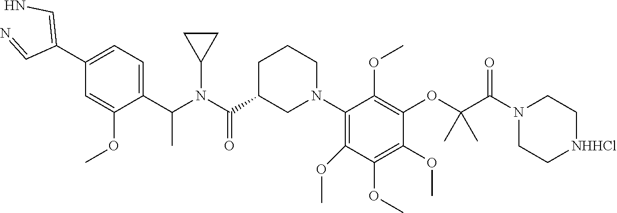 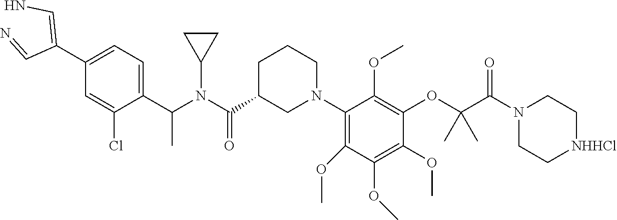 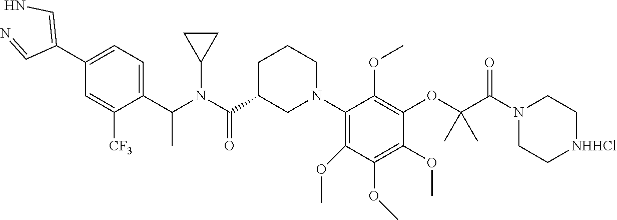 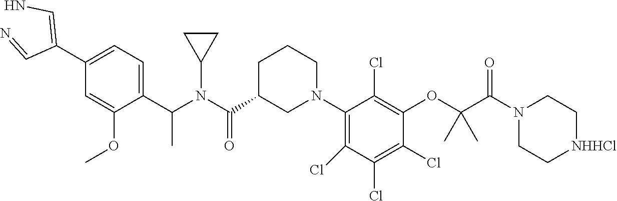 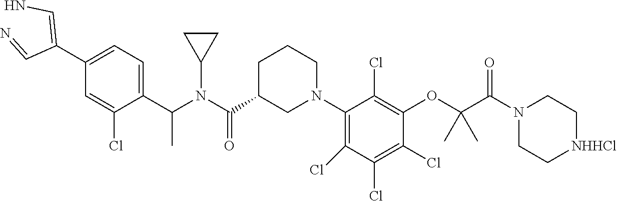 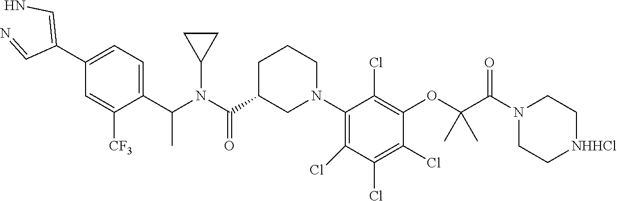 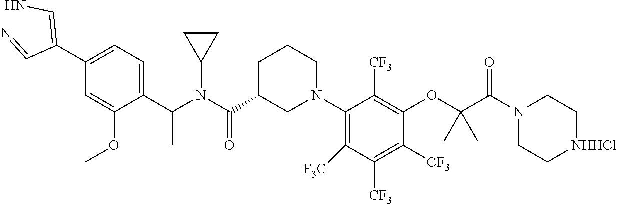 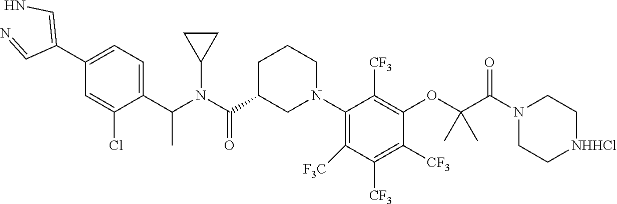 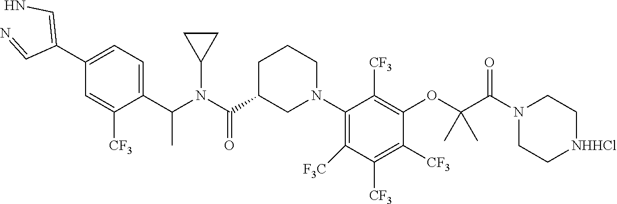 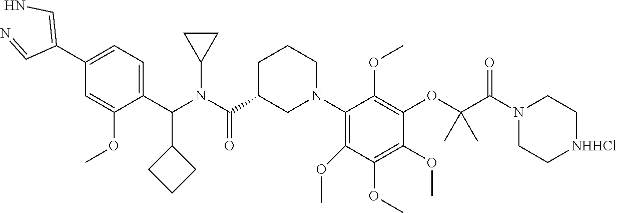 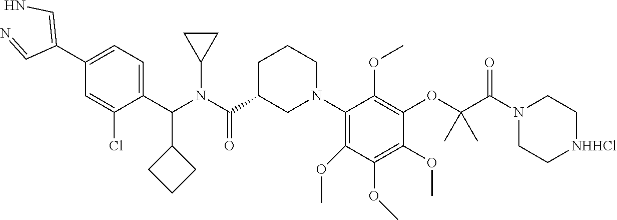 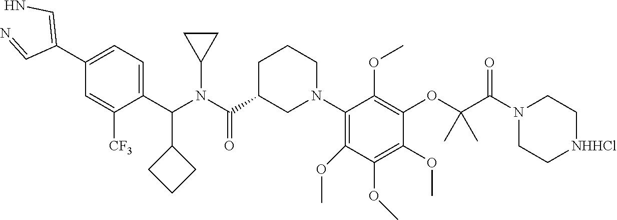 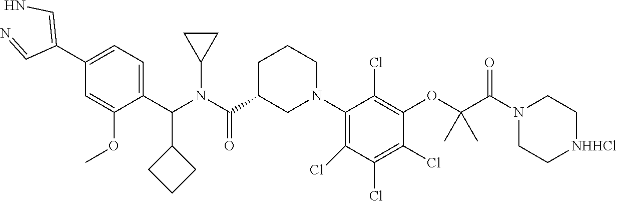 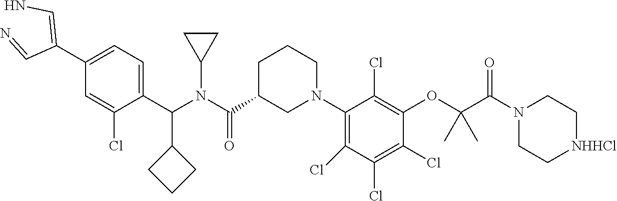 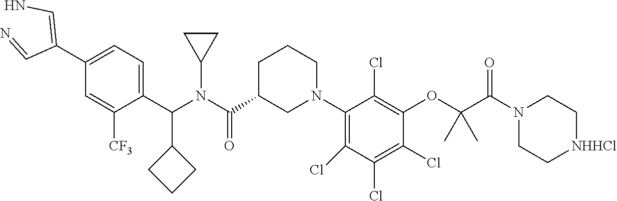 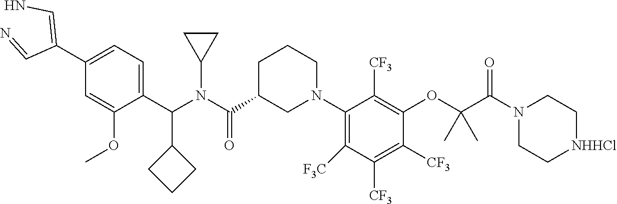 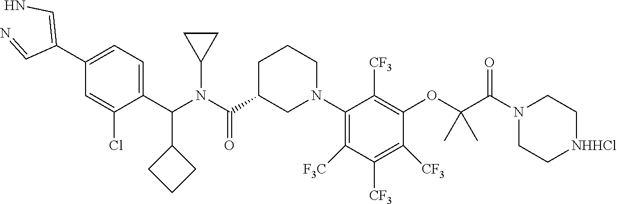 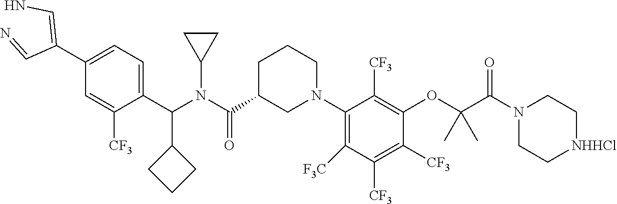
TABLE C

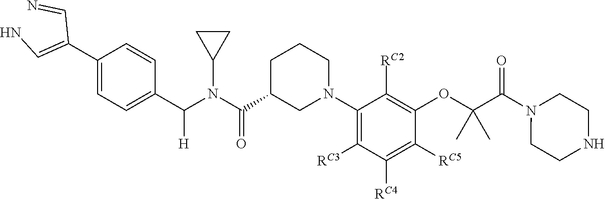

| | RC2 | RC3 | RC4 | RC5 |
|---|---|---|---|---|
| C020 | Me | H | H | H |
| C021 | H | Me | H | H |
| C022 | H | H | Me | H |
| C023 | H | H | H | Me |
| C024 | OMe | H | H | H |
| C025 | H | OMe | H | H |
| C026 | H | H | OMe | H |
| C027 | H | H | H | OMe |
| C028 | CF3 | H | H | H |
| C029 | H | CF3 | H | H |
| C030 | H | H | CF3 | H |
| C031 | H | H | H | CF3 |
| C032 | OCF3 | H | HH | H |
| C033 | H | OCF3 | H | H |
| C034 | H | H | OCF3 | H |
| C035 | H | H | H | OCF3 |
| C036 | Cl | H | H | H |
| C037 | H | Cl | H | H |

or a pharmaceutically acceptable salt thereof, or an isomer, solvate, crystal form or a prodrug thereof.

10. A pharmaceutical composition, wherein comprising:
(i) the compound of claim 1 or the pharmaceutically acceptable salt thereof, or the isomer, solvate, crystal form, or prodrug thereof; and
(ii) a pharmaceutically acceptable carrier or excipient.

11. A method for treating or preventing a disease associated with BCL9/β-catenin interaction, comprising a step of administering an effective amount of the compound of claim 1, or the pharmaceutically acceptable salt thereof, or the isomer, solvate, crystal form, or prodrug thereof, or administering a pharmaceutical composition comprising the compound of claim 1 or the pharmaceutically acceptable salt thereof.

12. The method of claim 11, wherein the disease associated with BCL9/β-catenin interaction is cancer, tumor, or a combination thereof.

13. A method for treating or preventing fibrosis or a related disease thereof, comprising a step of administering an effective amount of the compound of claim 1, or the pharmaceutically acceptable salt thereof, or the isomer, solvate, crystal form, or prodrug thereof, or administering a pharmaceutical composition comprising the compound of claim 1 or the pharmaceutically acceptable salt thereof.

14. The method of claim 13, wherein the fibrosis or the related disease thereof is: pulmonary fibrosis, hepatic fibrosis, non-alcoholic hepatic steatohepatitis, bone fibrosis, or a combination thereof.

15. The method of claim 13, wherein L¹ is —CH(R⁸)—N(R¹)—C(O)—NH—, wherein the CH(R⁸)— terminal is attached to Ring A.

16. The compound of claim 1 or the pharmaceutically acceptable salt thereof, or the isomer, solvate, crystal form or prodrug thereof, wherein, R⁶ is —NR⁴R⁵; wherein,
R⁴ and R⁵ are independently selected from the group consisting of: H and optionally substituted C-₁₋₆alkyl group; and wherein the optionally substituted means that one hydrogen in the group is substituted with a substituent selected from the group consisting of: —OR′ and —NR′R″; wherein R′ is independently selected from the group consisting of: H, D, and C₁₋₆ alkyl, and R″ is selected from the group consisting of: H, D, and C₁₋₄ alkyl; or, —NR⁴R⁵ is 4 to 10-membered heterocycloalkyl with at least one —O— present on the ring; or, —NR⁴R⁵ is 4 to 10 membered heterocycloalkyl with at least one —NH— or —NH²⁺— present on the ring.

17. The compound of claim 3 or the pharmaceutically acceptable salt thereof, or the isomer, solvate, crystal form or prodrug thereof, wherein R^{A1} is halogen.

18. The compound of claim 3 or the pharmaceutically acceptable salt thereof, or the isomer, solvate, crystal form or prodrug thereof, wherein R^{B1} is selected from the group consisting of: cyclohexyl and phenyl.

19. The compound of claim 3 or the pharmaceutically acceptable salt thereof, or the isomer, solvate, crystal form or prodrug thereof, wherein R^{C1} is a halogen.

20. The compound of claim 3 or the pharmaceutically acceptable salt thereof, or the isomer, solvate, crystal form or prodrug thereof, wherein both R² and R³ are methyl.
